(19) United States
(12) Patent Application Publication
(10) Pub. No.: US 2024/0270848 A1
(43) Pub. Date: Aug. 15, 2024

(54) COMBINATION THERAPIES TARGETING PD-1, TIM-3, AND LAG-3

(71) Applicant: LES LABORATOIRES SERVIER, Suresnes Cedex (FR)

(72) Inventors: Trine Lindsted, Farum (DK); Michael Monrad Grandal, Ballerup (DK); Eva Maria Carlsen Melander, Malmö (SE); Camilla Fröhlich, København Ø (DK); Mikkel Wandahl Pedersen, Allerød (DK); Michael Kragh, Copenhagen N (DK); Johan Lantto, Lund (SE); Monika Gad, Allerød (DK); Ivan David Horak, West Orange, NJ (US)

(73) Assignee: LES LABORATOIRES SERVIER, Suresnes Cedex (FR)

(21) Appl. No.: 18/444,187

(22) Filed: Feb. 16, 2024

Related U.S. Application Data
(63) Continuation of application No. 16/500,918, filed on Oct. 4, 2019, now Pat. No. 11,939,380.
(60) Provisional application No. 62/481,973, filed on Apr. 5, 2017.

(51) Int. Cl. C07K 16/28; A61K 39/00; A61K 45/06; A61P 35/00

(52) U.S. Cl. CPC C07K 16/2818; A61P 35/00; A61K 2039/507; A61K 45/06; C07K 2317/31; C07K 2317/565; C07K 2317/92

(57) ABSTRACT

This invention relates to combination therapies targeting two or all of PD-1, TIM-3, and LAG-3 using antibodies specific for these targets in patients who are in need of enhanced immunity. Also included in the invention are compositions useful in the therapies. The therapies are useful in treating diseases such as cancers.

### CROSS-REFERENCE TO RELATED APPLICATIONS

This application is a continuation of U.S. patent application Ser. No. 16/500,918, filed on Oct. 4, 2019, which is a national stage application under 35 U.S.C. § 371 of International Patent Application No. PCT/EP2018/058752, filed Apr. 5, 2018, which claims priority from U.S. Provisional Patent Application 62/481,973, filed Apr. 5, 2017. The disclosures of those applications are incorporated by reference herein in their entirety.

### SEQUENCE LISTING

The instant application contains a Sequence Listing which has been submitted electronically in ASCII format and is hereby incorporated by reference in its entirety. The copy of the Sequence Listing, created on Jan. 23, 2023, is named 022675.C1058.xml and is 519,202 bytes in size.

### BACKGROUND OF THE INVENTION

PD-1, also known as Programmed Cell Death Protein 1 and CD279, is a 268 amino acid cell surface receptor that belongs to the immunoglobulin superfamily. PD-1 is a member of the CD28 family of T cell regulators and is expressed on T cells, B cells and macrophages. It binds ligands PD-L1 (also known as B7 homolog) and PD-L2 (also known as B7-DC). PD-1 is a type I membrane protein whose structure includes an extracellular IgV domain, a transmembrane region and an intracellular tail containing two phosphorylation sites. Known as an immune checkpoint protein, PD-1 functions as an inducible immune modulatory receptor, playing a role in, e.g., negative regulation of T cell responses to antigen stimulation.

PD-L1 is the predominant ligand for PD-1. Binding of PD-L1 to PD-1 inhibits T cell activity, reducing cytokine production and suppressing T cell proliferation. Cancer cells that express PD-L1 are able to exploit this mechanism to inactivate the anti-tumor activity of T cells via binding of PD-L1 to the PD-1 receptor. In view of its immune response regulatory properties, PD-1 has been investigated as a potential target for immunotherapy, including treatment of cancer and autoimmune diseases. Two anti-PD-1 antibodies, pembrolizumab and nivolumab, have been approved in the United States and Europe for treating certain cancers.

Other immune checkpoint proteins include TIM-3 (T-cell immunoglobulin and mucin-domain containing 3) and LAG-3 (lymphocyte-activation gene 3). TIM-3, also known as HAVCR2 (hepatitis A virus cellular receptor 2) or CD366, is a member of the T-cell immunoglobulin and mucin domain protein family. TIM-3 is encoded in humans by the Havcr2 gene and is a 33 kDa type I glycoprotein with a membrane distal IgV domain and a membrane proximal mucin-domain. It contains a conserved region of five Tyr residues in the intracellular domain, which are phosphorylated upon ligand binding. TIM-3 is expressed by a range of different cells originating from both the adaptive and innate arms of the immune system, including T-cells, dendritic cells, macrophages, and natural killer (NK) cells. TIM-3 expression is low on naïve T cells but becomes highly upregulated upon T cell activation. In contrast to T-cells, innate cells such as dendritic cells, NK cells and monocytes have high basal TIM-3 expression. TIM-3 has been associated with several, mostly promiscuous, ligands, including galectin-9, phosphatidylserine, CEACAM-1 and HMGB-1, but the exact roles of these ligands are currently unknown.

Although TIM-3 has been suggested to be a checkpoint inhibitor, there is relatively sparse evidence to support the idea that TIM-3 directly mediates suppression of T cell activation or cytokine secretion in a manner similar to, e.g., PD-1. Furthermore, and in contrast to PD-1, TIM-3 appears to play a role in regulation of cells of the innate system, and in particular dendritic cells. The majority of functional data related to TIM-3 and its role in tumor immunology comes from in vivo studies using various antibodies. In most of these studies, due to poor antibody validation, it is not clear whether the effects of the TIM-3 antibodies are mediated by inhibition of ligand binding or by an agonistic effect on the target. In view of its immune response regulatory properties, TIM-3 has been investigated as a potential target for immunotherapy, including for treatment of cancer and autoimmune diseases. A single anti-TIM-3 antibody is currently in clinical development, but there are currently no approved anti-TIM-3 antibodies.

LAG-3, also known as CD223, is an immunoglobulin superfamily protein that functions as an immune checkpoint receptor. The mature protein is a 503-amino acid type I transmembrane protein with four extracellular Ig-like domains. It is expressed on various types of cells including activated T cells, T regulatory (Treg) cells, natural killer cells, B cells and plasmacytoid dendritic cells. Information on sequence data, exon/intron organization, and the chromosomal localization of LAG-3 indicates that it is closely related to CD4. Similar to CD4, LAG-3 binds MHC class II molecules, although with a higher affinity and at a distinct site compared to CD4.

LAG-3 is a co-inhibitory receptor that is thought to regulate T cell proliferation, activation and homeostasis in a manner similar to CTLA-4 and PD-1. Upon ligand binding to the extracellular domain, LAG-3 exerts its effect through subsequent signaling via the cytoplasmic domain. The best characterized ligand for LAG-3 is MHC class II (MHCII), but other LAG-3 ligands have been described, including LSECtin. LAG-3 has no classical ITIM or ITSM motifs, but has a conserved KIEELE motif (SEQ ID NO: 397) which is thought to be indispensable for accomplishing its inhibitory effect on T-cell activity. The exact mechanism by which LAG-3 affects T-cell activity is poorly understood. LAG-3 inhibits T cell expansion by blocking entry of activated T-cells into the growth phase of the cell cycle, resulting in the accumulation of cells in the S-phase. LAG-3 is also thought to play a role in enhancing the suppressive activity of regulatory T-cells and in modulating dendritic cell function. Cancer cells have the ability to upregulate expression of MHCII, which binds LAG-3 on effector T-cells, thus inhibiting their activity and inducing tumor immune escape.

In view of the critical role of PD-1, LAG-3, and TIM-3 as immune modulators, there is a need for new and improved combination therapies that target these receptors to treat cancers and certain disorders of the immune system.

### SUMMARY OF THE INVENTION

The present invention is based on the discovery that the immunity-enhancing efficacy of an anti-PD-1 antibody, such as those described herein, is significantly increased when the antibody is used in combination with an anti-TIM-3 antibody and/or an anti-LAG-3 antibody. The present inventors have found that the combination therapies of the present invention are particularly effective in treating cancer in a human patient by activating the patient's own anti-cancer immunity. Compared to currently available treatments for cancer, including antibody treatments, it is contemplated that the combination therapies of the invention may provide a superior clinical response.

Accordingly, the present invention provides a method of enhancing immunity in a human patient in need thereof, such as a cancer patient, by administering to the patient (1) an anti-PD-1 antibody, or an antigen-binding portion thereof, that competes for binding to human PD-1 with, or binds to the same epitope of human PD-1 as, an antibody selected from the group consisting of 12819.15384, 12748.15381, 12748.16124, 12865.15377, 12892.15378, 12796.15376, 12777.15382, 12760.15375 and 13112.15380; and (2) an anti-TIM-3 antibody or an antigen-binding portion thereof and/or an anti-LAG-3 antibody or an antigen-binding portion thereof. In certain embodiments, the method comprises administering the anti-PD-1 antibody or antigen-binding portion thereof, the anti-TIM-3 antibody or antigen-binding portion thereof, and the anti-LAG-3 antibody or antigen-binding portion thereof.

In some embodiments, the anti-PD-1 antibody binds to an epitope of human PD-1 comprising:
a) residues V64, L128, P130, K131, and A132 of SEQ ID NO: 388;
b) residues V44 and T145 of SEQ ID NO: 388;
c) residues K131 and E136 of SEQ ID NO: 388; or
d) residues V44 and T145 of SEQ ID NO: 388.

In some embodiments, the anti-PD-1 antibody binds to an epitope of human PD-1 comprising:
a) residues 56-64, 69-90, and 122-140 of SEQ ID NO: 388;
b) residues 69-90 and 122-140 of SEQ ID NO: 388;
c) residues 69-75 of SEQ ID NO: 388;
d) residues 136-140 of SEQ ID NO: 388; or
e) residues 69-75 and 136-140 of SEQ ID NO: 388.

In some embodiments, the anti-PD-1 antibody has at least one of the following properties:
a) binds to human PD-1 with a K_{D} of 750 pM or less;
b) binds to cynomolgus PD-1 with a K_{D} of 7 nM or less;
c) binds to mouse PD-1 with a K_{D} of 1 nM or less;
d) does not bind to rat PD-1;
e) increases IL-2 secretion in a Staphylococcal enterotoxin B (SEB) whole blood assay;
f) increases IFN-γ secretion in a one-way mixed lymphocyte reaction assay;
g) inhibits the interaction of PD-1 with PD-L1 by at least 60% at a concentration of 10 μg/mL in a flow cytometric competition assay;
h) blocks binding of PD-L1 and PD-L2 to PD-1 by at least 90% at a concentration of 10 μg/mL as determined by Bio-Layer Interferometry analysis; and
i) inhibits tumor growth in vivo.

In certain embodiments, the anti-PD-1 antibody has all of said properties.

In some embodiments, the heavy chain complementarity-determining regions (H-CDR) 1-3 and light chain complementarity-determining regions (L-CDR) 1-3 of the anti-PD-1 antibody comprise the amino acid sequences of:
a) SEQ ID NOs: 228-233, respectively;
b) SEQ ID NOs: 238-243, respectively;
c) SEQ ID NOs: 248-253, respectively;
d) SEQ ID NOs: 258-263, respectively;
e) SEQ ID NOs: 268-273, respectively;
f) SEQ ID NOs: 278-283, respectively;
g) SEQ ID NOs: 288-293, respectively; or
h) SEQ ID NOs: 298-303, respectively.

In some embodiments, the heavy and light chain variable domains of the anti-PD-1 antibody comprise the amino acid sequences of:
a) SEQ ID NOs: 226 and 227, respectively;
b) SEQ ID NOs: 236 and 237, respectively;
c) SEQ ID NOs: 236 and 392, respectively;
d) SEQ ID NOs: 246 and 247, respectively;
e) SEQ ID NOs: 256 and 257, respectively;
f) SEQ ID NOs: 266 and 267, respectively;
g) SEQ ID NOs: 276 and 277, respectively;
h) SEQ ID NOs: 286 and 287, respectively; or
i) SEQ ID NOs: 296 and 297, respectively.

In some embodiments, the anti-PD-1 antibody comprises:
a) a heavy chain (HC) comprising the amino acid sequence of SEQ ID NO: 226 and the amino acid sequence of SEQ ID NO: 375, and a light chain (LC) comprising the amino acid sequence of SEQ ID NO: 227 and the amino acid sequence of SEQ ID NO: 379;
b) an HC comprising the amino acid sequence of SEQ ID NO: 236 and the amino acid sequence of SEQ ID NO: 375, and an LC comprising the amino acid sequence of SEQ ID NO: 237 and the amino acid sequence of SEQ ID NO: 379;
c) an HC comprising the amino acid sequence of SEQ ID NO: 236 and the amino acid sequence of SEQ ID NO: 375, and an LC comprising the amino acid sequence of SEQ ID NO: 392 and the amino acid sequence of SEQ ID NO: 379;
d) an HC comprising the amino acid sequence of SEQ ID NO: 246 and the amino acid sequence of SEQ ID NO: 375, and an LC comprising the amino acid sequence of SEQ ID NO: 247 and the amino acid sequence of SEQ ID NO: 379;
e) an HC comprising the amino acid sequence of SEQ ID NO: 256 and the amino acid sequence of SEQ ID NO: 375, and an LC comprising the amino acid sequence of SEQ ID NO: 257 and the amino acid sequence of SEQ ID NO: 379;
f) an HC comprising the amino acid sequence of SEQ ID NO: 266 and the amino acid sequence of SEQ ID NO: 375, and an LC comprising the amino acid sequence of SEQ ID NO: 267 and the amino acid sequence of SEQ ID NO: 379;
g) an HC comprising the amino acid sequence of SEQ ID NO: 276 and the amino acid sequence of SEQ ID NO: 375, and an LC comprising the amino acid sequence of SEQ ID NO: 277 and the amino acid sequence of SEQ ID NO: 379;
h) an HC comprising the amino acid sequence of SEQ ID NO: 286 and the amino acid sequence of SEQ ID NO: 375, and an LC comprising the amino acid sequence of SEQ ID NO: 287 and the amino acid sequence of SEQ ID NO: 379; or
i) an HC comprising the amino acid sequence of SEQ ID NO: 296 and the amino acid sequence of SEQ ID NO: 375, and an LC comprising the amino acid sequence of SEQ ID NO: 297 and the amino acid sequence of SEQ ID NO: 379.

In some embodiments, the anti-TIM-3 antibody competes for binding to human TIM-3 with, or binds to the same epitope of human TIM-3 as, an antibody selected from the group consisting of 15086.17145, 15086.15086, 15086.16837, 15086.17144, 20131, 20293, 15105, 15107, 15109, 15174, 15175, 15260, 15284, 15299, 15353, 15354, 17244, 17245, 19324, 19416, 19568, 20185, 20300, 20362, and 20621.

In some embodiments, the anti-TIM-3 antibody binds to an epitope of human TIM-3 comprising:
a) residues P50, V60, F61, E62, G64, R69, 1117, M118, and D120 of SEQ ID NO: 389;
b) residues F61, R69, and 1117 of SEQ ID NO: 389; or
c) residues P50, F61, E62, 1117, M118, and D120 of SEQ ID NO: 389.

In some embodiments, the anti-TIM-3 antibody binds to an epitope of human TIM-3 comprising:
a) residues 62-67 of SEQ ID NO: 389; or
b) residues 114-117 of SEQ ID NO: 389.

In some embodiments, the anti-TIM-3 antibody has at least one of the following properties:
a) binds to human TIM-3 with a K_{D} of 23 nM or less as measured by surface plasmon resonance;
b) binds to cynomolgus TIM-3 with a K_{D} of 22 nM or less as measured by surface plasmon resonance;
c) binds to human TIM-3 with an EC50 of 1.2 nM or less as measured by ELISA; d) binds to cynomolgus TIM-3 with an EC50 of 46 nM or less as measured by ELISA;
e) increases IFN-γ secretion in a one-way mixed lymphocyte reaction assay; f) increases IFN-γ secretion in a two-way mixed lymphocyte reaction assay; g) increases TNF-α secretion in a one-way mixed lymphocyte reaction assay; h) increases TNF-α secretion from dendritic cells; and
i) inhibits interaction of TIM-3 with phosphatidylserine.

In certain embodiments, the anti-TIM-3 antibody has at least properties a), c), d), e), g), h), and i).

In some embodiments, the H-CDR1-3 and L-CDR1-3 of the anti-TIM-3 antibody comprise the amino acid sequences of:
a) SEQ ID NOs: 8-13, respectively;
b) SEQ ID NOs: 18-23, respectively;
c) SEQ ID NOs: 28-33, respectively;
d) SEQ ID NOs: 38-43, respectively;
e) SEQ ID NOs: 48-53, respectively;
f) SEQ ID NOs: 58-63, respectively;
g) SEQ ID NOs: 68-73, respectively;
h) SEQ ID NOs: 78-83, respectively;
i) SEQ ID NOs: 88-93, respectively;
j) SEQ ID NOs: 98-103, respectively;
k) SEQ ID NOs: 108-113, respectively;
l) SEQ ID NOs: 118-123, respectively;
m) SEQ ID NOs: 128-133, respectively;
n) SEQ ID NOs: 138-143, respectively;
o) SEQ ID NOs: 148-153, respectively;
p) SEQ ID NOs: 158-163, respectively;
q) SEQ ID NOs: 168-173, respectively;
r) SEQ ID NOs: 178-183, respectively;
s) SEQ ID NOs: 188-193, respectively;
t) SEQ ID NOs: 198-203, respectively;
u) SEQ ID NOs: 208-213, respectively; or
v) SEQ ID NOs: 218-223, respectively.

In some embodiments, the heavy and light chain variable domains of the anti-TIM-3 antibody comprise the amino acid sequences of:
a) SEQ ID NOs: 7 and 4, respectively;
b) SEQ ID NOs: 3 and 4, respectively;
c) SEQ ID NOs: 16 and 17, respectively;
d) SEQ ID NOs: 26 and 27, respectively;
e) SEQ ID NOs: 36 and 37, respectively;
f) SEQ ID NOs: 46 and 47, respectively;
g) SEQ ID NOs: 56 and 57, respectively;
h) SEQ ID NOs: 66 and 67, respectively;
i) SEQ ID NOs: 76 and 77, respectively;
j) SEQ ID NOs: 86 and 87, respectively;
k) SEQ ID NOs: 96 and 97, respectively;
l) SEQ ID NOs: 106 and 107, respectively;
m) SEQ ID NOs: 116 and 117, respectively;
n) SEQ ID NOs: 126 and 127, respectively;
o) SEQ ID NOs: 136 and 137, respectively;
p) SEQ ID NOs: 146 and 147, respectively;
q) SEQ ID NOs: 156 and 157, respectively;
r) SEQ ID NOs: 166 and 167, respectively;
s) SEQ ID NOs: 176 and 177, respectively;
t) SEQ ID NOs: 186 and 187, respectively;
u) SEQ ID NOs: 196 and 197, respectively;
v) SEQ ID NOs: 206 and 207, respectively; or
w) SEQ ID NOs: 216 and 217, respectively.

In some embodiments, the anti-TIM-3 antibody comprises:
a) an HC comprising the amino acid sequence of SEQ ID NO: 7 and the amino acid sequence of SEQ ID NO: 374, 375, 376, or 377, and an LC comprising the amino acid sequence of SEQ ID NO: 4 and the amino acid sequence of SEQ ID NO: 378;
b) an HC comprising the amino acid sequence of SEQ ID NO: 3 and the amino acid sequence of SEQ ID NO: 374, 375, 376, or 377, and an LC comprising the amino acid sequence of SEQ ID NO: 4 and the amino acid sequence of SEQ ID NO: 378;
c) an HC comprising the amino acid sequence of SEQ ID NO: 16 and the amino acid sequence of SEQ ID NO: 374, 375, 376, or 377, and an LC comprising the amino acid sequence of SEQ ID NO: 17 and the amino acid sequence of SEQ ID NO: 378;
d) an HC comprising the amino acid sequence of SEQ ID NO: 26 and the amino acid sequence of SEQ ID NO: 374, 375, 376, or 377, and an LC comprising the amino acid sequence of SEQ ID NO: 27 and the amino acid sequence of SEQ ID NO: 378;
e) an HC comprising the amino acid sequence of SEQ ID NO: 36 and the amino acid sequence of SEQ ID NO: 374, 375, 376, or 377, and an LC comprising the amino acid sequence of SEQ ID NO: 37 and the amino acid sequence of SEQ ID NO: 378;
f) an HC comprising the amino acid sequence of SEQ ID NO: 46 and the amino acid sequence of SEQ ID NO: 374, 375, 376, or 377, and an LC comprising the amino acid sequence of SEQ ID NO: 47 and the amino acid sequence of SEQ ID NO: 378;
g) an HC comprising the amino acid sequence of SEQ ID NO: 56 and the amino acid sequence of SEQ ID NO: 374, 375, 376, or 377, and an LC comprising the amino acid sequence of SEQ ID NO: 57 and the amino acid sequence of SEQ ID NO: 378;
h) an HC comprising the amino acid sequence of SEQ ID NO: 66 and the amino acid sequence of SEQ ID NO: 374, 375, 376, or 377, and an LC comprising the amino acid sequence of SEQ ID NO: 67 and the amino acid sequence of SEQ ID NO: 378;
i) an HC comprising the amino acid sequence of SEQ ID NO: 76 and the amino acid sequence of SEQ ID NO: 374, 375, 376, or 377, and an LC comprising the amino acid sequence of SEQ ID NO: 77 and the amino acid sequence of SEQ ID NO: 378;
j) an HC comprising the amino acid sequence of SEQ ID NO: 86 and the amino acid sequence of SEQ ID NO: 374, 375, 376, or 377, and an LC comprising the amino acid sequence of SEQ ID NO: 87 and the amino acid sequence of SEQ ID NO: 378;
k) an HC comprising the amino acid sequence of SEQ ID NO: 96 and the amino acid sequence of SEQ ID NO: 374, 375, 376, or 377, and an LC comprising the amino acid sequence of SEQ ID NO: 97 and the amino acid sequence of SEQ ID NO: 378;
l) an HC comprising the amino acid sequence of SEQ ID NO: 106 and the amino acid sequence of SEQ ID NO: 374, 375, 376, or 377, and an LC comprising the amino acid sequence of SEQ ID NO: 107 and the amino acid sequence of SEQ ID NO: 378;
m) an HC comprising the amino acid sequence of SEQ ID NO: 116 and the amino acid sequence of SEQ ID NO: 374, 375, 376, or 377, and an LC comprising the amino acid sequence of SEQ ID NO: 117 and the amino acid sequence of SEQ ID NO: 378;
n) an HC comprising the amino acid sequence of SEQ ID NO: 126 and the amino acid sequence of SEQ ID NO: 374, 375, 376, or 377, and an LC comprising the amino acid sequence of SEQ ID NO: 127 and the amino acid sequence of SEQ ID NO: 378;
o) an HC comprising the amino acid sequence of SEQ ID NO: 136 and the amino acid sequence of SEQ ID NO: 374, 375, 376, or 377, and an LC comprising the amino acid sequence of SEQ ID NO: 137 and the amino acid sequence of SEQ ID NO: 378;
p) an HC comprising the amino acid sequence of SEQ ID NO: 146 and the amino acid sequence of SEQ ID NO: 374, 375, 376, or 377, and an LC comprising the amino acid sequence of SEQ ID NO: 147 and the amino acid sequence of SEQ ID NO: 378;
q) an HC comprising the amino acid sequence of SEQ ID NO: 156 and the amino acid sequence of SEQ ID NO: 374, 375, 376, or 377, and an LC comprising the amino acid sequence of SEQ ID NO: 157 and the amino acid sequence of SEQ ID NO: 378;
r) an HC comprising the amino acid sequence of SEQ ID NO: 166 and the amino acid sequence of SEQ ID NO: 374, 375, 376, or 377, and an LC comprising the amino acid sequence of SEQ ID NO: 167 and the amino acid sequence of SEQ ID NO: 378;
s) an HC comprising the amino acid sequence of SEQ ID NO: 176 and the amino acid sequence of SEQ ID NO: 374, 375, 376, or 377, and an LC comprising the amino acid sequence of SEQ ID NO: 177 and the amino acid sequence of SEQ ID NO: 378;
t) an HC comprising the amino acid sequence of SEQ ID NO: 186 and the amino acid sequence of SEQ ID NO: 374, 375, 376, or 377, and an LC comprising the amino acid sequence of SEQ ID NO: 187 and the amino acid sequence of SEQ ID NO: 378;
u) an HC comprising the amino acid sequence of SEQ ID NO: 196 and the amino acid sequence of SEQ ID NO: 374, 375, 376, or 377, and an LC comprising the amino acid sequence of SEQ ID NO: 197 and the amino acid sequence of SEQ ID NO: 378;
v) an HC comprising the amino acid sequence of SEQ ID NO: 206 and the amino acid sequence of SEQ ID NO: 374, 375, 376, or 377, and an LC comprising the amino acid sequence of SEQ ID NO: 207 and the amino acid sequence of SEQ ID NO: 378; or
w) an HC comprising the amino acid sequence of SEQ ID NO: 216 and the amino acid sequence of SEQ ID NO: 374, 375, 376, or 377, and an LC comprising the amino acid sequence of SEQ ID NO: 217 and the amino acid sequence of SEQ ID NO: 378.

In some embodiments, the anti-LAG-3 antibody competes for binding to human LAG-3 with, or binds to the same epitope of human LAG-3 as, an antibody selected from the group consisting of 15532, 15646, 15723, 15595, 15431, 15572, and 15011.

In some embodiments, the anti-LAG-3 antibody binds to an epitope of human LAG-3 comprising:
a) residues H85, P86, A87, P89, S91, W92, and G93 of SEQ ID NO: 68; b) residues A40, Q41, P43, P46, P49, D52, T62, Q64, H65, Q66, P67, D68, G93, P94, P96, R98, Y99, T100, V101, P106, G107, R119, E124, R129, G130, D131, S133, R137, P138, D143, R148, and R163 of SEQ ID NO: 68; c) residues A40, Q41, P43, P46, P49, D52, T62, Q64, H65, Q66, P67, D68, P96, Y99, T100, V101, P106, G107, R119, E124, R129, G130, D131, S133, R137, P138, D143, R148, and R163 of SEQ ID NO: 68; or
d) residues G107, L109, R110, and S111 of SEQ ID NO: 68.

In some embodiments, the anti-LAG-3 antibody binds to an epitope of human LAG-3 comprising:
a) residues 98-105 of SEQ ID NO: 68;
b) residues 78-105 and 123-131 of SEQ ID NO: 68;
c) residues 23-30, 40-66, 88-105, 123-137, and 148-152 of SEQ ID NO: 68; or d) residues 23-30, 40-66, 98-105, 118-137, and 148-161 of SEQ ID NO: 68.

In some embodiments, the anti-LAG-3 antibody has at least one of the following properties:
a) at a concentration of 20 μg/mL, reduces the binding of human LAG-3 to human MHC class II on A375 cells by greater than 85% compared to a negative control antibody as determined by a flow cytometric competition assay;
b) at a concentration of 20 μg/mL, reduces the binding of human LAG-3 to human MHC class II on A375 cells to between 35% and 85% compared to a negative control antibody as determined by a flow cytometric competition assay;
c) blocks binding between human LAG-3 expressed on Jurkat cells and human MHC class II expressed on Raji cells;
d) binds to human LAG-3 with an EC50 of 0.1 nM or less as measured by flow cytometry;
e) binds to cynomolgus LAG-3 with an EC50 of 0.3 nM or less as measured by flow cytometry;
f) binds to human LAG-3 with a K_{D} of 3.0×10⁻⁸ or less as measured by surface plasmon resonance;
g) binds to cynomolgus LAG-3 with a K_{D} of 1.5×10⁻⁷ or less as measured by surface plasmon resonance;
h) binds to mouse LAG-3 with a K_{D} of 3.5×10⁻⁸ or less as measured by surface plasmon resonance;
i) stimulates IL-2 production in Staphylococcalenterotoxin B (SEB) treated human peripheral blood mononuclear cells (PBMCs);
j) reduces cellular levels of LAG-3 in human T cells;
k) reduces soluble levels of LAG-3 in the culture of human T cells;
l) induces tumor growth regression in vivo;
m) delays tumor growth in vivo; and
n) does not bind to the same epitope of human LAG-3 as antibody 25F7-Lag3.5. In certain embodiments, the anti-LAG-3 antibody has at least properties a), c), d), e), f), g), i), j), k), m), and n).

In some embodiments, the H-CDR1-3 and L-CDR1-3 of the anti-LAG-3 antibody comprise the amino acid sequences of:
a) SEQ ID NOs: 318-323, respectively;
b) SEQ ID NOs: 308-313, respectively;
c) SEQ ID NOs: 328-333, respectively;
d) SEQ ID NOs: 338-343, respectively;
e) SEQ ID NOs: 348-353, respectively;
f) SEQ ID NOs: 358-363, respectively; or
g) SEQ ID NOs: 368-373, respectively.

In some embodiments, the heavy and light chain variable domains of the anti-LAG-3 antibody comprise the amino acid sequences of:
a) SEQ ID NOs: 316 and 317, respectively;
b) SEQ ID NOs: 306 and 307, respectively;
c) SEQ ID NOs: 326 and 327, respectively;
d) SEQ ID NOs: 336 and 337, respectively;
e) SEQ ID NOs: 346 and 347, respectively;
f) SEQ ID NOs: 356 and 357, respectively; or
g) SEQ ID NOs: 366 and 367, respectively.

In some embodiments, the anti-LAG-3 antibody comprises:
a) an HC comprising the amino acid sequence of SEQ ID NO: 316 and the amino acid sequence of SEQ ID NO: 375, and an LC comprising the amino acid sequence of SEQ ID NO: 317 and the amino acid sequence of SEQ ID NO: 378; b) an HC comprising the amino acid sequence of SEQ ID NO: 306 and the amino acid sequence of SEQ ID NO: 375, and an LC comprising the amino acid sequence of SEQ ID NO: 307 and the amino acid sequence of SEQ ID NO: 378; c) an HC comprising the amino acid sequence of SEQ ID NO: 326 and the amino acid sequence of SEQ ID NO: 375, and an LC comprising the amino acid sequence of SEQ ID NO: 327 and the amino acid sequence of SEQ ID NO: 378; d) an HC comprising the amino acid sequence of SEQ ID NO: 336 and the amino acid sequence of SEQ ID NO: 375, and an LC comprising the amino acid sequence of SEQ ID NO: 337 and the amino acid sequence of SEQ ID NO: 378; e) an HC comprising the amino acid sequence of SEQ ID NO: 346 and the amino acid sequence of SEQ ID NO: 375, and an LC comprising the amino acid sequence of SEQ ID NO: 347 and the amino acid sequence of SEQ ID NO: 378; f) an HC comprising the amino acid sequence of SEQ ID NO: 356 and the amino acid sequence of SEQ ID NO: 375, and an LC comprising the amino acid sequence of SEQ ID NO: 357 and the amino acid sequence of SEQ ID NO: 378; or g) an HC comprising the amino acid sequence of SEQ ID NO: 366 and the amino acid sequence of SEQ ID NO: 375, and an LC comprising the amino acid sequence of SEQ ID NO: 367 and the amino acid sequence of SEQ ID NO: 379.

In some embodiments, the method comprises administering to the patient:
a) an anti-PD-1 antibody whose H-CDR1-3 and L-CDR1-3 comprise the amino acid sequences of SEQ ID NOs: 228-233, respectively; and an anti-TIM-3 antibody whose H-CDR1-3 and L-CDR1-3 comprise the amino acid sequences of SEQ ID NOs: 8-13, respectively;
b) an anti-PD-1 antibody whose VH and VL comprise the amino acid sequences of SEQ ID NOs: 226 and 227, respectively; and an anti-TIM-3 antibody whose VH and VL comprise the amino acid sequences of SEQ ID NOs: 7 and 4, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; and an anti-TIM-3 antibody comprising an HC that comprises the amino acid sequences of SEQ ID NOs: 7 and 377 and an LC that comprises the amino acid sequences of SEQ ID NOs: 4 and 378.

In some embodiments, the method comprises administering to the patient:
a) an anti-PD-1 antibody whose H-CDR1-3 and L-CDR1-3 comprise the amino acid sequences of SEQ ID NOs: 228-233, respectively; and an anti-LAG-3 antibody whose H-CDR1-3 and L-CDR1-3 comprise the amino acid sequences of SEQ ID NOs: 318-323, respectively;
b) an anti-PD-1 antibody whose VH and VL comprise the amino acid sequences of SEQ ID NOs: 226 and 227, respectively; and an anti-LAG-3 antibody whose VH and VL comprise the amino acid sequences of SEQ ID NOs: 316 and 317, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; and an anti-LAG-3 antibody comprising an HC that comprises the amino acid sequences of SEQ ID NOs: 316 and 375 and an LC that comprises the amino acid sequences of SEQ ID NOs: 317 and 378.

In some embodiments, the method comprises administering to the patient:
a) an anti-PD-1 antibody whose H-CDR1-3 and L-CDR1-3 comprise the amino acid sequences of SEQ ID NOs: 228-233, respectively; an anti-TIM-3 antibody whose H-CDR1-3 and L-CDR1-3 comprise the amino acid sequences of SEQ ID NOs: 8-13, respectively; and an anti-LAG-3 antibody whose H-CDR1-3 and L-CDR1-3 comprise the amino acid sequences of SEQ ID NOs: 318-323, respectively;
b) an anti-PD-1 antibody whose VH and VL comprise the amino acid sequences of SEQ ID NOs: 226 and 227, respectively; an anti-TIM-3 antibody whose VH and VL comprise the amino acid sequences of SEQ ID NOs: 7 and 4, respectively; and an anti-LAG-3 antibody whose VH and VL comprise the amino acid sequences of SEQ ID NOs: 316 and 317, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; an anti-TIM-3 antibody comprising an HC that comprises the amino acid sequences of SEQ ID NOs: 7 and 377 and an LC that comprises the amino acid sequences of SEQ ID NOs: 4 and 378; and an anti-LAG-3 antibody comprising an HC that comprises the amino acid sequences of SEQ ID NOs: 316 and 375 and an LC that comprises the amino acid sequences of SEQ ID NOs: 317 and 378.

The antibodies or antigen-binding portions may be administered to the patient concurrently (e.g., in a single pharmaceutical composition) or sequentially.

The therapies of the present invention are useful in treating a patient who has cancer, such as a hematological malignancy (e.g., leukemia, Hodgkin's lymphoma, or non-Hodgkin's lymphoma), or a solid tumor. In some embodiments, the patient may have melanoma, non-small cell lung cancer, bladder cancer, head and neck squamous cell carcinoma, ovarian cancer, colorectal cancer, renal cell carcinoma, Merkel-cell carcinoma, fibrosarcoma, gliosarcoma, or glioblastoma. The therapies of the present invention can additionally include radiation, or at least one of a chemotherapeutic agent, an anti-neoplastic agent, and an anti-angiogenic agent.

Also provided in the present invention is a multi-specific (e.g., bi-specific or tri-specific) antibody that specifically binds to: a) human PD-1 and human TIM-3; b) human PD-1 and human LAG-3; or c) human PD-1, human anti-TIM-3, and human LAG-3. In certain embodiments, the multi-specific antibody comprises an antigen-binding portion of an anti-PD-1 antibody as described herein, an antigen-binding portion of an anti-TIM-3 antibody as described herein, and/or an antigen-binding portion of an anti-LAG-3 antibody as described herein.

Also provided in the present invention is a pharmaceutical composition comprising (1) an anti-PD-1 antibody or an antigen-binding portion thereof as described herein, (2) an anti-TIM-3 antibody or an antigen-binding portion thereof and/or an anti-LAG-3 antibody or an antigen-binding portion thereof, and (3) a pharmaceutically acceptable excipient. The anti-TIM-3 antibody and the anti-LAG-3 antibody can be selected from those as described herein.

In some embodiments, the pharmaceutical composition comprises:
a) an anti-PD-1 antibody whose H-CDR1-3 and L-CDR1-3 comprise the amino acid sequences of SEQ ID NOs: 228-233, respectively; an anti-TIM-3 antibody whose H-CDR1-3 and L-CDR1-3 comprise the amino acid sequences of SEQ ID NOs: 8-13, respectively; and an anti-LAG-3 antibody whose H-CDR1-3 and L-CDR1-3 comprise the amino acid sequences of SEQ ID NOs: 318-323, respectively;
b) an anti-PD-1 antibody whose VH and VL comprise the amino acid sequences of SEQ ID NOs: 226 and 227, respectively; an anti-TIM-3 antibody whose VH and VL comprise the amino acid sequences of SEQ ID NOs: 7 and 4, respectively; and an anti-LAG-3 antibody whose VH and VL comprise the amino acid sequences of SEQ ID NOs: 316 and 317, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; an anti-TIM-3 antibody comprising an HC that comprises the amino acid sequences of SEQ ID NOs: 7 and 377 and an LC that comprises the amino acid sequences of SEQ ID NOs: 4 and 378; and an anti-LAG-3 antibody comprising an HC that comprises the amino acid sequences of SEQ ID NOs: 316 and 375 and an LC that comprises the amino acid sequences of SEQ ID NOs: 317 and 378.

The antibodies in the composition may be present in equal amounts. In some embodiments, the pharmaceutical composition is for use in treating a human patient in a method described herein. In particular embodiments, the pharmaceutical composition is for use in enhancing immunity in a human patient in need thereof, e.g., for treating cancer.

The present invention also provides an anti-PD-1 antibody or an antigen-binding portion thereof as described herein for use in a treatment method described herein, e.g., enhancing immunity and/or treating cancer in a human patient in need thereof, in combination with an anti-TIM-3 antibody or an antigen-binding portion thereof and/or an anti-LAG-3 antibody or an antigen-binding portion thereof. The anti-TIM-3 antibody and the anti-LAG-3 antibody can be selected from those as described herein.

The present invention also provides an anti-PD-1 antibody or an antigen-binding portion thereof as described herein for use in treating a human patient in a method described herein.

The present invention also provides the use of an anti-PD-1 antibody or an antigen-binding portion thereof as described herein for the manufacture of a medicament for enhancing immunity and/or treating cancer in a patient in need thereof (e.g., a human patient), in combination with an anti-TIM-3 antibody or an antigen-binding portion thereof and/or an anti-LAG-3 antibody or an antigen-binding portion thereof. In some embodiments, the present invention provides the use of an anti-PD-1 antibody or an antigen-binding portion thereof as described herein, and an anti-TIM-3 antibody or an antigen-binding portion thereof and/or an anti-LAG-3 antibody or an antigen-binding portion thereof, for the manufacture of a medicament for enhancing immunity in a human patient in need thereof, e.g., for treating cancer. The anti-TIM-3 antibody and the anti-LAG-3 antibody can be selected from those as described herein.

The present invention also provides the use of an anti-PD-1 antibody or an antigen-binding portion thereof as described herein for the manufacture of a medicament for treating a human patient in a method described herein.

The present invention further provides an article of manufacture comprising an anti-PD-1 antibody or an antigen-binding portion thereof as described herein, in combination with an anti-TIM-3 antibody or an antigen-binding portion thereof and/or an anti-LAG-3 antibody or an antigen-binding portion thereof, wherein said article of manufacture is suitable for enhancing immunity and/or treating cancer in a patient (such as a human patient), e.g., in a treatment method described herein. The anti-TIM-3 antibody and the anti-LAG-3 antibody can be selected from those as described herein.

### BRIEF DESCRIPTION OF THE DRAWINGS

FIG. 1 is a set of graphs showing the percent increase in IFN-γ levels in three donor pairs (Panels A-C) after treatment with 10 μg/mL of control antibody (IgG1 LALA or IgG2), anti-PD-1 antibody 12819, anti-TIM-3 antibody 15086.17145, or a combination of antibodies 12819 and 15086.17145 at a 1:1 ratio, after 5 days of culture in a one-way MLR (mixed lymphocyte reaction) assay. Each dot in the graph represents a replicate and the mean is indicated by a horizontal bar.

FIG. 2 is a set of graphs showing the percent increase in IFN-γ levels in three donor pairs (Panels A-C) after treatment with 10 μg/mL of control antibody (IgG1 LALA or IgG2), anti-PD-1 antibody 12819, anti-TIM-3 antibody 15086.17145, or a combination of antibodies 12819 and 15086.17145 at a 1:1 ratio, after 5 days of culture in a two-way MLR assay. Each dot in the graph represents a replicate and the mean is indicated by a horizontal bar.

FIG. 3 is a pair of graphs showing the effect of anti-TIM-3 antibody 15086.17145 in comparison to positive control anti-PD-1 antibody 12819 on T-cell proliferation in the one-way MLR assay. The antibodies were added to a final concentration of 25 μg/mL and incubated without (Panel A) or with (Panel B) soluble anti-CD3 for 5 days prior to adding 1 μCi/well 3H-thymidine for an additional 18 hours. 3H-thymidine incorporation in the harvested cells was determined by liquid scintillation counting.

FIG. 4 is a graph showing the effect of anti-TIM-3 antibody 15086.17145 on IL-12p40 secretion from monocyte derived dendritic cells incubated for 5 days with 10 μg/mL of antibody. IL-12p40 levels in cell supernatants were determined using ELISA.

FIG. 5 is a pair of graphs showing expression levels of selected activation markers on monocyte derived dendritic cells after treatment with 25 μg/mL anti-TIM-3 antibody 15086.17145 for 24 hours. Panel A shows gene expression analysis of several activation markers and co-simulatory molecules. Panel B shows validation of increased CD80 and Cd86 levels using FACS. The histogram overlays shown are representative of CD11c+ dendritic cells, and numbers adjacent to the histograms denote MFI (mean fluorescence intensity) values.

FIG. 6 is a graph showing the effect of TIM-3 targeting on tumor growth in a CD34⁺ humanized NSG-SGM3 mouse model engrafted with the human lung patient-derived xenograft (PDX) model LG1306. Mice were treated with anti-TIM-3 antibody 15086.17145 at an initial dose of 10 mg/kg followed by 5 mg/kg 5× Q5D. The grey area denotes the treatment period. Data are presented as means±SEM. **p<0.01.

FIG. 7 is a pair of graphs showing the enhanced effect on IL-2 levels of combining anti-PD-1 antibody 12819 with anti-LAG-3 antibody 15532 and/or anti-TIM-3 antibody 15086.17145 in a SEB (Staphylococcal Enterotoxin B)+PBMC (peripheral blood mononuclear cell) assay for two donor pairs (Panels A and B). The bars indicate IL-2 secretion from PBMCs treated with 10 μg/mL of the indicated antibodies or antibody mixtures, and SEB, for 48 hours. Signs above the bars indicate single treatments that are significantly different (p<0.05) from control antibody (*) or mixtures of two antibodies that are significantly different from the constituent single antibody treatments (#).

FIG. 8 is a pair of graphs showing the effect on tumor growth of 10 mg/kg of anti-PD-1 antibody 12819, anti-LAG-3 antibody C9B7W (which is reactive with mouse LAG-3), or the combination of anti-PD-1 and anti-LAG-3 antibodies, or vehicle treatment, in two syngeneic mouse tumor models: MC38 (colon cancer, Panel A) and ASB-XIV (lung cancer, Panel B). The grey area denotes the treatment period. Data are presented as means±SEM (standard error of the mean). **p<0.01, ***p<0.001, ****p<0.0001.

FIG. 9 is a graph showing the effect on tumor growth of 10 mg/kg of anti-PD-1 antibody 12819, anti-LAG-3 antibody 15011, or the combination of anti-PD-1 and anti-LAG-3 antibodies, or vehicle treatment, in the ASB-XIV syngeneic mouse tumor model. The grey area denotes the treatment period. Data are presented as means±SEM. *p<0.05, ****p<0.0001.

FIG. 10 is a pair of graphs showing the effect on tumor growth of anti-PD-1 antibody 12819, anti-TIM-3 antibody 5D12, or the combination of anti-PD-1 and anti-TIM-3 antibodies, or vehicle treatment, on tumor growth in two syngeneic mouse tumor models: ASB-XIV (lung cancer, Panel A) and Sa1N (fibrosarcoma, Panel B). The antibody treatments were administered at a dose of 10 mg/kg/target in mice with ASB-XIV tumors. Mice with Sa1N tumors were dosed with anti-PD-1 and anti-TIM-3 antibodies at 1 mg/kg and 10 mg/kg, respectively. The grey area denotes the treatment period. Data are presented as means±SEM. **p<0.01, ***p<0.001, and ****p<0.0001.

FIG. 11 is a set of graphs showing the effect on tumor growth of 10 mg/kg anti-PD-1 antibody 12819, anti-TIM-3 antibody 15086, or the combination of anti-PD-1 and anti-TIM-3 antibodies, or vehicle treatment, in a human xenograft tumor model, where the human melanoma cell line A375 was engrafted in mice reconstituted with human PBMC. One human PBMC donor was used in each experiment and the three graphs represent three different donors (Panels A-C). The grey area denotes the treatment period. Data are presented as means±SEM. *p<0.05 and **p<0.01.

FIG. 12 is a set of graphs showing the effect of single and dual targeting of PD-1 and TIM-3 on percent survival (Panel A) and tumor growth (Panels B-E) in NOD-scidmice engrafted with a mixture of human PBMCs and A375 melanoma cells. The mice were treated with anti-PD-1 antibody 12819, anti-TIM-3 antibody 15086.17145, or a combination of the anti-PD-1 and anti-TIM-3 antibodies. The antibody treatments were administered at a dose of 10 mg/kg three times weekly for each antibody. The grey area denotes the treatment period. Data in Panel A are presented as percentage of mice with tumor size <400 mm³ in each treatment group over time.

FIG. 13 is a set of graphs showing the effect of single, dual, and triple targeting of PD-1, LAG-3 and TIM-3 on percent survival (Panel A) and tumor growth (Panels B-H) in the ASB-XIV syngeneic tumor model using anti-PD-1 antibody 12819, anti-LAG-3 antibody C9B7W, anti-TIM-3 antibody 5D12, the combination of anti-PD1 and anti-LAG3 antibodies, the combination of anti-PD1 and anti-TIM-3 antibodies, or the “triple combo” (which refers to the combination of the anti-PD-1, anti-LAG-3, and anti-TIM-3 antibodies). The antibody treatments were administered at a dose of 10 mg/kg for each antibody. The grey area denotes the treatment period.

### DETAILED DESCRIPTION OF THE INVENTION

The present invention provides new combination therapies and compositions that target human PD-1, human TIM-3, and/or human LAG-3 by using antibodies that bind these targets. The therapies and compositions can be used to enhance the immune system in a human patient, such as a cancer patient. Unless otherwise stated, as used herein, “PD-1” refers to human PD-1. A human PD-1 polypeptide sequence is available under Uniprot Accession No. Q15116, shown here as SEQ ID NO: 388. Unless otherwise stated, as used herein, “TIM-3” refers to human TIM-3. A human TIM-3 polypeptide sequence is available under Uniprot Accession No. Q8TDQ0, shown here as SEQ ID NO: 389. Unless otherwise stated, as used herein, “LAG-3” refers to human LAG-3. A human LAG-3 polypeptide sequence is available under Uniprot Accession No. P18627, shown here as SEQ ID NO: 390.

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acid numbers in the heavy or light chain may be in accordance with IMGT© definitions (Lefranc et al., Dev Comp Immunol 27(1):55-77 (2003)); or the definitions of Kabat, Sequences of Proteins of Immunological Interest (National Institutes of Health, Bethesda, MD (1987 and 1991)); Chothia & Lesk, J. Mol. Biol. 196:901-917 (1987); or Chothia et al., Nature 342:878-883 (1989). Unless otherwise indicated, all antibody amino acid residue numbers referred to in this disclosure are those under the IMGT® numbering scheme.

The term “recombinant antibody” refers to an antibody that is expressed from a cell or cell line comprising the nucleotide sequence(s) that encode the antibody, wherein said nucleotide sequence(s) are not naturally associated with the cell.

The term “isolated protein”, “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between an antigen and an antibody. The intrinsic attractiveness of the antibody for the antigen is typically expressed as the binding affinity equilibrium constant (K_{D}) of a particular antibody-antigen interaction. An antibody is said to specifically bind to an antigen when the K_{D} is s 1 mM, preferably s 100 nM. A K_{D} binding affinity constant can be measured, e.g., by surface plasmon resonance (SPR) (BIAcore™) or Bio-Layer Interferometry, for example using the IBIS MX96 SPR system from IBIS Technologies, the ProteOn™ XPR36 SPR system from Bio-Rad, or the Octet™ system from ForteBio.

The term “k_{off}” refers to the dissociation rate constant of a particular antibody-antigen interaction. A kott dissociation rate constant can be measured, e.g., by Bio-Layer Interferometry, for example using one of the systems listed above.

The term “epitope” as used herein refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest or a relevant portion thereof, then screening for binding to the epitope.

One can determine whether an antibody binds to the same epitope as, or competes for binding with, an antibody as described herein by using methods known in the art, including, without limitation, competition assays, epitope binning, and alanine scanning. In some embodiments, the test antibody and an antibody as described herein bind to at least one common residue (e.g., at least two, three, four, five, six, seven, eight, or nine residues) on the target protein (i.e., TIM-3, PD-1, or LAG-3). In further embodiments, the contact residues on the target protein are completely identical between the test antibody and the antibody as described herein. In one embodiment, one allows the antibody as described herein to bind to the target protein under saturating conditions and then measures the ability of the test antibody to bind to the target protein. If the test antibody is able to bind to the target protein at the same time as the reference antibody, then the test antibody binds to a different epitope than the reference antibody. However, if the test antibody is not able to bind to the target protein at the same time, then the test antibody binds to the same epitope, an overlapping epitope, or an epitope that is in close proximity to the epitope bound by the antibody as described herein. This experiment can be performed using, e.g., ELISA, RIA, BIACORE™, SPR, Bio-Layer Interferometry or flow cytometry. To test whether an antibody cross-competes with another antibody, one may use the competition method described above in two directions, i.e., determining if the known antibody blocks the test antibody and vice versa. Such cross-competition experiments may be performed e.g. using an IBIS MX96 SPR instrument or the Octet™ system.

The term “chimeric antibody” refers in its broadest sense to an antibody that contains one or more regions from one antibody and one or more regions from one or more other antibodies, typically an antibody that is partially of human origin and partially of non-human origin, i.e., derived in part from a non-human animal, for example a mouse, rat or other rodent, or an avian such as a chicken. Chimeric antibodies are preferred over non-human antibodies in order to reduce the risk of a human anti-antibody response, e.g., a human anti-mouse antibody response in the case of a murine antibody. An example of a typical chimeric antibody is one in which the variable domain sequences are murine while the constant region sequences are human. In the case of a chimeric antibody, the non-human parts may be subjected to further alteration in order to humanize the antibody. The chimeric antibodies described herein have chicken variable domain sequences and human constant region sequences.

The term “humanize” refers to the fact that where an antibody is wholly or partially of non-human origin (for example, a murine or chicken antibody obtained from immunization of mice or chickens, respectively, with an antigen of interest, or a chimeric antibody based on such a murine or chicken antibody), it is possible to replace certain amino acids, in particular in the framework regions and constant regions of the heavy and light chains, in order to avoid or minimize an immune response in humans. Although it is not possible to precisely predict the immunogenicity and thereby the human anti-antibody response of a particular antibody, non-human antibodies tend to be more immunogenic in humans than human antibodies. Chimeric antibodies, where the foreign (e.g., rodent or avian) constant regions have been replaced with sequences of human origin, have been shown to be generally less immunogenic than antibodies of fully foreign origin, and the trend in therapeutic antibodies is towards humanized or fully human antibodies. Chimeric antibodies or other antibodies of non-human origin thus can be humanized to reduce the risk of a human anti-antibody response.

For chimeric antibodies, humanization typically involves modification of the framework regions of the variable domain sequences. Amino acid residues that are part of complementarity determining regions (CDRs) most often will not be altered in connection with humanization, although in certain cases it may be desirable to alter individual CDR amino acid residues, for example to remove a glycosylation site, a deamidation site, an aspartate isomerization site or an undesired cysteine or methionine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, preferably by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues.

Numerous methods for humanization of an antibody sequence are known in the art; see, e.g., the review by Almagro & Fransson, Front Biosci. 13:1619-1633 (2008). One commonly used method is CDR grafting, which for, e.g., a murine-derived chimeric antibody involves identification of human germline gene counterparts to the murine variable domain genes and grafting of the murine CDR sequences into this framework. The specificity of an antibody's interaction with a target antigen resides primarily in the amino acid residues located in the six CDRs of the heavy and light chain. The amino acid sequences within CDRs are therefore much more variable between individual antibodies than sequences outside of CDRs. Because CDR sequences are responsible for most antibody-antigen interactions, it is possible to express recombinant antibodies that mimic the properties of a specific naturally occurring antibody, or more generally any specific antibody with a given amino acid sequence, e.g., by constructing expression vectors that express CDR sequences from the specific antibody grafted into framework sequences from a different antibody. As a result, it is possible to “humanize” a non-human antibody and still substantially maintain the binding specificity and affinity of the original antibody. CDR grafting may be based on the Kabat CDR definitions, although a more recent publication (Magdelaine-Beuzelin et al., Crit Rev. Oncol Hematol. 64:210-225 (2007)) has suggested that the IMGT© definition (the international ImMunoGeneTics information system@, www.imgt.org) may improve the result of the humanization (see Lefranc et al., Dev. Comp Immunol. 27:55-77 (2003)).

In some cases, CDR grafting may reduce the binding specificity and affinity, and thus the biological activity, of a CDR-grafted non-human antibody as compared to the parent antibody from which the CDRs are obtained. Back mutations (sometimes referred to as “framework repair”) may be introduced at selected positions of the CDR-grafted antibody, typically in the framework regions, in order to reestablish the binding specificity and affinity of the parent antibody. Positions for possible back mutations can be identified using information available in the literature and in antibody databases. Amino acid residues that are candidates for back mutations are typically those that are located at the surface of an antibody molecule, while residues that are buried or that have a low degree of surface exposure will not normally be altered.

An alternative humanization technique to CDR grafting and back mutation is resurfacing, in which non-surface exposed residues of non-human origin are retained, while surface residues are altered to human residues.

In certain cases, it may be desirable to alter one or more CDR amino acid residues in order to improve binding affinity to the target epitope. This is known as “affinity maturation.” Various affinity maturation methods are known in the art, for example the in vitro scanning saturation mutagenesis method described by Burks et al., Proc Natl Acad Sci USA, 94:412-417 (1997), and the stepwise in vitro affinity maturation method of Wu et al., Proc Natl Acad Sci USA 95:6037-6042 (1998).

The term “human antibody” refers to an antibody in which the variable domain and constant region sequences are derived from human sequences. The term encompasses antibodies with sequences that are derived from human genes but have been modified, e.g., to decrease immunogenicity, increase affinity, and/or increase stability. Further, the term encompasses antibodies produced recombinantly using human-derived sequences in nonhuman cells, which may impart glycosylation not typical of human cells. The term also encompasses antibodies produced in transgenic nonhuman organisms with human antibody genes (e.g., OmniRat® rats).

The term “antigen-binding portion” of an antibody (or simply “antibody portion”), as used herein, refers to one or more portions or fragments of an antibody that retain the ability to specifically bind to an antigen (e.g., human TIM-3, human PD-1, or human LAG-3, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, C_{L} and C_{H}1 domains; (ii) a F(ab′)₂ fragment: a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment, which consists of a V_{H} domain; and (vi) an isolated complementarity determining region (CDR) capable of specifically binding to an antigen. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} domains pair to form monovalent molecules (known as single chain Fv (scFv)). Also within the invention are antigen-binding molecules comprising a V_{H} and/or a V_{L}. In the case of a V_{H}, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bi-specific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesion molecules can be obtained using standard recombinant DNA techniques, e.g., as described herein.

The class (isotype) and subclass of antibodies described herein may be determined by any method known in the art. In general, the class and subclass of an antibody may be determined using antibodies that are specific for a particular class and subclass of antibody. Such antibodies are available commercially. The class and subclass can be determined by ELISA and Western Blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies. A preferred isotype of the present invention is an IgG isotype.

When referring to particular amino acid residues in a given position of an antibody sequence, an indication of, e.g., “35S” refers to the position and residue, i.e., in this case indicating that a serine residue (S) is present in position 35 of the sequence. Similarly, an indication of, e.g., “13Q+35S” refers to the two residues in the respective positions. Unless otherwise indicated, all antibody amino acid residue numbers referred to in this disclosure are those under the IMGT® numbering scheme.

### Anti-PD-1 Antibodies

In some embodiments, the anti-PD-1 antibodies disclosed herein may be chimeric, with variable domains derived from chickens and human constant regions, or may be humanized.

The anti-PD-1 antibodies disclosed herein may be referred to by either a 5-digit number, e.g., “12819,” or by a 10-digit number, e.g., “12819.15384.” As used herein, the 5-digit number refers to all antibodies having the heavy and light chain CDR1-3 sequences shown for that number, whereas the use of a 10-digit number refers to a particular humanized variant. For example, 12819.15384 is a particular humanized variant having the CDR sequences of a 12819 antibody. The 5-digit number encompasses, for example, 10-digit variants that are identical except for some changes in the FRs (e.g., lacking residues SY at the N-terminus of the mature light chain, or having residues SS in lieu of SY). These modifications do not change the functional (e.g., antigen-binding) properties of the antibodies.

In some embodiments, the combination therapy or composition comprises an anti-PD-1 antibody or an antigen-binding portion thereof, wherein the anti-PD-1 antibody is the antibody referred to herein as antibody 12819.15384, 12748.15381, 12748.16124, 12865.15377, 12892.15378, 12796.15376, 12777.15382, 12760.15375 or 13112.15380 or a variant of any of these, where the variant may, e.g., contain certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of the antibody.

In some embodiments, the anti-PD-1 antibody competes for binding to human PD-1 with, or binds to the same epitope of human PD-1 as, any one of antibodies 12819.15384, 12748.15381, 12748.16124, 12865.15377, 12892.15378, 12796.15376, 12777.15382, 12760.15375 and 13112.15380.

In some embodiments, any of the anti-PD-1 antibodies or antigen-binding portions described herein may compete or cross-compete for binding to PD-1 with 12865, 12892, and 12777 antibodies (e.g., antibodies 12865.15377, 12892.15378, and 12777.15382). In some embodiments, any of the anti-PD-1 antibodies or antigen-binding portions described herein may compete or cross-compete for binding to PD-1 with a 12819 antibody (e.g., antibody 12819.15384). In some embodiments, any of the anti-PD-1 antibodies or antigen-binding portions described herein may compete or cross-compete for binding to PD-1 with 12760 and 13112 antibodies (e.g., antibodies 12760.15375 and 13112.15380). In some embodiments, the antibody has an IgG1 or IgG2 format. In certain embodiments, the antibody has an IgG1 format.

In some embodiments, the anti-PD-1 antibody competes or cross-competes for binding to human PD-1 with, or binds to the same epitope of human PD-1 as, an antibody whose heavy chain (H) CDR1-3 and light chain (L) CDR1-3 comprise, respectively, SEQ ID NOs: 228-233, 238-243, 248-253, 258-263, 268-273, 278-283, 288-293, or 298-303.

In some embodiments, the anti-PD-1 antibody comprises an H-CDR3 comprising the H-CDR3 amino acid sequence of SEQ ID NO: 230, 240, 250, 260, 270, 280, 290, or 300.

In some embodiments, the anti-PD-1 antibody comprises H-CDR1-3 comprising the H-CDR1-3 amino acid sequences, respectively, of SEQ ID NOs: 228-230, 238-240, 248-250, 258-260, 268-270, 278-280, 288-290, or 298-300.

In some embodiments, the anti-PD-1 antibody has a VH that is at least 90% (e.g., at least 92%, at least 95%, at least 96%, at least 97%, at least 98%, or at least 99%) identical in amino acid sequence to SEQ ID NO: 226, 236, 246, 256, 266, 276, 286, or 296.

In some embodiments, the anti-PD-1 antibody has a VH that comprises SEQ ID NO: 226, 236, 246, 256, 266, 276, 286, or 296.

In some embodiments, the anti-PD-1 antibody has a VH that is at least 90% (e.g., at least 92%, at least 95%, at least 96%, at least 97%, at least 98%, or at least 99%) identical in sequence to SEQ ID NO: 226, 236, 246, 256, 266, 276, 286, or 296; and a CH that is at least 90% (e.g., at least 92%, at least 95%, at least 96%, at least 97%, at least 98%, or at least 99%) identical in sequence to SEQ ID NO: 375.

In some embodiments, the anti-PD-1 antibody has an HC that comprises the VH amino acid sequence of SEQ ID NO: 226, 236, 246, 256, 266, 276, 286, or 296 and the CH amino acid sequence of SEQ ID NO: 375.

In some embodiments, the anti-PD-1 antibody has an L-CDR3 comprising the L-CDR3 amino acid sequence of SEQ ID NO: 233, 243, 253, 263, 273, 283, 293, or 303.

In some embodiments, the anti-PD-1 antibody comprises L-CDR1-3 comprising the L-CDR1-3 amino acid sequences, respectively, of SEQ ID NOs: 231-233, 241-243, 251-253, 261-263, 271-273, 281-283, 291-293, or 301-303.

In some embodiments, the anti-PD-1 antibody has a VL that is at least 90% (e.g., at least 92%, at least 95%, at least 96%, at least 97%, at least 98%, or at least 99%) identical in sequence to the VL amino acid sequence of SEQ ID NO: 227, 237, 247, 257, 267, 277, 287, 297, or 392.

In some embodiments, the anti-PD-1 antibody has a VL that comprises the VL amino acid sequence of SEQ ID NO: 227, 237, 247, 257, 267, 277, 287, 297, or 392.

In some embodiments, the anti-PD-1 antibody has a VL that is at least 90% (e.g., at least 92%, at least 95%, at least 96%, at least 97%, at least 98%, or at least 99%) identical in sequence to the VL amino acid sequence of SEQ ID NO: 227, 237, 247, 257, 267, 277, 287, 297, or 392; and a CL that is at least 90% (e.g., at least 92%, at least 95%, at least 96%, at least 97%, at least 98%, or at least 99%) identical in sequence to SEQ ID NO: 379.

In some embodiments, the anti-PD-1 antibody has a LC that comprises the VL amino acid sequence of SEQ ID NO: 227, 237, 247, 257, 267, 277, 287, 297, or 392 and the CL amino acid sequence of SEQ ID NO: 379.

In some embodiments, the anti-PD-1 antibody comprises any of the above heavy chain sequences and any of the above light chain sequences.

In some embodiments, the anti-PD-1 antibody comprises an H-CDR3 and L-CDR3 comprising the H-CDR3 and L-CDR3 amino acid sequences, respectively, of SEQ ID NOs: 230 and 233, 240 and 243, 250 and 253, 260 and 263, 270 and 273, 280 and 283, 290 and 293, or 300 and 303.

In some embodiments, the anti-PD-1 antibody comprises H-CDR1-3 and L-CDR1-3 comprising the H-CDR1-3 and L-CDR1-3 sequences, respectively, of SEQ ID NOs: 228-233, 238-243, 248-253, 258-263, 268-273, 278-283, 288-293, or 298-303.

In some embodiments, the anti-PD-1 antibody comprises a VH that is at least 90% (e.g., at least 92%, at least 95%, at least 96%, at least 97%, at least 98%, or at least 99%) identical in sequence to the amino acid sequence of SEQ ID NO: 226, 236, 246, 256, 266, 276, 286, or 296, and a VL that is at least 90% (e.g., at least 92%, at least 95%, at least 96%, at least 97%, at least 98%, or at least 99%) identical in sequence to the amino acid sequence of SEQ ID NO: 227, 237, 247, 257, 267, 277, 287, 297, or 392.

In some embodiments, the anti-PD-1 antibody has a VH that comprises the amino acid sequence of SEQ ID NO: 226, 236, 246, 256, 266, 276, 286, or 296, and a VL that comprises the amino acid sequence of SEQ ID NO: 227, 237, 247, 257, 267, 277, 287, 297, or 392.

In some embodiments, the anti-PD-1 antibody has an HC that comprises the amino acid sequence of SEQ ID NO: 226, 236, 246, 256, 266, 276, 286, or 296 and the amino acid sequence of SEQ ID NO: 375; and an LC that comprises the amino acid sequence of SEQ ID NO: 227, 237, 247, 257, 267, 277, 287, 297, or 392 and the amino acid sequence of SEQ ID NO: 379.

In some embodiments, the anti-PD-1 antibody comprises the H-CDR1-3 and L-CDR1-3 amino acid sequences of:
a) SEQ ID NOs: 228-233, respectively;
b) SEQ ID NOs: 238-243, respectively;
c) SEQ ID NOs: 248-253, respectively;
d) SEQ ID NOs: 258-263, respectively;
e) SEQ ID NOs: 268-273, respectively;
f) SEQ ID NOs: 278-283, respectively;
g) SEQ ID NOs: 288-293, respectively; or
h) SEQ ID NOs: 298-303, respectively.

In some embodiments, the anti-PD-1 antibody comprises a VH and a VL having the amino acid sequences of:
a) SEQ ID NOs: 226 and 227, respectively;
b) SEQ ID NOs: 236 and 237, respectively;
c) SEQ ID NOs: 236 and 392, respectively;
d) SEQ ID NOs: 246 and 247, respectively;
e) SEQ ID NOs: 256 and 257, respectively;
f) SEQ ID NOs: 266 and 267, respectively;
g) SEQ ID NOs: 276 and 277, respectively;
h) SEQ ID NOs: 286 and 287, respectively; or
i) SEQ ID NOs: 296 and 297, respectively.

In some embodiments, the anti-PD-1 antibody comprises:
a) an HC comprising the amino acid sequence of SEQ ID NO: 226 and the amino acid sequence of SEQ ID NO: 375, and an LC comprising the amino acid sequence of SEQ ID NO: 227 and the amino acid sequence of SEQ ID NO: 379;
b) an HC comprising the amino acid sequence of SEQ ID NO: 236 and the amino acid sequence of SEQ ID NO: 375, and an LC comprising the amino acid sequence of SEQ ID NO: 237 and the amino acid sequence of SEQ ID NO: 379;
c) an HC comprising the amino acid sequence of SEQ ID NO: 236 and the amino acid sequence of SEQ ID NO: 375, and an LC comprising the amino acid sequence of SEQ ID NO: 392 and the amino acid sequence of SEQ ID NO: 379;
d) an HC comprising the amino acid sequence of SEQ ID NO: 246 and the amino acid sequence of SEQ ID NO: 375, and an LC comprising the amino acid sequence of SEQ ID NO: 247 and the amino acid sequence of SEQ ID NO: 379;
e) an HC comprising the amino acid sequence of SEQ ID NO: 256 and the amino acid sequence of SEQ ID NO: 375, and an LC comprising the amino acid sequence of SEQ ID NO: 257 and the amino acid sequence of SEQ ID NO: 379;
f) an HC comprising the amino acid sequence of SEQ ID NO: 266 and the amino acid sequence of SEQ ID NO: 375, and an LC comprising the amino acid sequence of SEQ ID NO: 267 and the amino acid sequence of SEQ ID NO: 379;
g) an HC comprising the amino acid sequence of SEQ ID NO: 276 and the amino acid sequence of SEQ ID NO: 375, and an LC comprising the amino acid sequence of SEQ ID NO: 277 and the amino acid sequence of SEQ ID NO: 379;
h) an HC comprising the amino acid sequence of SEQ ID NO: 286 and the amino acid sequence of SEQ ID NO: 375, and an LC comprising the amino acid sequence of SEQ ID NO: 287 and the amino acid sequence of SEQ ID NO: 379; or
i) an HC comprising the amino acid sequence of SEQ ID NO: 296 and the amino acid sequence of SEQ ID NO: 375, and an LC comprising the amino acid sequence of SEQ ID NO: 297 and the amino acid sequence of SEQ ID NO: 379.

In some embodiments, the anti-PD-1 antibody is selected from the group consisting of:
a) an antibody whose H-CDR1-3 comprise the amino acid sequences of SEQ ID NOs: 228-230, respectively;
b) an antibody whose VH is at least 90% identical in sequence to the amino acid sequence of SEQ ID NO: 226;
c) an antibody whose VH comprises the amino acid sequence of SEQ ID NO: 226;
d) an antibody whose HC comprises the amino acid sequences of SEQ ID NOs: 226 and 375;
e) an antibody whose L-CDR1-3 comprise the amino acid sequences of SEQ ID NOs: 231-233, respectively;
f) an antibody whose VL is at least 90% identical in sequence to the amino acid sequence of SEQ ID NO: 227;
g) an antibody whose VL comprises the amino acid sequence of SEQ ID NO: 227;
h) an antibody whose LC comprises the amino acid sequences of SEQ ID NOs: 227 and 379;
i) an antibody whose H-CDR1-3 and L-CDR1-3 comprise the amino acid sequences of SEQ ID NOs: 228-233, respectively;
j) an antibody whose VH is at least 90% identical in sequence to the amino acid sequence of SEQ ID NO: 226 and whose VL is at least 90% identical in sequence to the amino acid sequence of SEQ ID NO: 227;
k) an antibody whose VH comprises the amino acid sequence of SEQ ID NO: 226 and whose VL comprises the amino acid sequence of SEQ ID NO: 227; and
l) an antibody whose HC comprises the amino acid sequence of SEQ ID NO: 226 and the amino acid sequence of SEQ ID NO: 375; and whose LC comprises the amino acid sequences of SEQ ID NOs: 227 and 379.

In some embodiments, the anti-PD-1 antibody is selected from the group consisting of:
a) an antibody whose H-CDR1-3 comprise the amino acid sequences of SEQ ID NOs: 238-240, respectively;
b) an antibody whose VH is at least 90% identical in sequence to the amino acid sequence of SEQ ID NO: 236;
c) an antibody whose VH comprises the amino acid sequence of SEQ ID NO: 236;
d) an antibody whose HC comprises the amino acid sequences of SEQ ID NOs: 236 and 375;
e) an antibody whose L-CDR1-3 comprise the amino acid sequences of SEQ ID NOs: 241-243, respectively;
f) an antibody whose VL is at least 90% identical in sequence to the amino acid sequence of SEQ ID NO: 237;
g) an antibody whose VL comprises the amino acid sequence of SEQ ID NO: 237;
h) an antibody whose LC comprises the amino acid sequences of SEQ ID NOs: 237 and 379;
i) an antibody whose H-CDR1-3 and L-CDR1-3 comprise the amino acid sequences of SEQ ID NOs: 238-243, respectively;
j) an antibody whose VH is at least 90% identical in sequence to the amino acid sequence of SEQ ID NO: 236 and whose VL is at least 90% identical in sequence to the amino acid sequence of SEQ ID NO: 237;
k) an antibody whose VH comprises the amino acid sequence of SEQ ID NO: 236 and whose VL comprises the amino acid sequence of SEQ ID NO: 237; and
l) an antibody whose HC comprises the amino acid sequence of SEQ ID NO: 236 and the amino acid sequence of SEQ ID NO: 375; and whose LC comprises the amino acid sequences of SEQ ID NOs: 237 and 379.

In some embodiments, the anti-PD-1 antibody is selected from the group consisting of:
a) an antibody whose H-CDR1-3 comprise the amino acid sequences of SEQ ID NOs: 238-240, respectively;
b) an antibody whose VH is at least 90% identical in sequence to the amino acid sequence of SEQ ID NO: 236;
c) an antibody whose VH comprises the amino acid sequence of SEQ ID NO: 236;
d) an antibody whose HC comprises the amino acid sequences of SEQ ID NOs: 236 and 375;
e) an antibody whose L-CDR1-3 comprise the amino acid sequences of SEQ ID NOs: 241-243, respectively;
f) an antibody whose VL is at least 90% identical in sequence to the amino acid sequence of SEQ ID NO: 392;
g) an antibody whose VL comprises the amino acid sequence of SEQ ID NO: 392;
h) an antibody whose LC comprises the amino acid sequences of SEQ ID NOs: 392 and 379;
i) an antibody whose H-CDR1-3 and L-CDR1-3 comprise the amino acid sequences of SEQ ID NOs: 238-243, respectively;
j) an antibody whose VH is at least 90% identical in sequence to the amino acid sequence of SEQ ID NO: 236 and whose VL is at least 90% identical in sequence to the amino acid sequence of SEQ ID NO: 392;
k) an antibody whose VH comprises the amino acid sequence of SEQ ID NO: 236 and whose VL comprises the amino acid sequence of SEQ ID NO: 392; and
l) an antibody whose HC comprises the amino acid sequence of SEQ ID NO: 236 and the amino acid sequence of SEQ ID NO: 375; and whose LC comprises the amino acid sequences of SEQ ID NOs: 392 and 379.

In some embodiments, the anti-PD-1 antibody is selected from the group consisting of:
a) an antibody whose H-CDR1-3 comprise the amino acid sequences of SEQ ID NOs: 248-250, respectively;
b) an antibody whose VH is at least 90% identical in sequence to the amino acid sequence of SEQ ID NO: 246;
c) an antibody whose VH comprises the amino acid sequence of SEQ ID NO: 246;
d) an antibody whose HC comprises the amino acid sequences of SEQ ID NOs: 246 and 375;
e) an antibody whose L-CDR1-3 comprise the amino acid sequences of SEQ ID NOs: 251-253, respectively;
f) an antibody whose VL is at least 90% identical in sequence to the amino acid sequence of SEQ ID NO: 247;
g) an antibody whose VL comprises the amino acid sequence of SEQ ID NO: 247;
h) an antibody whose LC comprises the amino acid sequences of SEQ ID NOs: 247 and 379;
i) an antibody whose H-CDR1-3 and L-CDR1-3 comprise the amino acid sequences of SEQ ID NOs: 248-253, respectively;
j) an antibody whose VH is at least 90% identical in sequence to the amino acid sequence of SEQ ID NO: 246 and whose VL is at least 90% identical in sequence to the amino acid sequence of SEQ ID NO: 247;
k) an antibody whose VH comprises the amino acid sequence of SEQ ID NO: 246 and whose VL comprises the amino acid sequence of SEQ ID NO: 247; and
l) an antibody whose HC comprises the amino acid sequence of SEQ ID NO: 246 and the amino acid sequence of SEQ ID NO: 375; and whose LC comprises the amino acid sequences of SEQ ID NOs: 247 and 379.

In some embodiments, the anti-PD-1 antibody is selected from the group consisting of:
a) an antibody whose H-CDR1-3 comprise the amino acid sequences of SEQ ID NOs: 258-260, respectively;
b) an antibody whose VH is at least 90% identical in sequence to the amino acid sequence of SEQ ID NO: 256;
c) an antibody whose VH comprises the amino acid sequence of SEQ ID NO: 256;
d) an antibody whose HC comprises the amino acid sequences of SEQ ID NOs: 256 and 375;
e) an antibody whose L-CDR1-3 comprise the amino acid sequences of SEQ ID NOs: 261-263, respectively;
f) an antibody whose VL is at least 90% identical in sequence to the amino acid sequence of SEQ ID NO: 257;
g) an antibody whose VL comprises the amino acid sequence of SEQ ID NO: 257;
h) an antibody whose LC comprises the amino acid sequences of SEQ ID NOs: 257 and 379;
i) an antibody whose H-CDR1-3 and L-CDR1-3 comprise the amino acid sequences of SEQ ID NOs: 258-263, respectively;
j) an antibody whose VH is at least 90% identical in sequence to the amino acid sequence of SEQ ID NO: 256 and whose VL is at least 90% identical in sequence to the amino acid sequence of SEQ ID NO: 257;
k) an antibody whose VH comprises the amino acid sequence of SEQ ID NO: 256 and whose VL comprises the amino acid sequence of SEQ ID NO: 257; and
l) an antibody whose HC comprises the amino acid sequence of SEQ ID NO: 256 and the amino acid sequence of SEQ ID NO: 375; and whose LC comprises the amino acid sequences of SEQ ID NOs: 257 and 379.

In some embodiments, the anti-PD-1 antibody is selected from the group consisting of:
a) an antibody whose H-CDR1-3 comprise the amino acid sequences of SEQ ID NOs: 268-270, respectively;
b) an antibody whose VH is at least 90% identical in sequence to the amino acid sequence of SEQ ID NO: 266;
c) an antibody whose VH comprises the amino acid sequence of SEQ ID NO: 266;
d) an antibody whose HC comprises the amino acid sequences of SEQ ID NOs: 266 and 375;
e) an antibody whose L-CDR1-3 comprise the amino acid sequences of SEQ ID NOs: 271-273, respectively;
f) an antibody whose VL is at least 90% identical in sequence to the amino acid sequence of SEQ ID NO: 267;
g) an antibody whose VL comprises the amino acid sequence of SEQ ID NO: 267;
h) an antibody whose LC comprises the amino acid sequences of SEQ ID NOs: 267 and 379;
i) an antibody whose H-CDR1-3 and L-CDR1-3 comprise the amino acid sequences of SEQ ID NOs: 268-273, respectively;
j) an antibody whose VH is at least 90% identical in sequence to the amino acid sequence of SEQ ID NO: 266 and whose VL is at least 90% identical in sequence to the amino acid sequence of SEQ ID NO: 267;
k) an antibody whose VH comprises the amino acid sequence of SEQ ID NO: 266 and whose VL comprises the amino acid sequence of SEQ ID NO: 267; and
l) an antibody whose HC comprises the amino acid sequence of SEQ ID NO: 266 and the amino acid sequence of SEQ ID NO: 375; and whose LC comprises the amino acid sequences of SEQ ID NOs: 267 and 379.

In some embodiments, the anti-PD-1 antibody is selected from the group consisting of:
a) an antibody whose H-CDR1-3 comprise the amino acid sequences of SEQ ID NOs: 278-280, respectively;
b) an antibody whose VH is at least 90% identical in sequence to the amino acid sequence of SEQ ID NO: 276;
c) an antibody whose VH comprises the amino acid sequence of SEQ ID NO: 276;
d) an antibody whose HC comprises the amino acid sequences of SEQ ID NOs: 276 and 375;
e) an antibody whose L-CDR1-3 comprise the amino acid sequences of SEQ ID NOs: 281-283, respectively;
f) an antibody whose VL is at least 90% identical in sequence to the amino acid sequence of SEQ ID NO: 277;
g) an antibody whose VL comprises the amino acid sequence of SEQ ID NO: 277;
h) an antibody whose LC comprises the amino acid sequences of SEQ ID NOs: 277 and 379;
i) an antibody whose H-CDR1-3 and L-CDR1-3 comprise the amino acid sequences of SEQ ID NOs: 278-283, respectively;
j) an antibody whose VH is at least 90% identical in sequence to the amino acid sequence of SEQ ID NO: 276 and whose VL is at least 90% identical in sequence to the amino acid sequence of SEQ ID NO: 277;
k) an antibody whose VH comprises the amino acid sequence of SEQ ID NO: 276 and whose VL comprises the amino acid sequence of SEQ ID NO: 277; and
l) an antibody whose HC comprises the amino acid sequence of SEQ ID NO: 276 and the amino acid sequence of SEQ ID NO: 375; and whose LC comprises the amino acid sequences of SEQ ID NOs: 277 and 379.

In some embodiments, the anti-PD-1 antibody is selected from the group consisting of:
a) an antibody whose H-CDR1-3 comprise the amino acid sequences of SEQ ID NOs: 288-290, respectively;
b) an antibody whose VH is at least 90% identical in sequence to the amino acid sequence of SEQ ID NO: 286;
c) an antibody whose VH comprises the amino acid sequence of SEQ ID NO: 286;
d) an antibody whose HC comprises the amino acid sequences of SEQ ID NOs: 286 and 375;
e) an antibody whose L-CDR1-3 comprise the amino acid sequences of SEQ ID NOs: 291-293, respectively;
f) an antibody whose VL is at least 90% identical in sequence to the amino acid sequence of SEQ ID NO: 287;
g) an antibody whose VL comprises the amino acid sequence of SEQ ID NO: 287;
h) an antibody whose LC comprises the amino acid sequences of SEQ ID NOs: 287 and 379;
i) an antibody whose H-CDR1-3 and L-CDR1-3 comprise the amino acid sequences of SEQ ID NOs: 288-293, respectively;
j) an antibody whose VH is at least 90% identical in sequence to the amino acid sequence of SEQ ID NO: 286 and whose VL is at least 90% identical in sequence to the amino acid sequence of SEQ ID NO: 287;
k) an antibody whose VH comprises the amino acid sequence of SEQ ID NO: 286 and whose VL comprises the amino acid sequence of SEQ ID NO: 287; and
l) an antibody whose HC comprises the amino acid sequence of SEQ ID NO: 286 and the amino acid sequence of SEQ ID NO: 375; and whose LC comprises the amino acid sequences of SEQ ID NOs: 287 and 379.

In some embodiments, the anti-PD-1 antibody is selected from the group consisting of:
a) an antibody whose H-CDR1-3 comprise the amino acid sequences of SEQ ID NOs: 298-300, respectively;
b) an antibody whose VH is at least 90% identical in sequence to the amino acid sequence of SEQ ID NO: 296;
c) an antibody whose VH comprises the amino acid sequence of SEQ ID NO: 296;
d) an antibody whose HC comprises the amino acid sequences of SEQ ID NOs: 296 and 375;
e) an antibody whose L-CDR1-3 comprise the amino acid sequences of SEQ ID NOs: 301-303, respectively;
f) an antibody whose VL is at least 90% identical in sequence to the amino acid sequence of SEQ ID NO: 297;
g) an antibody whose VL comprises the amino acid sequence of SEQ ID NO: 297;
h) an antibody whose LC comprises the amino acid sequences of SEQ ID NOs: 297 and 379;
i) an antibody whose H-CDR1-3 and L-CDR1-3 comprise the amino acid sequences of SEQ ID NOs: 298-303, respectively;
j) an antibody whose VH is at least 90% identical in sequence to the amino acid sequence of SEQ ID NO: 296 and whose VL is at least 90% identical in sequence to the amino acid sequence of SEQ ID NO: 297;
k) an antibody whose VH comprises the amino acid sequence of SEQ ID NO: 296 and whose VL comprises the amino acid sequence of SEQ ID NO: 297; and
l) an antibody whose HC comprises the amino acid sequence of SEQ ID NO: 296 and the amino acid sequence of SEQ ID NO: 375; and whose LC comprises the amino acid sequences of SEQ ID NOs: 297 and 379.

In some embodiments, any of the anti-PD-1 antibodies or antigen-binding portions described herein may bind to human PD-1 with a K_{D} of at least 900, at least 850, at least 800, at least 750, at least 700, at least 650, at least 600, at least 550, at least 500, at least 450, at least 400, at least 350, at least 300, at least 250, at least 200, at least 150, at least 100, at least 50, at least 40, at least 30, or at least 20 pM. In certain embodiments, the K_{D} is determined using surface plasmon resonance. In particular embodiments, the anti-PD-1 antibodies or antigen-binding portions bind to human PD-1 with a higher affinity than nivolumab, pembrolizumab, or both.

In some embodiments, any of the anti-PD-1 antibodies or antigen-binding portions described herein may bind to cynomolgus PD-1 with a K_{D} of at least 9000, at least 8000, at least 7000, at least 6000, at least 5000, at least 4000, at least 3000, at least 2500, at least 2000, at least 1500, at least 1000, at least 900, at least 800, at least 700, at least 600, at least 500, at least 400, at least 300, at least 200, at least 100, at least 75, at least 50, at least 25, at least 20, at least 15, at least 10, or at least 5 pM. In certain embodiments, the K_{D} is determined using surface plasmon resonance.

In some embodiments, any of the anti-PD-1 antibodies or antigen-binding portions described herein may bind to mouse PD-1 with a K_{D} of at least 1000, at least 950, at least 900, or at least 850 pM. In certain embodiments, the K_{D} is determined using surface plasmon resonance.

In some embodiments, any of the anti-PD-1 antibodies or antigen-binding portions described herein may inhibit the interaction of PD-1 with PD-L1 by at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% at a concentration of 10 μg/mL in a flow cytometric competition assay. In certain embodiments, the anti-PD-1 antibodies or antigen-binding portions may inhibit the interaction of PD-1 with PD-L1 by at least 83%.

In some embodiments, any of the anti-PD-1 antibodies or antigen-binding portions described herein may block binding of PD-L1 and PD-L2 to PD-1 by at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or 100% at a concentration of 10 μg/mL as determined by Bio-Layer Interferometry analysis. In certain embodiments, the anti-PD-1 antibodies or antigen-binding portions block binding of PD-L1 and PD-L2 to PD-1 by at least 90%.

In some embodiments, the anti-PD-1 antibody or antigen-binding portion described herein has at least one of the following properties:
a) binds to human PD-1 with a K_{D} of 750 pM or less;
b) binds to cynomolgus PD-1 with a K_{D} of 7 nM or less;
c) binds to mouse PD-1 with a K_{D} of 1 nM or less;
d) does not bind to rat PD-1;
e) increases IL-2 secretion in an SEB whole blood assay;
f) increases IFN-γ secretion in a one-way mixed lymphocyte reaction assay; g) inhibits the interaction of PD-1 with PD-L1 by at least 60% at a concentration of 10 μg/mL in a flow cytometric competition assay;
h) blocks binding of PD-L1 and PD-L2 to PD-1 by at least 90% at a concentration of 10 μg/mL as determined by Bio-Layer Interferometry analysis; and
i) inhibits tumor growth in vivo.

Examples of such an antibody include, without limitation, a 12819 antibody (having properties a-i); 12748, 12892, and 12777 antibodies (having at least properties a, b, and e-h); 12865 and 12796 antibodies (having at least properties a, b, e, f, and h), and 12760 and 13112 antibodies (having at least properties a, b, e, and f). In some embodiments, the anti-PD-1 antibody or antigen-binding portion has all of said properties. In some embodiments, the anti-PD-1 antibody or antigen-binding portion has at least properties a, b, and e-h. In some embodiments, the anti-PD-1 antibody or antigen-binding portion has at least properties a, b, e, f, and h. In some embodiments, the anti-PD-1 antibody or antigen-binding portion has at least properties a, b, e, and f.

In some embodiments, an anti-PD-1 antibody or an antigen-binding portion thereof as described herein binds to an epitope of PD-1 that includes at least one (e.g., at least one, at least two, at least three, at least four, or at least five) of the following residues of SEQ ID NO: 388: V44, V64, L128, P130, K131, A132, E136, and T145. In certain embodiments, the antibody or antigen-binding portion binds to an epitope of PD-1 that includes residues V64, L128, P130, K131, and A132 of SEQ ID NO: 388 (such as a 12819 antibody, e.g., antibody 12819.15384). In certain embodiments, the antibody or antigen-binding portion binds to an epitope of PD-1 that includes residues K131 and E136 of SEQ ID NO: 388 (such as a 12865 antibody, e.g., antibody 12865.15377). In certain embodiments, the antibody or antigen-binding portion binds to an epitope of PD-1 that includes residues V44 and T145 of SEQ ID NO: 388 (such as a 13112 antibody, e.g., antibody 13112.15380).

In some embodiments, the combination therapy or composition comprises an anti-PD-1 antibody or an antigen-binding portion thereof that binds to an epitope of PD-1 comprising amino acid residue K131 of SEQ ID NO: 388 (e.g., a 12819 or 12865 antibody). In some embodiments, the epitope further comprises amino acid residues P130 and A132, and may additionally comprise amino acid residues V64 and L128 (e.g., a 12819 antibody). In some embodiments, the epitope further comprises amino acid residue E136 (e.g., a 12865 antibody).

In some embodiments, an anti-PD-1 antibody or an antigen-binding portion thereof as described herein binds to an epitope of PD-1 that comprises residues 56-64, 69-90, and/or 122-140 of SEQ ID NO: 388. In certain embodiments, the antibody or antigen-binding portion binds to an epitope of PD-1 that comprises residues 69-90 and 122-140 of SEQ ID NO: 388 (such as 12819 and 12865 antibodies, e.g., antibodies 12819.15384 and 12865.15377). In certain embodiments, the antibody or antigen-binding portion binds to an epitope of PD-1 that comprises residues 56-64, 69-90, and 122-140 of SEQ ID NO: 388 (e.g., a 12819 antibody). In certain embodiments, the antibody or antigen-binding portion binds to an epitope of PD-1 that comprises residues 69-90 and 122-140 of SEQ ID NO: 388 (e.g., a 12865 antibody). In some embodiments, the antibody or portion binds to residues 69-75 (or a fragment thereof, such as a one, two, three, four, five, or six residue fragment), of SEQ ID NO: 388 (such as 12819 and 12865 antibodies, e.g., antibodies 12819.15384 and 12865.15377). In some embodiments, the antibody or portion binds to residues 136-140 (or a fragment thereof, such as a one, two, three, or four residue fragment) of SEQ ID NO: 388 (such as 12819 and 12865 antibodies, e.g., antibodies 12819.15384 and 12865.15377). In some embodiments, the antibody or portion binds to residues 69-75 (or a fragment thereof) and residues 136-140 (or a fragment thereof) of SEQ ID NO: 388, (such as 12819 and 12865 antibodies, e.g., antibodies 12819.15384 and 12865.15377). An epitope with any combination of the above residues is also contemplated.

In some embodiments, an anti-PD-1 antibody or an antigen-binding portion thereof as described herein is an anti-PD-1 antibody or antigen-binding portion described in PCT Patent Publication WO 2017/055547 or PCT Patent Application PCT/EP2017/079615, which are incorporated by reference in their entirety herein.

### Anti-TIM-3 Antibodies

In a particular embodiment, the anti-TIM-3 antibodies disclosed herein are human antibodies generated from transgenic rats that are able to generate antibodies with human idiotypes.

The anti-TIM-3 antibodies disclosed herein may be referred to by either a 5-digit number, e.g. “20131”, or by a 10-digit number, e.g. “15086.16837”. 10-digit numbers with the same first five digits are derived from the same parent antibody, as in the case of antibodies 15086.15086, 15086.16837, 15086.17145, 15086.17144. Such antibodies, which share the same six CDRs, are expected to have the same or substantially the same target binding properties. As will be apparent from the protein and DNA sequences provided herein, the 15086.16837, 15086.17145, and 15086.17144 variants have only a single amino acid difference in the VH sequence compared to the parent 15086 antibody (“15086.15086”), namely E, rather than Q, in position 6, whereas the VL amino acid sequences are identical. It will also be apparent that these variants differ primarily by their antibody format/subclass, i.e.:
15086.15086: IgG1
15086.16837: IgG1 LALA
15086.17145: IgG2
15086.17144: IgG4
   The VH sequence of the IgG2 and IgG4 subclass antibodies is the same as that of the IgG1 LALA variant. The VL sequence is the same for all four of the antibody subclasses.

In some embodiments, the combination therapy or composition comprises an anti-TIM-3 antibody or an antigen-binding portion thereof, wherein the anti-TIM-3 antibody is the antibody referred to herein as antibody 15086.17145, 15086.15086, 15086.16837, 15086.17144, 20131, 20293, 15105, 15107, 15109, 15174, 15175, 15260, 15284, 15299, 15353, 15354, 17244, 17245, 19324, 19416, 19568, 20185, 20300, 20362, or 20621 or a variant of any of these, where the variant may, e.g., contain certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of antibody.

In some embodiments, the anti-TIM-3 antibody competes or cross-competes for binding to human TIM-3 with, or binds to the same epitope of human TIM-3 as, antibody 15086.15086 having the IgG1 format, antibody 15086.16837 having the IgG1 LALA format, antibody 15086.17145 having the IgG2 format, or antibody 15086.17144 having the IgG4 format. In some embodiments, the antibody has an IgG1 or IgG2 format. In certain embodiments, the antibody has an IgG2 format.

In some embodiments, the anti-TIM-3 antibody competes or cross-competes for binding to human TIM-3 with, or binds to the same epitope of human TIM-3 as, antibody 20131, 20293, 15105, 15107, 15109, 15174, 15175, 15260, 15284, 15299, 15353, 15354, 17244, 17245, 19324, 19416, 19568, 20185, 20300, 20362, or 20621. In some embodiments, the antibody has an IgG1 or IgG2 format. In certain embodiments, the antibody has an IgG2 format.

In some embodiments, the anti-TIM-3 antibody competes or cross-competes for binding to human TIM-3 with, or binds to the same epitope of human TIM-3 as, an antibody whose heavy chain (H) CDR1-3 and light chain (L) CDR1-3 comprise, respectively, SEQ ID NOs: 8-13, 18-23, 28-33, 38-43, 48-53, 58-63, 68-73, 78-83, 88-93, 98-103, 108-113, 118-123, 128-133, 138-143, 148-153, 158-163, 168-173, 178-183, 188-193, 198-203, 208-213, or 218-223.

In some embodiments, the anti-TIM-3 antibody comprises an H-CDR3 comprising the H-CDR3 amino acid sequence of SEQ ID NO: 10, 20, 30, 40, 50, 60, 70, 80, 90, 100, 110, 120, 130, 140, 150, 160, 170, 180, 190, 200, 210, or 220.

In some embodiments, the anti-TIM-3 antibody comprises H-CDR1-3 comprising the H-CDR1-3 amino acid sequences, respectively, of SEQ ID NOs: 8-10, 18-20, 28-30, 38-40, 48-50, 58-60, 68-70, 78-80, 88-90, 98-100, 108-110, 118-120, 128-130, 138-140, 148-150, 158-160, 168-170, 178-180, 188-190, 198-200, 208-210, or 218-220.

In some embodiments, the anti-TIM-3 antibody has a VH that is at least 90% (e.g., at least 92%, at least 95%, at least 96%, at least 97%, at least 98%, or at least 99%) identical in amino acid sequence to SEQ ID NO: 3, 7, 16, 26, 36, 46, 56, 66, 76, 86, 96, 106, 116, 126, 136, 146, 156, 166, 176, 186, 196, 206, or 216.

In some embodiments, the anti-TIM-3 antibody has a VH that comprises SEQ ID NO: 3, 7, 16, 26, 36, 46, 56, 66, 76, 86, 96, 106, 116, 126, 136, 146, 156, 166, 176, 186, 196, 206, or 216.

In some embodiments, the anti-TIM-3 antibody has a VH that is at least 90% (e.g., at least 92%, at least 95%, at least 96%, at least 97%, at least 98%, or at least 99%) identical in sequence to SEQ ID NO: 3, 16, 26, 36, 46, 56, 66, 76, 86, 96, 106, 116, 126, 136, 146, 156, 166, 176, 186, 196, 206, or 216; and a CH that is at least 90% (e.g., at least 92%, at least 95%, at least 96%, at least 97%, at least 98%, or at least 99%) identical in sequence to SEQ ID NO: 374.

In some embodiments, the anti-TIM-3 antibody has an HC that comprises the VH amino acid sequence of SEQ ID NO: 3, 16, 26, 36, 46, 56, 66, 76, 86, 96, 106, 116, 126, 136, 146, 156, 166, 176, 186, 196, 206, or 216 and the CH amino acid sequence of SEQ ID NO: 374.

In some embodiments, the anti-TIM-3 antibody has a VH that is at least 90% (e.g., at least 92%, at least 95%, at least 96%, at least 97%, at least 98%, or at least 99%) identical in sequence to SEQ ID NO: 7, 16, 26, 36, 46, 56, 66, 76, 86, 96, 106, 116, 126, 136, 146, 156, 166, 176, 186, 196, 206, or 216; and a CH that is at least 90% (e.g., at least 92%, at least 95%, at least 96%, at least 97%, at least 98%, or at least 99%) identical in sequence to SEQ ID NO: 375, 376, or 377.

In some embodiments, the anti-TIM-3 antibody has a HC that comprises the VH amino acid sequence of SEQ ID NO: 7, 16, 26, 36, 46, 56, 66, 76, 86, 96, 106, 116, 126, 136, 146, 156, 166, 176, 186, 196, 206, or 216 and the CH amino acid sequence of SEQ ID NO: 375, 376, or 377. In certain embodiments, the CH amino acid sequence is SEQ ID NO: 377.

In some embodiments, the anti-TIM-3 antibody comprises an L-CDR3 comprising the L-CDR3 amino acid sequence of SEQ ID NO: 13, 23, 33, 43, 53, 63, 73, 83, 93, 103, 113, 123, 133, 143, 153, 163, 173, 183, 193, 203, 213, or 223.

In some embodiments, the anti-TIM-3 antibody comprises L-CDR1-3 comprising the L-CDR1-3 amino acid sequences, respectively, of SEQ ID NOs: 11-13, 21-23, 31-33, 41-43, 51-53, 61-63, 71-73, 81-83, 91-93, 101-103, 111-113, 121-123, 131-133, 141-143, 151-153, 161-163, 171-173, 181-183, 191-193, 201-203, 211-213, or 221-223.

In some embodiments, the anti-TIM-3 antibody has a VL that is at least 90% (e.g., at least 92%, at least 95%, at least 96%, at least 97%, at least 98%, or at least 99%) identical in sequence to the VL amino acid sequence of SEQ ID NO: 4, 17, 27, 37, 47, 57, 67, 77, 87, 97, 107, 117, 127, 137, 147, 157, 167, 177, 187, 197, 207, or 217.

In some embodiments, the anti-TIM-3 antibody has a VL that comprises the VL amino acid sequence of SEQ ID NO: 4, 17, 27, 37, 47, 57, 67, 77, 87, 97, 107, 117, 127, 137, 147, 157, 167, 177, 187, 197, 207, or217.

In some embodiments, the anti-TIM-3 antibody has a VL that is at least 90% (e.g., at least 92%, at least 95%, at least 96%, at least 97%, at least 98%, or at least 99%) identical in sequence to the VL amino acid sequence of SEQ ID NO: 4, 17, 27, 37, 47, 57, 67, 77, 87, 97, 107, 117, 127, 137, 147, 157, 167, 177, 187, 197, 207, or 217; and a CL that is at least 90% (e.g., at least 92%, at least 95%, at least 96%, at least 97%, at least 98%, or at least 99%) identical in sequence to SEQ ID NO: 378.

In some embodiments, the anti-TIM-3 antibody has a LC that comprises the VL amino acid sequence of SEQ ID NO: 4, 17, 27, 37, 47, 57, 67, 77, 87, 97, 107, 117, 127, 137, 147, 157, 167, 177, 187, 197, 207, or 217 and the CL amino acid sequence of SEQ ID NO: 378.

In some embodiments, the anti-TIM-3 antibody comprises any of the above heavy chain sequences and any of the above light chain sequences.

In some embodiments, the anti-TIM-3 antibody comprises an H-CDR3 and L-CDR3 comprising the H-CDR3 and L-CDR3 amino acid sequences, respectively, of SEQ ID NOs: 10 and 13, 20 and 23, 30 and 33, 40 and 43, 50 and 53, 60 and 63, 70 and 73, 80 and 83, 90 and 93, 100 and 103, 110 and 113, 120 and 123, 130 and 133, 140 and 143, 150 and 153, 160 and 163, 170 and 173, 180 and 183, 190 and 193, 200 and 203, 210 and 213, or 220 and 223.

In some embodiments, the anti-TIM-3 antibody comprises H-CDR1-3 and L-CDR1-3 comprising the H-CDR1-3 and L-CDR1-3 sequences, respectively, of SEQ ID NOs: 8-13, 18-23, 28-33, 38-43, 48-53, 58-63, 68-73, 78-83, 88-93, 98-103, 108-113, 118-123, 128-133, 138-143, 148-153, 158-163, 168-173, 178-183, 188-193, 198-203, 208-213, or 218-223.

In some embodiments, the anti-TIM-3 antibody has a VH that is at least 90% (e.g., at least 92%, at least 95%, at least 96%, at least 97%, at least 98%, or at least 99%) identical in sequence to the amino acid sequence of SEQ ID NO: 3, 7, 16, 26, 36, 46, 56, 66, 76, 86, 96, 106, 116, 126, 136, 146, 156, 166, 176, 186, 196, 206, or 216, and a VL that is at least 90% (e.g., at least 92%, at least 95%, at least 96%, at least 97%, at least 98%, or at least 99%) identical in sequence to the amino acid sequence of SEQ ID NO: 4, 17, 27, 37, 47, 57, 67, 77, 87, 97, 107, 117, 127, 137, 147, 157, 167, 177, 187, 197, 207, or217.

In some embodiments, the anti-TIM-3 antibody has a VH that comprises the amino acid sequence of SEQ ID NO: 3, 7, 16, 26, 36, 46, 56, 66, 76, 86, 96, 106, 116, 126, 136, 146, 156, 166, 176, 186, 196, 206, or 216, and a VL that comprises the amino acid sequence of SEQ ID NO: 4, 17, 27, 37, 47, 57, 67, 77, 87, 97, 107, 117, 127, 137, 147, 157, 167, 177, 187, 197, 207, or217.

In some embodiments, the anti-TIM-3 antibody has an HC that comprises the amino acid sequence of SEQ ID NO: 3, 7, 16, 26, 36, 46, 56, 66, 76, 86, 96, 106, 116, 126, 136, 146, 156, 166, 176, 186, 196, 206, or 216 and the amino acid sequence of SEQ ID NO: 378; and an LC that comprises the amino acid sequence of SEQ ID NO: 4, 17, 27, 37, 47, 57, 67, 77, 87, 97, 107, 117, 127, 137, 147, 157, 167, 177, 187, 197, 207, or 217 and the amino acid sequence of SEQ ID NO: 374, 375, 376, or 377.

In some embodiments, the anti-TIM-3 antibody comprises the H-CDR1-3 and L-CDR1-3 amino acid sequences of:
a) SEQ ID NOs: 8-13, respectively;
b) SEQ ID NOs: 18-23, respectively;
c) SEQ ID NOs: 28-33, respectively;
d) SEQ ID NOs: 38-43, respectively;
e) SEQ ID NOs: 48-53, respectively;
f) SEQ ID NOs: 58-63, respectively;
g) SEQ ID NOs: 68-73, respectively;
h) SEQ ID NOs: 78-83, respectively;
i) SEQ ID NOs: 88-93, respectively;
j) SEQ ID NOs: 98-103, respectively;
k) SEQ ID NOs: 108-113, respectively;
l) SEQ ID NOs: 118-123, respectively;
m) SEQ ID NOs: 128-133, respectively;
n) SEQ ID NOs: 138-143, respectively;
o) SEQ ID NOs: 148-153, respectively;
p) SEQ ID NOs: 158-163, respectively;
q) SEQ ID NOs: 168-173, respectively;
r) SEQ ID NOs: 178-183, respectively;
s) SEQ ID NOs: 188-193, respectively;
t) SEQ ID NOs: 198-203, respectively;
u) SEQ ID NOs: 208-213, respectively; or
v) SEQ ID NOs: 218-223, respectively.

In some embodiments, the anti-TIM-3 antibody comprises a heavy chain variable domain and a light chain variable domain having the amino acid sequences of:
a) SEQ ID NOs: 7 and 4, respectively;
b) SEQ ID NOs: 3 and 4, respectively;
c) SEQ ID NOs: 16 and 17, respectively;
d) SEQ ID NOs: 26 and 27, respectively;
e) SEQ ID NOs: 36 and 37, respectively;
f) SEQ ID NOs: 46 and 47, respectively;
g) SEQ ID NOs: 56 and 57, respectively;
h) SEQ ID NOs: 66 and 67, respectively;
i) SEQ ID NOs: 76 and 77, respectively;
j) SEQ ID NOs: 86 and 87, respectively;
k) SEQ ID NOs: 96 and 97, respectively;
l) SEQ ID NOs: 106 and 107, respectively;
m) SEQ ID NOs: 116 and 117, respectively;
n) SEQ ID NOs: 126 and 127, respectively;
o) SEQ ID NOs: 136 and 137, respectively;
p) SEQ ID NOs: 146 and 147, respectively;
q) SEQ ID NOs: 156 and 157, respectively;
r) SEQ ID NOs: 166 and 167, respectively;
s) SEQ ID NOs: 176 and 177, respectively;
t) SEQ ID NOs: 186 and 187, respectively;
u) SEQ ID NOs: 196 and 197, respectively;
v) SEQ ID NOs: 206 and 207, respectively; or
w) SEQ ID NOs: 216 and 217, respectively.

In some embodiments, the anti-TIM-3 antibody comprises:
a) an HC comprising the amino acid sequence of SEQ ID NO: 7 and the amino acid sequence of SEQ ID NO: 374, 375, 376, or 377, and an LC comprising the amino acid sequence of SEQ ID NO: 4 and the amino acid sequence of SEQ ID NO: 378;
b) an HC comprising the amino acid sequence of SEQ ID NO: 3 and the amino acid sequence of SEQ ID NO: 374, 375, 376, or 377, and an LC comprising the amino acid sequence of SEQ ID NO: 4 and the amino acid sequence of SEQ ID NO: 378;
c) an HC comprising the amino acid sequence of SEQ ID NO: 16 and the amino acid sequence of SEQ ID NO: 374, 375, 376, or 377, and an LC comprising the amino acid sequence of SEQ ID NO: 17 and the amino acid sequence of SEQ ID NO: 378;
d) an HC comprising the amino acid sequence of SEQ ID NO: 26 and the amino acid sequence of SEQ ID NO: 374, 375, 376, or 377, and an LC comprising the amino acid sequence of SEQ ID NO: 27 and the amino acid sequence of SEQ ID NO: 378;
e) an HC comprising the amino acid sequence of SEQ ID NO: 36 and the amino acid sequence of SEQ ID NO: 374, 375, 376, or 377, and an LC comprising the amino acid sequence of SEQ ID NO: 37 and the amino acid sequence of SEQ ID NO: 378;
f) an HC comprising the amino acid sequence of SEQ ID NO: 46 and the amino acid sequence of SEQ ID NO: 374, 375, 376, or 377, and an LC comprising the amino acid sequence of SEQ ID NO: 47 and the amino acid sequence of SEQ ID NO: 378;
g) an HC comprising the amino acid sequence of SEQ ID NO: 56 and the amino acid sequence of SEQ ID NO: 374, 375, 376, or 377, and an LC comprising the amino acid sequence of SEQ ID NO: 57 and the amino acid sequence of SEQ ID NO: 378;
h) an HC comprising the amino acid sequence of SEQ ID NO: 66 and the amino acid sequence of SEQ ID NO: 374, 375, 376, or 377, and an LC comprising the amino acid sequence of SEQ ID NO: 67 and the amino acid sequence of SEQ ID NO: 378;
i) an HC comprising the amino acid sequence of SEQ ID NO: 76 and the amino acid sequence of SEQ ID NO: 374, 375, 376, or 377, and an LC comprising the amino acid sequence of SEQ ID NO: 77 and the amino acid sequence of SEQ ID NO: 378;
j) an HC comprising the amino acid sequence of SEQ ID NO: 86 and the amino acid sequence of SEQ ID NO: 374, 375, 376, or 377, and an LC comprising the amino acid sequence of SEQ ID NO: 87 and the amino acid sequence of SEQ ID NO: 378;
k) an HC comprising the amino acid sequence of SEQ ID NO: 96 and the amino acid sequence of SEQ ID NO: 374, 375, 376, or 377, and an LC comprising the amino acid sequence of SEQ ID NO: 97 and the amino acid sequence of SEQ ID NO: 378;
l) an HC comprising the amino acid sequence of SEQ ID NO: 106 and the amino acid sequence of SEQ ID NO: 374, 375, 376, or 377, and an LC comprising the amino acid sequence of SEQ ID NO: 107 and the amino acid sequence of SEQ ID NO: 378;
m) an HC comprising the amino acid sequence of SEQ ID NO: 116 and the amino acid sequence of SEQ ID NO: 374, 375, 376, or 377, and an LC comprising the amino acid sequence of SEQ ID NO: 117 and the amino acid sequence of SEQ ID NO: 378;
n) an HC comprising the amino acid sequence of SEQ ID NO: 126 and the amino acid sequence of SEQ ID NO: 374, 375, 376, or 377, and an LC comprising the amino acid sequence of SEQ ID NO: 127 and the amino acid sequence of SEQ ID NO: 378;
o) an HC comprising the amino acid sequence of SEQ ID NO: 136 and the amino acid sequence of SEQ ID NO: 374, 375, 376, or 377, and an LC comprising the amino acid sequence of SEQ ID NO: 137 and the amino acid sequence of SEQ ID NO: 378;
p) an HC comprising the amino acid sequence of SEQ ID NO: 146 and the amino acid sequence of SEQ ID NO: 374, 375, 376, or 377, and an LC comprising the amino acid sequence of SEQ ID NO: 147 and the amino acid sequence of SEQ ID NO: 378;
q) an HC comprising the amino acid sequence of SEQ ID NO: 156 and the amino acid sequence of SEQ ID NO: 374, 375, 376, or 377, and an LC comprising the amino acid sequence of SEQ ID NO: 157 and the amino acid sequence of SEQ ID NO: 378;
r) an HC comprising the amino acid sequence of SEQ ID NO: 166 and the amino acid sequence of SEQ ID NO: 374, 375, 376, or 377, and an LC comprising the amino acid sequence of SEQ ID NO: 167 and the amino acid sequence of SEQ ID NO: 378;
s) an HC comprising the amino acid sequence of SEQ ID NO: 176 and the amino acid sequence of SEQ ID NO: 374, 375, 376, or 377, and an LC comprising the amino acid sequence of SEQ ID NO: 177 and the amino acid sequence of SEQ ID NO: 378;
t) an HC comprising the amino acid sequence of SEQ ID NO: 186 and the amino acid sequence of SEQ ID NO: 374, 375, 376, or 377, and an LC comprising the amino acid sequence of SEQ ID NO: 187 and the amino acid sequence of SEQ ID NO: 378;
u) an HC comprising the amino acid sequence of SEQ ID NO: 196 and the amino acid sequence of SEQ ID NO: 374, 375, 376, or 377, and an LC comprising the amino acid sequence of SEQ ID NO: 197 and the amino acid sequence of SEQ ID NO: 378;
v) an HC comprising the amino acid sequence of SEQ ID NO: 206 and the amino acid sequence of SEQ ID NO: 374, 375, 376, or 377, and an LC comprising the amino acid sequence of SEQ ID NO: 207 and the amino acid sequence of SEQ ID NO: 378; or
w) an HC comprising the amino acid sequence of SEQ ID NO: 216 and the amino acid sequence of SEQ ID NO: 374, 375, 376, or 377, and an LC comprising the amino acid sequence of SEQ ID NO: 217 and the amino acid sequence of SEQ ID NO: 378.

In some embodiments, the anti-TIM-3 antibody is selected from the group consisting of:
a) an antibody whose H-CDR1-3 comprise the amino acid sequences of SEQ ID NOs: 8-10, respectively;
b) an antibody whose VH is at least 90% identical in sequence to the amino acid sequence of SEQ ID NO: 3 or 7;
c) an antibody whose VH comprises the amino acid sequence of SEQ ID NO: 3 or 7;
d) an antibody whose HC comprises the amino acid sequences of SEQ ID NOs: 3 and 374, or the amino acid sequence of SEQ ID NO: 7 and the amino acid sequence of SEQ ID NO: 375, 376, or 377;
e) an antibody whose L-CDR1-3 comprise the amino acid sequences of SEQ ID NOs: 11-13, respectively;
f) an antibody whose VL is at least 90% identical in sequence to the amino acid sequence of SEQ ID NO: 4;
g) an antibody whose VL comprises the amino acid sequence of SEQ ID NO: 4; h) an antibody whose LC comprises the amino acid sequences of SEQ ID NOs: 4 and 378;
i) an antibody whose H-CDR1-3 and L-CDR1-3 comprise the amino acid sequences of SEQ ID NOs: 8-13, respectively;
j) an antibody whose VH is at least 90% identical in sequence to the amino acid sequence of SEQ ID NO: 3 or 7 and whose VL is at least 90% identical in sequence to the amino acid sequence of SEQ ID NO: 4;
k) an antibody whose VH comprises the amino acid sequence of SEQ ID NO: 3 or 15 and whose VL comprises the amino acid sequence of SEQ ID NO: 4; and
l) an antibody whose HC comprises the amino acid sequences of SEQ ID NOs: 3 and 374, or the amino acid sequence of SEQ ID NO: 7 and the amino acid sequence of SEQ ID NO: 375, 376, or 377; and whose LC comprises the amino acid sequences of SEQ ID NOs: 4 and 378.

In some embodiments, the anti-TIM-3 antibody is selected from the group consisting of:
a) an antibody whose H-CDR1-3 comprise the amino acid sequences of SEQ ID NOs: 18-20, respectively;
b) an antibody whose VH is at least 90% identical in sequence to the amino acid sequence of SEQ ID NO: 16;
c) an antibody whose VH comprises the amino acid sequence of SEQ ID NO: 16;
d) an antibody whose HC comprises the amino acid sequences of SEQ ID NO: 16 and SEQ ID NO: 374, 375, 376, or 377;
e) an antibody whose L-CDR1-3 comprise the amino acid sequences of SEQ ID NOs: 21-23, respectively;
f) an antibody whose VL is at least 90% identical in sequence to the amino acid sequence of SEQ ID NO: 17;
g) an antibody whose VL comprises the amino acid sequence of SEQ ID NO: 17; h) an antibody whose LC comprises the amino acid sequences of SEQ ID NOs: 17 and 378;
i) an antibody whose H-CDR1-3 and L-CDR1-3 comprise the amino acid sequences of SEQ ID NOs: 18-23, respectively;
j) an antibody whose VH is at least 90% identical in sequence to the amino acid sequence of SEQ ID NO: 16 and whose VL is at least 90% identical in sequence to the amino acid sequence of SEQ ID NO: 17;
k) an antibody whose VH comprises the amino acid sequence of SEQ ID NO: 16 and whose VL comprises the amino acid sequence of SEQ ID NO: 17; and
l) an antibody whose HC comprises the amino acid sequence of SEQ ID NO: 16 and the amino acid sequence of SEQ ID NO: 374, 375, 376, or 377; and whose LC comprises the amino acid sequences of SEQ ID NOs: 17 and 378.

In some embodiments, the anti-TIM-3 antibody is selected from the group consisting of:
a) an antibody whose H-CDR1-3 comprise the amino acid sequences of SEQ ID NOs: 28-30, respectively;
b) an antibody whose VH is at least 90% identical in sequence to the amino acid sequence of SEQ ID NO: 26;
c) an antibody whose VH comprises the amino acid sequence of SEQ ID NO: 26;
d) an antibody whose HC comprises the amino acid sequences of SEQ ID NO: 26 and SEQ ID NO: 374, 375, 376, or 377;
e) an antibody whose L-CDR1-3 comprise the amino acid sequences of SEQ ID NOs: 31-33, respectively;
f) an antibody whose VL is at least 90% identical in sequence to the amino acid sequence of SEQ ID NO: 27;
g) an antibody whose VL comprises the amino acid sequence of SEQ ID NO: 27; h) an antibody whose LC comprises the amino acid sequences of SEQ ID NOs: 27 and 378;
i) an antibody whose H-CDR1-3 and L-CDR1-3 comprise the amino acid sequences of SEQ ID NOs: 28-33, respectively;
j) an antibody whose VH is at least 90% identical in sequence to the amino acid sequence of SEQ ID NO: 26 and whose VL is at least 90% identical in sequence to the amino acid sequence of SEQ ID NO: 27;
k) an antibody whose VH comprises the amino acid sequence of SEQ ID NO: 26 and whose VL comprises the amino acid sequence of SEQ ID NO: 27; and
l) an antibody whose HC comprises the amino acid sequence of SEQ ID NO: 26 and the amino acid sequence of SEQ ID NO: 374, 375, 376, or 377; and whose LC comprises the amino acid sequences of SEQ ID NOs: 27 and 378.

In some embodiments, the anti-TIM-3 antibody is selected from the group consisting of:
a) an antibody whose H-CDR1-3 comprise the amino acid sequences of SEQ ID NOs: 38-40, respectively;
b) an antibody whose VH is at least 90% identical in sequence to the amino acid sequence of SEQ ID NO: 36;
c) an antibody whose VH comprises the amino acid sequence of SEQ ID NO: 36;
d) an antibody whose HC comprises the amino acid sequences of SEQ ID NO: 36 and SEQ ID NO: 374, 375, 376, or 377;
e) an antibody whose L-CDR1-3 comprise the amino acid sequences of SEQ ID NOs: 41-43, respectively;
f) an antibody whose VL is at least 90% identical in sequence to the amino acid sequence of SEQ ID NO: 37;
g) an antibody whose VL comprises the amino acid sequence of SEQ ID NO: 37; h) an antibody whose LC comprises the amino acid sequences of SEQ ID NOs: 37 and 378;
i) an antibody whose H-CDR1-3 and L-CDR1-3 comprise the amino acid sequences of SEQ ID NOs: 38-43, respectively;
j) an antibody whose VH is at least 90% identical in sequence to the amino acid sequence of SEQ ID NO: 36 and whose VL is at least 90% identical in sequence to the amino acid sequence of SEQ ID NO: 37;
k) an antibody whose VH comprises the amino acid sequence of SEQ ID NO: 36 and whose VL comprises the amino acid sequence of SEQ ID NO: 37; and
l) an antibody whose HC comprises the amino acid sequence of SEQ ID NO: 36 and the amino acid sequence of SEQ ID NO: 374, 375, 376, or 377; and whose LC comprises the amino acid sequences of SEQ ID NOs: 37 and 378.

In some embodiments, the anti-TIM-3 antibody is selected from the group consisting of:
a) an antibody whose H-CDR1-3 comprise the amino acid sequences of SEQ ID NOs: 48-50, respectively;
b) an antibody whose VH is at least 90% identical in sequence to the amino acid sequence of SEQ ID NO: 46;
c) an antibody whose VH comprises the amino acid sequence of SEQ ID NO: 46;
d) an antibody whose HC comprises the amino acid sequences of SEQ ID NO: 46 and SEQ ID NO: 374, 375, 376, or 377;
e) an antibody whose L-CDR1-3 comprise the amino acid sequences of SEQ ID NOs: 51-53, respectively;
f) an antibody whose VL is at least 90% identical in sequence to the amino acid sequence of SEQ ID NO: 47;
g) an antibody whose VL comprises the amino acid sequence of SEQ ID NO: 47; h) an antibody whose LC comprises the amino acid sequences of SEQ ID NOs: 47 and 378;
i) an antibody whose H-CDR1-3 and L-CDR1-3 comprise the amino acid sequences of SEQ ID NOs: 48-53, respectively;
j) an antibody whose VH is at least 90% identical in sequence to the amino acid sequence of SEQ ID NO: 46 and whose VL is at least 90% identical in sequence to the amino acid sequence of SEQ ID NO: 47;
k) an antibody whose VH comprises the amino acid sequence of SEQ ID NO: 46 and whose VL comprises the amino acid sequence of SEQ ID NO: 47; and
l) an antibody whose HC comprises the amino acid sequence of SEQ ID NO: 46 and the amino acid sequence of SEQ ID NO: 374, 375, 376, or 377; and whose LC comprises the amino acid sequences of SEQ ID NOs: 47 and 378.

In some embodiments, the anti-TIM-3 antibody is selected from the group consisting of:
a) an antibody whose H-CDR1-3 comprise the amino acid sequences of SEQ ID NOs: 58-60, respectively;
b) an antibody whose VH is at least 90% identical in sequence to the amino acid sequence of SEQ ID NO: 56;
c) an antibody whose VH comprises the amino acid sequence of SEQ ID NO: 56;
d) an antibody whose HC comprises the amino acid sequences of SEQ ID NO: 56 and SEQ ID NO: 374, 375, 376, or 377;
e) an antibody whose L-CDR1-3 comprise the amino acid sequences of SEQ ID NOs: 61-63, respectively;
f) an antibody whose VL is at least 90% identical in sequence to the amino acid sequence of SEQ ID NO: 57;
g) an antibody whose VL comprises the amino acid sequence of SEQ ID NO: 57; h) an antibody whose LC comprises the amino acid sequences of SEQ ID NOs: 57 and 378;
i) an antibody whose H-CDR1-3 and L-CDR1-3 comprise the amino acid sequences of SEQ ID NOs: 58-63, respectively;
j) an antibody whose VH is at least 90% identical in sequence to the amino acid sequence of SEQ ID NO: 56 and whose VL is at least 90% identical in sequence to the amino acid sequence of SEQ ID NO: 57;
k) an antibody whose VH comprises the amino acid sequence of SEQ ID NO: 56 and whose VL comprises the amino acid sequence of SEQ ID NO: 57; and
l) an antibody whose HC comprises the amino acid sequence of SEQ ID NO: 56 and the amino acid sequence of SEQ ID NO: 374, 375, 376, or 377; and whose LC comprises the amino acid sequences of SEQ ID NOs: 57 and 378.

In some embodiments, the anti-TIM-3 antibody is selected from the group consisting of:
a) an antibody whose H-CDR1-3 comprise the amino acid sequences of SEQ ID NOs: 68-70, respectively;
b) an antibody whose VH is at least 90% identical in sequence to the amino acid sequence of SEQ ID NO: 66;
c) an antibody whose VH comprises the amino acid sequence of SEQ ID NO: 66;
d) an antibody whose HC comprises the amino acid sequences of SEQ ID NO: 66 and SEQ ID NO: 374, 375, 376, or 377;
e) an antibody whose L-CDR1-3 comprise the amino acid sequences of SEQ ID NOs: 71-73, respectively;
f) an antibody whose VL is at least 90% identical in sequence to the amino acid sequence of SEQ ID NO: 67;
g) an antibody whose VL comprises the amino acid sequence of SEQ ID NO: 67; h) an antibody whose LC comprises the amino acid sequences of SEQ ID NOs: 67 and 378;
i) an antibody whose H-CDR1-3 and L-CDR1-3 comprise the amino acid sequences of SEQ ID NOs: 68-73, respectively;
j) an antibody whose VH is at least 90% identical in sequence to the amino acid sequence of SEQ ID NO: 66 and whose VL is at least 90% identical in sequence to the amino acid sequence of SEQ ID NO: 67;
k) an antibody whose VH comprises the amino acid sequence of SEQ ID NO: 66 and whose VL comprises the amino acid sequence of SEQ ID NO: 67; and
l) an antibody whose HC comprises the amino acid sequence of SEQ ID NO: 66 and the amino acid sequence of SEQ ID NO: 374, 375, 376, or 377; and whose LC comprises the amino acid sequences of SEQ ID NOs: 67 and 378.

In some embodiments, the anti-TIM-3 antibody is selected from the group consisting of:
a) an antibody whose H-CDR1-3 comprise the amino acid sequences of SEQ ID NOs: 78-80, respectively;
b) an antibody whose VH is at least 90% identical in sequence to the amino acid sequence of SEQ ID NO: 76;
c) an antibody whose VH comprises the amino acid sequence of SEQ ID NO: 76;
d) an antibody whose HC comprises the amino acid sequences of SEQ ID NO: 76 and SEQ ID NO: 374, 375, 376, or 377;
e) an antibody whose L-CDR1-3 comprise the amino acid sequences of SEQ ID NOs: 81-83, respectively;
f) an antibody whose VL is at least 90% identical in sequence to the amino acid sequence of SEQ ID NO: 77;
g) an antibody whose VL comprises the amino acid sequence of SEQ ID NO: 77; h) an antibody whose LC comprises the amino acid sequences of SEQ ID NOs: 77 and 378;
i) an antibody whose H-CDR1-3 and L-CDR1-3 comprise the amino acid sequences of SEQ ID NOs: 78-83, respectively;
j) an antibody whose VH is at least 90% identical in sequence to the amino acid sequence of SEQ ID NO: 76 and whose VL is at least 90% identical in sequence to the amino acid sequence of SEQ ID NO: 77;
k) an antibody whose VH comprises the amino acid sequence of SEQ ID NO: 76 and whose VL comprises the amino acid sequence of SEQ ID NO: 77; and
l) an antibody whose HC comprises the amino acid sequence of SEQ ID NO: 76 and the amino acid sequence of SEQ ID NO: 374, 375, 376, or 377; and whose LC comprises the amino acid sequences of SEQ ID NOs: 77 and 378.

In some embodiments, the anti-TIM-3 antibody is selected from the group consisting of:
a) an antibody whose H-CDR1-3 comprise the amino acid sequences of SEQ ID NOs: 88-90, respectively;
b) an antibody whose VH is at least 90% identical in sequence to the amino acid sequence of SEQ ID NO: 86;
c) an antibody whose VH comprises the amino acid sequence of SEQ ID NO: 86;
d) an antibody whose HC comprises the amino acid sequences of SEQ ID NO: 86 and SEQ ID NO: 374, 375, 376, or 377;
e) an antibody whose L-CDR1-3 comprise the amino acid sequences of SEQ ID NOs: 91-93, respectively;
f) an antibody whose VL is at least 90% identical in sequence to the amino acid sequence of SEQ ID NO: 87;
g) an antibody whose VL comprises the amino acid sequence of SEQ ID NO: 87; h) an antibody whose LC comprises the amino acid sequences of SEQ ID NOs: 87 and 378;
i) an antibody whose H-CDR1-3 and L-CDR1-3 comprise the amino acid sequences of SEQ ID NOs: 88-93, respectively;
j) an antibody whose VH is at least 90% identical in sequence to the amino acid sequence of SEQ ID NO: 86 and whose VL is at least 90% identical in sequence to the amino acid sequence of SEQ ID NO: 87;
k) an antibody whose VH comprises the amino acid sequence of SEQ ID NO: 86 and whose VL comprises the amino acid sequence of SEQ ID NO: 87; and
l) an antibody whose HC comprises the amino acid sequence of SEQ ID NO: 86 and the amino acid sequence of SEQ ID NO: 374, 375, 376, or 377; and whose LC comprises the amino acid sequences of SEQ ID NOs: 87 and 378.

In some embodiments, the anti-TIM-3 antibody is selected from the group consisting of:
a) an antibody whose H-CDR1-3 comprise the amino acid sequences of SEQ ID NOs: 98-100, respectively;
b) an antibody whose VH is at least 90% identical in sequence to the amino acid sequence of SEQ ID NO: 96;
c) an antibody whose VH comprises the amino acid sequence of SEQ ID NO: 96;
d) an antibody whose HC comprises the amino acid sequences of SEQ ID NO: 96 and SEQ ID NO: 374, 375, 376, or 377;
e) an antibody whose L-CDR1-3 comprise the amino acid sequences of SEQ ID NOs: 101-103, respectively;
f) an antibody whose VL is at least 90% identical in sequence to the amino acid sequence of SEQ ID NO: 97;
g) an antibody whose VL comprises the amino acid sequence of SEQ ID NO: 97; h) an antibody whose LC comprises the amino acid sequences of SEQ ID NOs: 97 and 378;
i) an antibody whose H-CDR1-3 and L-CDR1-3 comprise the amino acid sequences of SEQ ID NOs: 98-103, respectively;
j) an antibody whose VH is at least 90% identical in sequence to the amino acid sequence of SEQ ID NO: 96 and whose VL is at least 90% identical in sequence to the amino acid sequence of SEQ ID NO: 97;
k) an antibody whose VH comprises the amino acid sequence of SEQ ID NO: 96 and whose VL comprises the amino acid sequence of SEQ ID NO: 97; and
l) an antibody whose HC comprises the amino acid sequence of SEQ ID NO: 96 and the amino acid sequence of SEQ ID NO: 374, 375, 376, or 377; and whose LC comprises the amino acid sequences of SEQ ID NOs: 97 and 378.

In some embodiments, the anti-TIM-3 antibody is selected from the group consisting of:
a) an antibody whose H-CDR1-3 comprise the amino acid sequences of SEQ ID NOs: 108-110, respectively;
b) an antibody whose VH is at least 90% identical in sequence to the amino acid sequence of SEQ ID NO: 106;
c) an antibody whose VH comprises the amino acid sequence of SEQ ID NO: 106;
d) an antibody whose HC comprises the amino acid sequences of SEQ ID NO: 106 and SEQ ID NO: 374, 375, 376, or 377;
e) an antibody whose L-CDR1-3 comprise the amino acid sequences of SEQ ID NOs: 111-113, respectively;
f) an antibody whose VL is at least 90% identical in sequence to the amino acid sequence of SEQ ID NO: 107;
g) an antibody whose VL comprises the amino acid sequence of SEQ ID NO: 107;
h) an antibody whose LC comprises the amino acid sequences of SEQ ID NOs: 107 and 378;
i) an antibody whose H-CDR1-3 and L-CDR1-3 comprise the amino acid sequences of SEQ ID NOs: 108-113, respectively;
j) an antibody whose VH is at least 90% identical in sequence to the amino acid sequence of SEQ ID NO: 106 and whose VL is at least 90% identical in sequence to the amino acid sequence of SEQ ID NO: 107;
k) an antibody whose VH comprises the amino acid sequence of SEQ ID NO: 106 and whose VL comprises the amino acid sequence of SEQ ID NO: 107; and
l) an antibody whose HC comprises the amino acid sequence of SEQ ID NO: 106 and the amino acid sequence of SEQ ID NO: 374, 375, 376, or 377; and whose LC comprises the amino acid sequences of SEQ ID NOs: 107 and 378.

In some embodiments, the anti-TIM-3 antibody is selected from the group consisting of:
a) an antibody whose H-CDR1-3 comprise the amino acid sequences of SEQ ID NOs: 118-120, respectively;
b) an antibody whose VH is at least 90% identical in sequence to the amino acid sequence of SEQ ID NO: 116;
c) an antibody whose VH comprises the amino acid sequence of SEQ ID NO: 116;
d) an antibody whose HC comprises the amino acid sequences of SEQ ID NO: 116 and SEQ ID NO: 374, 375, 376, or 377;
e) an antibody whose L-CDR1-3 comprise the amino acid sequences of SEQ ID NOs: 121-123, respectively;
f) an antibody whose VL is at least 90% identical in sequence to the amino acid sequence of SEQ ID NO: 117;
g) an antibody whose VL comprises the amino acid sequence of SEQ ID NO: 117;
h) an antibody whose LC comprises the amino acid sequences of SEQ ID NOs: 117 and 378;
i) an antibody whose H-CDR1-3 and L-CDR1-3 comprise the amino acid sequences of SEQ ID NOs: 118-123, respectively;
j) an antibody whose VH is at least 90% identical in sequence to the amino acid sequence of SEQ ID NO: 116 and whose VL is at least 90% identical in sequence to the amino acid sequence of SEQ ID NO: 117;
k) an antibody whose VH comprises the amino acid sequence of SEQ ID NO: 116 and whose VL comprises the amino acid sequence of SEQ ID NO: 117; and
l) an antibody whose HC comprises the amino acid sequence of SEQ ID NO: 116 and the amino acid sequence of SEQ ID NO: 374, 375, 376, or 377; and whose LC comprises the amino acid sequences of SEQ ID NOs: 117 and 378.

In some embodiments, the anti-TIM-3 antibody is selected from the group consisting of:
a) an antibody whose H-CDR1-3 comprise the amino acid sequences of SEQ ID NOs: 128-130, respectively;
b) an antibody whose VH is at least 90% identical in sequence to the amino acid sequence of SEQ ID NO: 126;
c) an antibody whose VH comprises the amino acid sequence of SEQ ID NO: 126;
d) an antibody whose HC comprises the amino acid sequences of SEQ ID NO: 126 and SEQ ID NO: 374, 375, 376, or 377;
e) an antibody whose L-CDR1-3 comprise the amino acid sequences of SEQ ID NOs: 131-133, respectively;
f) an antibody whose VL is at least 90% identical in sequence to the amino acid sequence of SEQ ID NO: 127;
g) an antibody whose VL comprises the amino acid sequence of SEQ ID NO: 127;
h) an antibody whose LC comprises the amino acid sequences of SEQ ID NOs: 127 and 378;
i) an antibody whose H-CDR1-3 and L-CDR1-3 comprise the amino acid sequences of SEQ ID NOs: 128-133, respectively;
j) an antibody whose VH is at least 90% identical in sequence to the amino acid sequence of SEQ ID NO: 126 and whose VL is at least 90% identical in sequence to the amino acid sequence of SEQ ID NO: 127;
k) an antibody whose VH comprises the amino acid sequence of SEQ ID NO: 126 and whose VL comprises the amino acid sequence of SEQ ID NO: 127; and
l) an antibody whose HC comprises the amino acid sequence of SEQ ID NO: 126 and the amino acid sequence of SEQ ID NO: 374, 375, 376, or 377; and whose LC comprises the amino acid sequences of SEQ ID NOs: 127 and 378.

In some embodiments, the anti-TIM-3 antibody is selected from the group consisting of:
a) an antibody whose H-CDR1-3 comprise the amino acid sequences of SEQ ID NOs: 138-140, respectively;
b) an antibody whose VH is at least 90% identical in sequence to the amino acid sequence of SEQ ID NO: 136;
c) an antibody whose VH comprises the amino acid sequence of SEQ ID NO: 136;
d) an antibody whose HC comprises the amino acid sequences of SEQ ID NO: 136 and SEQ ID NO: 374, 375, 376, or 377;
e) an antibody whose L-CDR1-3 comprise the amino acid sequences of SEQ ID NOs: 141-143, respectively;
f) an antibody whose VL is at least 90% identical in sequence to the amino acid sequence of SEQ ID NO: 137;
g) an antibody whose VL comprises the amino acid sequence of SEQ ID NO: 137;
h) an antibody whose LC comprises the amino acid sequences of SEQ ID NOs: 137 and 378;
i) an antibody whose H-CDR1-3 and L-CDR1-3 comprise the amino acid sequences of SEQ ID NOs: 138-143, respectively;
j) an antibody whose VH is at least 90% identical in sequence to the amino acid sequence of SEQ ID NO: 136 and whose VL is at least 90% identical in sequence to the amino acid sequence of SEQ ID NO: 137;
k) an antibody whose VH comprises the amino acid sequence of SEQ ID NO: 136 and whose VL comprises the amino acid sequence of SEQ ID NO: 137; and
l) an antibody whose HC comprises the amino acid sequence of SEQ ID NO: 136 and the amino acid sequence of SEQ ID NO: 374, 375, 376, or 377; and whose LC comprises the amino acid sequences of SEQ ID NOs: 137 and 378.

In some embodiments, the anti-TIM-3 antibody is selected from the group consisting of:
a) an antibody whose H-CDR1-3 comprise the amino acid sequences of SEQ ID NOs: 148-150, respectively;
b) an antibody whose VH is at least 90% identical in sequence to the amino acid sequence of SEQ ID NO: 146;
c) an antibody whose VH comprises the amino acid sequence of SEQ ID NO: 146;
d) an antibody whose HC comprises the amino acid sequences of SEQ ID NO: 146 and SEQ ID NO: 374, 375, 376, or 377;
e) an antibody whose L-CDR1-3 comprise the amino acid sequences of SEQ ID NOs: 151-153, respectively;
f) an antibody whose VL is at least 90% identical in sequence to the amino acid sequence of SEQ ID NO: 147;
g) an antibody whose VL comprises the amino acid sequence of SEQ ID NO: 147;
h) an antibody whose LC comprises the amino acid sequences of SEQ ID NOs: 147 and 378;
i) an antibody whose H-CDR1-3 and L-CDR1-3 comprise the amino acid sequences of SEQ ID NOs: 148-53, respectively;
j) an antibody whose VH is at least 90% identical in sequence to the amino acid sequence of SEQ ID NO: 146 and whose VL is at least 90% identical in sequence to the amino acid sequence of SEQ ID NO: 147;
k) an antibody whose VH comprises the amino acid sequence of SEQ ID NO: 146 and whose VL comprises the amino acid sequence of SEQ ID NO: 147; and
l) an antibody whose HC comprises the amino acid sequence of SEQ ID NO: 146 and the amino acid sequence of SEQ ID NO: 374, 375, 376, or 377; and whose LC comprises the amino acid sequences of SEQ ID NOs: 147 and 378.

In some embodiments, the anti-TIM-3 antibody is selected from the group consisting of:
a) an antibody whose H-CDR1-3 comprise the amino acid sequences of SEQ ID NOs: 158-160, respectively;
b) an antibody whose VH is at least 90% identical in sequence to the amino acid sequence of SEQ ID NO: 156;
c) an antibody whose VH comprises the amino acid sequence of SEQ ID NO: 156;
d) an antibody whose HC comprises the amino acid sequences of SEQ ID NO: 156 and SEQ ID NO: 374, 375, 376, or 377;
e) an antibody whose L-CDR1-3 comprise the amino acid sequences of SEQ ID NOs: 161-163, respectively;
f) an antibody whose VL is at least 90% identical in sequence to the amino acid sequence of SEQ ID NO: 157;
g) an antibody whose VL comprises the amino acid sequence of SEQ ID NO: 157;
h) an antibody whose LC comprises the amino acid sequences of SEQ ID NOs: 157 and 378;
i) an antibody whose H-CDR1-3 and L-CDR1-3 comprise the amino acid sequences of SEQ ID NOs: 158-163, respectively;
j) an antibody whose VH is at least 90% identical in sequence to the amino acid sequence of SEQ ID NO: 156 and whose VL is at least 90% identical in sequence to the amino acid sequence of SEQ ID NO: 157;
k) an antibody whose VH comprises the amino acid sequence of SEQ ID NO: 156 and whose VL comprises the amino acid sequence of SEQ ID NO: 157; and
l) an antibody whose HC comprises the amino acid sequence of SEQ ID NO: 156 and the amino acid sequence of SEQ ID NO: 374, 375, 376, or 377; and whose LC comprises the amino acid sequences of SEQ ID NOs: 157 and 378.

In some embodiments, the anti-TIM-3 antibody is selected from the group consisting of:
a) an antibody whose H-CDR1-3 comprise the amino acid sequences of SEQ ID NOs: 168-170, respectively;
b) an antibody whose VH is at least 90% identical in sequence to the amino acid sequence of SEQ ID NO: 166;
c) an antibody whose VH comprises the amino acid sequence of SEQ ID NO: 166;
d) an antibody whose HC comprises the amino acid sequences of SEQ ID NO: 166 and SEQ ID NO: 374, 375, 376, or 377;
e) an antibody whose L-CDR1-3 comprise the amino acid sequences of SEQ ID NOs: 171-173, respectively;
f) an antibody whose VL is at least 90% identical in sequence to the amino acid sequence of SEQ ID NO: 167;
g) an antibody whose VL comprises the amino acid sequence of SEQ ID NO: 167;
h) an antibody whose LC comprises the amino acid sequences of SEQ ID NOs: 167 and 378;
i) an antibody whose H-CDR1-3 and L-CDR1-3 comprise the amino acid sequences of SEQ ID NOs: 168-173, respectively;
j) an antibody whose VH is at least 90% identical in sequence to the amino acid sequence of SEQ ID NO: 166 and whose VL is at least 90% identical in sequence to the amino acid sequence of SEQ ID NO: 167;
k) an antibody whose VH comprises the amino acid sequence of SEQ ID NO: 166 and whose VL comprises the amino acid sequence of SEQ ID NO: 167; and
l) an antibody whose HC comprises the amino acid sequence of SEQ ID NO: 166 and the amino acid sequence of SEQ ID NO: 374, 375, 376, or 377; and whose LC comprises the amino acid sequences of SEQ ID NOs: 167 and 378.

In some embodiments, the anti-TIM-3 antibody is selected from the group consisting of:
a) an antibody whose H-CDR1-3 comprise the amino acid sequences of SEQ ID NOs: 178-80, respectively;
b) an antibody whose VH is at least 90% identical in sequence to the amino acid sequence of SEQ ID NO: 176;
c) an antibody whose VH comprises the amino acid sequence of SEQ ID NO: 176;
d) an antibody whose HC comprises the amino acid sequences of SEQ ID NO: 176 and SEQ ID NO: 374, 375, 376, or 377;
e) an antibody whose L-CDR1-3 comprise the amino acid sequences of SEQ ID NOs: 181-183, respectively;
f) an antibody whose VL is at least 90% identical in sequence to the amino acid sequence of SEQ ID NO: 177;
g) an antibody whose VL comprises the amino acid sequence of SEQ ID NO: 177;
h) an antibody whose LC comprises the amino acid sequences of SEQ ID NOs: 177 and 378;
i) an antibody whose H-CDR1-3 and L-CDR1-3 comprise the amino acid sequences of SEQ ID NOs: 178-183, respectively;
j) an antibody whose VH is at least 90% identical in sequence to the amino acid sequence of SEQ ID NO: 176 and whose VL is at least 90% identical in sequence to the amino acid sequence of SEQ ID NO: 177;
k) an antibody whose VH comprises the amino acid sequence of SEQ ID NO: 176 and whose VL comprises the amino acid sequence of SEQ ID NO: 177; and
l) an antibody whose HC comprises the amino acid sequence of SEQ ID NO: 176 and the amino acid sequence of SEQ ID NO: 374, 375, 376, or 377; and whose LC comprises the amino acid sequences of SEQ ID NOs: 177 and 378.

In some embodiments, the anti-TIM-3 antibody is selected from the group consisting of:
a) an antibody whose H-CDR1-3 comprise the amino acid sequences of SEQ ID NOs: 188-190, respectively;
b) an antibody whose VH is at least 90% identical in sequence to the amino acid sequence of SEQ ID NO: 186;
c) an antibody whose VH comprises the amino acid sequence of SEQ ID NO: 186;
d) an antibody whose HC comprises the amino acid sequences of SEQ ID NO: 186 and SEQ ID NO: 374, 375, 376, or 377;
e) an antibody whose L-CDR1-3 comprise the amino acid sequences of SEQ ID NOs: 191-193, respectively;
f) an antibody whose VL is at least 90% identical in sequence to the amino acid sequence of SEQ ID NO: 187;
g) an antibody whose VL comprises the amino acid sequence of SEQ ID NO: 187;
h) an antibody whose LC comprises the amino acid sequences of SEQ ID NOs: 187 and 378;
i) an antibody whose H-CDR1-3 and L-CDR1-3 comprise the amino acid sequences of SEQ ID NOs: 188-193, respectively;
j) an antibody whose VH is at least 90% identical in sequence to the amino acid sequence of SEQ ID NO: 186 and whose VL is at least 90% identical in sequence to the amino acid sequence of SEQ ID NO: 187;
k) an antibody whose VH comprises the amino acid sequence of SEQ ID NO: 186 and whose VL comprises the amino acid sequence of SEQ ID NO: 187; and
l) an antibody whose HC comprises the amino acid sequence of SEQ ID NO: 186 and the amino acid sequence of SEQ ID NO: 374, 375, 376, or 377; and whose LC comprises the amino acid sequences of SEQ ID NOs: 187 and 378.

In some embodiments, the anti-TIM-3 antibody is selected from the group consisting of:
a) an antibody whose H-CDR1-3 comprise the amino acid sequences of SEQ ID NOs: 198-200, respectively;
b) an antibody whose VH is at least 90% identical in sequence to the amino acid sequence of SEQ ID NO: 196;
c) an antibody whose VH comprises the amino acid sequence of SEQ ID NO: 196;
d) an antibody whose HC comprises the amino acid sequences of SEQ ID NO: 196 and SEQ ID NO: 374, 375, 376, or 377;
e) an antibody whose L-CDR1-3 comprise the amino acid sequences of SEQ ID NOs: 201-203, respectively;
f) an antibody whose VL is at least 90% identical in sequence to the amino acid sequence of SEQ ID NO: 197;
g) an antibody whose VL comprises the amino acid sequence of SEQ ID NO: 197;
h) an antibody whose LC comprises the amino acid sequences of SEQ ID NOs: 197 and 378;
i) an antibody whose H-CDR1-3 and L-CDR1-3 comprise the amino acid sequences of SEQ ID NOs: 198-203, respectively;
j) an antibody whose VH is at least 90% identical in sequence to the amino acid sequence of SEQ ID NO: 196 and whose VL is at least 90% identical in sequence to the amino acid sequence of SEQ ID NO: 197;
k) an antibody whose VH comprises the amino acid sequence of SEQ ID NO: 196 and whose VL comprises the amino acid sequence of SEQ ID NO: 197; and
l) an antibody whose HC comprises the amino acid sequence of SEQ ID NO: 196 and the amino acid sequence of SEQ ID NO: 374, 375, 376, or 377; and whose LC comprises the amino acid sequences of SEQ ID NOs: 197 and 378.

In some embodiments, the anti-TIM-3 antibody is selected from the group consisting of:
a) an antibody whose H-CDR1-3 comprise the amino acid sequences of SEQ ID NOs: 208-210, respectively;
b) an antibody whose VH is at least 90% identical in sequence to the amino acid sequence of SEQ ID NO: 206;
c) an antibody whose VH comprises the amino acid sequence of SEQ ID NO: 206;
d) an antibody whose HC comprises the amino acid sequences of SEQ ID NO: 206 and SEQ ID NO: 374, 375, 376, or 377;
e) an antibody whose L-CDR1-3 comprise the amino acid sequences of SEQ ID NOs: 211-213, respectively;
f) an antibody whose VL is at least 90% identical in sequence to the amino acid sequence of SEQ ID NO: 207;
g) an antibody whose VL comprises the amino acid sequence of SEQ ID NO: 207;
h) an antibody whose LC comprises the amino acid sequences of SEQ ID NOs: 207 and 378;
i) an antibody whose H-CDR1-3 and L-CDR1-3 comprise the amino acid sequences of SEQ ID NOs: 208-213, respectively;
j) an antibody whose VH is at least 90% identical in sequence to the amino acid sequence of SEQ ID NO: 206 and whose VL is at least 90% identical in sequence to the amino acid sequence of SEQ ID NO: 207;
k) an antibody whose VH comprises the amino acid sequence of SEQ ID NO: 206 and whose VL comprises the amino acid sequence of SEQ ID NO: 207; and
l) an antibody whose HC comprises the amino acid sequence of SEQ ID NO: 206 and the amino acid sequence of SEQ ID NO: 374, 375, 376, or 377; and whose LC comprises the amino acid sequences of SEQ ID NOs: 207 and 378.

In some embodiments, the anti-TIM-3 antibody is selected from the group consisting of:
a) an antibody whose H-CDR1-3 comprise the amino acid sequences of SEQ ID NOs: 218-220, respectively;
b) an antibody whose VH is at least 90% identical in sequence to the amino acid sequence of SEQ ID NO: 216;
c) an antibody whose VH comprises the amino acid sequence of SEQ ID NO: 216;
d) an antibody whose HC comprises the amino acid sequences of SEQ ID NO: 216 and SEQ ID NO: 374, 375, 376, or 377;
e) an antibody whose L-CDR1-3 comprise the amino acid sequences of SEQ ID NOs: 221-223, respectively;
f) an antibody whose VL is at least 90% identical in sequence to the amino acid sequence of SEQ ID NO: 217;
g) an antibody whose VL comprises the amino acid sequence of SEQ ID NO: 217;
h) an antibody whose LC comprises the amino acid sequences of SEQ ID NOs: 217 and 378;
i) an antibody whose H-CDR1-3 and L-CDR1-3 comprise the amino acid sequences of SEQ ID NOs: 218-223, respectively;
j) an antibody whose VH is at least 90% identical in sequence to the amino acid sequence of SEQ ID NO: 216 and whose VL is at least 90% identical in sequence to the amino acid sequence of SEQ ID NO: 217;
k) an antibody whose VH comprises the amino acid sequence of SEQ ID NO: 216 and whose VL comprises the amino acid sequence of SEQ ID NO: 217; and
1) an antibody whose HC comprises the amino acid sequence of SEQ ID NO: 216 and the amino acid sequence of SEQ ID NO: 374, 375, 376, or 377; and whose LC comprises the amino acid sequences of SEQ ID NOs: 217 and 378.

In some embodiments, any of the anti-TIM-3 antibodies or antigen-binding portions described herein may inhibit binding of ligands such as galectin-9, CEACAMI, HMGB-1, and phosphatidylserine to TIM-3.

In some embodiments, any of the anti-TIM-3 antibodies or antigen-binding portions described herein may increase the activity of NK cells. In some embodiments, this activity can mediate ADCC.

In some embodiments, administration of an anti-TIM-3 antibody or an antigen-binding portion thereof as described herein may activate dendritic cells, causing their maturation and thereby their ability to stimulate T-cells. While not wishing to be bound by any particular theory, it is believed that the anti-TIM-3 antibodies described herein function as TIM-3 dendritic cell activators, whereby their effect on dendritic cells serves to stimulate T cells. In a tumor-related setting, the anti-TIM-3 antibodies thus would cause maturation and activation of tumor associated dendritic cells, resulting in activation of tumor specific T-cells.

In some embodiments, administration of an anti-TIM-3 antibody or an antigen-binding portion thereof as described herein may directly activate T cells.

In some embodiments, the anti-TIM-3 antibody or antigen-binding portion described herein has at least one of the following properties:
a) binds to human TIM-3 with a K_{D} of 23 nM or less as measured by surface plasmon resonance;
b) binds to cynomolgus TIM-3 with a K_{D} of 22 nM or less as measured by surface plasmon resonance;
c) binds to human TIM-3 with an EC50 of 1.2 nM or less as measured by ELISA; d) binds to cynomolgus TIM-3 with an EC50 of 46 nM or less as measured by ELISA;
e) increases IFN-γ secretion in a one-way mixed lymphocyte reaction assay; f) increases IFN-γ secretion in a two-way mixed lymphocyte reaction assay; g) increases TNF-α secretion in a one-way mixed lymphocyte reaction assay; h) increases TNF-α secretion from dendritic cells; and
i) inhibits interaction of TIM-3 with phosphatidylserine.

Examples of such an antibody include, without limitation, antibody 15086.15086 (having at least properties a, c, d, e, g, and h); antibody 15086.17145 (having at least properties a, c, d, e, g, h, and i), antibody 15086.16837 or 15086.17144 (having at least properties a, c, and d), antibody 20293 or 20131 (having at least properties a, b, c, d, e, f, and h), antibody 20362 (having at least properties c, e, f, and h), and antibody 19324, 19416, 19568, 20185, 20300, or 20621 (having at least properties c, d, e, f, and h). In some embodiments, the anti-TIM-3 antibody or antigen-binding portion has all of said properties. In some embodiments, the anti-TIM-3 antibody or antigen-binding portion has at least properties a, c, d, e, g, and h. In some embodiments, the anti-TIM-3 antibody or antigen-binding portion has at least properties a, c, d, e, g, h, and i. In some embodiments, the anti-TIM-3 antibody or antigen-binding portion has at least properties a, c, and d. In some embodiments, the anti-TIM-3 antibody or antigen-binding portion has at least properties a, b, c, d, e, f, and h. In some embodiments, the anti-TIM-3 antibody or antigen-binding portion has at least properties c, e, f, and h. In some embodiments, the anti-TIM-3 antibody or antigen-binding portion has at least properties c, d, e, f, and h.

In some embodiments, an anti-TIM-3 antibody or an antigen-binding portion thereof as described herein binds to an epitope of TIM-3 that includes at least one (e.g., at least two, at least three, at least four, at least five, at least six, at least seven, at least eight, or at least nine) of the following residues of SEQ ID NO: 389: P50, V60, F61, E62, G64, R69, 1117, M118, and D120. In certain embodiments, the antibody or antigen-binding portion binds to an epitope of TIM-3 that includes residues P50, V60, F61, E62, G64, R69, 1117, M118, and D120 of SEQ ID NO: 389 (such as antibody 15086.15086, 15086.16837, 15086.17145, or 15086.17144). In certain embodiments, the antibody or antigen-binding portion binds to an epitope of TIM-3 that includes residues F61, R69, and 1117 of SEQ ID NO: 389 (such as antibody 20293). In certain embodiments, the antibody or antigen-binding portion binds to an epitope of TIM-3 that includes residues P50, F61, E62, 1117, M118, and D120 of SEQ ID NO: 389 (such as antibody 20131).

In some embodiments, the anti-TIM-3 antibody or antigen-binding portion thereof binds to an epitope of TIM-3 comprising amino acid residues F61 and 1117 of SEQ ID NO: 389 (e.g., antibody 15086.15086, 15086.16837, 15086.17145, 15086.17144, 20293, or 20131). In some embodiments, the epitope further comprises amino acid residue R69 (e.g., antibody 15086.15086, 15086.16837, 15086.17145, 15086.17144, or 20293). In some embodiments, the epitope further comprises P50, E62, M118, and D120 (e.g., antibody 15086.15086, 15086.16837, 15086.17145, 15086.17144, or 20131) and may additionally comprise amino acid residues V60 and G64 (e.g., antibody 15086.15086, 15086.16837, 15086.17145, or 15086.17144).

In some embodiments, an anti-TIM-3 antibody or an antigen-binding portion thereof as described herein binds to an epitope of TIM-3 that includes at least one (e.g., at least one, at least two, at least three, at least four, at least five, at least six, at least seven, at least eight, or at least nine) of the following residues of SEQ ID NO: 236: P50, V60, F61, E62, G64, R69, 1117, M118, and D120. An epitope with any combination of the above residues is contemplated.

In some embodiments, an anti-TIM-3 antibody or an antigen-binding portion thereof as described herein binds to an epitope of TIM-3 that comprises residues 62-67 and/or 114-117 of SEQ ID NO: 389. In some embodiments, the antibody or portion binds to residues 62-67 (or a fragment thereof, such as a one, two, three, four, or five residue fragment), of SEQ ID NO: 389 (e.g., antibodies 15086.15086, 15086.16837, 15086.17145, 15086.17144, and 20293). In some embodiments, the antibody or portion binds to residues 114-117 (or a fragment thereof, such as a one, two, or three residue fragment) of SEQ ID NO: 389 (e.g., antibody 20131). An epitope with any combination of the above residues is also contemplated.

In some embodiments, the anti-TIM-3 antibody or antigen-binding portion thereof does not compete for binding to TIM-3 with ABTIM3 (from PCT Patent Publication WO 2015/117002) and/or mAb15 (from PCT Patent Publication WO 2016/111947). In some embodiments, the anti-TIM-3 antibody or antigen-binding portion does not bind to the same epitope as ABTIM3 and/or mAB15; for example, the antibody or portion binds to one or more residues on TIM-3 that are not bound by ABTIM3 and/or mAb15.

In some embodiments, an anti-TIM-3 antibody or an antigen-binding portion thereof as described herein is an anti-TIM-3 antibody or antigen-binding portion described in PCT Patent Publication WO 2017/178493, which is incorporated by reference in its entirety herein.

### Anti-LAG-3 Antibodies

In some embodiments, the anti-LAG-3 antibodies disclosed herein are human antibodies generated from transgenic rats that are able to generate antibodies with human idiotypes. In another embodiment, the antibodies are chicken-derived chimeric antibodies comprising chicken CDR sequences and human framework regions, where the framework regions have been subjected to humanization.

One advantage of the novel anti-LAG-3 antibodies described herein is that they are able to enhance activity of T-cells as measured by increased IL-2 production. While not wishing to be bound by any particular theory, it is believed that the anti-LAG-3 antibodies are able to block the interaction of LAG-3 with its putative ligands such as MHCII and LSECtin. The antibodies may accomplish this directly via blocking of the ligand binding region or via induction of LAG-3 internalization. Another potential advantage of the anti-LAG-3 antibodies described herein is a low level of secondary effector functions in antibodies having the “LALA” mutations (L234A/L235A), which hinder significant antibody binding to human FcgR (Fc gamma receptors) and hence depletion of effector T-cells.

In some embodiments, the combination therapy or composition comprises an anti-LAG-3 antibody or an antigen-binding portion thereof, wherein the anti-LAG-3 antibody is the antibody referred to herein as antibody 15646, 15532, 15723, 15595, 15431, 15572 or 15011 or a variant of any of these, where the variant may, e.g., contain certain minimum amino acid changes relative to said antibody (e.g., 1, 2, 3, 4, 5, 6, 7, 8, 9, or 10 amino acid changes, which may be, e.g., in the framework regions) without losing the antigen-binding specificity of antibody.

In some embodiments, the anti-LAG-3 antibody, or an antigen-binding portion thereof, competes or cross-competes for binding to human LAG-3 with, or binds to the same epitope of human LAG-3 as, antibody 15646, 15532, 15723, 15595, 15431, 15572 or 15011. In some embodiments, the antibody has an IgG1 or IgG2 format. In certain embodiments, the antibody has an IgG2 format.

In some embodiments, the anti-LAG-3 antibody competes or cross-competes for binding to human LAG-3 with, or binds to the same epitope of human LAG-3 as, an antibody whose heavy chain (H) CDR1-3 and light chain (L) CDR1-3 comprise, respectively, SEQ ID NOs: 308-313, 318-323, 328-333, 338-343, 348-353, 358-363, or 368-373.

In some embodiments, the anti-LAG-3 antibody comprises an H-CDR3 comprising the H-CDR3 amino acid sequence of SEQ ID NO: 310, 320, 330, 340, 350, 360, or 370.

In some embodiments, the anti-LAG-3 antibody comprises H-CDR1-3 comprising the H-CDR1-3 amino acid sequences, respectively, of SEQ ID NOs: 308-310, 318-320, 328-330, 338-340, 348-350, 358-360, or 368-370.

In some embodiments, the anti-LAG-3 antibody has a VH that is at least 90% (e.g., at least 92%, at least 95%, at least 96%, at least 97%, at least 98%, or at least 99%) identical in amino acid sequence to SEQ ID NO: 306, 316, 326, 336, 346, 356, or 366.

In some embodiments, the anti-LAG-3 antibody has a VH that comprises SEQ ID NO: 306, 316, 326, 336, 346, 356, or 366.

In some embodiments, the anti-LAG-3 antibody has a VH that is at least 90% (e.g., at least 92%, at least 95%, at least 96%, at least 97%, at least 98%, or at least 99%) identical in sequence to SEQ ID NO: 306, 316, 326, 336, 346, 356, or 366; and a CH that is at least 90% (e.g., at least 92%, at least 95%, at least 96%, at least 97%, at least 98%, or at least 99%) identical in sequence to SEQ ID NO: 375.

In some embodiments, the anti-LAG-3 antibody has a HC that comprises the VH amino acid sequence of SEQ ID NO: 306, 316, 326, 336, 346, 356, or 366 and the CH amino acid sequence of SEQ ID NO: 375.

In some embodiments, the anti-LAG-3 antibody comprises an L-CDR3 comprising the L-CDR3 amino acid sequence of SEQ ID NO: 313, 323, 333, 343, 353, 363, or 373.

In some embodiments, the anti-LAG-3 antibody comprises L-CDR1-3 comprising the L-CDR1-3 amino acid sequences, respectively, of SEQ ID NOs: 311-313, 321-323, 331-333, 341-343, 351-353, 361-363, or 371-373.

In some embodiments, the anti-LAG-3 antibody has a VL that is at least 90% (e.g., at least 92%, at least 95%, at least 96%, at least 97%, at least 98%, or at least 99%) identical in sequence to the VL amino acid sequence of SEQ ID NO: 307, 317, 327, 337, 347, 357, or 367.

In some embodiments, the anti-LAG-3 antibody has a VL that comprises the VL amino acid sequence of SEQ ID NO: 307, 317, 327, 337, 347, 357, or 367.

In some embodiments, the anti-LAG-3 antibody has a VL that is at least 90% (e.g., at least 92%, at least 95%, at least 96%, at least 97%, at least 98%, or at least 99%) identical in sequence to the VL amino acid sequence of SEQ ID NO: 307, 317, 327, 337, 347, or 357; and a CL that is at least 90% (e.g., at least 92%, at least 95%, at least 96%, at least 97%, at least 98%, or at least 99%) identical in sequence to SEQ ID NO: 378.

In some embodiments, the anti-LAG-3 antibody has a LC that comprises the VL amino acid sequence of SEQ ID NO: 307, 317, 327, 337, 347, or 357 and the CL amino acid sequence of SEQ ID NO: 378. In some embodiments, the anti-LAG-3 antibody has an LC that comprises the VL amino acid sequence of SEQ ID NO: 367 and the CL amino acid sequence of SEQ ID NO: 379.

In some embodiments, the anti-LAG-3 antibody comprises any of the above heavy chain sequences and any of the above light chain sequences.

In some embodiments, the anti-LAG-3 antibody comprises an H-CDR3 and L-CDR3 comprising the H-CDR3 and L-CDR3 amino acid sequences, respectively, of SEQ ID NOs: 310 and 313, 320 and 323, 330 and 333, 340 and 343, 350 and 353, 360 and 363, or 370 and 373.

In some embodiments, the anti-LAG-3 antibody comprises H-CDR1-3 and L-CDR1-3 comprising the H-CDR1-3 and L-CDR1-3 sequences, respectively, of SEQ ID NOs: 308-313, 318-323, 328-333, 338-343, 348-353, 358-363, or 368-373.

In some embodiments, the anti-LAG-3 antibody has a VH that is at least 90% (e.g., at least 92%, at least 95%, at least 98%, or at least 99%) identical in sequence to the amino acid sequence of SEQ ID NO: 306, 316, 326, 336, 346, 356, or 366, and a VL that is at least 90% (e.g., at least 92%, at least 95%, at least 98%, or at least 99%) identical in sequence to the amino acid sequence of SEQ ID NO: 307, 317, 327, 337, 347, 357, or 367.

In some embodiments, the anti-LAG-3 antibody has a VH that comprises the amino acid sequence of SEQ ID NO: 306, 316, 326, 336, 346, 356, or 366, and a VL that comprises the amino acid sequence of SEQ ID NO: 307, 317, 327, 337, 347, 357, or 367.

In some embodiments, the anti-LAG-3 antibody has an HC that comprises the amino acid sequence of SEQ ID NO: 306, 316, 326, 336, 346, 356, or 366 and the amino acid sequence of SEQ ID NO: 375; and an LC that comprises the amino acid sequence of SEQ ID NO: 307, 317, 327, 337, 347, or 357 and the amino acid sequence of SEQ ID NO: 378.

In some embodiments, the anti-LAG-3 antibody has an HC that comprises the amino acid sequence of SEQ ID NO: 306, 316, 326, 336, 346, 356, or 366 and the amino acid sequence of SEQ ID NO: 375; and an LC that comprises the amino acid sequence of SEQ ID NO: 367 and the amino acid sequence of SEQ ID NO: 379.

In some embodiments, the anti-LAG-3 antibody comprises the H-CDR1-3 and L-CDR1-3 amino acid sequences of:
a) SEQ ID NOs: 308-313, respectively;
b) SEQ ID NOs: 318-323, respectively;
c) SEQ ID NOs: 328-333, respectively;
d) SEQ ID NOs: 338-343, respectively;
e) SEQ ID NOs: 348-353, respectively;
f) SEQ ID NOs: 358-363, respectively; or
g) SEQ ID NOs: 368-373, respectively.

In some embodiments, the anti-LAG-3 antibody comprises a heavy chain variable domain and a light chain variable domain having the amino acid sequences of:
a) SEQ ID NOs: 306 and 307, respectively;
b) SEQ ID NOs: 316 and 317, respectively;
c) SEQ ID NOs: 326 and 327, respectively;
d) SEQ ID NOs: 336 and 337, respectively;
e) SEQ ID NOs: 346 and 347, respectively;
f) SEQ ID NOs: 356 and 357, respectively; or
g) SEQ ID NOs: 366 and 367, respectively.

In some embodiments, the anti-LAG-3 antibody comprises:
a) an HC comprising the amino acid sequence of SEQ ID NO: 306 and the amino acid sequence of SEQ ID NO: 375, and an LC comprising the amino acid sequence of SEQ ID NO: 307 and the amino acid sequence of SEQ ID NO: 378;
b) an HC comprising the amino acid sequence of SEQ ID NO: 316 and the amino acid sequence of SEQ ID NO: 375, and an LC comprising the amino acid sequence of SEQ ID NO: 317 and the amino acid sequence of SEQ ID NO: 378;
c) an HC comprising the amino acid sequence of SEQ ID NO: 326 and the amino acid sequence of SEQ ID NO: 375, and an LC comprising the amino acid sequence of SEQ ID NO: 327 and the amino acid sequence of SEQ ID NO: 378;
d) an HC comprising the amino acid sequence of SEQ ID NO: 336 and the amino acid sequence of SEQ ID NO: 375, and an LC comprising the amino acid sequence of SEQ ID NO: 337 and the amino acid sequence of SEQ ID NO: 378;
e) an HC comprising the amino acid sequence of SEQ ID NO: 346 and the amino acid sequence of SEQ ID NO: 375, and an LC comprising the amino acid sequence of SEQ ID NO: 347 and the amino acid sequence of SEQ ID NO: 378;
f) an HC comprising the amino acid sequence of SEQ ID NO: 356 and the amino acid sequence of SEQ ID NO: 375, and an LC comprising the amino acid sequence of SEQ ID NO: 357 and the amino acid sequence of SEQ ID NO: 378; or
g) an HC comprising the amino acid sequence of SEQ ID NO: 366 and the amino acid sequence of SEQ ID NO: 375, and an LC comprising the amino acid sequence of SEQ ID NO: 367 and the amino acid sequence of SEQ ID NO: 379.

In some embodiments, the anti-LAG-3 antibody is selected from the group consisting of:
a) an antibody whose H-CDR1-3 comprise the amino acid sequences of SEQ ID NOs: 308-310, respectively;
b) an antibody whose VH is at least 90% identical in sequence to the amino acid sequence of SEQ ID NO: 306;
c) an antibody whose VH comprises the amino acid sequence of SEQ ID NO: 306;
d) an antibody whose HC comprises the amino acid sequences of SEQ ID NOs: 306 and 375;
e) an antibody whose L-CDR1-3 comprise the amino acid sequences of SEQ ID NOs: 311-313, respectively;
f) an antibody whose VL is at least 90% identical in sequence to the amino acid sequence of SEQ ID NO: 307;
g) an antibody whose VL comprises the amino acid sequence of SEQ ID NO: 307;
h) an antibody whose LC comprises the amino acid sequences of SEQ ID NOs: 307 and 378;
i) an antibody whose H-CDR1-3 and L-CDR1-3 comprise the amino acid sequences of SEQ ID NOs: 308-313, respectively;
j) an antibody whose VH is at least 90% identical in sequence to the amino acid sequence of SEQ ID NO: 306 and whose VL is at least 90% identical in sequence to the amino acid sequence of SEQ ID NO: 307;
k) an antibody whose VH comprises the amino acid sequence of SEQ ID NO: 306 and whose VL comprises the amino acid sequence of SEQ ID NO: 307; and
l) an antibody whose HC comprises the amino acid sequences of SEQ ID NOs: 306 and 375; and whose LC comprises the amino acid sequences of SEQ ID NOs: 307 and 378.

In some embodiments, the anti-LAG-3 antibody is selected from the group consisting of:
a) an antibody whose H-CDR1-3 comprise the amino acid sequences of SEQ ID NOs: 318-320, respectively;
b) an antibody whose VH is at least 90% identical in sequence to the amino acid sequence of SEQ ID NO: 316;
c) an antibody whose VH comprises the amino acid sequence of SEQ ID NO: 316;
d) an antibody whose HC comprises the amino acid sequences of SEQ ID NOs: 316 and 375;
e) an antibody whose L-CDR1-3 comprise the amino acid sequences of SEQ ID NOs: 321-323, respectively;
f) an antibody whose VL is at least 90% identical in sequence to the amino acid sequence of SEQ ID NO: 317;
g) an antibody whose VL comprises the amino acid sequence of SEQ ID NO: 317;
h) an antibody whose LC comprises the amino acid sequences of SEQ ID NOs: 317 and 378;
i) an antibody whose H-CDR1-3 and L-CDR1-3 comprise the amino acid sequences of SEQ ID NOs: 318-323, respectively;
j) an antibody whose VH is at least 90% identical in sequence to the amino acid sequence of SEQ ID NO: 316 and whose VL is at least 90% identical in sequence to the amino acid sequence of SEQ ID NO: 317;
k) an antibody whose VH comprises the amino acid sequence of SEQ ID NO: 316 and whose VL comprises the amino acid sequence of SEQ ID NO: 317; and
l) an antibody whose HC comprises the amino acid sequences of SEQ ID NOs: 316 and 375; and whose LC comprises the amino acid sequences of SEQ ID NOs: 317 and 378.

In some embodiments, the anti-LAG-3 antibody is selected from the group consisting of:
a) an antibody whose H-CDR1-3 comprise the amino acid sequences of SEQ ID NOs: 328-330, respectively;
b) an antibody whose VH is at least 90% identical in sequence to the amino acid sequence of SEQ ID NO: 326;
c) an antibody whose VH comprises the amino acid sequence of SEQ ID NO: 326;
d) an antibody whose HC comprises the amino acid sequences of SEQ ID NOs: 326 and 375;
e) an antibody whose L-CDR1-3 comprise the amino acid sequences of SEQ ID NOs: 331-333, respectively;
f) an antibody whose VL is at least 90% identical in sequence to the amino acid sequence of SEQ ID NO: 327;
g) an antibody whose VL comprises the amino acid sequence of SEQ ID NO: 327;
h) an antibody whose LC comprises the amino acid sequences of SEQ ID NOs: 327 and 378;
i) an antibody whose H-CDR1-3 and L-CDR1-3 comprise the amino acid sequences of SEQ ID NOs: 328-333, respectively;
j) an antibody whose VH is at least 90% identical in sequence to the amino acid sequence of SEQ ID NO: 326 and whose VL is at least 90% identical in sequence to the amino acid sequence of SEQ ID NO: 327;
k) an antibody whose VH comprises the amino acid sequence of SEQ ID NO: 326 and whose VL comprises the amino acid sequence of SEQ ID NO: 327; and
l) an antibody whose HC comprises the amino acid sequences of SEQ ID NOs: 326 and 375; and whose LC comprises the amino acid sequences of SEQ ID NOs: 327 and 378.

In some embodiments, the anti-LAG-3 antibody is selected from the group consisting of:
a) an antibody whose H-CDR1-3 comprise the amino acid sequences of SEQ ID NOs: 338-340, respectively;
b) an antibody whose VH is at least 90% identical in sequence to the amino acid sequence of SEQ ID NO: 336;
c) an antibody whose VH comprises the amino acid sequence of SEQ ID NO: 336;
d) an antibody whose HC comprises the amino acid sequences of SEQ ID NOs: 336 and 375;
e) an antibody whose L-CDR1-3 comprise the amino acid sequences of SEQ ID NOs: 341-343, respectively;
f) an antibody whose VL is at least 90% identical in sequence to the amino acid sequence of SEQ ID NO: 337;
g) an antibody whose VL comprises the amino acid sequence of SEQ ID NO: 337;
h) an antibody whose LC comprises the amino acid sequences of SEQ ID NOs: 337 and 378;
i) an antibody whose H-CDR1-3 and L-CDR1-3 comprise the amino acid sequences of SEQ ID NOs: 338-343, respectively;
j) an antibody whose VH is at least 90% identical in sequence to the amino acid sequence of SEQ ID NO: 336 and whose VL is at least 90% identical in sequence to the amino acid sequence of SEQ ID NO: 337;
k) an antibody whose VH comprises the amino acid sequence of SEQ ID NO: 336 and whose VL comprises the amino acid sequence of SEQ ID NO: 337; and
l) an antibody whose HC comprises the amino acid sequences of SEQ ID NOs: 336 and 375; and whose LC comprises the amino acid sequences of SEQ ID NOs: 337 and 378.

In some embodiments, the anti-LAG-3 antibody is selected from the group consisting of:
a) an antibody whose H-CDR1-3 comprise the amino acid sequences of SEQ ID NOs: 348-350, respectively;
b) an antibody whose VH is at least 90% identical in sequence to the amino acid sequence of SEQ ID NO: 346;
c) an antibody whose VH comprises the amino acid sequence of SEQ ID NO: 346;
d) an antibody whose HC comprises the amino acid sequences of SEQ ID NOs: 346 and 375;
e) an antibody whose L-CDR1-3 comprise the amino acid sequences of SEQ ID NOs: 351-353, respectively;
f) an antibody whose VL is at least 90% identical in sequence to the amino acid sequence of SEQ ID NO: 347;
g) an antibody whose VL comprises the amino acid sequence of SEQ ID NO: 347;
h) an antibody whose LC comprises the amino acid sequences of SEQ ID NOs: 347 and 378;
i) an antibody whose H-CDR1-3 and L-CDR1-3 comprise the amino acid sequences of SEQ ID NOs: 348-353, respectively;
j) an antibody whose VH is at least 90% identical in sequence to the amino acid sequence of SEQ ID NO: 346 and whose VL is at least 90% identical in sequence to the amino acid sequence of SEQ ID NO: 347;
k) an antibody whose VH comprises the amino acid sequence of SEQ ID NO: 346 and whose VL comprises the amino acid sequence of SEQ ID NO: 347; and
l) an antibody whose HC comprises the amino acid sequences of SEQ ID NOs: 346 and 375; and whose LC comprises the amino acid sequences of SEQ ID NOs: 347 and 378.

In some embodiments, the anti-LAG-3 antibody is selected from the group consisting of:
a) an antibody whose H-CDR1-3 comprise the amino acid sequences of SEQ ID NOs: 358-360, respectively;
b) an antibody whose VH is at least 90% identical in sequence to the amino acid sequence of SEQ ID NO: 356;
c) an antibody whose VH comprises the amino acid sequence of SEQ ID NO: 356;
d) an antibody whose HC comprises the amino acid sequences of SEQ ID NOs: 356 and 375;
e) an antibody whose L-CDR1-3 comprise the amino acid sequences of SEQ ID NOs: 361-363, respectively;
f) an antibody whose VL is at least 90% identical in sequence to the amino acid sequence of SEQ ID NO: 357;
g) an antibody whose VL comprises the amino acid sequence of SEQ ID NO: 357;
h) an antibody whose LC comprises the amino acid sequences of SEQ ID NOs: 357 and 378;
i) an antibody whose H-CDR1-3 and L-CDR1-3 comprise the amino acid sequences of SEQ ID NOs: 358-363, respectively;
j) an antibody whose VH is at least 90% identical in sequence to the amino acid sequence of SEQ ID NO: 356 and whose VL is at least 90% identical in sequence to the amino acid sequence of SEQ ID NO: 357;
k) an antibody whose VH comprises the amino acid sequence of SEQ ID NO: 356 and whose VL comprises the amino acid sequence of SEQ ID NO: 357; and
l) an antibody whose HC comprises the amino acid sequences of SEQ ID NOs: 356 and 375; and whose LC comprises the amino acid sequences of SEQ ID NOs: 357 and 378.

In some embodiments, the anti-LAG-3 antibody is selected from the group consisting of:
a) an antibody whose H-CDR1-3 comprise the amino acid sequences of SEQ ID NOs: 368-370, respectively;
b) an antibody whose VH is at least 90% identical in sequence to the amino acid sequence of SEQ ID NO: 366;
c) an antibody whose VH comprises the amino acid sequence of SEQ ID NO: 366;
d) an antibody whose HC comprises the amino acid sequences of SEQ ID NOs: 366 and 375;
e) an antibody whose L-CDR1-3 comprise the amino acid sequences of SEQ ID NOs: 371-373, respectively;
f) an antibody whose VL is at least 90% identical in sequence to the amino acid sequence of SEQ ID NO: 367;
g) an antibody whose VL comprises the amino acid sequence of SEQ ID NO: 367;
h) an antibody whose LC comprises the amino acid sequences of SEQ ID NOs: 367 and 379;
i) an antibody whose H-CDR1-3 and L-CDR1-3 comprise the amino acid sequences of SEQ ID NOs: 368-373, respectively;
j) an antibody whose VH is at least 90% identical in sequence to the amino acid sequence of SEQ ID NO: 366 and whose VL is at least 90% identical in sequence to the amino acid sequence of SEQ ID NO: 367;
k) an antibody whose VH comprises the amino acid sequence of SEQ ID NO: 366 and whose VL comprises the amino acid sequence of SEQ ID NO: 367; and
l) an antibody whose HC comprises the amino acid sequences of SEQ ID NOs: 366 and 375; and whose LC comprises the amino acid sequences of SEQ ID NOs: 367 and 379.

In some embodiments, any of the anti-LAG-3 antibodies or antigen-binding portions described herein may bind to human LAG-3 with an EC50 of, for example, 0.2 nM or less, 0.15 nM or less, 0.1 nM or less, 0.09 nM or less, 0.08 nM or less, 0.07 nM or less, 0.06 nM or less, 0.05 nM or less, or 0.04 nM or less. In some embodiments, any of the anti-LAG-3 antibodies or antigen-binding portions described herein may bind to cynomolgus LAG-3 with, for example, an EC50 of 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, or 0.03 nM or less. In particular embodiments, any of the anti-LAG-3 antibodies or antigen-binding portions described herein may bind to human LAG-3 with, for example, an EC50 of 0.1 nM or less and cynomolgus LAG-3 with, for example, an EC50 of 0.3 nM or less.

In some embodiments, any of the anti-LAG-3 antibodies or antigen-binding portions described herein may bind to human LAG-3 with an EC50 of, for example, 0.1 nM or less. In some embodiments, any of the anti-LAG-3 antibodies or antigen-binding portions described herein may bind to cynomolgus LAG-3 with, for example, an EC50 of 0.3 nM or less. In particular embodiments, any of the anti-LAG-3 antibodies or antigen-binding portions described herein may bind to human LAG-3 with, for example, an EC50 of 0.1 nM or less and cynomolgus LAG-3 with, for example, an EC50 of 0.3 nM or less.

In some embodiments, any of the anti-LAG-3 antibodies or antigen-binding portions described herein may inhibit binding of ligands such as MHC class II (MHCII) or LSECtin to LAG-3. For example, at 20 μg/mL, the anti-LAG-3 antibody or antigen-binding portion may reduce the binding of LAG-3 to MHCII by at least 35%, at least 40%, at least 45%, at least 50%, at least 55%, at least 60%, at least 65%, at least 70%, at least 75%, at least 80%, at least 85%, at least 89%, at least 90%, at least 91%, at least 92%, at least 93%, at least 94%, at least 95%, at least 96%, at least 97%, at least 98%, at least 99%, or 100% compared to binding in the presence of a negative control antibody. In one embodiment, the anti-LAG-3 antibody or antigen-binding protein may reduce the binding of LAG-3 to MHCII by greater than 85% compared to the negative control. In one embodiment, the anti-LAG-3 antibody or antigen-binding protein may reduce the binding of LAG-3 to MHCII by between about 25% and 95%, 30% and 90%, or 35% and 85%, compared to the negative control.

In some embodiments, any of the anti-LAG-3 antibodies or antigen-binding portions described herein may block binding between LAG-3 and MHC class II, e.g., human LAG-3 expressed on Jurkat cells and human MHC class II expressed on Raji cells (for example, at a concentration of 0.1 μg/mL, 0.5 μg/mL, 1 μg/mL, 5 μg/mL, 10 μg/mL, 20 μg/mL, 30 μg/mL, 40 μg/mL, or 50 μg/mL).

In some embodiments, any of the anti-LAG-3 antibodies or antigen-binding portions described herein may bind to human LAG-3 with a K_{D} of 5.0×10⁻⁸ or less, 4.0×10⁻⁸ or less, 3.0×10⁻⁸ or less, 2.0×10⁻⁸ or less, 1.0×10⁻⁸ or less, 9.0×10⁻⁹ or less, 8.0×10⁻⁹ or less, 7.0×10⁻⁹ or less, 6.0×10⁻⁹ or less, 5.0×10⁻⁹ or less, 4.0×10⁻⁹ or less, 3.0×10⁻⁹ or less, 2.0×10⁻⁹ or less, or 1.0×10⁻⁹ or less, as measured by surface plasmon resonance.

In some embodiments, any of the anti-LAG-3 antibodies or antigen-binding portions described herein may bind to cynomolgus LAG-3 with a K_{D} of 1.5×10⁻⁷ or less, 1.0×10⁻⁷ or less, 9.0×10⁻⁸ or less, 8.0×10⁻⁸ or less, 7.0×10⁻⁸ or less, 6.0×10⁻⁸ or less, 5.0×10⁻⁸ or less, 4.0×10⁻⁸ or less, 3.0×10⁻⁸ or less, 2.0×10⁻⁸ or less, or 1.0×10⁻⁸ or less, as measured by surface plasmon resonance.

In some embodiments, any of the anti-LAG-3 antibodies or antigen-binding portions described herein may bind to mouse LAG-3 with a K_{D} of 5.0×10⁻⁸ or less, 4.5×10⁻⁸ or less, 4.0×10⁻⁸ or less, 3.5×10⁻⁸ or less, or 3.0×10⁻⁸ or less, as measured by surface plasmon resonance.

In some embodiments, any of the anti-LAG-3 antibodies or antigen-binding portions described herein may stimulate IL-2 production, e.g., from SEB-stimulated PBMCs.

In some embodiments, any of the anti-LAG-3 antibodies or antigen-binding portions described herein may reduce cellular and/or soluble levels of LAG-3, e.g., in a human T cell line (such as a human T cell line overexpressing LAG-3).

In some embodiments, any of the anti-LAG-3 antibodies or antigen-binding portions described herein may induce tumor growth regression and/or delay tumor growth in vivo.

In some embodiments, any of the anti-LAG-3 antibodies or antigen-binding portions described herein may bind to a different epitope of human LAG-3 than antibody 25F7-Lag3.5.

In some embodiments, any of the anti-LAG-3 antibodies or antigen-binding portions described herein may activate T-cells, causing enhanced anti-tumor activity.

In some embodiments, the anti-LAG-3 antibody or antigen-binding portion described herein has at least one of the following properties:
a) at a concentration of 20 μg/mL, reduces the binding of human LAG-3 to human MHC class II on A375 cells by greater than 85% compared to a negative control antibody as determined by a flow cytometric competition assay;
b) at a concentration of 20 μg/mL, reduces the binding of human LAG-3 to human MHC class II on A375 cells to between 35% and 85% compared to a negative control antibody as determined by a flow cytometric competition assay;
c) blocks binding between human LAG-3 expressed on Jurkat cells and human MHC class II expressed on Raji cells;
d) binds to human LAG-3 with an EC50 of 0.1 nM or less as measured by flow cytometry;
e) binds to cynomolgus LAG-3 with an EC50 of 0.3 nM or less as measured by flow cytometry;
f) binds to human LAG-3 with a K_{D} of 3.0×10⁻⁸ or less as measured by surface plasmon resonance;
g) binds to cynomolgus LAG-3 with a K_{D} of 1.5×10⁻⁷ or less as measured by surface plasmon resonance;
h) binds to mouse LAG-3 with a K_{D} of 3.5×10⁻⁸ or less as measured by surface plasmon resonance;
i) stimulates IL-2 production in Staphylococcalenterotoxin B (SEB) treated human peripheral blood mononuclear cells (PBMCs);
j) reduces cellular levels of LAG-3 in human T cells;
k) reduces soluble levels of LAG-3 in the culture of human T cells;
l) induces tumor growth regression in vivo;
m) delays tumor growth in vivo; and
n) does not bind to the same epitope of human LAG-3 as antibody 25F7-Lag3.5.
   Examples of such an antibody include, without limitation, antibody 15646 (having at least properties b, c, d, e, i, and n), antibody 15532 (having at least properties a, c, d, e, f, g, i, j, k, m, and n), antibody 15723 (having at least properties b, c, d, e, i, and n), antibody 15595 (having at least properties a, c, d, e, i, and n), antibody 15431 (having at least properties a, c, d, e, f, g, i, and n), antibody 15572 (having at least properties b, c, d, e, f, g, i, and n), and antibody 15011 (having at least properties a, c, d, e, f, g, h, i, j, k, l, m, and n). In some embodiments, the anti-TIM-3 antibody or antigen-binding portion of the invention has at least 13, 12, 11, 10, 9, 8, 7, 6, 5, 4, 3, 2, or 1 of said properties. In some embodiments, the anti-TIM-3 antibody or antigen-binding portion of the invention has at least properties b, c, d, e, i, and n; at least properties a, c, d, e, f, g, i, j, k, m, and n; at least properties a, c, d, e, i, and n; at least properties a, c, d, e, f, g, i, and n; at least properties b, c, d, e, f, g, i, and n; or at least properties a, c, d, e, f, g, h, i, j, k, l, m, and n.

In some embodiments, the anti-LAG-3 antibody or antigen-binding portion of the invention competes for binding to human LAG-3 with antibody 15011, 15572, and/or 15431.

In some embodiments, the anti-LAG-3 antibody or antigen-binding portion of the invention binds to an epitope of human LAG-3 having:
a) amino acid residues H85, P86, A87, P89, S91, W92, and G93 of SEQ ID NO: 68;
b) amino acid residues A40, Q41, P43, P46, P49, D52, T62, Q64, H65, Q66, P67, D68, G93, P94, P96, R98, Y99, T100, V101, P106, G107, R119, E124, R129, G130, D131, S133, R137, P138, D143, R148, and R163 of SEQ ID NO: 68;
c) amino acid residues A40, Q41, P43, P46, P49, D52, T62, Q64, H65, Q66, P67, D68, P96, Y99, T100, V101, P106, G107, R119, E124, R129, G130, D131, S133, R137, P138, D143, R148, and R163 of SEQ ID NO: 68; or
d) amino acid residues G107, L109, R110, and S111 of SEQ ID NO: 68.

In some embodiments, the anti-LAG-3 antibody or antigen-binding portion of the invention binds to an epitope having amino acid residues 98-105 of SEQ ID NO: 68. Examples of such an antibody include, without limitation, antibodies 15532, 15431, 15572, and 15011.

In some embodiments, the anti-LAG-3 antibody or antigen-binding portion of the invention binds to an epitope having:
a) amino acid residues 78-105 and 123-131 of SEQ ID NO: 68;
b) amino acid residues 23-30, 40-66, 88-105, 123-137, and 148-152 of SEQ ID NO: 68; or
c) amino acid residues 23-30, 40-66, 98-105, 118-137, and 148-161 of SEQ ID NO: 68

In some embodiments, an anti-LAG-3 antibody or an antigen-binding portion thereof as described herein is an anti-LAG-3 antibody or antigen-binding portion described in PCT Patent Application PCT/EP2017/076188, which is incorporated by reference in its entirety herein.

The class of an antibody described herein may be changed or switched with another class or subclass. In one aspect, a nucleic acid molecule encoding VL or VH is isolated using methods well-known in the art such that it does not include nucleic acid sequences encoding CL or CH. The nucleic acid molecules encoding VL or VH then are operatively linked to a nucleic acid sequence encoding a CL or CH, respectively, from a different class of immunoglobulin molecule. This may be achieved using a vector or nucleic acid molecule that comprises a CL or CH chain, as described above. For example, an antibody that was originally IgM may be class switched to IgG. Further, the class switching may be used to convert one IgG subclass to another, e.g., from IgG1 to IgG2. A κ light chain constant region can be changed, e.g., to a λ light chain constant region. A preferred method for producing an antibody as described herein with a desired Ig isotype comprises the steps of isolating a nucleic acid molecule encoding the heavy chain of an antibody and a nucleic acid molecule encoding the light chain of an antibody, obtaining the variable domain of the heavy chain, ligating the variable domain of the heavy chain with the constant region of a heavy chain of the desired isotype, expressing the light chain and the ligated heavy chain in a cell, and collecting the antibody with the desired isotype.

An antibody described herein can be an IgG, an IgM, an IgE, an IgA, or an IgD molecule, but is typically of the IgG isotype, e.g. of IgG subclass IgG1, IgG2a or IgG2b, IgG3 or IgG4. In one embodiment, the antibody is an IgG1. In another embodiment, the antibody is an IgG2.

In one embodiment, the antibody may comprise at least one mutation in the Fc region. A number of different Fc mutations are known, where these mutations provide altered effector function. For example, in many cases it will be desirable to reduce or eliminate effector function, e.g., where ligand/receptor interactions are undesired or in the case of antibody-drug conjugates.

In one embodiment, the antibody comprises at least one mutation in the Fc region that reduces effector function. Fc region amino acid positions that may be advantageous to mutate in order to reduce effector function include one or more of positions 228, 233, 234 and 235, where amino acid positions are numbered according to the IMGT® numbering scheme.

In some embodiments, one or both of the amino acid residues at positions 234 and 235 may be mutated, for example, from Leu to Ala (L234A/L235A). These mutations reduce effector function of the Fc region of IgG1 antibodies. Additionally or alternatively, the amino acid residue at position 228 may be mutated, for example to Pro. In some embodiments, the amino acid residue at position 233 may be mutated, e.g., to Pro, the amino acid residue at position 234 may be mutated, e.g., to Val, and/or the amino acid residue at position 235 may be mutated, e.g., to Ala. The amino acid positions are numbered according to the IMGT® numbering scheme.

In some embodiments, where the antibody is of the IgG4 subclass, it may comprise the mutation S228P, i.e., having a proline in position 228, where the amino acid position is numbered according to the Eu IMGT® numbering scheme. This mutation is known to reduce undesired Fab arm exchange (Angal et al., Mol Immunol. 30:105-8 (1993)).

In certain embodiments, an antibody or antigen-binding portion thereof as described herein may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov et al., Human Antibodies and Hybridomas 6:93-101 (1995)) and use of a cysteine residue, a marker peptide and a C-terminal polyhistidine tag to make bivalent and biotinylated scFv molecules (Kipriyanov et al., Mol. Immunol. 31:1047-1058 (1994)). Other examples include where one or more CDRs from an antibody are incorporated into a molecule either covalently or noncovalently to make it an immunoadhesin that specifically binds to an antigen of interest. In such embodiments, the CDR(s) may be incorporated as part of a larger polypeptide chain, may be covalently linked to another polypeptide chain, or may be incorporated noncovalently.

In another embodiment, a fusion antibody or immunoadhesin may be made that comprises all or a portion of an antibody described herein linked to another polypeptide. In certain embodiments, only the variable domains of the antibody are linked to the polypeptide. In certain embodiments, the VH domain of an antibody is linked to a first polypeptide, while the VL domain of an antibody is linked to a second polypeptide that associates with the first polypeptide in a manner such that the VH and VL domains can interact with one another to form an antigen-binding site. In another preferred embodiment, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bi-specific antibody.

To create a single chain antibody (scFv), the VH- and VL-encoding DNA fragments are operatively linked to another fragment encoding a flexible linker, e.g., encoding the amino acid sequence (Gly4-Ser)3 (SEQ ID NO: 396), such that the VH and VL sequences can be expressed as a contiguous single-chain protein, with the VL and VH domains joined by the flexible linker. See, e.g., Bird et al., Science 242:423-426 (1988); Huston et al., Proc. Natl. Acad. Sci. USA 85:5879-5883 (1988); and McCafferty et al., Nature 348:552-554 (1990). The single chain antibody may be monovalent, if only a single VH and VL are used; bivalent, if two VH and VL are used; or polyvalent, if more than two VH and VL are used. Bi-specific or polyvalent antibodies may be generated that bind specifically to human PD-1, TIM-3, or LAG-3 and to another molecule, for instance. In some embodiments, the bi-specific or polyvalent antibodies may bind to PD-1 and TIM-3, PD-1 and LAG-3, TIM-3 and LAG-3, or PD-1, TIM-3, and LAG-3.

In other embodiments, other modified antibodies may be prepared using antibody-encoding nucleic acid molecules. For instance, “kappa bodies” (III et al., Protein Eng. 10:949-57 (1997)), “minibodies” (Martin et al., EMBO J. 13:5303-9 (1994)), “diabodies” (Holliger et al., Proc. Natl. Acad. Sci. USA 90:6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

An antibody or antigen-binding portion as described herein can be derivatized or linked to another molecule (e.g., another peptide or protein). In general, the antibodies or portions thereof are derivatized such that antigen binding is not affected adversely by the derivatization or labeling. Accordingly, the antibodies and antibody portions that may be used in the combination therapies and compositions of the invention are intended to include both intact and modified forms of the antibodies described herein. For example, an antibody or antibody portion as described herein can be functionally linked (by chemical coupling, genetic fusion, noncovalent association or otherwise) to one or more other molecular entities, such as another antibody (e.g., a bi-specific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bi-specific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available, e.g., from Pierce Chemical Company, Rockford, II.

An antibody can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody as described herein may also be labeled. As used herein, the terms “label” or “labeled” refer to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies described herein may be present in a neutral form (including zwitter ionic forms) or as a positively or negatively-charged species. In some embodiments, the antibodies may be complexed with a counterion to form a pharmaceutically acceptable salt.

The term “pharmaceutically acceptable salt” refers to a complex comprising one or more antibodies and one or more counterions, wherein the counterions are derived from pharmaceutically acceptable inorganic and organic acids and bases.

### Combination Therapies

The present invention provides a combination therapy (e.g., a composition) that comprises any (e.g., any one) of the anti-PD-1 antibodies or antigen-binding portions thereof described herein and any (e.g., any one) of the anti-TIM-3 antibodies or antigen-binding portions thereof described herein. In some embodiments, the combination therapy comprises any (e.g., any one) of the anti-PD-1 antibodies or antigen-binding portions thereof described herein and any (e.g., any one) of the anti-LAG-3 antibodies or antigen-binding portions thereof described herein. In some embodiments, the combination therapy comprises any (e.g., any one) of the anti-TIM-3 antibodies or antigen-binding portions thereof described herein and any (e.g., any one) of the anti-LAG-3 antibodies or antigen-binding portions thereof described herein. In particular embodiments, the combination therapy comprises any (e.g., any one) of the anti-PD-1 antibodies or antigen-binding portions thereof described herein, any (e.g., any one) of the anti-TIM-3 antibodies or antigen-binding portions thereof described herein, and any (e.g., any one) of the anti-LAG-3 antibodies or antigen-binding portions thereof described herein. The combination therapy may take the form of, e.g., a method for treatment using said antibodies or antigen-binding portions or a pharmaceutical composition comprising said antibodies or antigen-binding portions.

In certain embodiments, the combination therapy or composition of the invention comprises anti-PD-1 antibody 12819 and anti-TIM-3 antibody 15086.17145. In certain embodiments, the combination therapy or composition of the invention comprises anti-PD-1 antibody 12819 and anti-LAG-3 antibody 15532. In certain embodiments, the combination therapy or composition of the invention comprises anti-PD-1 antibody 12819, anti-TIM-3 antibody 15086.17145, and anti-LAG-3 antibody 15532.

In certain embodiments, the combination therapy or composition of the invention comprises:
an anti-PD-1 antibody or an antigen-binding portion thereof comprising the H-CDR1-3 and L-CDR1-3 amino acid sequences of SEQ ID NOs: 228-233, respectively; and an anti-TIM-3 antibody or an antigen-binding portion thereof comprising the H-CDR1-3 and L-CDR1-3 amino acid sequences of SEQ ID NOs: 8-13, respectively;
an anti-PD-1 antibody or an antigen-binding portion thereof comprising the VH and VL amino acid sequences of SEQ ID NOs: 226 and 227, respectively; and an anti-TIM-3 antibody or an antigen-binding portion thereof comprising the VH and VL amino acid sequences of SEQ ID NOs: 7 and 4, respectively; or
an anti-PD-1 antibody comprising an HC that comprises the amino acid sequences of SEQ ID NOs: 226 and 375 and an LC that comprises the amino acid sequences of SEQ ID NOs: 227 and 379; and an anti-TIM-3 antibody comprising an HC that comprises the amino acid sequences of SEQ ID NOs: 7 and 377 and an LC that comprises the amino acid sequences of SEQ ID NOs: 4 and 378.

In certain embodiments, the combination therapy or composition of the invention comprises:
an anti-PD-1 antibody or an antigen-binding portion thereof comprising the H-CDR1-3 and L-CDR1-3 amino acid sequences of SEQ ID NOs: 228-233, respectively; and an anti-LAG-3 antibody or an antigen-binding portion thereof comprising the H-CDR1-3 and L-CDR1-3 amino acid sequences of SEQ ID NOs: 318-323, respectively;
an anti-PD-1 antibody or an antigen-binding portion thereof comprising the VH and VL amino acid sequences of SEQ ID NOs: 226 and 227, respectively; and an anti-LAG-3 antibody or an antigen-binding portion thereof comprising the VH and VL amino acid sequences of SEQ ID NOs: 316 and 317, respectively; or
an anti-PD-1 antibody comprising an HC that comprises the amino acid sequences of SEQ ID NOs: 226 and 375 and an LC that comprises the amino acid sequences of SEQ ID NOs: 227 and 379; and an anti-LAG-3 antibody comprising an HC that comprises the amino acid sequences of SEQ ID NOs: 316 and 375 and an LC that comprises the amino acid sequences of SEQ ID NOs: 317 and 378.

In certain embodiments, the combination therapy or composition of the invention comprises:
an anti-TIM-3 antibody or an antigen-binding portion thereof comprising the H-CDR1-3 and L-CDR1-3 amino acid sequences of SEQ ID NOs: 8-13, respectively; and an anti-LAG-3 antibody or an antigen-binding portion thereof comprising the H-CDR1-3 and L-CDR1-3 amino acid sequences of SEQ ID NOs: 318-323, respectively;
an anti-TIM-3 antibody or an antigen-binding portion thereof comprising the VH and VL amino acid sequences of SEQ ID NOs: 7 and 4, respectively; and an anti-LAG-3 antibody or an antigen-binding portion thereof comprising the VH and VL amino acid sequences of SEQ ID NOs: 316 and 317, respectively; or
an anti-TIM-3 antibody comprising an HC that comprises the amino acid sequences of SEQ ID NOs: 7 and 377 and an LC that comprises the amino acid sequences of SEQ ID NOs: 4 and 378; and an anti-LAG-3 antibody comprising an HC that comprises the amino acid sequences of SEQ ID NOs: 316 and 375 and an LC that comprises the amino acid sequences of SEQ ID NOs: 317 and 378.

In certain embodiments, the combination therapy or composition of the invention comprises:
an anti-PD-1 antibody or an antigen-binding portion thereof comprising the H-CDR1-3 and L-CDR1-3 amino acid sequences of SEQ ID NOs: 228-233, respectively; an anti-TIM-3 antibody or an antigen-binding portion thereof comprising the H-CDR1-3 and L-CDR1-3 amino acid sequences of SEQ ID NOs: 8-13, respectively; and an anti-TIM-3 antibody or an antigen-binding portion thereof comprising the H-CDR1-3 and L-CDR1-3 amino acid sequences of SEQ ID NOs: 318-323, respectively;
an anti-PD-1 antibody or an antigen-binding portion thereof comprising the VH and VL amino acid sequences of SEQ ID NOs: 226 and 227, respectively; an anti-TIM-3 antibody or an antigen-binding portion thereof comprising the VH and VL amino acid sequences of SEQ ID NOs: 7 and 4, respectively; and an anti-LAG-3 antibody or an antigen-binding portion thereof comprising the VH and VL amino acid sequences of SEQ ID NOs: 316 and 317, respectively; or
an anti-PD-1 antibody comprising an HC that comprises the amino acid sequences of SEQ ID NOs: 226 and 375 and an LC that comprises the amino acid sequences of SEQ ID NOs: 227 and 379; an anti-TIM-3 antibody comprising an HC that comprises the amino acid sequences of SEQ ID NOs: 7 and 377 and an LC that comprises the amino acid sequences of SEQ ID NOs: 4 and 378; and an anti-LAG-3 antibody comprising an HC that comprises the amino acid sequences of SEQ ID NOs: 316 and 375 and an LC that comprises the amino acid sequences of SEQ ID NOs: 317 and 378.

In some embodiments, any or all of the antibodies in the combination therapy or composition may be an IgG, for example, IgG1 or IgG2.

### Multi-Specific Binding Molecules

In a further aspect, the invention provides a multi-specific binding molecule having the binding specificity (e.g., comprising the antigen-binding portions, such as antigen-binding portions comprising the six CDRs) of:
an anti-PD-1 antibody as described herein and an anti-TIM-3 antibody as described herein;
an anti-PD-1 antibody as described herein and an anti-LAG-3 antibody as described herein; or
an anti-TIM-3 antibody as described herein and an anti-LAG-3 antibody as described herein.
   In some embodiments, the anti-PD-1 antibody, anti-TIM-3 antibody, and/or anti-LAG-3 antibody are selected from the antibodies described herein. In certain embodiments, the multi-specific binding molecule has the binding specificity of an anti-PD-1 antibody described herein, an anti-TIM-3 antibody described herein, and an anti-LAG-3 antibody described herein. Multi-specific binding molecules are known in the art, and examples of different types of multi-specific binding molecules are given elsewhere herein. Such multi-specific (e.g., bi-specific or trispecific) binding molecules are encompassed by the combination therapies of the invention.

In a further aspect, the invention provides combination therapy with two or more of a bi-specific binding molecule targeting PD-1, a bi-specific molecule targeting TIM-3, and a bi-specific binding molecule targeting LAG-3. A bi-specific binding molecule targeting PD-1, TIM-3, or LAG-3 may have the binding specificity of an antibody targeting said antigen as described herein and the binding specificity of another antibody targeting the same antigen (e.g., another antibody as described herein) or an antibody that targets a different protein, such as another immune checkpoint protein, a cancer antigen, or another cell surface molecule whose activity mediates a disease condition such as cancer. Such bi-specific binding molecules are known in the art, and examples of different types of bi-specific binding molecules are given elsewhere herein.

### Nucleic Acid Molecules and Vectors

Also described are nucleic acid molecules and sequences encoding anti-PD-1, anti-TIM-3, and/or anti-LAG-3 antibodies or antigen-binding portions thereof described herein. In some embodiments, different nucleic acid molecules encode the heavy chain and light chain amino acid sequences of the anti-PD-1 antibody or antigen-binding portion thereof, anti-TIM-3 antibody or antigen-binding portion thereof, or anti-LAG-3 antibody or antigen-binding portion thereof. In other embodiments, the same nucleic acid molecule encodes the heavy chain and light chain amino acid sequences of the anti-PD-1 antibody or antigen-binding portion thereof, anti-TIM-3 antibody or antigen-binding portion thereof, or anti-LAG-3 antibody or antigen-binding portion thereof.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single and double stranded forms.

In some embodiments, the nucleotide sequences are at least 70%, 75%, 80%, 85%, 90%, 95%, 97%, 98% or 99% identical to one or more nucleotide sequences recited herein, e.g., to a nucleotide sequence encoding an amino acid sequence selected from the group consisting of SEQ ID NOs: 1, 2, 5, 6, 14, 15, 24, 25, 34, 35, 44, 45, 54, 55, 64, 65, 74, 75, 84, 85, 94, 95, 104, 105, 114, 115, 124, 125, 134, 135, 144, 145, 154, 155, 164, 165, 174, 175, 184, 185, 194, 195, 204, 205, 214, 215, 224, 225, 234, 235, 244, 245, 254, 255, 264, 265, 274, 275, 284, 285, 294, 295, 304, 305, 314, 315, 324, 325, 334, 335, 344, 345, 354, 355, 364, 365 or 391. The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wisconsin. FASTA, which includes, e.g., the programs FASTA2 and FASTA3, provides alignments and percent sequence identity of the regions of the best overlap between the query and search sequences (see, e.g., Pearson, Methods Enzymol. 183:63-98 (1990); Pearson, Methods Mol. Biol. 132:185-219 (2000); Pearson, Methods Enzymol. 266:227-258 (1996); and Pearson, J. Mol. Biol. 276:71-84 (1998); incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

In some embodiments, the nucleic acid molecule comprising one or more nucleotide sequences selected from the group consisting of SEQ ID NOs: 1, 2, 5, 6, 14, 15, 24, 25, 34, 35, 44, 45, 54, 55, 64, 65, 74, 75, 84, 85, 94, 95, 104, 105, 114, 115, 124, 125, 134, 135, 144, 145, 154, 155, 164, 165, 174, 175, 184, 185, 194,195, 204, 205, 214, 215, 224, 225, 234, 235, 244, 245, 254, 255, 264, 265, 274, 275, 284, 285, 294, 295, 304, 305, 314, 315, 324, 325, 334, 335, 344, 345, 354, 355, 364, 365 or 391.

In any of the above embodiments, the nucleic acid molecules may be isolated. A nucleic acid molecule encoding the heavy and/or light chain of an antibody or antigen-binding portion thereof described herein can be isolated from any source that produces such an antibody or portion. In various embodiments, the nucleic acid molecules are isolated from B cells that express an antibody isolated from an animal immunized with a PD-1, TIM-3, or LAG-3 antigen, or from an immortalized cell produced from such a B cell. Methods of isolating nucleic acids encoding an antibody are well-known in the art. mRNA may be isolated and used to produce cDNA for use in polymerase chain reaction (PCR) or cDNA cloning of antibody genes. In certain embodiments, a nucleic acid molecule as described herein can be synthesized rather than isolated.

In some embodiments, a nucleic acid molecule as described herein can comprise a nucleotide sequence encoding a VH domain from an antibody or antigen-binding portion described herein joined in-frame to a nucleotide sequence encoding a heavy chain constant region from any source. Similarly, a nucleic acid molecule of as described herein can comprise a nucleotide sequence encoding a VL domain from an antibody or antigen-binding portion described herein joined in-frame to a nucleotide sequence encoding a light chain constant region from any source.

In a further aspect, nucleic acid molecules encoding the variable domain of the heavy (VH) and/or light (VL) chains may be “converted” to full-length antibody genes. In one embodiment, nucleic acid molecules encoding the VH or VL domains are converted to full-length antibody genes by insertion into an expression vector already encoding heavy chain constant (CH) or light chain constant (CL) regions, respectively, such that the VH segment is operatively linked to the CH segment(s) within the vector, and/or the VL segment is operatively linked to the CL segment within the vector. In another embodiment, nucleic acid molecules encoding the VH and/or VL domains are converted into full-length antibody genes by linking, e.g., ligating, a nucleic acid molecule encoding a VH and/or VL domains to a nucleic acid molecule encoding a CH and/or CL region using standard molecular biological techniques. Nucleic acid molecules encoding the full-length heavy and/or light chains may then be expressed from a cell into which they have been introduced and the antibody isolated.

The nucleic acid molecules may be used to recombinantly express large quantities of antibodies. The nucleic acid molecules also may be used to produce chimeric antibodies, bi-specific antibodies, single chain antibodies, immunoadhesins, diabodies, mutated antibodies and antibody derivatives, as described herein.

Also described herein is a vector suitable for expressing one or both of the chains of an anti-PD-1 antibody or antigen-binding portion thereof, an anti-TIM-3 antibody or antigen-binding portion thereof, and/or an anti-LAG-3 antibody or antigen-binding portion thereof, as described herein. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

In some embodiments, the vectors comprise nucleic acid molecules that encode the heavy chain, the light chain, or both the heavy and light chains, of an antibody described herein or an antigen-binding portion thereof. In some embodiments, the vectors comprise nucleic acid molecules encoding fusion proteins, modified antibodies, antibody fragments, and probes thereof.

In some embodiments, the anti-PD-1, anti-TIM-3, or anti-LAG-3 antibodies or antigen-binding portions thereof are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody coding sequence may be ligated into a vector such that transcriptional and translational control sequences within the vector serve their intended function of regulating the transcription and translation of the antibody coding sequence. The expression vector and expression control sequences may be chosen to be compatible with the expression host cell used. The antibody light chain coding sequence and the antibody heavy chain coding sequence can be inserted into separate vectors, and may be operatively linked to the same or different expression control sequences (e.g., promoters). In one embodiment, both coding sequences are inserted into the same expression vector, and may be operatively linked to the same expression control sequences (e.g., a common promoter), to separate identical expression control sequences (e.g., promoters), or to different expression control sequences (e.g., promoters). The antibody coding sequences may be inserted into the expression vector by standard methods (e.g., ligation of complementary restriction sites on the antibody gene fragment and vector, or blunt end ligation if no restriction sites are present).

A convenient vector is one that encodes a functionally complete human CH or CL immunoglobulin sequence, with appropriate restriction sites engineered so that any VH or VL sequence can easily be inserted and expressed, as described above. The HC- and LC-encoding genes in such vectors may contain intron sequences that will result in enhanced overall antibody protein yields by stabilizing the related mRNA. The intron sequences are flanked by splice donor and splice acceptor sites, which determine where RNA splicing will occur. Location of intron sequences can be either in variable or constant regions of the antibody chains, or in both variable and constant regions when multiple introns are used. Polyadenylation and transcription termination may occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors may carry regulatory sequences that control the expression of the antibody chain genes in a host cell. It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods for expressing antibodies in plants, including a description of promoters and vectors, as well as transformation of plants, are known in the art. See, e.g., U.S. Pat. No. 6,517,529. Methods of expressing polypeptides in bacterial cells or fungal cells, e.g., yeast cells, are also well known in the art.

In addition to the antibody chain genes and regulatory sequences, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. For example, selectable marker genes include the dihydrofolate reductase (DHFR) gene (for use in dhfr-host cells with methotrexate selection/amplification), the neo gene (for G418 selection), and the glutamate synthetase gene.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

### Host Cells and Methods of Antibody and Antibody Composition Production

Also described are methods for producing the combination therapies (e.g., compositions) of the invention. One embodiment relates to a method for producing antibodies as described herein, comprising providing recombinant host cells capable of expressing the antibodies, culturing said host cells under conditions suitable for expression of the antibodies, and isolating the resulting antibodies. Antibodies produced by such expression in such recombinant host cells are referred to herein as “recombinant antibodies.” Also described are progeny cells of such host cells, and antibodies produced by same.

The term “recombinant host cell” (or simply “host cell”), as used herein, means a cell into which a recombinant expression vector has been introduced. The host cell may comprise, e.g., one or more vectors as described herein. The host cells may comprise, e.g., a nucleotide sequence encoding the heavy chain or an antigen-binding portion thereof, a nucleotide sequence encoding the light chain or an antigen-binding portion thereof, or both, of an anti-PD-1, anti-TIM-3, and/or anti-LAG-3 antibody or antigen-binding portion thereof as described herein. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-PD-1, anti-TIM-3, and/or anti-LAG-3 antibodies or antigen-binding portions thereof and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455. Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected by determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris.

Further, expression of antibodies or antigen-binding portions thereof as described herein from production cell lines can be enhanced using a number of known techniques. For example, the glutamine synthetase gene expression system (the GS system) is a common approach for enhancing expression under certain conditions. The GS system is discussed in whole or part in connection with EP Patents 0 216 846, 0 256 055, 0 323 997 and 0 338 841.

It is likely that antibodies expressed by different cell lines or in transgenic animals will have different glycosylation patterns from each other. However, all antibodies encoded by the nucleic acid molecules provided herein, or comprising the amino acid sequences provided herein, are part of the instant invention, regardless of the glycosylation state of the antibodies, and more generally, regardless of the presence or absence of post-translational modification(s).

In some embodiments, the invention relates to a method for producing an antibody composition comprising an anti-PD-1 antibody and an anti-TIM-3 antibody, the method comprising:
providing first and second host cells, wherein the first host cell is capable of expressing an anti-PD-1 antibody as described herein and the second host cell is capable of expressing an anti-TIM-3 antibody as described herein,
cultivating the first and second and host cells under conditions suitable for expression of the anti-PD-1 antibody and the anti-TIM-3 antibody, and
isolating the resulting antibodies.

In some embodiments, the invention relates to a method for producing an antibody composition comprising an anti-PD-1 antibody and an anti-LAG-3 antibody, the method comprising:
providing first and second host cells, wherein the first host cell is capable of expressing an anti-PD-1 antibody as described herein and the second host cell is capable of expressing an anti-LAG-3 antibody as described herein,
cultivating the first and second host cells under conditions suitable for expression of the anti-PD-1 antibody and the anti-LAG-3 antibody, and
isolating the resulting antibodies.

In some embodiments, the invention relates to a method for producing an antibody composition comprising an anti-TIM-3 antibody and an anti-LAG-3 antibody, the method comprising:
providing first and second host cells, wherein the first host cell is capable of expressing an anti-TIM-3 antibody as described herein and the second host cell is capable of expressing an anti-LAG-3 antibody as described herein,
cultivating the first and second host cells under conditions suitable for expression of the anti-TIM-3 antibody and the anti-LAG-3 antibody, and
isolating the resulting antibodies.

In some embodiments, the invention relates to a method for producing an antibody composition comprising an anti-PD-1 antibody, an anti-TIM-3 antibody, and an anti-LAG-3 antibody, the method comprising:
providing first, second, and third host cells, wherein the first host cell is capable of expressing an anti-PD-1 antibody as described herein, the second host cell is capable of expressing an anti-TIM-3 antibody as described herein, and the third host cell is capable of expressing an anti-LAG-3 antibody as described herein,
cultivating the first, second, and third host cells under conditions suitable for expression of the anti-PD-1 antibody, the anti-TIM-3 antibody, and the anti-LAG-3 antibody, and
isolating the resulting antibodies.

For production of an antibody composition of the invention, the antibodies directed to different targets may be produced separately, i.e., each antibody being produced in a separate bioreactor, or the individual antibodies may be produced together in a single bioreactor. If the antibody composition is produced in more than one bioreactor, the purified antibody composition can be obtained by pooling the antibodies obtained from individually purified supernatants from each bioreactor. Various approaches for production of a polyclonal antibody composition in multiple bioreactors, where the cell lines or antibody preparations are combined at a later point upstream or prior to or during downstream processing, are described in PCT Publication WO 2009/129814.

In the case of producing individual antibodies in a single bioreactor, this may be performed, e.g., as described in PCT Publication WO 2004/061104 or WO 2008/145133. The method described in WO 2004/061104 is based on site-specific integration of the antibody coding sequence into the genome of the individual host cells, while the method of WO 2008/145133 involves an alternative approach using random integration to produce antibodies in a single bioreactor.

Further information regarding methods suitable for preparing the antibody compositions of the invention may be found in PCT Publications WO 2012/059857 and WO 2013/164689.

The present invention also provides a polyclonal cell line that produces:
at least one anti-PD-1 antibody or antigen-binding portion thereof as described herein and at least one anti-TIM-3 antibody or antigen-binding portion thereof as described herein;
at least one anti-PD-1 antibody or antigen-binding portion thereof as described herein and at least one anti-LAG-3 antibody or antigen-binding portion thereof as described herein;
at least one anti-TIM-3 antibody or antigen-binding portion thereof as described herein and at least one anti-LAG-3 antibody or antigen-binding portion thereof as described herein; or
at least one anti-PD-1 antibody or antigen-binding portion thereof as described herein, at least one anti-TIM-3 antibody or antigen-binding portion thereof as described herein, and at least one anti-LAG-3 antibody or antigen-binding portion thereof as described herein.

The present invention also provides a method for producing the above polyclonal cell line, comprising providing host cells that each comprise a nucleotide sequence that encodes the heavy chain or an antigen-binding portion thereof and a nucleotide sequence that encodes the light chain or an antigen-binding portion thereof of at least one of the antibodies or portions produced by the polyclonal cell line.

The present invention also provide host cells comprising:
a nucleotide sequence that encodes the heavy chain or an antigen-binding portion thereof, a nucleotide sequence that encodes the light chain or an antigen-binding portion thereof, or both, of an anti-PD-1 antibody as described herein, and a nucleotide sequence that encodes the heavy chain or an antigen-binding portion thereof, a nucleotide sequence that encodes the light chain or an antigen-binding portion thereof, or both, of an anti-TIM-3 antibody as described herein;
a nucleotide sequence that encodes the heavy chain or an antigen-binding portion thereof, a nucleotide sequence that encodes the light chain or an antigen-binding portion thereof, or both, of an anti-PD-1 antibody as described herein; and a nucleotide sequence that encodes the heavy chain or an antigen-binding portion thereof, a nucleotide sequence that encodes the light chain or an antigen-binding portion thereof, or both, of an anti-LAG-3 antibody as described herein;
a nucleotide sequence that encodes the heavy chain or an antigen-binding portion thereof, a nucleotide sequence that encodes the light chain or an antigen-binding portion thereof, or both, of an anti-TIM-3 antibody as described herein; and a nucleotide sequence that encodes the heavy chain or an antigen-binding portion thereof, a nucleotide sequence that encodes the light chain or an antigen-binding portion thereof, or both, of an anti-LAG-3 antibody as described herein; or
a nucleotide sequence that encodes the heavy chain or an antigen-binding portion thereof, a nucleotide sequence that encodes the light chain or an antigen-binding portion thereof, or both, of an anti-PD-1 antibody as described herein; a nucleotide sequence that encodes the heavy chain or an antigen-binding portion thereof, a nucleotide sequence that encodes the light chain or an antigen-binding portion thereof, or both, of an anti-TIM-3 antibody as described herein; and a nucleotide sequence that encodes the heavy chain or an antigen-binding portion thereof, a nucleotide sequence that encodes the light chain or an antigen-binding portion thereof, or both, of an anti-LAG-3 antibody as described herein.

### Pharmaceutical Compositions

Another aspect of the invention is a pharmaceutical composition comprising as active ingredients (e.g., as the sole active ingredients):
at least one anti-PD-1 antibody or antigen-binding portion thereof as described herein and at least one anti-TIM-3 antibody or antigen-binding portion thereof as described herein;
at least one anti-PD-1 antibody or antigen-binding portion thereof as described herein and at least one anti-LAG-3 antibody or antigen-binding portion thereof as described herein;
at least one anti-TIM-3 antibody or antigen-binding portion thereof as described herein and at least one anti-LAG-3 antibody or antigen-binding portion thereof as described herein; or
at least one anti-PD-1 antibody or antigen-binding portion thereof as described herein, at least one anti-TIM-3 antibody or antigen-binding portion thereof as described herein, and at least one anti-LAG-3 antibody or antigen-binding portion thereof as described herein.
   In some aspects, the pharmaceutical composition comprises a multi-specific binding molecule (e.g., a multi-specific binding molecule that has the binding specificity of an anti-PD-1 antibody as described herein and an anti-TIM-3 or anti-LAG-3 antibody as described herein; or an anti-PD1 antibody, an anti-TIM-3 antibody, and an anti-LAG-3 antibody as described herein).

In some embodiments, the pharmaceutical composition may further comprise one or more additional antibodies that target one or more relevant cell surface receptors, e.g., one or more cancer-relevant receptors.

In some embodiments, the pharmaceutical composition is intended for amelioration, prevention, and/or treatment of a disorder, disease, or condition that improves, or slows down in its progression, by modulation of PD-1, TIM-3, and/or LAG-3. In some embodiments, the pharmaceutical composition is intended for amelioration, prevention, and/or treatment of cancer. In some embodiments, the pharmaceutical composition is intended for activation of the immune system.

The ratio between the antibodies of antigen-binding portions thereof in a pharmaceutical composition of the invention (or of individual antibodies or portions described herein being administered simultaneously, sequentially or separately) will often be such that the antibodies are administered in equal amounts, but this need not necessarily be the case. Thus, a composition of the invention comprising an anti-PD-1 antibody and an anti-TIM-3 antibody, an anti-PD-1 antibody and an anti-LAG-3 antibody, or an anti-TIM-3 antibody and an anti-LAG-3 antibody may contain said antibodies in approximately a 1:1 ratio. A composition of the invention comprising an anti-PD-1 antibody, an anti-TIM-3 antibody, and an anti-LAG-3 antibody may contain said antibodies in approximately a 1:1:1: ratio (i.e., in equal amounts). Depending on the characteristics of the individual antibodies, however, it may be desirable to use non-equal amounts of the different antibodies. Suitable ratios for the different antibodies in compositions of the invention may be determined as described in PCT Publication WO 2010/040356, which describes methods for identifying and selecting the optimal stoichiometric ratio between chemical entities in a combinatorial drug product, e.g. a polyclonal antibody composition, to obtain a combinatorial drug with optimal potency and efficacy.

Generally, the pharmaceutical compositions described herein are suitable to be administered as a formulation in association with one or more pharmaceutically acceptable excipient(s), e.g., as described below.

The term “excipient” is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and antigen-binding portions described herein.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Particular embodiments include the intravenous and the subcutaneous routes.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the compositions in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin, and/or by using modified-release coatings (e.g., slow-release coatings).

### Therapeutic Uses of Combination Therapies and Compositions of the Invention

In one aspect, the combination therapies and compositions of the invention are used to enhance or activate the immune system in a patient (e.g., a human) in need thereof. In some embodiments, the patient is immune-suppressed. In some embodiments, a physician can boost the anti-cancer activity of a patient's own immune system by administering a combination therapy or composition of the present invention, alone or in combination with other therapeutic agents (sequentially or concurrently). The combination therapy or composition modulates the activity of PD-1, TIM-3, and/or LAG-3 in immune cells, resulting in enhancement of anti-cancer immunity. In certain embodiments, the combination therapies and compositions of the invention are for use in the treatment of cancer, e.g., cancers that originate in tissues such as skin, lung, intestine, colon, ovary, brain, prostate, kidney, soft tissues, the hematopoietic system, head and neck, liver, bladder, breast, stomach, uterus and pancreas, and any cancers or other conditions which rely on PD-1, TIM-3, and/or LAG-3 activity and/or in which the patient expresses or overexpresses a ligand of any of these.

In some embodiments, cancers treated by the combination therapies and compositions of the invention may include, e.g., melanoma (e.g., advanced or metastatic melanoma), non-small cell lung cancer, head and neck squamous cell cancer, renal cell carcinoma, Hodgkin's lymphoma, non-Hodgkin's lymphoma, glioblastoma, glioma, squamous cell lung cancer, small-cell lung cancer, hepatocellular carcinoma, bladder cancer, upper urinary tract cancer, esophageal cancer, gastroesophageal junction cancer, gastric cancer, liver cancer, colon cancer, colorectal carcinoma, multiple myeloma, sarcomas, acute myeloid leukemia, chronic myeloid leukemia, myelodysplastic syndrome, nasopharyngeal cancer, chronic lymphocytic leukemia, acute lymphoblastic leukemia, small lymphocytic lymphoma, ovarian cancer, gastrointestinal cancer, primary peritoneal cancer, fallopian tube cancer, urothelial cancer, HTLV-associated T-cell leukemia/lymphoma, prostate cancer, genitourinary cancer, meningioma, adrenocortical cancer, gliosarcoma, fibrosarcoma, kidney cancer, breast cancer, pancreatic cancer, endometrial cancer, skin basal cell cancer, cancer of the appendix, biliary tract cancer, salivary gland cancer, advanced Merkel cell cancer, diffuse large B cell lymphoma, follicular lymphoma, mesothelioma, neuroendocrine tumors, urological cancer, bone cancer, thoracic cancer, respiratory tract cancer, adenoid cystic carcinoma, cervical cancer, astrocytoma, chordoma, neuroblastoma, oral cavity cancer, cutaneous squamous cell carcinoma, thyroid cancer, Kaposi sarcoma, anal cancer, gallbladder cancer, thymic cancer, uterine cancer, and solid tumors. The cancer may be, e.g., at an early, intermediate, advanced, or metastatic stage.

In particular embodiments, cancers treated by the combination therapies and compositions of the invention may include, e.g., melanoma (e.g., advanced melanoma, or unresectable or metastatic melanoma), non-small cell lung cancer (e.g., advanced non-small cell lung cancer), lung carcinoma, head and neck squamous cell carcinoma, glioblastoma (e.g., recurrent glioblastoma), gliosarcoma, Merkel-cell carcinoma, fibrosarcoma, ovarian cancer, bladder cancer, renal cell carcinoma, colorectal cancer, Hodgkin's lymphoma, non-Hodgkin's lymphoma, leukemia (e.g., acute myeloid leukemia), hematologic malignancies, solid tumors (e.g., advanced or metastatic solid tumors), MSI high tumors, HPV and HIV associated malignancies, and tumors with BRAC1 and BRAC2 mutations.

In certain embodiments, the pharmaceutical compositions of the invention are intended for treatment of an immune-mediated disorder such as psoriasis, systemic lupus erythematosis, MLS (sclerosis), Crohn's disease, diabetes mellitus, and/or colitis ulcerotis.

In some embodiments, the combination therapy or composition is for use in treating viral and/or parasitic infections, e.g., where the pathogens inhibit the host immune response. For example, the pathogen may be, e.g., HIV, hepatitis (A, B, or C), human papilloma virus (HPV), lymphocytic choriomeningitis virus (LCMV), adenovirus, flavivirus, echovirus, rhinovirus, coxsackie virus, cornovirus, respiratory syncytial virus, mumps virus, rotavirus, measles virus, rubella virus, parvovirus, vaccinia virus, human T-cell lymphotrophic virus (HTLV), human cytomegalovirus (HCMV), dengue virus, molluscum virus, poliovirus, rabies virus, John Cunningham (JC) virus, arboviral encephalitis virus, simian immunodeficiency virus (SIV), influenza, herpes, Giardia, malaria, Leishmania, Staphylococcus aureus, or Pseudomonas aeruginosa.

In some embodiments, the combination therapies and compositions of the invention may be used to treat a patient who is, or is at risk of being, immunocompromised (e.g., due to chemotherapeutic or radiation therapy).

In some embodiments, the combination therapies and compositions of the invention may be used for ex vivo activation and expansion of antigen-specific T cells.

In some embodiments, the patient may have been treated previously for a condition characterized by overexpression or overactivity of PD-1, TIM-3, and/or LAG-3 or any of their ligands (e.g., cancer or an immune disorder). For example, the patient may have been treated with one or more drugs targeting PD-1, TIM-3, and/or LAG-3 and may have acquired resistance to said drug(s).

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disorder being treated. A therapeutically effective amount of an anti-cancer therapeutic may, for example, result in tumor shrinkage, increased survival, elimination of cancer cells, decreased disease progression, reversal of metastasis, or other clinical endpoints desired by healthcare professionals.

In some embodiments, the antibodies, antigen-binding portions, or multi-specific binding molecules in the combination therapy of the invention are administered in a single composition. In other embodiments, the antibodies, antigen-binding portions, or multi-specific binding molecules are administered in more than one composition. For example, a combination therapy comprising an anti-PD-1 antibody, an anti-TIM-3 antibody, and an anti-LAG-3 antibody may involve administration of a single composition comprising all three antibodies, a composition comprising two of the antibodies and a composition comprising one of the antibodies, or a separate composition for each antibody. In a case where there is more than one composition, the compositions can be administered simultaneously, sequentially, separately, or any combination thereof.

The combination therapies and compositions of the invention may be administered alone or in combination with one or more other drugs or antibodies (or as any combination thereof). The pharmaceutical compositions, methods and uses of the invention thus also encompass embodiments of combinations (co-administration) with other active agents, as detailed below.

As used herein, the terms “co-administration”, “co-administered” and “in combination with,” referring to the combination therapies or compositions of the invention with one or more other therapeutic agents, is intended to mean, and does refer to and include the following:
simultaneous administration of such combination therapy (or a component thereof) or composition and therapeutic agent(s) to a patient in need of treatment, when such components are formulated together into a single dosage form which releases said components at substantially the same time to said patient,
substantially simultaneous administration of such combination therapy (or a component thereof) or composition and therapeutic agent(s) to a patient in need of treatment, when such components are formulated apart from each other into separate dosage forms which are taken at substantially the same time by said patient, whereupon said components are released at substantially the same time to said patient,
sequential administration of such combination therapy (or a component thereof) or composition and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination therapy (or a component thereof) or composition and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

The combination therapies and compositions of the invention may be administered without additional therapeutic treatments, i.e., as a stand-alone therapy (i.e., monotherapy). Alternatively, treatment with a combination therapy or composition of the invention may include at least one additional therapeutic treatment, e.g., another immunostimulatory agent, an anti-cancer agent, an anti-viral agent, or a vaccine (e.g., a tumor vaccine). In some embodiments, the combination therapy or composition may be co-administered or formulated with another medication/drug for the treatment of cancer. The additional therapeutic treatment may comprise, e.g., a chemotherapeutic, anti-neoplastic, or anti-angiogenic agent, a different anti-cancer antibody, and/or radiation therapy.

By combining the combination therapies and compositions of the invention with agents known to induce terminal differentiation of cancer cells, the effect may be improved further. Such compounds may, for example, be selected from the group consisting of retinoic acid, trans-retinoic acids, cis-retinoic acids, phenylbutyrate, nerve growth factor, dimethyl sulfoxide, active form vitamin D3, peroxisome proliferator-activated receptor gamma, 12-O-tetradecanoylphorbol 13-acetate, hexamethylene-bis-acetamide, transforming growth factor-beta, butyric acid, cyclic AMP, and vesnarinone. In some embodiments, the compound is selected from the group consisting of retinoic acid, phenylbutyrate, all-trans-retinoic acid and active form vitamin D.

Pharmaceutical articles comprising a combination therapy or composition of the invention and at least one other agent (e.g., a chemotherapeutic, anti-neoplastic, or anti-angiogenic agent) may be used as a combination treatment for simultaneous, separate or successive administration in cancer therapy. The other agent may be any agent suitable for treatment of the particular cancer in question, for example, an agent selected from the group consisting of alkylating agents, e.g., platinum derivatives such as cisplatin, carboplatin and/or oxaliplatin; plant alkoids, e.g., paclitaxel, docetaxel and/or irinotecan; antitumor antibiotics, e.g., doxorubicin (adriamycin), daunorubicin, epirubicin, idarubicin mitoxantrone, dactinomycin, bleomycin, actinomycin, luteomycin, and/or mitomycin; topoisomerase inhibitors such as topotecan; and/or antimetabolites, e.g., fluorouracil and/or other fluoropyrimidines. In some embodiments, the other agent is dacarbazine or gemcitabine.

A combination therapy or composition of the invention may also be used in combination with other anti-cancer therapies such as vaccines, cytokines, enzyme inhibitors, immunostimulatory compounds, and T cell therapies. In the case of a vaccine, it may, e.g., be a protein, peptide or DNA vaccine containing one or more antigens which are relevant for the cancer being treated, or a vaccine comprising dendritic cells along with an antigen. Suitable cytokines include, for example, IL-2, IFN-gamma and GM-CSF. An example of a type of enzyme inhibitor that has anti-cancer activity is an indoleamine-2,3-dioxygenase (IDO) inhibitor, for example 1-methyl-D-tryptophan (1-D-MT). Adoptive T cell therapy refers to various immunotherapy techniques that involve expanding or engineering patients' own T cells to recognize and attack their tumors.

It is also contemplated that a combination therapy or composition of the invention may be used in adjunctive therapy in connection with tyrosine kinase inhibitors. These are synthetic, mainly quinazoline-derived, low molecular weight molecules that interact with the intracellular tyrosine kinase domain of receptors and inhibit ligand-induced receptor phosphorylation by competing for the intracellular Mg-ATP binding site.

In some embodiments, the combination therapy or composition may be used in combination with another medication/drug that mediates immune system activation, including, but not limited to, an agent that modulates the expression or activity of A2AR, BTLA, B7-H3, B7-H4, CTLA-4, CD27, CD28, CD39, CD40, CD47, CD55, CD73, CD122, CD137, CD160, CGEN-15049, LY108, CHK1, CHK2, CTLA-3, CEACAM (e.g., CEACAM-1 and/or CEACAM-5), GAL9, GITR, HVEM, ICOS, IDO, KIR, LAIR1, NKG2A, OX40, PD-L1/PD-L2, LILRB2, CMTM6, TIGIT, TGFR-beta, TNFR2, VISTA and/or 2B4. In certain embodiments, the agent is an antibody or an antigen-binding fragment thereof that binds to one of the above molecules. In particular embodiments, the antibody or antigen-binding portion thereof, composition, or bi-specific binding molecule of the invention may be administered in combination with a CTLA-4 inhibitor (e.g., an anti-CTLA-4 antibody such as tremelimumab or ipilimumab). In one embodiment, the antibody or antigen-binding portion thereof, composition, or bi-specific binding molecule of the invention may be administered in combination with ipilimumab. It is also contemplated that the combination therapy or composition of the invention may be used in combination with a cytokine (e.g., IL-1, IL-2, IL-12, IL-15 or IL-21), an EGFR inhibitor, a VEGF inhibitor, etc.

In certain aspects, the combination therapies and compositions of the invention may be administered in combination with another inhibitor of the PD-1, TIM-3, or LAG-3 pathway, which may target PD-1, TIM-3, LAG-3, or one or more of ligands of any of these targets. Examples of such inhibitors include:
other anti-PD-1 antibodies and antibodies that target PD-1 ligands and/or co-receptors such as PD-L1 or PD-L2 (e.g., pembrolizumab and/or nivolumab);
other anti-TIM-3 antibodies and antibodies that target TIM-3 ligands and/or co-receptors such as galectin-9, HMGB-1, phosphatidylserine lipids, CEACAMI, LILRA1-6, or LILRB1-5 (e.g., MGB453, TSR-022, and/or LY3321367); and
other anti-LAG-3 antibodies and antibodies that target LAG-3 ligands and/or co-receptors such as MHCII, Galectin-3, and LSECtin (e.g., BMS-986016, GSK2831781, REGN3767, BAP050 or BAP050-chi, or LAG525).

It is understood that the combination therapies and compositions of the invention may be used in a method of treatment as described herein, may be for use in a treatment as described herein, and/or may be for use in the manufacture of a medicament for a treatment as described herein. The invention also provides kits and articles of manufacture comprising the combination therapies or compositions of the invention as described herein.

### Dose and Route of Administration

The combination therapies and compositions of the invention will be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied combination therapy. Further, the dosage regimen with the combination therapies and compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

It is contemplated that a suitable dose of an antibody in a combination therapy or composition of the invention will be in the range of 0.1-100 mg/kg, such as about 0.5-50 mg/kg, e.g., about 1-20 mg/kg. The antibody may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., once every week, once every two weeks, once every three weeks, or once every four weeks, and for as long as deemed appropriate by the responsible doctor, who may optionally increase or decrease the dosage as necessary.

An effective amount for tumor therapy may be measured by its ability to stabilize disease progression and/or ameliorate symptoms in a patient, and preferably to reverse disease progression, e.g., by reducing tumor size. The ability of a combination therapy of the invention to inhibit cancer may be evaluated by in vitro assays, e.g., as described in the Examples, as well as in suitable animal models that are predictive of the efficacy in human tumors (see, e.g., the Examples). Suitable dosage regimens will be selected in order to provide an optimum therapeutic response in each particular situation, for example, administered as a single bolus or as a continuous infusion, and with possible adjustment of the dosage as indicated by the exigencies of each case.

### Articles of Manufacture and Kits

The present invention also provides articles of manufacture comprising an anti-PD-1 antibody that competes for binding to human PD-1 with, or binds to the same epitope of human PD-1 as, an antibody selected from the group consisting of 12819.15384, 12748.15381, 12748.16124, 12865.15377, 12892.15378, 12796.15376, 12777.15382, 12760.15375 and 13112.15380; and an anti-TIM-3 antibody or an anti-LAG-3 antibody.

In some embodiments, the article of manufacture comprises:
at least one anti-PD-1 antibody or antigen-binding portion thereof as described herein and at least one anti-TIM-3 antibody or antigen-binding portion thereof as described herein;
at least one anti-PD-1 antibody or antigen-binding portion thereof as described herein and at least one anti-LAG-3 antibody or antigen-binding portion thereof as described herein;
at least one anti-TIM-3 antibody or antigen-binding portion thereof as described herein and at least one anti-LAG-3 antibody or antigen-binding portion thereof as described herein; or
at least one anti-PD-1 antibody or antigen-binding portion thereof as described herein, at least one anti-TIM-3 antibody or antigen-binding portion thereof as described herein, and at least one anti-LAG-3 antibody or antigen-binding portion thereof as described herein;
as well as methods for manufacturing said articles.

The present invention also provides kits comprising an anti-PD-1 antibody that competes for binding to human PD-1 with, or binds to the same epitope of human PD-1 as, an antibody selected from the group consisting of 12819.15384, 12748.15381, 12748.16124, 12865.15377, 12892.15378, 12796.15376, 12777.15382, 12760.15375 and 13112.15380; and an anti-TIM-3 antibody or an anti-LAG-3 antibody.

In some embodiments, the kit comprises:
at least one anti-PD-1 antibody or antigen-binding portion thereof as described herein and at least one anti-TIM-3 antibody or antigen-binding portion thereof as described herein;
at least one anti-PD-1 antibody or antigen-binding portion thereof as described herein and at least one anti-LAG-3 antibody or antigen-binding portion thereof as described herein;
at least one anti-TIM-3 antibody or antigen-binding portion thereof as described herein and at least one anti-LAG-3 antibody or antigen-binding portion thereof as described herein; or
at least one anti-PD-1 antibody or antigen-binding portion thereof as described herein, at least one anti-TIM-3 antibody or antigen-binding portion thereof as described herein, and at least one anti-LAG-3 antibody or antigen-binding portion thereof as described herein.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Enhanced In Vitro Activity of Combined PD-1/TIM-3 Targeting in One-Way MLR Assay

This example demonstrates that combined targeting of PD-1 and TIM-3 using the anti-PD-1 antibody 12819 and the anti-TIM-3 antibody 15086.17145 enhances IFN-γ secretion in a one-way mixed lymphocyte reaction (MLR) assay.

In the one-way MLR assay, dendritic cells (DCs) and CD4⁺ T-cells isolated from two different human healthy donors were co-cultured to induce an alloantigen specific reaction resulting in cytokine production and T-cell activation/proliferation. Dendritic cells (DCs) were differentiated from CD14⁺ monocytes by 6 days of culture with 20 ng/ml granulocyte-macrophage colony-stimulating factor (GM-CSF) and 20 ng/ml interleukin-4 (IL-4) and mixed in a 1:10 ratio with CD4⁺ T-cells isolated from peripheral blood mononuclear cells (PBMCs) from healthy human donor material. The indicated antibodies or antibody mixture were added to a final total concentration of 10 μg/mL. The antibody mixture contained a 1:1 ratio of anti-PD-1 and anti-TIM-3 antibodies. After 5 days of culture, supernatants were harvested and IFN-γ levels were determined using the Meso Scale electrochemiluminescence cytokine assay. Student's unpaired t-test was used for statistical analysis and Bonferroni correction was used to adjust for multiple comparisons. A corrected p-value<0.05 is considered statistically significant.

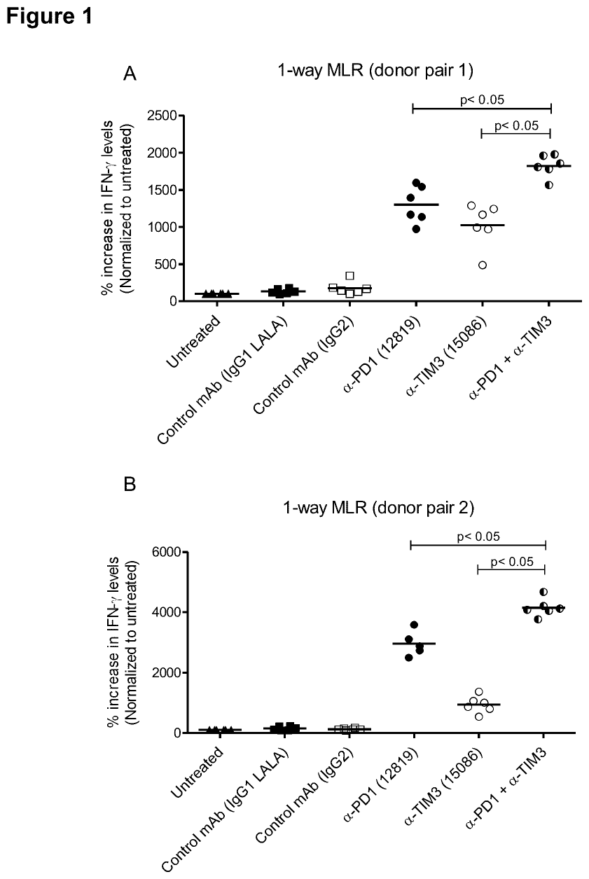

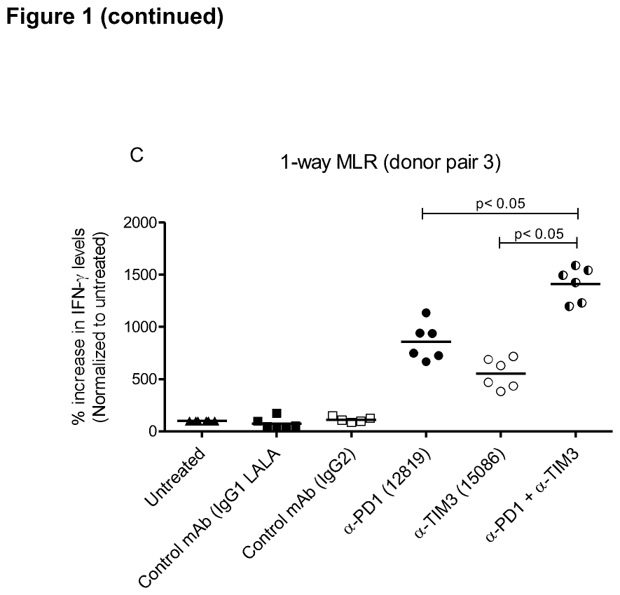

FIG. 1 shows the effect of anti-PD-1 and anti-TIM-3 antibodies on IFN-γ secretion in the one-way MLR assay in three independent donor-pairs. Both the anti-PD-1 (12819) and anti-TIM-3 (15086) antibodies were functional in the one-way MLR assay and increase IFN-γ secretion in all three donor pairs. Furthermore, the combination of anti-PD-1 and anti-TIM-3 antibodies shows enhanced activity by inducing a statistically significant increase in IFN-γ compared to the individual antibodies.

### Example 2: Enhanced In Vitro Activity of Combined PD-1/TIM-3 Targeting in Two-Way MLR Assay

This example demonstrates that combined targeting of PD-1 and TIM-3 using the anti-PD-1 antibody 12819 and the anti-TIM-3 antibody 15086.17145 enhances IFN-γ secretion in a two-way MLR assay.

In the two-way MLR assay, PBMCs from two different healthy human donors were co-cultured to induce an alloantigen specific reaction resulting in cytokine production and T-cell activation/proliferation. The PBMCs from the two different donors were mixed in a 1:1 ratio. The antibodies were tested at a final total antibody concentration of 10 μg/mL. After 5 days of culture, supernatants were harvested and IFN-γ levels were determined using the Meso Scale electrochemiluminescence cytokine assay. Student's unpaired t-test was used for statistical analysis and Bonferroni correction was used to adjust for multiple comparisons. A corrected p-value<0.05 is considered statistically significant.

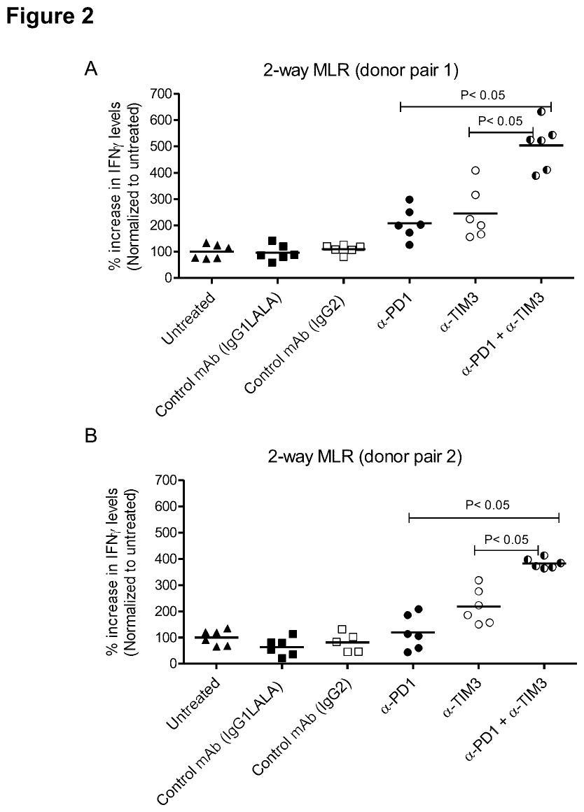

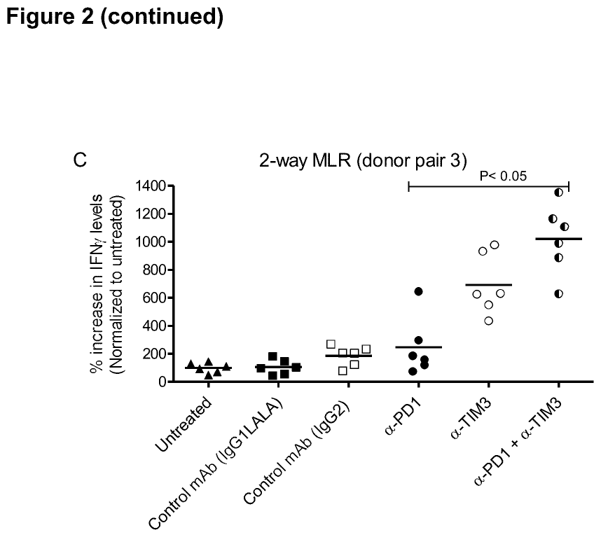

FIG. 2 shows the effect of anti-PD-1 and anti-TIM-3 antibodies on IFN-γ secretion in the two-way MLR assay in three independent human donor pairs. Anti-PD-1 and anti-TIM-3 antibodies both increased IFN-γ secretion in the two-way MLR assay. Additionally, the combination of anti-PD-1 and anti-TIM-3 antibodies showed enhanced activity by inducing increased IFN-γ secretion in comparison with the individual antibodies.

### Example 3: Anti-TIM-3 Antibody Induces T-Cell Proliferation In Vitro in One-Way MLR Assay

The ability of anti-TIM-3 antibody 15086.17145 to induce T-cell proliferation was investigated in the one-way MLR assay. The anti-TIM-3 antibody, a positive control antibody against PD-1 (12819), or a negative control IgG2 antibody were added to a final concentration of 25 μg/mL and incubated for 5 days prior to adding 1 μCi/well 3H-thymidine for an additional 18 hours. Cells were harvested, and 3H-thymidine incorporation determined by liquid scintillation counting (MicroBeta2).

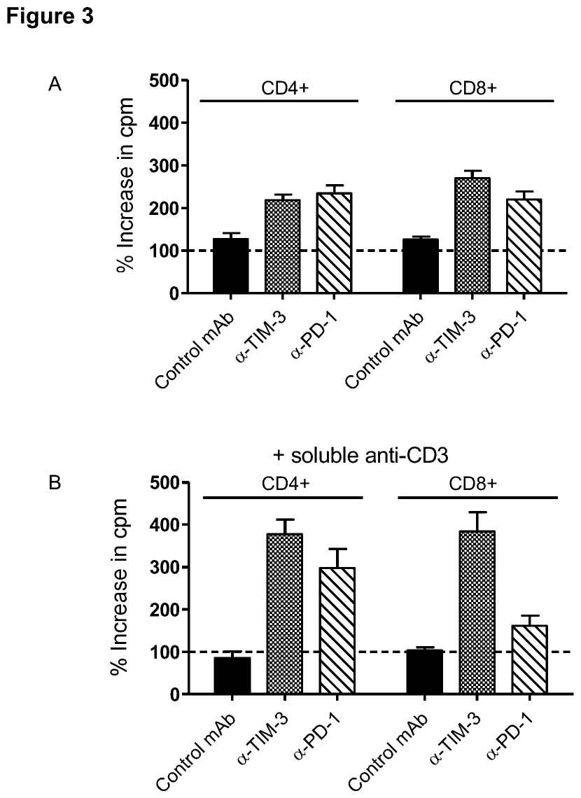

As shown in FIG. 3, anti-TIM-3 antibody 15086.17145 induced proliferation of both CD4+ and CD8+ T-cells to a similar degree as the positive control antibody against PD-1 (Panel A). Addition of soluble anti-CD3 enhanced the proliferation induced by anti-TIM-3 antibody (Panel B).

### Example 4: Anti-TIM-3 Antibody Induces IL-12p40 Secretion from Dendritic Cells

Monocyte derived dendritic cells were generated from healthy donor material as described previously in Example 1. The dendritic cells were incubated for 5 days with 10 μg/mL of anti-TIM-3 antibody 15086.17145 or a negative control IgG2 antibody, or without treatment, and IL-12p40 levels in the supernatants were determined using the standard ELISA cytokine assay.

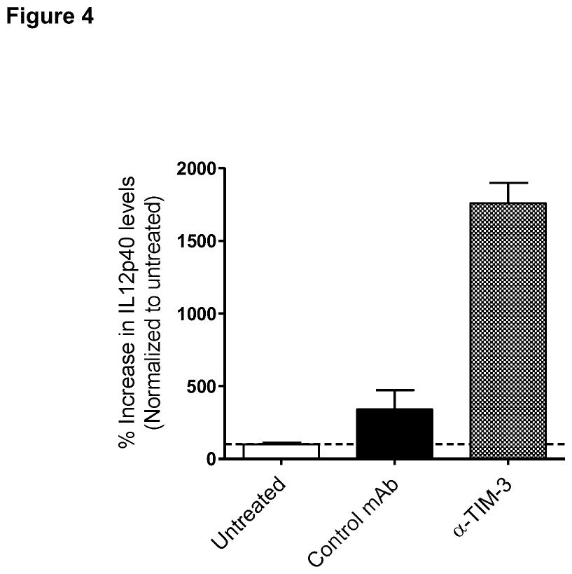

As shown in FIG. 4, ligation of TIM-3 by anti-TIM-3 antibody 15086.17145 resulted in an increase in IL-12p40 secretion from dendritic cells.

### Example 5: Anti-TIM-3 Antibody Induces Expression of Activation Markers in Dendritic Cells

To further investigate the functional role of targeting TIM-3, expression levels of selected activation markers on monocyte derived dendritic cells were determined after treatment with anti-TIM-3 antibody 15086.17145.

Dendritic cells were treated with 25 μg/mL anti-TIM-3 antibody for 24 hours and gene expression of co-stimulatory molecules was determined using NanoString Technologies. Gene expression levels were normalized to 30 housekeeping genes with uniform expression. Data for selected relevant genes are presented as fold change relative to untreated control cells.

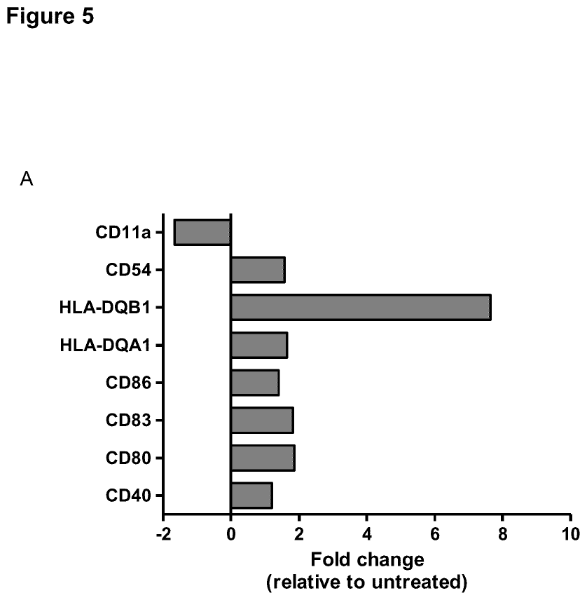

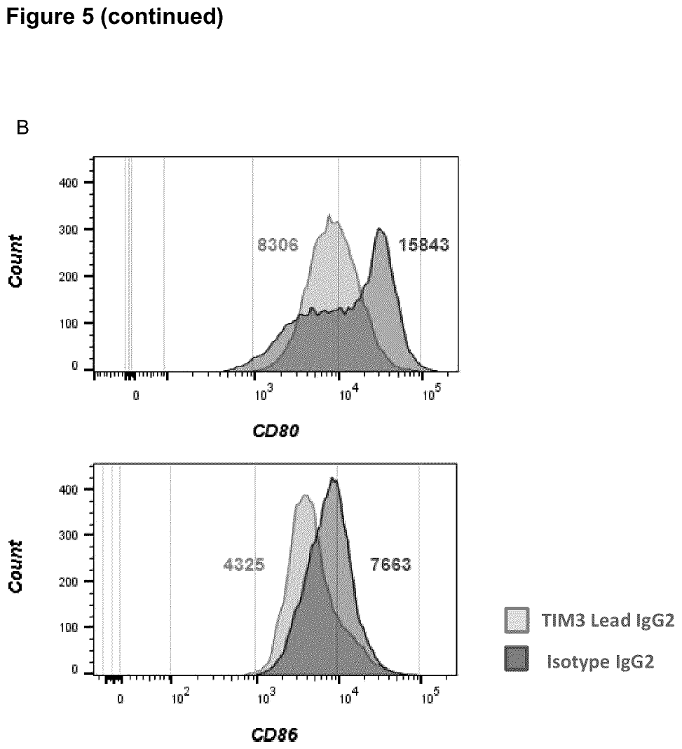

Gene expression analysis showed an upregulation of several activation markers and co-stimulatory molecules including MHC-II (HLA-DQB1 and HLA-DQA1), CD80 and CD86 (FIG. 5, Panel A). Increased cell surface expression of CD80 and CD86 upon treatment with anti-TIM-3 antibody 15086.17145 was validated using FACS analysis (FIG. 5, Panel B). The histogram overlays shown are representative of CD11c+ dendritic cells, and numbers adjacent to the histograms denote MFI values.

### Example 6: In Vivo Efficacy of Anti-TIM-3 Antibody 15086 on Human Lung PDX Tumor Growth

This example shows the in vivo efficacy of anti-TIM-3 antibody 15086.17145 on a human lung patient-derived xenograft (PDX) tumor model in CD34⁺ humanized NSG-SGM3 mice.

NSG-SGM3 mice were humanized using cord blood derived CD34⁺ cells and engrafted in the right flank with patient-derived lung tumor fragments (LG1306). At tumor sizes between 50-150 mm³, mice were randomized and treatment initiated. The mice received intraperitoneal injection of vehicle or anti-TIM-3 antibody 15086 with an initial dose of 10 mg/kg followed by 5 mg/kg 5× QSD. Tumors were measured three times weekly by caliper in two dimensions and tumor volume in mm³ was calculated according to the formula: (width)²×length×0.5. The grey area denotes the treatment period. Two-way ANOVA with Bonferroni's multiple comparisons test was applied to compare tumor volumes at each time-point between treatment groups. Data are presented as means±SEM, **p<0.01.

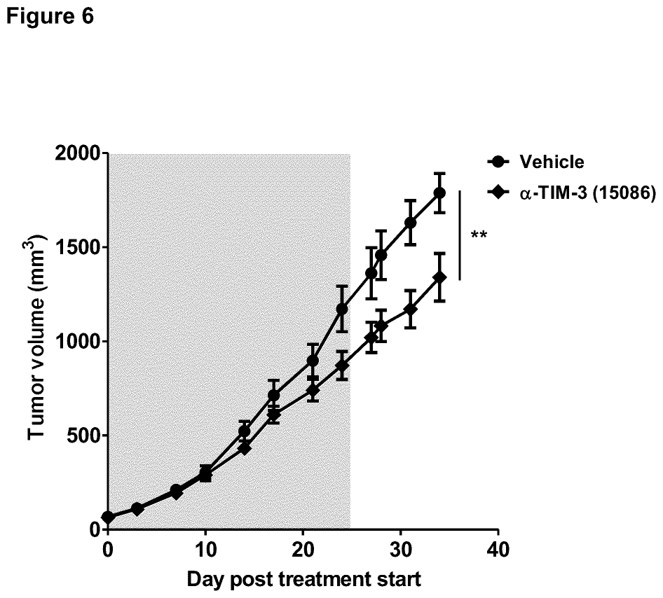

Treatment with anti-TIM-3 antibody 15086 resulted in a significant tumor growth inhibition in a CD34⁺ humanized NSG-SGM3 mouse human lung PDX model (FIG. 6).

### Example 7: Enhanced Effect of Combined PD-1/LAG-3, PD-1/TIM-3, or PD-1/TIM-3/LAG-3 Targeting in a PBMC+SEB Assay

This example describes the enhanced effect of combining anti-PD-1 antibody 12819 with anti-LAG-3 antibody 15532 or anti-TIM-3 antibody 15086.17145 in the PBMC+SEB (Staphylococcal Enterotoxin B) assay.

SEB is a super-antigen that binds to MHC class II molecules and specific Vβ regions of T cell receptors (TCR) and drives non-specific stimulation of T-cells. This results in polyclonal T cell activation/proliferation and release of cytokines, including IL-2. Human PBMCs isolated from buffy coats from healthy donors were seeded in 384-well plates, and left untreated or treated with 10 ng/mL SEB and 10 μg/mL of the indicated single antibodies or antibody mixtures. Combinations of antibodies were 1:1 or 1:1:1 mixtures of the indicated antibodies. After 48 hours in a humidified incubator at 37° C., supernatants were removed and analyzed for IL-2 levels using an IL-2 ELISA kit (Life Technologies). Data are presented as average±SEM. Significant differences were tested using Student's t-test with Bonferroni correction.

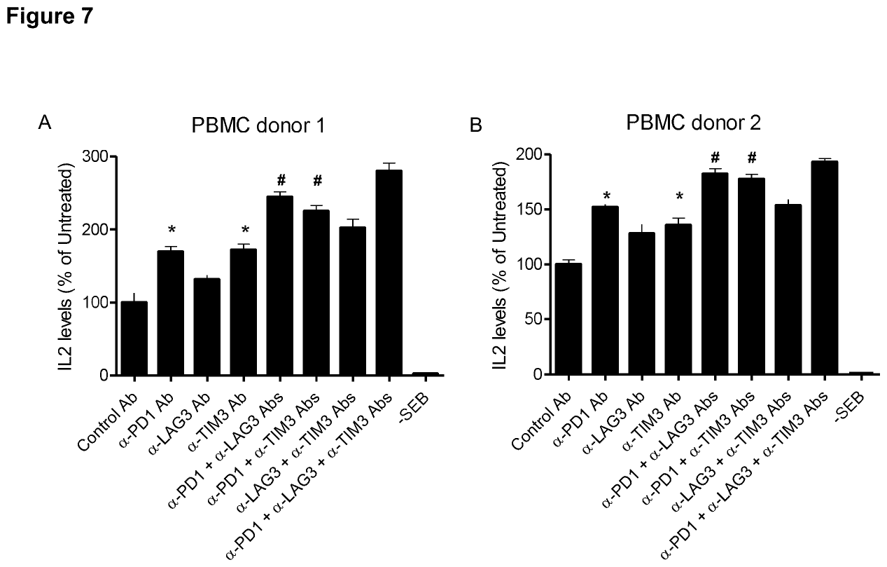

FIG. 7 shows an increase in IL-2 secretion after treatment with a single anti-PD-1, anti-LAG-3 or anti-TIM-3 antibody, mixtures of two of the antibodies, or the mixture of all three antibodies. The data demonstrated that IL-2 levels were increased by single treatment with the anti-PD-1, anti-LAG-3 or anti-TIM-3 antibody, whereas IL-2 secretion was increased further by treatment with a combination of anti-PD-1 and anti-LAG-3 antibodies, or anti-PD-1 and anti-TIM-3 antibodies. The triple combination of anti-PD-1, anti-LAG-3, and anti-TIM-3 antibodies increased IL-2 secretion more than any of the two-antibody combinations.

### Example 8: Enhanced In Vivo Efficacy of Combining Anti-PD-1 and Anti-LAG-3 Antibodies in Two Syngeneic Mouse Tumor Models

This example demonstrates the in vivo efficacy of combining anti-PD-1 antibody 12819 with anti-LAG-3 antibody C9B7W (reactive with mouse LAG-3; BioXcell) or anti-LAG-3 antibody 15011 in two syngeneic mouse tumor models.

0.5×10⁶ MC38 (colon carcinoma) or 5×10⁶ ASB-XIV (lung carcinoma) cells were inoculated subcutaneously into the flank of 6-8 week old female BALB/cAnNRj (ASB-XIV) or C57BL/6 (MC38) mice, respectively. Tumors were measured three times weekly by caliper in two dimensions and tumor volume in mm³ was calculated according to the formula: (width)²×length×0.5. On day 5 (ASB-XIV) or day 13 (MC38) post-inoculation at an average tumor size of 30-50 mm³, mice were randomized into four groups of ten animals and treatment was initiated. The mice were treated three times weekly with a total of six treatments by intraperitoneal injection of vehicle buffer, anti-PD-1 antibody 12819, anti-LAG-3 antibody C9B7W, anti-LAG-3 antibody 15011, or a combination of anti-PD-1 and anti-LAG-3 antibodies. The antibody treatments were administered at a dose of 10 mg/kg/target. Two-way ANOVA with Bonferroni's multiple comparisons test was applied to compare tumor volumes at each time-point between treatment groups. Statistical analyses were performed using GraphPad Prism version 5.0 (GraphPad Software, Inc.).

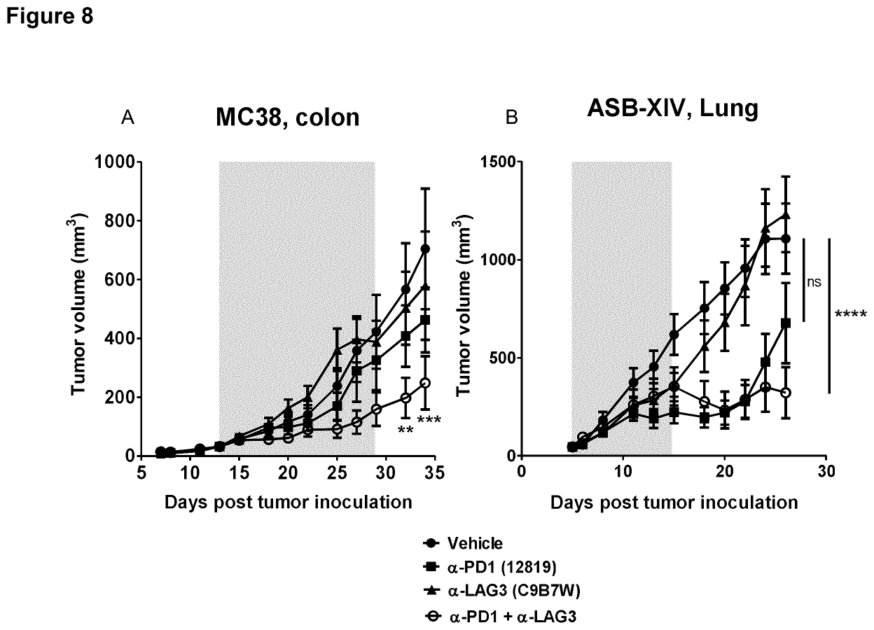

MC38 syngeneic tumors treated with monoclonal anti-PD-1 antibody 12819 displayed continuous tumor growth, albeit with slower growth kinetics than vehicle treated tumors (FIG. 8). No effect of treatment with anti-LAG-3 antibody C9B7W alone was observed. The combination of the anti-PD-1 antibody and the anti-LAG-3 antibody showed enhanced tumor growth inhibition compared to the anti-PD-1 treatment and significant tumor growth inhibition compared to vehicle treated tumors (p<0.001) (FIG. 8).

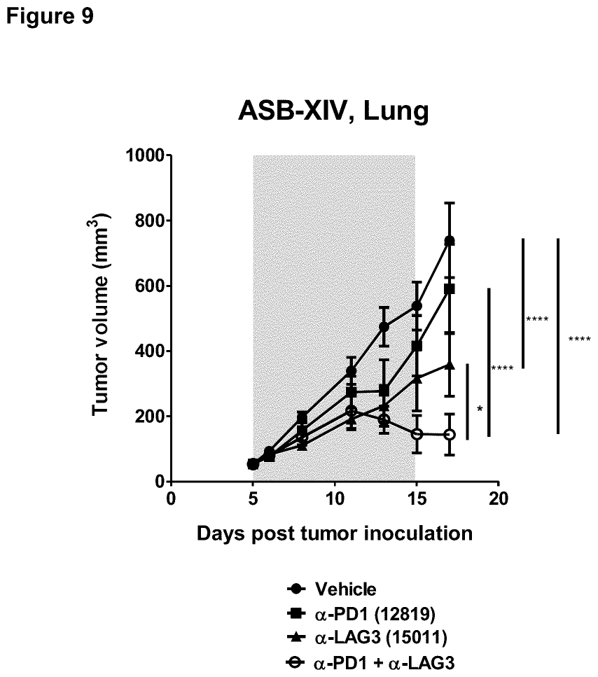

ASB-XIV syngeneic tumors treated with anti-PD-1 antibody 12819 showed delayed tumor growth compared to vehicle treated tumors (FIGS. 8 and 9). No effect of treatment with anti-LAG-3 antibody C9B7W alone was observed (FIG. 8), whereas anti-LAG-3 antibody 15011 significantly inhibited tumor growth compared to vehicle treatment (p<0.05) (FIG. 9). Combining the anti-PD-1 antibody and anti-LAG-3 antibody C9B7W significantly enhanced anti-tumor efficacy compared to vehicle treatment (FIG. 8). A significantly enhanced tumor inhibitory effect was also observed by a combination of the anti-PD-1 antibody and anti-LAG-3 antibody 15011 compared to single treatment with the anti-PD-1 antibody (p<0.0001) or the anti-LAG-3 antibody (p<0.05) (FIG. 9).

### Example 9: Enhanced In Vivo Efficacy of Combining Anti-PD-1 and Anti-TIM-3 Antibodies in Two Syngeneic Mouse Tumor Models

This example demonstrates the in vivo efficacy of combining anti-PD-1 antibody 12819 and anti-TIM-3 antibody 5D12 (reactive with mouse TIM-3; Anderson et al., Science 318:1141-43 (2007)) in two syngeneic mouse tumor models.

0.2×10⁶ Sa1N (fibrosarcoma) or 5×10⁶ ASB-XIV (lung carcinoma) cells were inoculated subcutaneously into the flank of 6-8 week old female A/J (Sa1N) and BALB/cAnNRj (ASB-XIV) mice, respectively. Tumors were measured three times weekly by caliper in two dimensions and tumor volume in mm³ was calculated according to the formula: (width)²×length×0.5. At an average tumor size of 60-110 mm³, mice were randomized and treatment initiated. The mice were treated with a single dose (Sa1N), or treated three times weekly with a total of six treatments (ASB-XIV), by intraperitoneal injection of vehicle buffer, anti-PD-1 antibody 12819, and/or anti-TIM-3 antibody 5D12. The antibody treatments were administered at a dose of 10 mg/kg/target in mice with ASB-XIV tumors. Mice with Sa1N tumors were dosed with anti-PD-1 and anti-TIM-3 antibodies at 1 mg/kg and 10 mg/kg, respectively. Two-way ANOVA with Bonferroni's multiple comparisons test was applied to compare tumor volumes at each time-point between treatment groups. Statistical analyses were performed using GraphPad Prism version 5.0 (GraphPad Software, Inc.).

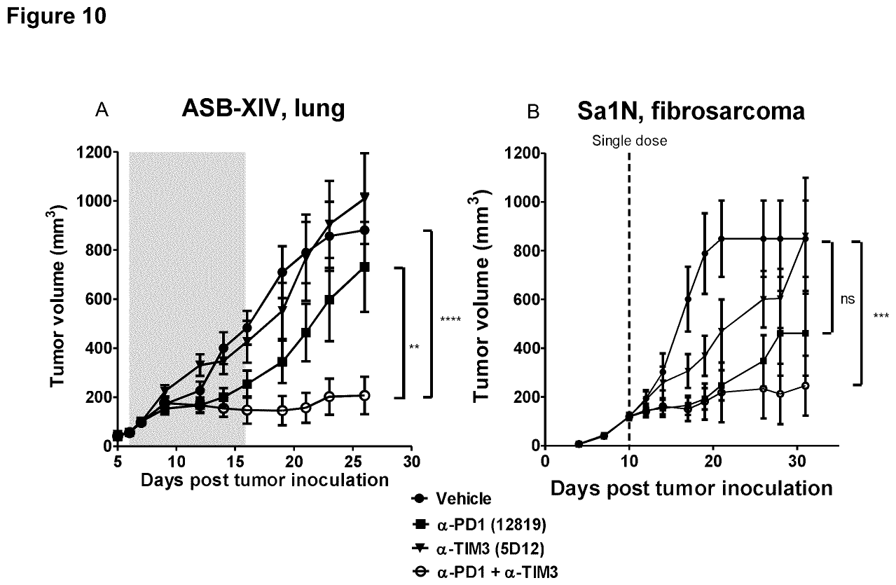

On day 6 post-inoculation of ASB-XIV tumor cells, at an average tumor size of 56 mm³, mice were randomized into four groups of ten animals and treatment was initiated. Treatment with anti-PD-1 antibody 12819 delayed tumor growth, whereas treatment with anti-TIM-3 antibody 5D12 had no effect on tumor growth compared to vehicle treatment. A pronounced tumor inhibitory effect was seen by combining the anti-PD-1 antibody with the anti-TIM-3 antibody, compared to single treatment with either antibody (p<0.001) (FIG. 10).

On day 13 post-inoculation of Sa1N tumor cells, at an average tumor size of 110 mm³, mice were randomized into four groups of ten animals and dosed with a single treatment of antibodies. The results showed an initial tumor growth delay from single antibody treatment with anti-PD-1 antibody 12819 or anti-TIM-3 antibody 5D12. An enhanced anti-tumor effect was observed by combining the anti-PD-1 antibody with the anti-TIM-3 antibody, compared to single antibody treatment. The combination treatment also significantly inhibited tumor growth compared to vehicle treatment (p<0.0001) (FIG. 10).

### Example 10: In Vivo Efficacy of Combining Anti-PD-1 and Anti-TIM-3 Antibodies in a Human Xenograft Tumor Model

This example demonstrates enhanced in vivo efficacy of combining anti-PD-1 antibody 12819 with anti-TIM-3 antibody 15086.17145 in a human xenograft tumor model, where A375 cells (human melanoma) were engrafted in mice reconstituted with human PBMCs.

Human PBMCs were interperitoneally injected into NOG (Donor 1 and Donor 2) or NOG-EXL (hGM-CSF/hlL-3-NOG) (Donor 3) mice one day prior to subcutaneous engraftment of the human A375 melanoma cells. Treatment was initiated on the day of PBMC injection and the mice were treated three times weekly with a total of six treatments by intraperitoneal injection of vehicle buffer, anti-PD-1 antibody 12819, anti-TIM-3 antibody 15086.17145, or a combination of the anti-PD-1 and anti-TIM-3 antibodies. The antibody treatments were administered at a dose of 10 mg/kg/target. Tumors were measured three times weekly by caliper in two dimensions and tumor volume in mm³ was calculated per the formula: (width)²×length×0.5. Two-way ANOVA with Bonferroni's multiple comparisons test was applied to compare tumor volumes at each time-point between treatment groups. Statistical analyses were performed using GraphPad Prism version 5.0 (GraphPad Software, Inc.).

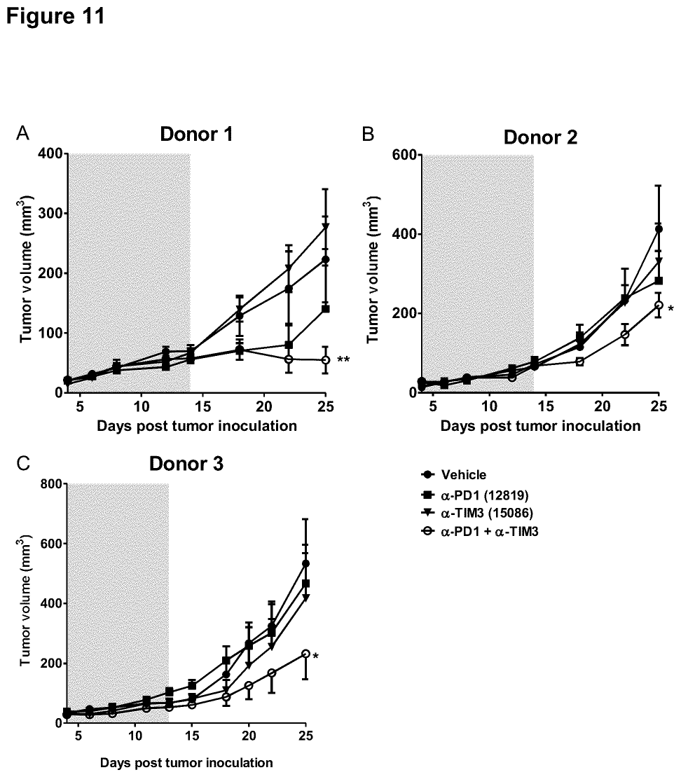

As shown in FIG. 11, treatment with the combination of the anti-PD-1 and anti-TIM-3 antibodies resulted in significant (and synergistic, in at least Donor 3) tumor growth delay (p<0.05 and p<0.01), whereas single antibody treatment (anti-PD-1 or anti-TIM-3) showed limited effect on tumor growth compared to the vehicle treated group.

### Example 11: Combined PD-1 and TIM-3 Targeting Leads to Increased Survival in Mice Co-Engrafted with Human Immune and Tumor Cells

This example demonstrates the in vivo efficacy of combining anti-PD-1 antibody 12819 with anti-TIM-3 antibody 15086.17145 in mouse models of human tumors.

2×10⁶ A375 (melanoma) cells were mixed with 2×10⁶ human PBMC and inoculated subcutaneously into the flank of 6-8 week old female NOD-scidmice. At the time of cell inoculation, treatment was initiated. The mice were treated three times weekly with a total of six treatments by intraperitoneal injection of vehicle buffer, anti-PD-1 antibody 12819, anti-TIM-3 antibody 15086.17145, or a combination of anti-PD-1 and anti-TIM-3. The antibody treatments were administered at a dose of 10 mg/kg for each antibody. Tumors were measured three times weekly by caliper in two dimensions and tumor volume in mm³ was calculated according to the formula: (width)²×length×0.5.

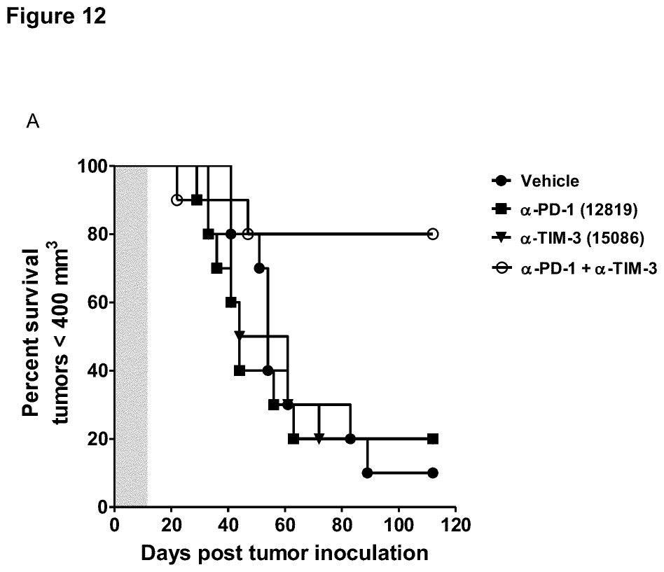

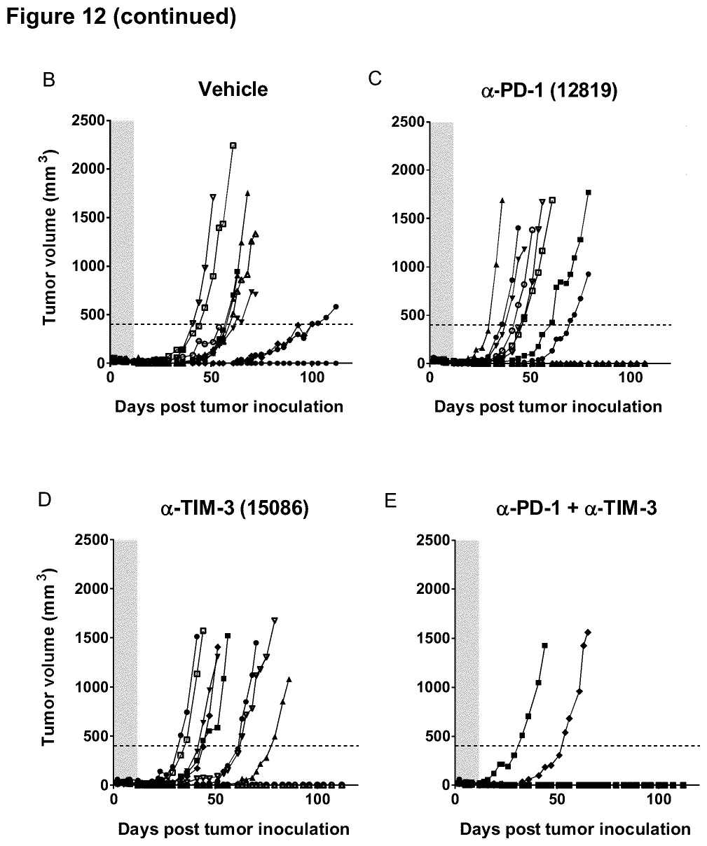

Survival was defined as having a tumor size<400 mm³. The results showed an increase in survival of mice treated with a dual combination of anti-PD-1 and anti-TIM-3 antibodies compared to any single antibody treatment (FIG. 12, Panels A-E)).

### Example 12: Combined PD-1, LAG-3, and TIM-3 Targeting Leads to Increased Survival in a Syngeneic Mouse Tumor Model

This example demonstrates the in vivo efficacy of combining anti-PD-1 antibody 12819 with anti-LAG-3 antibody C9B7W and anti-TIM-3 antibody 5D12 in a syngeneic mouse tumor model.

5×10⁶ ASB-XIV (lung carcinoma) cells were inoculated subcutaneously into the flank of 6-8 week old female BALB/cAnNRj mice. Tumors were measured three times weekly by caliper in two dimensions and tumor volume in mm³ was calculated according to the formula: (width)²×length×0.5. On day 5 post-inoculation, at an average tumor size of 50 mm³, mice were randomized into seven groups of ten animals and treatment was initiated. The mice were treated three times weekly with a total of six treatments by intraperitoneal injection of vehicle buffer, anti-PD-1 antibody 12819, anti-LAG-3 antibody C9B7W, anti-TIM-3 antibody 5D12, or a combination of anti-PD-1 and anti-LAG-3, anti-PD-1 and anti-TIM-3, or anti-PD-1, anti-LAG-3, and anti-TIM-3 antibodies. The antibody treatments were administered at a dose of 10 mg/kg for each antibody.

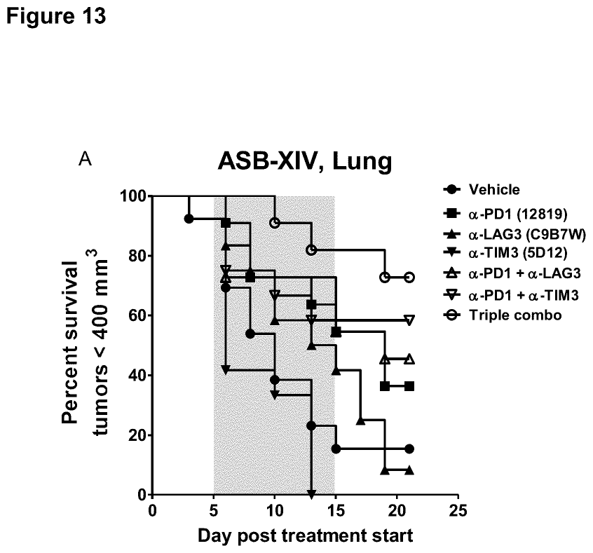

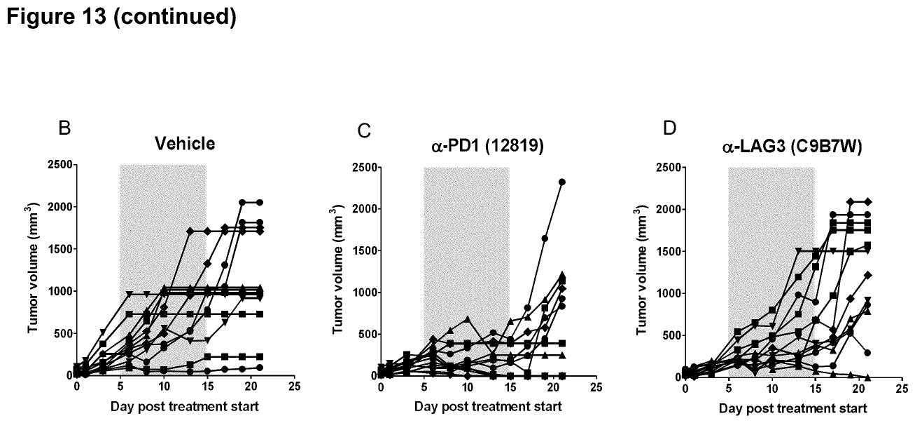

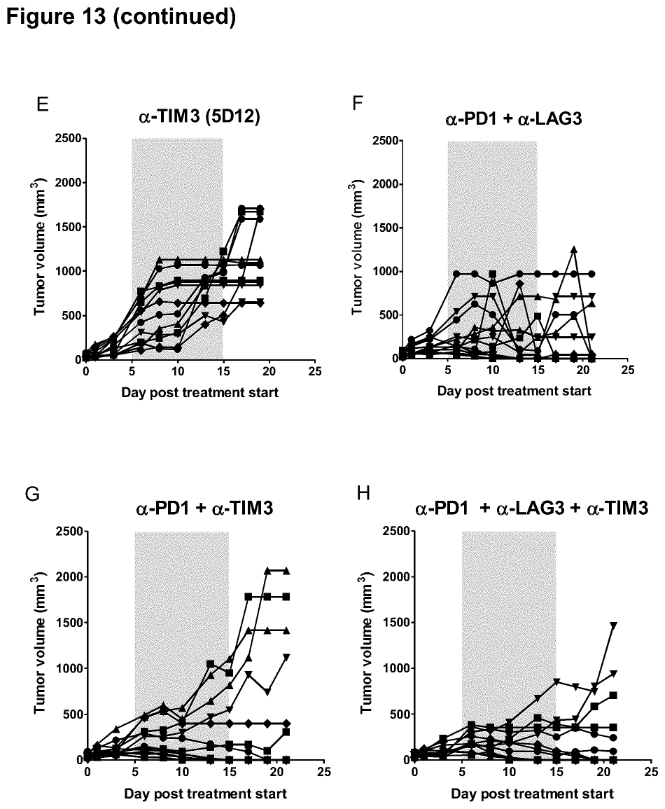

Survival was defined as having a tumor size<400 mm³. The results showed increased survival of mice treated with a triple combination of anti-PD-1, anti-LAG-3 and anti-TIM-3 antibodies compared to any single or dual antibody treatment (FIG. 13, Panels A-H).

TABLE 1
Antibody Sequence Identifiers

| | | DNA | | Protein | | | | | | | |
|---|---|---|---|---|---|---|---|---|---|---|---|
| Target | Ab | VH | VL | VH | VL | H-CDR1 | H-CDR2 | H-CDR3 | L-CDR1 | L-CDR2 | L-CDR3 |
| TIM-3 | 15086.15086 | 1 | 2 | 3 | 4 | 8 | 9 | 10 | 11 | 12 | 13 |
| | 15086.16837 | 5 | 6 | 7 | | | | | | | |
| | 15086.17145 | | | | | | | | | | |
| | 15086.17144 | | | | | | | | | | |
| | 15105 | 14 | 15 | 16 | 17 | 18 | 19 | 20 | 21 | 22 | 23 |
| | 15107 | 24 | 25 | 26 | 27 | 28 | 29 | 30 | 31 | 32 | 33 |
| | 15109 | 34 | 35 | 36 | 37 | 38 | 39 | 40 | 41 | 42 | 43 |
| | 15174 | 44 | 45 | 46 | 47 | 48 | 49 | 50 | 51 | 52 | 53 |
| | 15175 | 54 | 55 | 56 | 57 | 58 | 59 | 60 | 61 | 62 | 63 |
| | 15260 | 64 | 65 | 66 | 67 | 68 | 69 | 70 | 71 | 72 | 73 |
| | 15284 | 74 | 75 | 76 | 77 | 78 | 79 | 80 | 81 | 82 | 83 |
| | 15299 | 84 | 85 | 86 | 87 | 88 | 89 | 90 | 91 | 92 | 93 |
| | 15353 | 94 | 95 | 96 | 97 | 98 | 99 | 100 | 101 | 102 | 103 |
| | 15354 | 104 | 105 | 106 | 107 | 108 | 109 | 110 | 111 | 112 | 113 |
| | 17244 | 114 | 115 | 116 | 117 | 118 | 119 | 120 | 121 | 122 | 123 |
| | 17245 | 124 | 125 | 126 | 127 | 128 | 129 | 130 | 131 | 132 | 133 |
| | 19324 | 134 | 135 | 136 | 137 | 138 | 139 | 140 | 141 | 142 | 143 |
| | 19416 | 144 | 145 | 146 | 147 | 148 | 149 | 150 | 151 | 152 | 153 |
| | 19568 | 154 | 155 | 156 | 157 | 158 | 159 | 160 | 161 | 162 | 163 |
| | 20131 | 164 | 165 | 166 | 167 | 168 | 169 | 170 | 171 | 172 | 173 |
| | 20185 | 174 | 175 | 176 | 177 | 178 | 179 | 180 | 181 | 182 | 183 |
| | 20293 | 184 | 185 | 186 | 187 | 188 | 189 | 190 | 191 | 192 | 193 |
| | 20300 | 194 | 195 | 196 | 197 | 198 | 199 | 200 | 201 | 202 | 203 |
| | 20362 | 204 | 205 | 206 | 207 | 208 | 209 | 210 | 211 | 212 | 213 |
| | 20621 | 214 | 215 | 216 | 217 | 218 | 219 | 220 | 221 | 222 | 223 |
| PD-1 | 12819.15384 | 224 | 225 | 226 | 227 | 228 | 229 | 230 | 231 | 232 | 233 |
| | 12748.15381 | 234 | 235 | 236 | 237 | 238 | 239 | 240 | 241 | 242 | 243 |
| | 12865.15377 | 244 | 245 | 246 | 247 | 248 | 249 | 250 | 251 | 252 | 253 |
| | 12892.15378 | 254 | 255 | 256 | 257 | 258 | 259 | 260 | 261 | 262 | 263 |
| | 12796.15376 | 264 | 265 | 266 | 267 | 268 | 269 | 270 | 271 | 272 | 273 |
| | 12777.15382 | 274 | 275 | 276 | 277 | 278 | 279 | 280 | 281 | 282 | 283 |
| | 12760.15375 | 284 | 285 | 286 | 287 | 288 | 289 | 290 | 291 | 292 | 293 |
| | 13112.15380 | 294 | 295 | 296 | 297 | 298 | 299 | 300 | 301 | 302 | 303 |
| | 12748.16124 | 234 | 391 | 236 | 392 | 238 | 239 | 240 | 393 | 394 | 395 |
| LAG-3 | 15646 | 304 | 305 | 306 | 307 | 308 | 309 | 310 | 311 | 312 | 313 |
| | 15532 | 314 | 315 | 316 | 317 | 318 | 319 | 320 | 321 | 322 | 323 |
| | 15723 | 324 | 325 | 326 | 327 | 328 | 329 | 330 | 331 | 332 | 333 |
| | 15595 | 334 | 335 | 336 | 337 | 338 | 339 | 340 | 341 | 342 | 343 |
| | 15431 | 344 | 345 | 346 | 347 | 348 | 349 | 350 | 351 | 352 | 353 |
| | 15572 | 354 | 355 | 356 | 357 | 358 | 359 | 360 | 361 | 362 | 363 |
| | 15011 | 364 | 365 | 366 | 367 | 368 | 369 | 370 | 371 | 372 | 373 |

TABLE 2
Constant Region Sequences

| SEQ | | |
|---|---|---|
| ID | | |
| NO | Description | Sequence |
| 374 | IgG1 heavy chain | ASTKGPSVFPLAPSSKSTSGGTAALGCLVKDYFPEPVTVSWNSGALTSGVHTFPAVLQSSGLYSLSSVVTVPSS |
| | constant region | SLGTQTYICNVNHKPSNTKVDKRVEPKSCDKTHTCPPCPAPELLGGPSVFLFPPKPKDTLMISRTPEVTCVVVD |
| | protein sequence | VSHEDPEVKFNWYVDGVEVHNAKTKPREEQYNSTYRVVSVLTVLHQDWLNGKEYKCKVSNKALPAPIEKTISKA |
| | | KGQPREPQVYTLPPSREEMTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 375 | IgG1-LALA heavy | ASTKGPSVFPLAPSSKSTSGGTAALGCLVKDYFPEPVTVSWNSGALTSGVHTFPAVLQSSGLYSLSSVVTVPSS |
| | chain constant region | SLGTQTYICNVNHKPSNTKVDKRVEPKSCDKTHTCPPCPAPEAAGGPSVFLFPPKPKDTLMISRTPEVTCVVVD |
| | protein sequence | VSHEDPEVKFNWYVDGVEVHNAKTKPREEQYNSTYRVVSVLTVLHQDWLNGKEYKCKVSNKALPAPIEKTISKA |
| | | KGQPREPQVYTLPPSREEMTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 376 | IgG4 (S228P) heavy | ASTKGPSVFPLAPCSRSTSESTAALGCLVKDYFPEPVTVSWNSGALTSGVHTFPAVLQSSGLYSLSSVVTVPSS |
| | chain constant region | SLGTKTYTCNVDHKPSNTKVDKRVESKYGPPCPPCPAPEFLGGPSVFLFPPKPKDTLMISRTPEVTCVVVDVSQ |
| | protein sequence | EDPEVQFNWYVDGVEVHNAKTKPREEQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNYKTTPPVLDSDGSFFLYSRLTVDKSR |
| | | WQEGNVFSCSVMHEALHNHYTQKSLSLSLGK |
| 377 | IgG2 heavy chain | ASTKGPSVFPLAPCSRSTSESTAALGCLVKDYFPEPVTVSWNSGALTSGVHTFPAVLQSSGLYSLSSVVTVPSS |
| | constant region | NFGTQTYTCNVDHKPSNTKVDKTVERKCCVECPPCPAPPVAGPSVFLFPPKPKDTLMISRTPEVTCVVVDVSHE |
| | protein sequence | DPEVQFNWYVDGVEVHNAKTKPREEQFNSTFRVVSVLTVVHQDWLNGKEYKCKVSNKGLPAPIEKTISKTKGQP |
| | | REPQVYTLPPSREEMTKNQVSLTCLVKGFYPSDIAVEWESNGQPENNYKTTPPMLDSDGSFFLYSKLTVDKSRW |
| | | QQGNVFSCSVMHEALHNHYTQKSLSLSPGK |
| 378 | Kappa light chain | RTVAAPSVFIFPPSDEQLKSGTASVVCLLNNFYPREAKVQWKVDNALQSGNSQESVTEQDSKDSTYSLSSTLTL |
| | constant region | SKADYEKHKVYACEVTHQGLSSPVTKSFNRGEC |
| | protein sequence | |
| 379 | Lambda light chain | GQPKANPTVTLFPPSSEELQANKATLVCLISDFYPGAVTVAWKADGSPVKAGVETTKPSKQSNNKYAASSYLSL |
| | constant region | TPEQWKSHRSYSCQVTHEGSTVEKTVAPTECS |
| | protein sequence | |
| 380 | IgG1-LALA heavy | GCCTCCACCAAGGGCCCATCGGTCTTCCCCCTGGCACCCTCCTCCAAGAGCACCTCTGGGGGCACAGCGGCCCT |
| | chain constant region | GGGCTGCCTGGTCAAGGACTACTTCCCCGAACCGGTGACGGTGTCGTGGAACTCAGGCGCCCTGACCAGCGGCG |
| | DNA sequence | TGCACACCTTCCCGGCTGTCCTACAGTCCTCAGGACTCTACTCCCTCAGCAGCGTGGTGACCGTGCCCTCCAGC |
| | excluding introns | AGCTTGGGCACCCAGACCTACATCTGCAACGTGAATCACAAGCCCAGCAACACCAAGGTGGACAAGAGAGTTGA |
| | | GCCCAAATCTTGTGACAAAACTCACACATGCCCACCGTGCCCAGCACCTGAAGCCGCCGGGGGACCGTCAGTCT |
| | | TCCTCTTCCCCCCAAAACCCAAGGACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGTGGAGGTGCATAATGCCAAGACAAA |
| | | GCCGCGGGAGGAGCAGTACAACAGCACGTACCGTGTGGTCAGCGTCCTCACCGTCCTGCACCAGGACTGGCTGA |
| | | ATGGCAAGGAGTACAAGTGCAAGGTCTCCAACAAAGCCCTCCCAGCCCCCATCGAGAAAACCATCTCCAAAGCC |
| | | AAAGGGCAGCCCCGAGAACCACAGGTGTACACCCTGCCCCCATCCCGGGAGGAGATGACCAAGAACCAGGTCAG |
| | | CCTGACCTGCCTGGTCAAAGGCTTCTATCCCAGCGACATCGCCGTGGAGTGGGAGAGCAATGGGCAGCCGGAGA |
| | | ACAACTACAAGACCACGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCACAACCACTACACGCA |
| | | GAAGAGCCTCTCCCTGTCCCCGGGTAAA |
| 381 | IgG1-LALA heavy | GCCTCCACCAAGGGCCCATCGGTCTTCCCCCTGGCACCCTCCTCCAAGAGCACCTCTGGGGGCACAGCGGCCCT |
| | chain constant region | GGGCTGCCTGGTCAAGGACTACTTCCCCGAACCGGTGACGGTGTCGTGGAACTCAGGCGCCCTGACCAGCGGCG |
| | DNA sequence | TGCACACCTTCCCGGCTGTCCTACAGTCCTCAGGACTCTACTCCCTCAGCAGCGTGGTGACCGTGCCCTCCAGC |
| | including introns | AGCTTGGGCACCCAGACCTACATCTGCAACGTGAATCACAAGCCCAGCAACACCAAGGTGGACAAGAGAGTTGG |
| | | TGAGAGGCCAGCACAGGGAGGGAGGGTGTCTGCTGGAAGCCAGGCTCAGCGCTCCTGCCTGGACGCATCCCGGC |
| | | TATGCAGTCCCAGTCCAGGGCAGCAAGGCAGGCCCCGTCTGCCTCTTCACCCGGAGGCCTCTGCCCGCCCCACT |
| | | CATGCTCAGGGAGAGGGTCTTCTGGCTTTTTCCCCAGGCTCTGGGCAGGCACAGGCTAGGTGCCCCTAACCCAG |
| | | GCCCTGCACACAAAGGGGCAGGTGCTGGGCTCAGACCTGCCAAGAGCCATATCCGGGAGGACCCTGCCCCTGAC |
| | | CTAAGCCCACCCCAAAGGCCAAACTCTCCACTCCCTCAGCTCGGACACCTTCTCTCCTCCCAGATTCCAGTAAC |
| | | TCCCAATCTTCTCTCTGCAGAGCCCAAATCTTGTGACAAAACTCACACATGCCCACCGTGCCCAGGTAAGCCAG |
| | | CCCAGGCCTCGCCCTCCAGCTCAAGGCGGGACAGGTGCCCTAGAGTAGCCTGCATCCAGGGACAGGCCCCAGCC |
| | | GGGTGCTGACACGTCCACCTCCATCTCTTCCTCAGCACCTGAAgccgccGGGGGACCGTCAGTCTTCCTCTTCC |
| | | CCCCAAAACCCAAGGACACCCTCATGATCTCCCGGACCCCTGAGGTCACATGCGTGGTGGTGGACGTGAGCCAC |
| | | GAAGACCCTGAGGTCAAGTTCAACTGGTACGTGGACGGCGTGGAGGTGCATAATGCCAAGACAAAGCCGCGGGA |
| | | GGAGCAGTACAACAGCACGTACCGTGTGGTCAGCGTCCTCACCGTCCTGCACCAGGACTGGCTGAATGGCAAGG |
| | | AGTACAAGTGCAAGGTCTCCAACAAAGCCCTCCCAGCCCCCATCGAGAAAACCATCTCCAAAGCCAAAGGTGGG |
| | | ACCCGTGGGGTGCGAGGGCCACATGGACAGAGGCCGGCTCGGCCCACCCTCTGCCCTGAGAGTGACCGCTGTAC |
| | | CAACCTCTGTCCCTACAGGGCAGCCCCGAGAACCACAGGTGTACACCCTGCCCCCATCCCGGGAGGAGATGACC |
| | | AAGAACCAGGTCAGCCTGACCTGCCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGACGGCTCCTTCTTCCTCTATAGCA |
| | | AGCTCACCGTGGACAAGAGCAGGTGGCAGCAGGGGAACGTCTTCTCATGCTCCGTGATGCATGAGGCTCTGCAC |
| | | AACCACTACACGCAGAAGAGCCTCTCCCTGTCCCCGGGTAAA |
| 382 | IgG4 (S228P) heavy | GCTTCCACCAAGGGCCCATCGGTCTTCCCCCTGGCGCCCTGCTCCAGGAGCACCTCCGAGAGCACAGCCGCCCT |
| | chain constant region | GGGCTGCCTGGTCAAGGACTACTTCCCCGAACCGGTGACGGTGTCGTGGAACTCAGGCGCCCTGACCAGCGGCG |
| | DNA sequence | TGCACACCTTCCCGGCTGTCCTACAGTCCTCAGGACTCTACTCCCTCAGCAGCGTGGTGACCGTGCCCTCCAGC |
| | excluding introns | AGCTTGGGCACGAAGACCTACACCTGCAACGTAGATCACAAGCCCAGCAACACCAAGGTGGACAAGAGAGTTGA |
| | | GTCCAAATATGGTCCCCCATGCCCAcCATGCCCAGCACCTGAGTTCCTGGGGGGACCATCAGTCTTCCTGTTCC |
| | | CCCCAAAACCCAAGGACACTCTCATGATCTCCCGGACCCCTGAGGTCACGTGCGTGGTGGTGGACGTGAGCCAG |
| | | GAAGACCCCGAGGTCCAGTTCAACTGGTACGTGGATGGCGTGGAGGTGCATAATGCCAAGACAAAGCCGCGGGA |
| | | GGAGCAGTTCAACAGCACGTACCGTGTGGTCAGCGTCCTCACCGTCCTGCACCAGGACTGGCTGAACGGCAAGG |
| | | AGTACAAGTGCAAGGTCTCCAACAAAGGCCTCCCGTCCTCCATCGAGAAAACCATCTCCAAAGCCAAAGGGCAG |
| | | CCCCGAGAGCCACAGGTGTACACCCTGCCCCCATCCCAGGAGGAGATGACCAAGAACCAGGTCAGCCTGACCTG |
| | | CCTGGTCAAAGGCTTCTACCCCAGCGACATCGCCGTGGAGTGGGAGAGCAATGGGCAGCCGGAGAACAACTACA |
| | | AGACCACGCCTCCCGTGCTGGACTCCGACGGCTCCTTCTTCCTCTACAGCAGGCTCACCGTGGACAAGAGCAGG |
| | | TGGCAGGAGGGGAATGTCTTCTCATGCTCCGTGATGCATGAGGCTCTGCACAACCACTACACACAGAAGAGCCT |
| | | CTCCCTGTCTCTGGGTAAA |
| 383 | IgG4 (S228P) heavy | GCTTCCACCAAGGGCCCATCGGTCTTCCCCCTGGCGCCCTGCTCCAGGAGCACCTCCGAGAGCACAGCCGCCCT |
| | chain constant region | GGGCTGCCTGGTCAAGGACTACTTCCCCGAACCGGTGACGGTGTCGTGGAACTCAGGCGCCCTGACCAGCGGCG |
| | DNA sequence | TGCACACCTTCCCGGCTGTCCTACAGTCCTCAGGACTCTACTCCCTCAGCAGCGTGGTGACCGTGCCCTCCAGC |
| | including introns | AGCTTGGGCACGAAGACCTACACCTGCAACGTAGATCACAAGCCCAGCAACACCAAGGTGGACAAGAGAGTTGG |
| | | TGAGAGGCCAGCACAGGGAGGGAGGGTGTCTGCTGGAAGCCAGGCTCAGCCCTCCTGCCTGGACGCACCCCGGC |
| | | TGTGCAGCCCCAGCCCAGGGCAGCAAGGCAGGCCCCATCTGTCTCCTCACCCGGAGGCCTCTGACCACCCCACT |
| | | CATGCTCAGGGAGAGGGTCTTCTGGATTTTTCCACCAGGCTCCGGGCAGCCACAGGCTGGATGCCCCTACCCCA |
| | | GGCCCTGaGCATACAGGGGCAGGTGCTGCGCTCAGACCTGCCAAGAGCCATATCCGGGAGGACCCTGCCCCTGA |
| | | CCTAAGCCCACCCCAAAGGCCAAACTCTCCACTCCCTCAGCTCAGACACCTTCTCTCCTCCCAGATCTGAGTAA |
| | | CTCCCAATCTTCTCTCTGCAGAGTCCAAATATGGTCCCCCATGCCCAcCATGCCCAGGTAAGCCAACCCAGGCC |
| | | TCGCCCTCCAGCTCAAGGCGGGACAGGTGCCCTAGAGTAGCCTGCATCCAGGGACAGGCCCCAGCCGGGTGCTG |
| | | ACGCATCCACCTCCATCTCTTCCTCAGCACCTGAGTTCCTGGGGGGACCATCAGTCTTCCTGTTCCCCCCAAAA |
| | | CCCAAGGACACTCTCATGATCTCCCGGACCCCTGAGGTCACGTGCGTGGTGGTGGACGTGAGCCAGGAAGACCC |
| | | CGAGGTCCAGTTCAACTGGTACGTGGATGGCGTGGAGGTGCATAATGCCAAGACAAAGCCGCGGGAGGAGCAGT |
| | | TCAACAGCACGTACCGTGTGGTCAGCGTCCTCACCGTCCTGCACCAGGACTGGCTGAACGGCAAGGAGTACAAG |
| | | TGCAAGGTCTCCAACAAAGGCCTCCCGTCCTCCATCGAGAAAACCATCTCCAAAGCCAAAGGTGGGACCCACGG |
| | | GGTGCGAGGGCCACATGGACAGAGGTCAGCTCGGCCCACCCTCTGCCCTGGGAGTGACCGCTGTGCCAACCTCT |
| | | GTCCCTACAGGGCAGCCCCGAGAGCCACAGGTGTACACCCTGCCCCCATCCCAGGAGGAGATGACCAAGAACCA |
| | | GGTCAGCCTGACCTGCCTGGTCAAAGGCTTCTACCCCAGCGACATCGCCGTGGAGTGGGAGAGCAATGGGCAGC |
| | | CGGAGAACAACTACAAGACCACGCCTCCCGTGCTGGACTCCGACGGCTCCTTCTTCCTCTACAGCAGGCTCACC |
| | | GTGGACAAGAGCAGGTGGCAGGAGGGGAATGTCTTCTCATGCTCCGTGATGCATGAGGCTCTGCACAACCACTA |
| | | CACACAGAAGAGCCTCTCCCTGTCTCTGGGTAAA |
| 384 | IgG2 heavy chain | GCCTCCACCAAGGGCCCATCGGTCTTCCCCCTGGCGCCCTGCTCCAGGAGCACCTCCGAGAGCACAGCCGCCCT |
| | constant region DNA | GGGCTGCCTGGTCAAGGACTACTTCCCCGAACCGGTGACGGTGTCGTGGAACTCAGGCGCTCTGACCAGCGGCG |
| | sequence excluding | TGCACACCTTCCCAGCTGTCCTACAGTCCTCAGGACTCTACTCCCTCAGCAGCGTGGTGACCGTGCCCTCCAGC |
| | introns | AACTTCGGCACCCAGACCTACACCTGCAACGTAGATCACAAGCCCAGCAACACCAAGGTGGACAAGACAGTTGA |
| | | GCGCAAATGTTGTGTCGAGTGCCCACCGTGCCCAGCACCACCTGTGGCAGGACCGTCAGTCTTCCTCTTCCCCC |
| | | CAAAACCCAAGGACACCCTCATGATCTCCCGGACCCCTGAGGTCACGTGCGTGGTGGTGGACGTGAGCCACGAA |
| | | GACCCCGAGGTCCAGTTCAACTGGTACGTGGACGGCGTGGAGGTGCATAATGCCAAGACAAAGCCACGGGAGGA |
| | | GCAGTTCAACAGCACGTTCCGTGTGGTCAGCGTCCTCACCGTTGTGCACCAGGACTGGCTGAACGGCAAGGAGT |
| | | ACAAGTGCAAGGTCTCCAACAAAGGCCTCCCAGCCCCCATCGAGAAAACCATCTCCAAAACCAAAGGGCAGCCC |
| | | CGAGAACCACAGGTGTACACCCTGCCCCCATCCCGGGAGGAGATGACCAAGAACCAGGTCAGCCTGACCTGCCT |
| | | GGTCAAAGGCTTCTACCCCAGCGACATCGCCGTGGAGTGGGAGAGCAATGGGCAGCCGGAGAACAACTACAAGA |
| | | CCACACCTCCCATGCTGGACTCCGACGGCTCCTTCTTCCTCTACAGCAAGCTCACCGTGGACAAGAGCAGGTGG |
| | | CAGCAGGGGAACGTCTTCTCATGCTCCGTGATGCATGAGGCTCTGCACAACCACTACACGCAGAAGAGCCTCTC |
| | | CCTGTCTCCGGGTAAA |
| 385 | IgG2 heavy chain | GCCTCCACCAAGGGCCCATCGGTCTTCCCCCTGGCGCCCTGCTCCAGGAGCACCTCCGAGAGCACAGCCGCCCT |
| | constant region DNA | GGGCTGCCTGGTCAAGGACTACTTCCCCGAACCGGTGACGGTGTCGTGGAACTCAGGCGCTCTGACCAGCGGCG |
| | sequence including | TGCACACCTTCCCAGCTGTCCTACAGTCCTCAGGACTCTACTCCCTCAGCAGCGTGGTGACCGTGCCCTCCAGC |
| | introns | AACTTCGGCACCCAGACCTACACCTGCAACGTAGATCACAAGCCCAGCAACACCAAGGTGGACAAGACAGTTGG |
| | | TGAGAGGCCAGCTCAGGGAGGGAGGGTGTCTGCTGGAAGCCAGGCTCAGCCCTCCTGCCTGGACGCACCCCGGC |
| | | TGTGCAGCCCCAGCCCAGGGCAGCAAGGCAGGCCCCATCTGTCTCCTCACCCGGAGGCCTCTGCCCGCCCCACT |
| | | CATGCTCAGGGAGAGGGTCTTCTGGCTTTTTCCACCAGGCTCCAGGCAGGCACAGGCTGGGTGCCCCTACCCCA |
| | | GGCCCTTCACACACAGGGGCAGGTGCTTGGCTCAGACCTGCCAAAAGCCATATCCGGGAGGACCCTGCCCCTGA |
| | | CCTAAGCCGACCCCAAAGGCCAAACTGTCCACTCCCTCAGCTCGGACACCTTCTCTCCTCCCAGATCCGAGTAA |
| | | CTCCCAATCTTCTCTCTGCAGAGCGCAAATGTTGTGTCGAGTGCCCACCGTGCCCAGGTAAGCCAGCCCAGGCC |
| | | TCGCCCTCCAGCTCAAGGCGGGACAGGTGCCCTAGAGTAGCCTGCATCCAGGGACAGGCCCCAGCTGGGTGCTG |
| | | ACACGTCCACCTCCATCTCTTCCTCAGCACCACCTGTGGCAGGACCGTCAGTCTTCCTCTTCCCCCCAAAACCC |
| | | AAGGACACCCTCATGATCTCCCGGACCCCTGAGGTCACGTGCGTGGTGGTGGACGTGAGCCACGAAGACCCCGA |
| | | GGTCCAGTTCAACTGGTACGTGGACGGCGTGGAGGTGCATAATGCCAAGACAAAGCCACGGGAGGAGCAGTTCA |
| | | ACAGCACGTTCCGTGTGGTCAGCGTCCTCACCGTTGTGCACCAGGACTGGCTGAACGGCAAGGAGTACAAGTGC |
| | | AAGGTCTCCAACAAAGGCCTCCCAGCCCCCATCGAGAAAACCATCTCCAAAACCAAAGGTGGGACCCGCGGGGT |
| | | ATGAGGGCCACATGGACAGAGGCCGGCTCGGCCCACCCTCTGCCCTGGGAGTGACCGCTGTGCCAACCTCTGTC |
| | | CCTACAGGGCAGCCCCGAGAACCACAGGTGTACACCCTGCCCCCATCCCGGGAGGAGATGACCAAGAACCAGGT |
| | | CAGCCTGACCTGCCTGGTCAAAGGCTTCTACCCCAGCGACATCGCCGTGGAGTGGGAGAGCAATGGGCAGCCGG |
| | | AGAACAACTACAAGACCACACCTCCCATGCTGGACTCCGACGGCTCCTTCTTCCTCTACAGCAAGCTCACCGTG |
| | | GACAAGAGCAGGTGGCAGCAGGGGAACGTCTTCTCATGCTCCGTGATGCATGAGGCTCTGCACAACCACTACAC |
| | | GCAGAAGAGCCTCTCCCTGTCTCCGGGTAAA |
| 386 | Kappa light chain | CGTACGGTGGCTGCACCATCTGTCTTCATCTTCCCGCCATCTGATGAGCAGTTGAAATCTGGAACTGCCTCTGT |
| | constant region DNA | TGTGTGCCTGCTGAATAACTTCTATCCCAGAGAGGCCAAAGTACAGTGGAAGGTGGATAACGCCCTCCAATCGG |
| | sequence | GTAACTCCCAGGAGAGTGTCACAGAGCAGGACAGCAAGGACAGCACCTACAGCCTCAGCAGCACCCTGACGCTG |
| | | AGCAAAGCAGACTACGAGAAACACAAAGTCTACGCCTGCGAAGTCACCCATCAGGGCCTGAGCTCGCCCGTCAC |
| | | AAAGAGCTTCAACAGGGGAGAGTGT |
| 387 | Lambda light chain | CCTAGGTCAGCCCAAGGCCAACCCCACTGTCACTCTGTTCCCGCCCTCCTCTGAGGAGCTCCAAGCCAACAAGG |
| | constant region DNA | CCACACTAGTGTGTCTGATCAGTGACTTCTACCCGGGAGCTGTGACAGTGGCCTGGAAGGCAGATGGCAGCCCC |
| | sequence | GTCAAGGCGGGAGTGGAGACCACCAAACCCTCCAAACAGAGCAACAACAAGTACGCGGCCAGCAGCTACCTGAG |
| | | CCTGACGCCCGAGCAGTGGAAGTCCCACAGAAGCTACAGCTGCCAGGTCACGCATGAAGGGAGCACCGTGGAGA |
| | | AGACAGTGGCCCCTACAGAATGTTCATAA |

TABLE 3
Target Protein Sequences

| Protein | Amino Acid Sequence |
|---|---|
| Human PD-1 | MQIPQAPWPVVWAVLQLGWRPGWFLDSPDRPWNPPTFSPALLVVTEGDNATFTCSFSNTS |
| UniProt Q15116 | ESFVLNWYRMSPSNQTDKLAAFPEDRSQPGQDCRFRVTQLPNGRDFHMSVVRARRNDSGT |
| (SEQ ID NO: 388) | YLCGAISLAPKAQIKESLRAELRVTERRAEVPTAHPSPSPRPAGQFQTLVVGVVGGLLGS |
| | LVLLVWVLAVICSRAARGTIGARRTGQPLKEDPSAVPVFSVDYGELDFQWREKTPEPPVP |
| | CVPEQTEYATIVFPSGMGTSSPARRGSADGPRSAQPLRPEDGHCSWPL |
| Human TIM-3 | MFSHLPFDCVLLLLLLLLTRSSEVEYRAEVGQNAYLPCFYTPAAPGNLVPVCWGKGACPV |
| UniProt Q8TDQ0 | FECGNVVLRTDERDVNYWTSRYWLNGDFRKGDVSLTIENVTLADSGIYCCRIQIPGIMND |
| (SEQ ID NO: 389) | EKFNLKLVIKPAKVTPAPTRQRDFTAAFPRMLTTRGHGPAETQTLGSLPDINLTQISTLA |
| | NELRDSRLANDLRDSGATIRIGIYIGAGICAGLALALIFGALIFKWYSHSKEKIQNLSLI |
| | SLANLPPSGLANAVAEGIRSEENIYTIEENVYEVEEPNEYYCYVSSRQQPSQPLGCRFAM |
| | P |
| Human LAG-3 | MWEAQFLGLLFLQPLWVAPVKPLQPGAEVPVVWAQEGAPAQLPCSPTIPLQDLSLLRRAG |
| UniProt P18627 | VTWQHQPDSGPPAAAPGHPLAPGPHPAAPSSWGPRPRRYTVLSVGPGGLRSGRLPLQPRV |
| (SEQ ID NO: 390) | QLDERGRQRGDFSLWLRPARRADAGEYRAAVHLRDRALSCRLRLRLGQASMTASPPGSLR |
| | ASDWVILNCSFSRPDRPASVHWFRNRGQGRVPVRESPHHHLAESFLFLPQVSPMDSGPWG |
| | CILTYRDGFNVSIMYNLTVLGLEPPTPLTVYAGAGSRVGLPCRLPAGVGTRSFLTAKWTP |
| | PGGGPDLLVTGDNGDFTLRLEDVSQAQAGTYTCHIHLQEQQLNATVTLAIITVTPKSFGS |
| | PGSLGKLLCEVTPVSGQERFVWSSLDTPSQRSFSGPWLEAQEAQLLSQPWQCQLYQGERL |
| | LGAAVYFTELSSPGAQRSGRAPGALPAGHLLLFLILGVLSLLLLVTGAFGFHLWRRQWRP |
| | RRFSALEQGIHPPQAQSKIEELEQEPEPEPEPEPEPEPEPEPEQL |

TABLE 4
Anti-TIM-3 antibody heavy and light chain variable domain nucleotide
sequences

| Ab | Sequence (5′ to 3′) |
|---|---|
| 15086. | CAGGTGCAGCTACAGCAGTCGGGCCCAGGACTGGTGAAGCCTTCACAGACCCTGTCC |
| 15086 VH | CTCACCTGCACTGTCTCTGGTGGCTCCATCAGCAGTGGTGGTTACTACTGGAGTTGG |
| | ACCCGTCAGCACCCAGGGATGGGCCTGGAGTGGATTGGATACATCTCTTACAGTGGG |
| | AGTATCTATTACACTCCGTCCCTCAAGAGTCGACTTACCATATCAGTGGACACGTCT |
| | AAGAACCAGTTCTCCCTGAAGCTGAGCTCTGTGACTGCCGCGGACACGGCCGTATAT |
| | TACTGTGCGAGTTTGGATTCCTGGGGATCTAACCGTGACTACTGGGGCCAGGGAACC |
| | CTGGTCACCGTCTCGAGT (SEQ ID NO: 1) |
| 15086. | GAAATTGTGTTGACGCAGTCTCCAGCCACCCTGTCTTTGTCTCCAGGGGAAAGAGCC |
| 15086 VL | ACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCG |
| | CTCACTTTCGGCGGAGGGACCAAGGTGGAGATTAAG (SEQ ID NO: 2) |
| 15086. | CAGGTGCAGCTGCAGGAGAGTGGCCCCGGACTGGTCAAGCCTTCACAGACTCTGAGC |
| 16837/ | CTGACCTGCACAGTGTCTGGCGGAAGTATCAGCTCCGGGGGTTACTATTGGAGCTGG |
| 15086. | ACCCGACAGCACCCAGGAATGGGTCTGGAATGGATCGGGTACATTTCATATAGCGGC |
| 17145/ | TCCATCTACTATACACCCTCACTGAAAAGCAGGCTGACCATTTCCGTGGACACATCT |
| 15086. | AAGAACCAGTTCAGCCTGAAACTGTCTAGTGTGACAGCCGCTGATACTGCAGTCTAC |
| 17144 VH | TATTGTGCCTCCCTGGACTCTTGGGGCAGTAATAGAGATTACTGGGGCCAGGGAACT |
| | CTGGTCACCGTCTCGAGT (SEQ ID NO: 5) |
| 15086. | GAGATCGTGCTGACTCAGTCCCCAGCCACCCTGTCACTGAGCCCAGGAGAACGAGCA |
| 16837/ | ACCCTGTCTTGCAGGGCCTCCCAGTCTGTCAGCTCCTACCTGGCTTGGTATCAGCAG |
| 15086. | AAGCCCGGGCAGGCACCTCGACTGCTGATCTACGACGCCAGTAACAGAGCTACCGGT |
| 17145/ | ATTCCCGCCCGCTTCAGTGGTTCAGGCAGCGGAACAGACTTTACCCTGACAATCTCT |
| 15086. | AGTCTGGAGCCTGAAGATTTCGCCGTGTACTATTGTCAGCAGAGGTCTAATTGGCCA |
| 17144 VL | CTGACATTTGGCGGAGGGACTAAGGTCGAGATCAAG (SEQ ID NO: 6) |
| 15105 VH | CAGGTCACCTTGAAGGAGTGGGGCGCAGGACTGTTGAGGCCCTCGGAGACCCTGTCC |
| | CTCACCTGCGCTGTCTATGGTGGGTCCTTCAGTGGTTACTACTGGAGCTGGATCCGC |
| | CAGCCCCCAGGGAAGGGGCTGGAGTGGATAGGGGAAATCAATCATAGTGGAAGCACC |
| | AACTACAACCCGTCCCTCAAGAGTCGAGTCACCATATCAGTAGACGCGACCAAGAAA |
| | CAATTCTCCCTGAAGCTGACCTCTGTGACCGCCGCGGACACGGCTGTGTATTACTGT |
| | GCGAGATATTGGGAGCTCCCTGACTACTGGGGCCAGGGCACCCTGGTCACCGTCTCG |
| | AGT (SEQ ID NO: 14) |
| 15105 VL | GACATCCAGTTGACCCAGTCTCCATCCTCCCTGTCTGCATCTGTAGGAGACAGAGTC |
| | ACCATCACTTGCCGGGCAAGTCAGGGCATTAGAAATGATTTAGGCTGGTATCAGCAG |
| | AAACCAGGGAAAGCCCCTAAGCGCCTGATCTATGCTGCATCCAGTTTGCAAAGTGGG |
| | GTCCCATCAAGGTTCAGCGGCAGTGGATCTGGGACAGAATTCACTCTCACAATCAGC |
| | AGCCTGCAGCCTGAAGATTTTGCAACTTATTACTGTCTACAGCATAATAGTTACCCT |
| | CCGACGTTCGGCCAAGGGACCAAGGTGGAAATCAAG (SEQ ID NO: 15) |
| 15107 VH | CAGATGCAGCTGGTGCAGTGGGGCGCAGGACTGTTGAAGCCTTCGGAGACCCTGTCC |
| | CTCACCTGCGCTGTCTATGGTGGGTCCTTCAGTGGTTACTACTGGAGCTGGATCCGC |
| | CAGCCCCCAGGGAAGGGGCTGGAGTGGATTGGGGAAATCAATCATAGTGGAAGCACC |
| | AACTACAACCCGTCCCTCAAGAGTCGAGTCACCATGTCAGTTGACACGTCCAAGCAC |
| | CAGTTCTCCCTGAAGCTGAGCTCTGTGACCGCCGCGGACACGGCTGTGTATTACTGT |
| | GCGAGATGGTGGGAGCTTCCTGACTACTGGGGCCAGGGAACCCTGGTCACCGTCTCG |
| | AGT (SEQ ID NO: 24) |
| 15107 VL | GAAATTGTGTTGACGCAGTCTCCATCCTCCCTGTCTGCATCTGTAGGAGACAGAGTC |
| | ACCATCACTTGCCGGGCAAGTCAGGGCATTAGAAATGATTTAGGCTGGTATCAGCAG |
| | AAACCAGGGAAAGCCCCTAAGCGCCTGATCTATGCTGCATCCAGTTTGCAAAGTGGG |
| | GTCCCATCAAGGTTCAGCGGCAGTGGATCTGGGACAGAATTCACTCTCACAATCAGC |
| | AGCCTGCAGCCTGAAGATTTTGCAACTTATTACTGTCTACAGCATAATAGTTACCCG |
| | TGGACGTTCGGCCAAGGGACCAAGGTGGAAATCAAG (SEQ ID NO: 25) |
| 15109 VH | CAGATGCAGCTGGTGCAATGGGGCGCAGGACTGTTGAAGCCTTCGGAGACCCTGTCC |
| | CTCACCTGCGCTGTCTATGGTGGGTCCTTCAGTGGTTACTACTGGAGCTGGATCCGC |
| | CAGCCCCCAGGGAAGGGGCTGGAGTGGATTGGGGAAATCAATCATAGTGGAAGCACC |
| | AACTACAACCCGTCCCTCAAGAGTCGAGTCACCATATCAGTAGACACGTCCAAGAAC |
| | CAGTTCTCCCTGAAGCTGAGCTCTGTGACCGCCGCGGACACGGCTGTGTATTACTGT |
| | GCGAGGTTTTACTATGCTCCGAACTTTGACTACTGGGGCCAGGGCACCCTGGTCACC |
| | GTCTCGAGT (SEQ ID NO: 34) |
| 15109 VL | GAAATTGTGTTGACGCAGTCTCCTTCCACCCTGTCTGCATCTGTAGGAGACAGAGTC |
| | ACCATCACTTGCCGGGCCAGTCAGAGTATTAGTAGCTGGTTGGCCTGGTATCAGCAG |
| | AAACCAGGGACAGCCCCTAAGCTCCTGATCTATAAGGCGTCTAGTTTAGAAAGTGGG |
| | GTCCCATCAAGGTTCAGCGGCAGTGGATCTGGGACAGAATTCACTCTCACCATCAGC |
| | AGCCTGCAGCCTGATGATTTTGCAACTTATTACTGCCAACAGTATAATAGTTATTCC |
| | ACTTTCGGCGGAGGGACCAAGGTGGAGATCAAA (SEQ ID NO: 35) |
| 15174 VH | CAGGTGCAGCTGCAGCAGTCGGGCCCAGGACTGGTGAAGCCTTCGGAGACCCTGTCC |
| | CTCACCTGCACTGTCTCTGGTGGCTCCATCAGCAGTAGTAATTACTACTGGGGCTGG |
| | ATCCGCCAGCCCCCAGGGAAGGGGCTGGAGTGGATTGGGAGTATCTATTATAGTGGG |
| | AACACCTACTACAACCCGTCCCTCAAGAGTCGAGTCACCATATCCGTAGACACGTCC |
| | AAGAACCAGTTCTCCCTGAAGCTGAGTTCTGTGACCGCCGCAGACACGGCTGTGTAT |
| | TACTGTGCGAGACAGACAGTGGCTGGCCCCCTCTTTGACTACTGGGGCCAGGGAACC |
| | CTGGTCACCGTCTCGAGT (SEQ ID NO: 44) |
| 15174 VL | GAAATTGTGATGACGCAGTCTCCTTCCACCCTGTCTGCATCTGTAGGAGACAGAGTC |
| | ACCATCACTTGCCGGGCCAGTCAGAGTATTAGTAGCTGGTTGGCCTGGTATCAGCAG |
| | AAACCAGGGAAAGCCCCTAAGGTCCTGATCTATAAGGCGTCTAGTTTAGAAAGTGGG |
| | GTCCCATCAAGGTTCAGCGGCAGTGGATCTGGGACAGAACTCACTCTCACCATCAGC |
| | AGCCTGCAGCCTGATGATTTTGCAACTTATTACTGCCAACAGTATAATAGTTATTCA |
| | TTCACTTTCGGCCCTGGGACCAAAGTGGATATCAAG (SEQ ID NO: 45) |
| 15175 VH | CAGGTCCAGCTGGTGCAATCTGGGGCTGAGGTGAAGAAGCCTGGGGCCTCAGTGAAG |
| | GTCTCCTGCAAGGCTGCTGGATACACCTTAACCGGCTACTATATGCACTGGGTGCGA |
| | CAGGCCCCTGGACAAGGCCTTGAGTGGATGGGACGGATCAACCCTAACAGTGGTGGC |
| | TCAAACAATGCACAGAAGTTTCAGGGCAGGGTCACCATGACCAGGGACACGTCCATC |
| | AGCACAGCCTACATGGAGCTGAGCAGGCTGAGATCTGACGACACGGCCGTGTATTAC |
| | TGTGCGAGAGAGGGTCCCCTGTATAGCAGTGGCTGGTACGAGGGTGCTTTTGATATC |
| | TGGGGCCAAGGGACAATGGTCACCGTCTCGAGT (SEQ ID NO: 54) |
| 15175 VL | GAAATTGTGATGACGCAGTCTCCTTCCACCCTGTCTGCATCTGTAGGAGACAGAGTC |
| | ACCATCACTTGCCGGGCCAGTCAGAGTATTAGTAGTTGGTTGGCCTGGTATCAGCAG |
| | AAACCAGGGAAAGCCCCTAAGCTCCTGATCTATAAGGCGTCTAGTTTAGAAAGTGGG |
| | GTCCCATCAAGGTTCAGCGGCAGTGGATCTGGGACAGAATTCACTCTCACCATCAGC |
| | AGCCTGCAGCCTGATGATTTTGCAACTTATTACTGCCAACAGTATAATAGTTATTCT |
| | CCGGGGCTCACTTTCGGCGGAGGGACCAAGGTGGAGATCAAG (SEQ ID NO: |
| | 55) |
| 15260 VH | CAGATGCAGCTACAGCAGTCAGGTCCAGGACTGGTGAAGCCCTCGCAGACCCTCTCA |
| | CTCACCTGTGCCATCTCCGGGGACAGTGTCTCTAGCAACAGTGCTGCTTGGAACTGG |
| | ATCAGGCAGTCCCCATCGAGAGGCCTTGAGTGGCTGGGAAGGACATACTACAGGTCC |
| | AAGTGGTATTCTGCTTTTGCAGTATCTGTGAAAAGTCGAATAACCATCAACCCAGAC |
| | ACATCCAAGAACCAGTTCTCCCTGCAGCTGAACTCTGTGACTCCCGAGGACACGGCT |
| | GTGTATTACTGTGCAAGAGAGGGTAGCAGTGGCTGGTACGGATACGTCCACCACTGG |
| | GGCCAGGGCACCCTGGTCACCGTCTCGAGT (SEQ ID NO: 64) |
| 15260 VL | GAAATTGTGTTGACGCAGTCTCCAGCTTCCCTGTCTGTATCTCTGGGAGAAACTGTC |
| | ACCATCGAATGTCGAGCAAGTGAGGACATTTACAATGGTTTAGCATGGTATCAGCAG |
| | AAGCCAGGGAAATCTCCTCAGCTCCTGATCTATAATGCAAATAGCTTGCATACTGGG |
| | GTCCCATCACGGTTCAGTGGCAGTGGATCTGGTACACAGTATTCTCTCAAGATAAAC |
| | AGCCTGCAATCTGAAGATGTCGCAAGTTATTTCTGTCAACAGTATTACGATTATCCT |
| | CCGACGTTCGGCCAAGGGACCAAGGTGGAAATCAAA (SEQ ID NO: 65) |
| 15284 VH | CAGGTGCAGCTGCAGGAGTCGGGCCCAGGACTGGTGAAGCCTTCGGAGACCCTGTCC |
| | CTCACCTGCACTGTCTCTGGTGGCTCCTTCAGCAGTAGTAGTTACTACTGGGGCTGG |
| | ATCCGCCAGCCCCCTGGGAAGGGGCTGGAGTGGATTGGGATCTTCTATTATAGTGGG |
| | ACCACCTACTACAACCCGTCCCTCAAGAGTCGAGTCACCATATCCGCACACACGTCC |
| | AAGAGCCAGTTCTCCCTGAAGCTGAGCTCTGTGACCGCCGCAGACACGGCTGTGTAT |
| | TACTGTGCGAGAGGGGGAGAATATTTTGACCGGTTACTCCCCTTTGACTACTGGGGC |
| | CAGGGAACCCTGGTCACCGTCTCGAGT (SEQ ID NO: 74) |
| 15284 VL | GAAATTGTGATGACGCAGTCTCCATCCTTCCTGTCTGCATCTGTAGGAGACAGAGTC |
| | ACCATCACTTGCCGGGCCAGTCAGGGCATTAGCAGTTATTTAGCCTGGTATCAGCAA |
| | AAACCAGGGAAAGCCCCTAAGCTCCTGATCTATGCTGCATCCACTTTGGAAAGTGGG |
| | GTCCCATCAAGGTTCAGCGGCAGTGGATCTGGGACAGAATTCACTCTCACAATCAGC |
| | AGCCTGCAGCCTGAAGATTTTGCAACTTATTACTGTCAACAGCTTAATAGTTACCCA |
| | TTCACTTTCGGCCCTGGGACCAAAGTGGATATCAAA (SEQ ID NO: 75) |
| 15299 VH | CAGGTGCAGCTGGTGGAGTCTGGGGGAGGCTTGGTACAGCCGGGGGGGTCCCTGAGA |
| | CTCTCCTGTACAGCCTCTGGATTCACCTTTAGCAGCTATGCCATGAGCTGGGTCCGC |
| | CAGGCTCCAGGGAAGGGGCTGGAGTGGGTCTCAGCTATTGGTGGTAGTGGTGGTAGC |
| | ACATACTACGCAGACTCCGTGAAGGGCCGGTTCACCATCTCCAGAGACAATTCCAAG |
| | AACACGCTGTATCTGCAAATGAACAGCCTGAGAGCCGAGGACACGGCCGTATATTAC |
| | TGTGTGAAAGATGGGGCAGGAGGCTTTGACTACTGGGGCCAGGGAACCCTGGTCACC |
| | GTCTCGAGT (SEQ ID NO: 84) |
| 15299 VL | GATATTGTGATGACGCAGTCTTCATCCTCCCTGTCTGCATCTGTAGGAGACAGAGTC |
| | ACCATCACTTGCCGGGCAAGTCAGGGCATTATAAATCATTTAGGCTGGTATCAGCAT |
| | AAACCAGGGAAAGCCCCTAATCGCCTAATCTATGCTGCATCCAGTTTGCAAAGTGGG |
| | GTCCCATCAAGGTTCAGCGGCAGTGGATCTGGGACAGAATTCACTCTCACAATCAGC |
| | AGCCTGCAGCCTGAAGATTTTGCAACTTATTACTGTCTACGGCATAATAGTTACCCT |
| | CCGACGTTCGGCCAAGGGACCAAGGTGGAAATCAAG (SEQ ID NO: 85) |
| 15353 VH | CAGGTGCAGCTACAGCAGTCGGGCCCAGGACTGGTGAAGCCTTCACAGACCCTGTCC |
| | CTCACCTGCACTGTCTCTGGTGGCTCCATCAACAGTGGTGGTCACTACTGGAGCTGG |
| | ATCCGCCAGCACCCAGGGAGGGGCCTGGAGTGGATTGGGTACATCTATTACAGTGGG |
| | AGCATCTACTACAACCCGTCCCTCAAGAGTCGACTTACCATATCAGTAGACACGTCT |
| | AAGAACCAGTTCTCCCTGAAGCTGAGCTCTGTGACTGCCGCGGACACGGCCGTGTAT |
| | TACTGTGCGAGTTATTACTATGCCAGTAGTGGTGATGCTTTTGATATCTGGGGCCAA |
| | GGGACAATGGTCACCGTCTCGAGT |
| | (SEQ ID NO: 94) |
| 15353 VL | GAAACGACACTCACGCAGTCTCCAGCCACCCTGTCTTTGTCTCCAGGGGAAAGAGCC |
| | ACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCT |
| | CCGACGTTCGGCCAAGGGACCAAGGTGGAAATCAAA (SEQ ID NO: 95) |
| 15354 VH | CAGGTGCAGCTGCAGGAGTCTGGGGGAGGCTTGGTACAGCCGGGGGGGTCCCTGAGA |
| | CTCTCCTGTACAGCCTCTGGATTCACCTTTAGTAATTATGCCATGAGCTGGGTCCGC |
| | CAGGCTCCAGGGAAGGGGCTGGAGTGGGTCTCAGCTATTAGTGGTCGTGGTGGTAGC |
| | ACATTCTTCGCAGACTCCGTGAAGGGCCGGTTCACCATCTCCAGAGACAATTCCAAG |
| | AGCACGCTGTATCTGCAAACGAACAGCCTGAGAGCCGAGGACACGGCCGTATATTAC |
| | TGTGCGAAAGGGGGCCCGTTGTATAACTGGAACGACGGTGATGGTTTTGATATCTGG |
| | GGCCAAGGGACCACGGTCACAGTCTCGAGT (SEQ ID NO: 104) |
| 15354 VL | GAAATTGTGTTGACGCAGTCTCCAGCCACCCTGTCTGTGTCTCCAGGGGAAAGAGCC |
| | ACCCTCTCCTGCAGGGCCAGTCAGAGTGTTAGCAGCAACTTAGCCTGGTACCAGCAG |
| | AAACCTGGCCAGGCTCCCAGGCTCCTCATCTATGGTGCATCCACCAGGGCCACTGGT |
| | ATCCCAGCCAGGTTCAGTGGCACTGGGTCTGGGACAGAGTTCACTCTCACCATCAGC |
| | AGCCTGCAGTCTGAAGATTTTGCACTTTATTACTGTCAGCAGTATGATAACTGGCCT |
| | CCGTGGACGTTCGGCCAAGGGACCAAGGTGGAAATCAAA (SEQ ID NO: 105) |
| 17244 VH | CAGGTGCAGCTGCAGGAGTCTGGGGGAGGCTTGGTCAAGCCTGGAGGGTCCCTGAGA |
| | CTCTCCTGTGCAGCCTCTGGATTCACCTTCAGTGACTACTACATGACCTGGATCCGC |
| | CAGGCTCCAGGGAAGGGGCTGGAGTGGATTTCATACATTAGTGGTGGTGGTGGTTCC |
| | ATATACTACGCAGACTCTGTGAAGGGCCGATTCACCATCTCCAGGGACAACGCCAAG |
| | AACTCACTGTATCTGCAAATGAACAGCCTGAGAGCCGAGGACACGGCCGTTTATTTC |
| | TGTGCGAGAGGGAACTGGGGATCGGCGGCTCTTGATATCTGGGGCCAAGGGACAATG |
| | GTCACGGTCTCGAGT (SEQ ID NO: 114) |
| 17244 VL | GAAATTGTGTTGACGCAGTCTCCATCCTCACTGTCTGCATCTGTAGGAGACAGAGTC |
| | ACCATCACTTGTCGGGCGAGTCAGGGCATTAACAATTATTTAGCCTGGTTTCAGCAG |
| | AAACCAGGGAGAGCCCCTAAGTCCCTGATCTATGCTGCATCCAGTTTGCAAAGTGGG |
| | GTCCCATCGAAGTTCAGCGGCAGTGGATCTGGGACAGATTTCACTCTCACCATCAGC |
| | AGCCTGCAGCCTGAAGATTTTGCAACTTATTACTGCCAACAGTATAATAGTTACCCT |
| | CCAACTCTCGGCCCTGGGACCAACGTGGATATCAAA (SEQ ID NO: 115) |
| 17245 VH | CAGGTGCAGCTGGTGGAGTCTGGGGGAGGCTTGGTACAGCCGGGGGGGTCCCTGAGA |
| | CTCTCCTGTACAGCCTCTGGATTCACCTTTAGCAGCTATGCCATGAGCTGGGTCCGC |
| | CAGGCTCCAGGGAAGGGGCTGGAGTGGGTCTCAGCTATTGGTGGTAGTGGTGGTAGC |
| | GCATACTACGCAGACTCCGTGAAGGGCCGGTTCACCATCTCCAGAGACAATTCCAAG |
| | AACACGCTGTATCTGCAAATGAACAGCCTGAGAGCCGAGGACACGGCCGTATATTAC |
| | TGTGTGAAAGATGGGGCAGGAGGCTTTGACTACTGGGGCCAGGGCACCCTGGTCACC |
| | GTCTCGAGT (SEQ ID NO: 124) |
| 17245 VL | GACATCCAGTTGACCCAGTCCCCATCCTCCCTGTCTGCATCTGTAGGAGACAGAGTC |
| | ACCATCACTTGCCGGGCAAGTCAGGGCATTAGAAATCATTTAGGCTGGTATCAGCAG |
| | AAACCAGGGAAAGCCCCTAAGCGCCTAATCTATGCTGCATCCAGTTTGCAAAGTGGG |
| | GTCCCATCAAGGTTCAGCGGCAGTGGATCTGGGACAGAATTCACTCTCACAATCAGC |
| | AGCCTGCAGCCTGAAGATTTTGCAACTTATTACTGTCTACAGCATAATAGTTACCCT |
| | CCGACGTTCGGCCAAGGGACCAAGGTGGAAATCAAG (SEQ ID NO: 125) |
| 19324 VH | CAGATGCAGCTACAGCAGTCTGGGGGAGGCTTGGTACAGCCGGGGGGGTCCCTGAGA |
| | CTCTCCTGTGCAGCCTCTGGATTCACCGTTAGCAGCTATGCCATGAGCTGGGTCCGC |
| | CAGGCTCTAGGGAAGGGGCTGGAGTGGGTCTCAGGTATTAGTGGTAGTGGTGGTAGC |
| | ACATACTACGCAGACTCCGTGAAGGGCCGGTTCACCATCTCCAGAGACAATTCCAAG |
| | AATACGCTGTATCTGCAAATGAACAGCCTGAGAGCCGAGGACACGGCCGTATATTAC |
| | TGTGCGAAGATAGTGGGAGCTACCCACTTTGACTACTGGGGCCAGGGAACCCTGGTC |
| | ACGGTCTCGAGT (SEQ ID NO: 134) |
| 19324 VL | GAAATTGTGATGACACAGTCTCCAGACTCCCTGGCTGTGTCTCTGGGCGAGAGGGCC |
| | ACCATCAACTGCAAGTCCAGCCAGAGTGTTTTATACAGCTCCAACAATAAGAACTAC |
| | TTAGCTTGGTACCAGCAGAAACCAGGACAGCCTCCTAAGCTGCTCATTTACTGGGCA |
| | TCTACCCGGGAATCCGGGGTCCCTGACCGATTCAGTGGCAGCGGGTCTGGGACAGAT |
| | TTCACTCTCACCATCAGCAGCCTGCAGGCTGAAGATGTGGCAGTTTATTACTGTCAG |
| | CAATATTATAGTGGTCCGATCACCTTCGGCCAAGGGACACGACTGGAGATTAAG |
| | (SEQ ID NO: 135) |
| 19416 VH | CAGGTGCAGCTGGTGGAGTCGGGCCCAGGACTGGTGAAGCCTTCACAGACCCTGTCC |
| | CTCACCTGCACTGTCTCTGGTGGCTCCATCAACAGTGGTGGTTACTACTGGAGCTGG |
| | ATCCGCCAGCACCCAGGGAAGGGCCTGGAGTGGATTGGGTACATCTATTACAGTGGG |
| | AGCATCTACTACAACCCGTCCCTCAGGAGTCGACTTACCATATCAGTAGACACGTCT |
| | AAGAACCAGTTCTCCCTGAAGCTGAGCTCTGTGACTGCCGCGGACACGGCCGTTTAT |
| | TACTGTGCGACTCCTTATTACTATGGTTCGGGGAGTTATGGGGACTACTGGGGCCAG |
| | GGCACCCTGGTCACTGTCTCGAGT (SEQ ID NO: 144) |
| 19416 VL | GACATCCAGATGACCCAGTCTCCAGCCACCCTGTCTTTGTCTCCAGGGGAAAGAGCC |
| | ACCCTCTCCTGCAGGGCCAGTCAGAGTGTTAACAACTACTTAGCCTGGTACCAACAG |
| | AAACCTGGCCAGGCTCCCAGGCTCCTCATCTATGATGCATCCAACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCC |
| | ATCACCTTCGGCCAAGGGACACGACTGGAGATTAAA (SEQ ID NO: 145) |
| 19568 VH | CAGATGCAGCTGCAGGAGTCGGGCCCAGGACTGGTGAAGCCTTCTCAGACCCTGTCC |
| | CTCACCTGCACTGTGTCTGGTGGCTCCATCAGCAGTGTTGGTTACTACTGGAACTGG |
| | ATCCGCCAGCACCCAGGGAAGGGCCTGGAGTTCATTGGGTACATCTATTACAGTGGG |
| | AGCATCTACTACAATCCGTCCCTCAAGAGTCGAGTTACCATATCCGTAGACACGTCT |
| | AAGAACCAGTTCTCCCTGAAGCTGAGCTCTGTGACTGCCGCGGACACGGCCCTATAT |
| | TACTGTGCGAGCGTCGGTATAGTGGGAGCCTCCTACTTTGAGTACTGGGGCCAGGGA |
| | ACCCTGGTCACAGTCTCGAGT (SEQ ID NO: 154) |
| 19568 VL | GAAATTGTGATGACACAGTCTCCAGCCACCCTGTCTTTGTCTCCAGGGGAAAGAGCC |
| | ACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCT |
| | ATCACCTTCGGCCAAGGGACACGACTGGAGATCAAG (SEQ ID NO: 155) |
| 20131 VH | CAGGTGCAGCTACAGCAGTCTGGGGGAGGCTTGGTACAGCCTGGGGGGTCCCTGAGA |
| | CTCTCCTGTGCAGCCTCTGGATTCACCTTAAGCAGCTATGCCATGAGCTGGGTCCGC |
| | CAGGCTCCAGGGAAGGGGCTGGAGTGGGTCTCAGGTATTAGTGGTAGTGGTGGTAGC |
| | ACATACAACGCAGACTCCGTGAAGGGCCGGTTCACCATCTCCAGAGACAATTCCAAG |
| | AACACGCTGTTTCTGCAAATGAACAGCCTGAGAGCCGAGGACACGGCCGTATATTAC |
| | TGTGCGAAAATTTTTGGGTCCTACTACTTTGACTACTGGGGCCAGGGAACCCTGGTC |
| | ACAGTCTCGAGT (SEQ ID NO: 164) |
| 20131 VL | GAAATTGTGATGACACAGTCTCCAGACTCCCTGGCTGTGTCTCTGGGCGAGAGGGCC |
| | ACCATCAACTGCAAGTCCAGCCAGAGTGTTTTATACAGCTCCAACAATAAGAACTAC |
| | TTAGCTTGGTACCAGCAGAAACCAGGACAGCCTCCTAAGCTGCTCATTTACTGGACA |
| | TCTACCCGGGAATCCGGGGTCCCTAACCGATTCAGTGGCAGCGGGTCTGGGACAGAT |
| | TTCACTCTCACCATCAGCAGCCTGCAGGCTGAAGATGTGGCAGTTTATTACTGTCAG |
| | CAATATTATAGTGGTCCTCCGACGTTCGGCCAAGGGACCAAGGTGGAAATCAAA |
| | (SEQ ID NO: 165) |
| 20185 VH | CAGGTGCAGCTGGTGGAGTCTGGGGGAGGCTTGGTTCGGCCTGGGGGGTCCCTGAGA |
| | CTCTCCTGTGCAGTCTCTGGATTCACCTTTAGCAGCTATGCCATGAGCTGGGTCCGC |
| | CAGGCTCCAGGGAAGGGGCTGGAGTGGGTCTCAGGTATTAGTGGTAGTGGTGGTAGC |
| | ACATACAACGCAGACTCCGTGAAGGGCCGGTTCACCATCTCCAGAGACAATTCCAAG |
| | AACACGCTGTATCTTCAAATGAACAGCCTGAGAGCCGAGGACACGGCCGTATATTAC |
| | TGTGCGAAAATTTTTGGGTCCTACTACTTTGACTACTGGGGCCAGGGAACCCTGGTC |
| | ACCGTCTCGAGT (SEQ ID NO: 174) |
| 20185 VL | GAAATTGTGATGACACAGTCTCCAGACTCCCTGGCTGTGTCTCTGGGCGAGAGGGCC |
| | ACCATCAACTGCAAGTCCAGCCAGAGTGTTTTATACAGCTCCAATAATAAGAACTAC |
| | TTAGCTTGGTACCAGCAGAAATCAGGACAGCCTCCTAAGCTGCTCATTTACTGGGCA |
| | TCTACCCGGGAATCCGGGGTCCCTGACCGATTCAGTGGCAGCGGGTCTGGGACAGAT |
| | TTCACTCTCACCATCAGCAGCCTGCAGGCTGAAGATGTGGCAGTTTATTACTGTCAG |
| | CAATATTATAGTGGTCCACCGACGTTCGGCCAAGGGACCAAGGTGGAAATCAAA |
| | (SEQ ID NO: 175) |
| 20293 VH | ACGTGACAGGGCGCGCCCAGGTCCAGCTGCAGGAGAGCGGTCCCGGACTGGTGAAGC |
| | CATCCCAGACACTGAGCCTGACTTGTACTGTGAGCGGCGGTAGCATCTCCAGCGGCG |
| | GCTACTATTGGTCCTGGATCAGGCAGCACCCAGGCAAGGGCCTGGAGTGGATCGGCT |
| | ACATCTACTATAGCGGCTCTATCTACTATAACCCTTCCCTGAAGAGCCGGGTGACCA |
| | TCTCTGTGGACACATCCAAGAATCAGTTCTATCTGAAGCTGTCTTCCGTGACCGCCG |
| | CTGATACAGCCGTGTACTATTGCGCCTCACTGATGGTCTGGGGGGTCATGGGCGATT |
| | ACTGGGGGCAGGGCACACTGGTCACAGTCTCGAGT (SEQ ID NO: 184) |
| 20293 VL | GAGATTGTGCTGACCCAGTCTCCCGCCACCCTGTCTCTGAGTCCTGGCGAGAGAGCC |
| | ACCCTGAGCTGCAGAGCCTCTCAGTCCGTGTCCAGCTATCTGGCCTGGTATCAGCAG |
| | AAGCCCGGCCAGGCTCCCCGGCTGCTGATCTACGATGCCTCCAATAGAGCCACCGGC |
| | ATCCCTGCCAGATTCTCCGGCTCTGGCTCTGGCACCGACTTTACCCTGACCATCTCC |
| | AGCCTGGAACCCGAGGACTTCGCCGTGTACTACTGCCAGCAGCGGTCCGACTGGCCT |
| | CCTACATTTGGCCAAGGCACCAAGGTGGAAATCAAG (SEQ ID NO: 185) |
| 20300 VH | CAGGTCCAGCTACAGCAGTCTGGGGGAGGCTTGGTTCATCCTGGGGGGTCCCTAAGA |
| | CTCTCCTGTGCAGCCTCTGGATTCACCGTTGACACCTATGCCATGACCTGGGTCCGC |
| | CAGGCTCCAGGGAAGGGGCTGGAGTGGGTCTCAGGTATTAGCGGTAGTGGTGGTAGC |
| | ACATACTACGCAGACTCCGTGAAGGGCCGGTTCACCATCTCCAGAGACAACTCCAAG |
| | AACACGCTGTATCTGCAAATGAACAGCCTGAGAGACGAGGACACGGCCGTATATTAC |
| | TGTGCGAAGATAGTGGGAGTTACCCACTTTGACTACTGGGGCCAGGGCACCCTGGTC |
| | ACGGTCTCGAGT (SEQ ID NO: 194) |
| 20300 VL | GAAATTGTGATGACGCAGTCTCCAGACTCCCTGGCTGTGTCTCTGGGCGAGAGGGCC |
| | ACCATCAACTGCAAGTCCAGCCAGAGTGTTTTATACAGGTCCAACAATAAGAACTAT |
| | TTAGCTTGGTACCAGCAGAAACCAGGACAGCCTCCTAAGCTGCTCATTTACTGGGCA |
| | TCTACCCGGGAATCCGGGGTCCCTGACCGATTCAGTGGCAGCGGGTCTGGGACAGAT |
| | TTCACTCTCACCATCAGCAGCCTGCAGGCTGAAGATGTGGCAGTTTATTACTGTCAG |
| | CAATATTATAGTGGTCCGATCACCTTCGGCCAAGGGACACGACTGGAGATTAAG |
| | (SEQ ID NO: 195) |
| 20362 VH | CAGGTCACCTTGAAGGAGTCGGGCCCAGGACTGGTGAAGCCTTCACAGACCCTGTCC |
| | CTCACCTGCACTGTCTCTGGTGGCTCCATCAGCAGTGGTGGTCATTACTGGAGCTGG |
| | ATCCGCCAGCACCCAGGGAAGGGCCTGGAGTGGATTGGGTACATCTCTTACAGTGGG |
| | AGCACCTACTACAACCCGTCCCTCAAGAGTCGACTTACCATATCAGTAGACACGTCT |
| | AAGAACCAGTTCTCCCTGAAGCTGAGCTCTGTGACTGCCGCGGACACGGCCGTGTAT |
| | TACTGTGCGACCGCGTATTACGATATTTTGACTGGTTACCCTTTTGACTACTGGGGC |
| | CAGGGAACCCTGGTCACGGTCTCGAGT (SEQ ID NO: 204) |
| 20362 VL | GAAATTGTGATGACACAGTCTCCAGCCACCCTGTCTTTGTCTCCAGGGGAAAGAGCC |
| | ACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCGACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCG |
| | ATCACCTTCGGCCAAGGGACACGACTGGAGATCAAG (SEQ ID NO: 205) |
| 20621 VH | CAGGTGCAGCTACAGCAGTCGGGCCCAGGACTGGTGAAGCCTTCACAGACCCTGTCC |
| | CTCACCTGCACTGTCTCTGGTGGCTCCATCAGCAGTGGTGGTTACTACTGGAGCTGG |
| | ATCCGCCAGCACCCAGGGAAGGGCCTGGAGTGGATTGGGTACATCTCTTATAGTGGG |
| | AGTATCTACTACAACCCGTCCCTCAAGAGTCGAGTTACCATATCAGTAGACACGTCT |
| | AAGAACCAGTTCTCCCTGAAGCTGAGCTCTGTGACTGCCACGGACACGGCCGTGTAT |
| | TACTGTGCGACCGCGTATTACGATCTTTTGACTGGTTACCCTTTTGACTACTGGGGC |
| | CAGGGAACCCTGGTCACGGTCTCGAGT (SEQ ID NO: 214) |
| 20621 VL | GAAATTGTGATGACGCAGTCTCCAGCCACCCTGTCTTTGTCTCCAGGGGAAAGAGCC |
| | ACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGC |
| | ATCCCAGCCAGGTTCAGTGGCAGTGGGTCTGGGACAGACTTCACTCTCACCATCAGC |
| | AGCCTAGAGCCTGAAGATTTTGCAGTTTATTACTGTCAGCAGCGTAGCAACTGGCCG |
| | ATCACCTTCGGCCAAGGGACACGACTGGAGATTAAG (SEQ ID NO: 215) |

TABLE 5
Anti-TIM-3 antibody heavy and light chain variable domain amino acid
sequences

| Ab | Sequence (N-terminal to C-terminal) |
|---|---|
| 15086.15086 | QVQLQQSGPGLVKPSQTLSLTCTVS [symbol] WSWTRQHPGMGLEWIGY [symbol] |
| VH | [symbol] YYTPSLKSRLTISVDTSKNQFSLKLSSVTAADTAVYY [symbol] |
| | [symbol] GQGTLVTVSS (SEQ ID NO: 3) |
| 15086.16837 | QVQLQESGPGLVKPSQTLSLTCTVS [symbol] WSWTRQHPGMGLEWIGY [symbol] |
| 15086.17145 | [symbol] YYTPSLKSRLTISVDTSKNQFSLKLSSVTAADTAVYY [symbol] |
| 15086.17144 | [symbol] GQGTLVTVSS (SEQ ID NO: 7) |
| VH | |
| 15086.15086 | EIVLTQSPATLSLSPGERATLSCRAS [symbol] LAWYQQKPGQAPRLLIY [symbol] N |
| 15086.16837 | RATGIPARFSGSGSGTDFTLTISSLEPEDFAVYY [symbol] GGGTKVEI |
| 15086.17145 | K (SEQ ID NO: 4) |
| 15086.17144 | |
| VL | |
| 15105 VH | QVTLKEWGAGLLRPSETLSLTCAVY [symbol] WSWIRQPPGKGLEWIGE [symbol] |
| | [symbol] NYNPSLKSRVTISVDATKKQFSLKLTSVTAADTAVYY [symbol] G |
| | QGTLVTVSS (SEQ ID NO: 16) |
| 15105 VL | DIQLTQSPSSLSASVGDRVTITCRAS [symbol] LGWYQQKPGKAPKRLIY [symbol] S |
| | LQSGVPSRFSGSGSGTEFTLTISSLQPEDFATYY [symbol] GQGTKVEI |
| | K (SEQ ID NO: 17) |
| 15107 VH | QMQLVQWGAGLLKPSETLSLTCAVY [symbol] WSWIRQPPGKGLEWIGE [symbol] |
| | [symbol] NYNPSLKSRVTMSVDTSKHQFSLKLSSVTAADTAVYY [symbol] G |
| | QGTLVTVSS (SEQ ID NO: 26) |
| 15107 VL | EIVLTQSPSSLSASVGDRVTITCRAS [symbol] LGWYQQKPGKAPKRLIY [symbol] S |
| | LQSGVPSRFSGSGSGTEFTLTISSLQPEDFATYY [symbol] GQGTKVEI |
| | K (SEQ ID NO: 27) |
| 15109 VH | QMQLVQWGAGLLKPSETLSLTCAVY [symbol] WSWIRQPPGKGLEWIGE [symbol] |
| | [symbol] NYNPSLKSRVTISVDTSKNQFSLKLSSVTAADTAVYY [symbol] |
| | [symbol] WGQGTLVTVSS (SEQ ID NO: 36) |
| 15109 VL | EIVLTQSPSTLSASVGDRVTITCRAS [symbol] LAWYQQKPGTAPKLLIY [symbol] S |
| | LESGVPSRFSGSGSGTEFTLTISSLQPDDFATYY [symbol] GGGTKVEIK |
| | (SEQ ID NO: 37) |
| 15174 VH | QVQLQQSGPGLVKPSETLSLTCTVSGGSISSSNYYWGWIRQPPGKGLEWIGS [symbol] |
| | [symbol] YYNPSLKSRVTISVDTSKNQFSLKLSSVTAADTAVYY [symbol] |
| | [symbol] GQGTLVTVSS (SEQ ID NO: 46) |
| 15174 VL | EIVMTQSPSTLSASVGDRVTITCRAS [symbol] LAWYQQKPGKAPKVLIY [symbol] S |
| | LESGVPSRFSGSGSGTELTLTISSLQPDDFATYY [symbol] GPGTKVDI |
| | K (SEQ ID NO: 47) |
| 15175 VH | QVQLVQSGAEVKKPGASVKVSCKAA [symbol] MHWVRQAPGQGLEWMGR [symbol] |
| | [symbol] NNAQKFQGRVTMTRDTSISTAYMELSRLRSDDTAVYY [symbol] |
| | [symbol] GQGTMVTVSS (SEQ ID NO: 56) |
| 15175 VL | EIVMTQSPSTLSASVGDRVTITCRAS [symbol] LAWYQQKPGKAPKLLIY [symbol] S |
| | LESGVPSRFSGSGSGTEFTLTISSLQPDDFATYY [symbol] GGGTKV |
| | EIK (SEQ ID NO: 57) |
| 15260 VH | QMQLQQSGPGLVKPSQTLSLTCAIS [symbol] WNWIRQSPSRGLEWLGR [symbol] |
| | [symbol] [symbol] AFAVSVKSRITINPDTSKNQFSLQLNSVTPEDTAVYY [symbol] |
| | [symbol] GQGTLVTVSS (SEQ ID NO: 66) |
| 15260 VL | EIVLTQSPASLSVSLGETVTIECRAS [symbol] LAWYQQKPGKSPQLLIY [symbol] S |
| | LHTGVPSRFSGSGSGTQYSLKINSLQSEDVASYF [symbol] GQGTKVEI |
| | K (SEQ ID NO: 67) |
| 15284 VH | QVQLQESGPGLVKPSETLSLTCTVS [symbol] WGWIRQPPGKGLEWIGI [symbol] |
| | [symbol] YYNPSLKSRVTISAHTSKSQFSLKLSSVTAADTAVYY [symbol] |
| | [symbol] GQGTLVTVSS (SEQ ID NO: 76) |
| 15284 VL | EIVMTQSPSFLSASVGDRVTITCRAS [symbol] LAWYQQKPGKAPKLLIY [symbol] T |
| | LESGVPSRFSGSGSGTEFTLTISSLQPEDFATYY [symbol] GPGTKVDI |
| | K (SEQ ID NO: 77) |
| 15299 VH | QVQLVESGGGLVQPGGSLRLSCTAS [symbol] MSWVRQAPGKGLEWVSA [symbol] |
| | [symbol] YYADSVKGRFTISRDNSKNTLYLQMNSLRAEDTAVYY [symbol] |
| | [symbol] GQGTLVTVSS (SEQ ID NO: 86) |
| 15299 VL | DIVMTQSSSSLSASVGDRVTITCRAS [symbol] LGWYQHKPGKAPNRLIY [symbol] S |
| | LQSGVPSRFSGSGSGTEFTLTISSLQPEDFATYYCLRHNS [symbol] GQGTKVEI |
| | K (SEQ ID NO: 87) |
| 15353 VH | QVQLQQSGPGLVKPSQTLSLTCTVS [symbol] WSWIRQHPGRGLEWIGY [symbol] |
| | [symbol] YYNPSLKSRLTISVDTSKNQFSLKLSSVTAADTAVYY [symbol] |
| | [symbol] GQGTMVTVSS (SEQ ID NO: 96) |
| 15353 VL | ETTLTQSPATLSLSPGERATLSCRAS [symbol] LAWYQQKPGQAPRLLIY [symbol] N |
| | RATGIPARFSGSGSGTDFTLTISSLEPEDFAVYY [symbol] GQGTKVEI |
| | K (SEQ ID NO: 97) |
| 15354 VH | QVQLQESGGGLVQPGGSLRLSCTAS [symbol] MSWVRQAPGKGLEWVSA [symbol] |
| | [symbol] FFADSVKGRFTISRDNSKSTLYLQTNSLRAEDTAVYY [symbol] |
| | [symbol] GQGTTVTVSS (SEQ ID NO: 106) |
| 15354 VL | EIVLTQSPATLSVSPGERATLSCRAS [symbol] LAWYQQKPGQAPRLLIY [symbol] T |
| | RATGIPARFSGTGSGTEFTLTISSLQSEDFALYY [symbol] [symbol] GQGTKVE |
| | IK (SEQ ID NO: 107) |
| 17244 VH | QVQLQESGGGLVKPGGSLRLSCAAS [symbol] MTWIRQAPGKGLEW [symbol] |
| | [symbol] YYADSVKGRFTISRDNAKNSLYLQMNSLRAEDTAVYF [symbol] |
| | [symbol] GQGTMVTVSS (SEQ ID NO: 116) |
| 17244 VL | EIVLTQSPSSLSASVGDRVTITCRAS [symbol] LAWFQQKPGRAPKSLIY [symbol] S |
| | LQSGVPSKFSGSGSGTDFTLTISSLQPEDFATYY [symbol] GPGTNVDI |
| | K (SEQ ID NO: 117) |
| 17245 VH | QVQLVESGGGLVQPGGSLRLSCTAS [symbol] MSWVRQAPGKGLEWVSA [symbol] |
| | [symbol] YYADSVKGRFTISRDNSKNTLYLQMNSLRAEDTAVYY [symbol] |
| | [symbol] GQGTLVTVSS (SEQ ID NO: 126) |
| 17245 VL | DIQLTQSPSSLSASVGDRVTITCRAS [symbol] LGWYQQKPGKAPKRLIY [symbol] S |
| | LQSGVPSRFSGSGSGTEFTLTISSLQPEDFATYY [symbol] [symbol] GQGTKVEI |
| | K (SEQ ID NO: 127) |
| 19324 VH | QMQLQQSGGGLVQPGGSLRLSCAAS [symbol] MSWVRQALGKGLEWVSG [symbol] |
| | [symbol] YYADSVKGRFTISRDNSKNTLYLQMNSLRAEDTAVYY [symbol] |
| | [symbol] GQGTLVTVSS (SEQ ID NO: 136) |
| 19324 VL | EIVMTQSPDSLAVSLGERATINCKSS [symbol] LAWYQQKPGQPPKLL |
| | IY [symbol] TRESGVPDRFSGSGSGTDFTLTISSLQAEDVAVYY [symbol] GQ |
| | GTRLEIK (SEQ ID NO: 137) |
| 19416 VH | QVQLVESGPGLVKPSQTLSLTCTVS [symbol] WSWIRQHPGKGLEWIGY [symbol] |
| | [symbol] YYNPSLRSRLTISVDTSKNQFSLKLSSVTAADTAVYY [symbol] |
| | [symbol] GQGTLVTVSS (SEQ ID NO: 146) |
| 19416 VL | DIQMTQSPATLSLSPGERATLSCRAS [symbol] LAWYQQKPGQAPRLLIY [symbol] N |
| | RATGIPARFSGSGSGTDFTLTISSLEPEDFAVYY [symbol] GQGTRLEI |
| | K (SEQ ID NO: 147) |
| 19568 VH | QMQLQESGPGLVKPSQTLSLTCTVS [symbol] WNWIRQHPGKGLEFIGY [symbol] |
| | [symbol] YYNPSLKSRVTISVDTSKNQFSLKLSSVTAADTALYY [symbol] |
| | [symbol] GQGTLVTVSS (SEQ ID NO: 156) |
| 19568 VL | EIVMTQSPATLSLSPGERATLSCRAS [symbol] LAWYQQKPGQAPRLLIY [symbol] N |
| | RATGIPARFSGSGSGTDFTLTISSLEPEDFAVYY [symbol] GQGTRLEI |
| | K (SEQ ID NO: 157) |
| 20131 VH | QVQLQQSGGGLVQPGGSLRLSCAAS [symbol] MSWVRQAPGKGLEWVSG [symbol] |
| | [symbol] YNADSVKGRFTISRDNSKNTLFLQMNSLRAEDTAVYY [symbol] |
| | [symbol] GQGTLVTVSS (SEQ ID NO: 166) |
| 20131 VL | EIVMTQSPDSLAVSLGERATINCKSS [symbol] LAWYQQKPGQPPKLL |
| | IY [symbol] TRESGVPNRFSGSGSGTDFTLTISSLQAEDVAVYY [symbol] GQ |
| | GTKVEIK (SEQ ID NO: 167) |
| 20185 VH | QVQLVESGGGLVRPGGSLRLSCAVS [symbol] MSWVRQAPGKGLEWVSG [symbol] |
| | [symbol] YNADSVKGRFTISRDNSKNTLYLQMNSLRAEDTAVYY [symbol] |
| | [symbol] GQGTLVTVSS (SEQ ID NO: 176) |
| 20185 VL | EIVMTQSPDSLAVSLGERATINCKSS [symbol] LAWYQQKSGQPPKLL |
| | IY [symbol] TRESGVPDRFSGSGSGTDFTLTISSLQAEDVAVYY [symbol] GQ |
| | GTKVEIK (SEQ ID NO: 177) |
| 20293 VH | QVQLQESGPGLVKPSQTLSLTCTVS [symbol] WSWIRQHPGKGLEWIGY [symbol] I |
| | [symbol] YYNPSLKSRVTISVDTSKNQFYLKLSSVTAADTAVYY [symbol] |
| | [symbol] GQGTLVTVSS (SEQ ID NO: 186) |
| 20293 VL | EIVLTQSPATLSLSPGERATLSCRAS [symbol] LAWYQQKPGQAPRLLIY [symbol] N |
| | RATGIPARFSGSGSGTDFTLTISSLEPEDFAVYY [symbol] GQGTKVEI |
| | K (SEQ ID NO: 187) |
| 20300 VH | QVQLQQSGGGLVHPGGSLRLSCAAS [symbol] MTWVRQAPGKGLEWVSG [symbol] |
| | [symbol] YYADSVKGRFTISRDNSKNTLYLQMNSLRDEDTAVYY [symbol] |
| | [symbol] GQGTLVTVSS (SEQ ID NO: 196) |
| 20300 VL | EIVMTQSPDSLAVSLGERATINCKSS [symbol] LAWYQQKPGQPPKLL |
| | IY [symbol] TRESGVPDRFSGSGSGTDFTLTISSLQAEDVAVYY [symbol] GQ |
| | GTRLEIK (SEQ ID NO: 197) |
| 20362 VH | QVTLKESGPGLVKPSQTLSLTCTVS [symbol] WSWIRQHPGKGLEWIGY [symbol] |
| | [symbol] YYNPSLKSRLTISVDTSKNQFSLKLSSVTAADTAVYY [symbol] |
| | [symbol] GQGTLVTVSS (SEQ ID NO: 206) |
| 20362 VL | EIVMTQSPATLSLSPGERATLSCRAS [symbol] LAWYQQKPGQAPRLLIY [symbol] D |
| | RATGIPARFSGSGSGTDFTLTISSLEPEDFAVYY [symbol] GQGTRLEI |
| | K (SEQ ID NO: 207) |
| 20621 VH | QVQLQQSGPGLVKPSQTLSLTCTVS [symbol] WSWIRQHPGKGLEWIGY [symbol] |
| | [symbol] YYNPSLKSRVTISVDTSKNQFSLKLSSVTATDTAVYY [symbol] |
| | [symbol] GQGTLVTVSS (SEQ ID NO: 216) |
| 20621 VL | EIVMTQSPATLSLSPGERATLSCRAS [symbol] LAWYQQKPGQAPRLLIY [symbol] N |
| | RATGIPARFSGSGSGTDFTLTISSLEPEDFAVYY [symbol] GQGTRLEI |
| | K (SEQ ID NO: 217) |

(CDRs are bolded and italicized)

TABLE 6
Anti-PD-1 antibody heavy and light chain variable domain
nucleotide sequences

| Ab | Sequence (5′ to 3′) |
|---|---|
| 12819.15384 | GGCGCGCCGAGGTGCAGCTGCTGGAATCTGGAGGAGGACTGGTCCAGCCAGGT |
| VH | GGATCCCTGCGACTGAGCTGCGCCGCTTCTGGATTCACCTTTACAAGATACGA |
| | CATGGTGTGGGTCCGCCAGGCACCAGGAAAGGGACTGGAGTGGGTGGCTGGTA |
| | TCGGCGATAGTAACAAGATGACCCGCTACGCACCTGCCGTCAAAGGGAGGGCA |
| | ACAATTAGTCGGGACAACTCAAAGAATACTCTGTATCTGCAGATGAATTCCCT |
| | GCGAGCTGAGGATACAGCAGTGTACTATTGTGCCAAAGGTAGCTGCATCGCCT |
| | GTTGGGACGAAGCTGGCCGTATTGATGCATGGGGACAGGGGACTCTGGTGACC |
| | GTCTCGAG (SEQ ID NO: 224) |
| 12819.15384 | GCTAGCCTCTTACGAGCTGACTCAGGACCCTGCAGTGAGTGTCGCCCTGGGCC |
| VL | AGACAGTGAGAATCACTTGCTCCGGCGGAGGGAGCTACGATGGTTCCAGCTAC |
| | TATGGCTGGTATCAGCAGAAGCCAGGACAGGCACCTGTGACCGTCATCTATAA |
| | CAATAACAATAGGCCATCTGACATTCCCGATCGGTTCAGTGGATCTAGTTCAG |
| | GGAACACAGCTTCTCTGACCATTACAGGAGCCCAGGCTGAGGACGAAGCAGAT |
| | TACTATTGTGGGTCATACGACAGGCCAGAAACAAATTCCGATTATGTGGGAAT |
| | GTTTGGTAGCGGCACTAAAGTCACCGTCCTAGG (SEQ ID NO: 225) |
| 12748.15381 | GGCGCGCCGAGGTGCAGCTGCTGGAAAGCGGAGGAGGACTGGTCCAGCCAGGT |
| 12748.16124 | GGATCTCTGCGACTGAGTTGCGCCGCTTCAGGCTTCACATTTTCTGACTACGC |
| VH | CATGAACTGGGTGAGGCAGGCTCCTGGCAAGGGACTGGAGTGGGTCGCAGGAA |
| | TCGGGAACGATGGAAGTTACACTAATTATGGAGCAGCCGTGAAGGGGAGAGCT |
| | ACTATTTCCCGCGACAACAGCAAAAATACCCTGTACCTGCAGATGAACTCACT |
| | GAGAGCTGAAGATACCGCAGTGTACTATTGTGCCTCTGACATCAGGAGTCGGA |
| | ATGATTGCTCCTATTTCCTGGGAGGGTGTTCCAGCGGCTTTATTGACGTGTGG |
| | GGTCAGGGCACCCTGGTCACAGTCTCGAG (SEQ ID NO: 234) |
| 12748.15381 | GCTAGCCTCTTACGAGCTGACCCAGGACCCAGCAGTGTCCGTCGCCCTGGGCC |
| VL | AGACAGTGAGAATCACTTGCTCCGGCGGATCCAGCTACAGCTATGGGTGGTTC |
| | CAGCAGAAGCCCGGTCAGGCCCCTGTGACCGTCATCTATGAAAGTAACAATAG |
| | GCCATCAGACATTCCCGATCGGTTTTCTGGCTCTAGTTCAGGAAACACAGCTA |
| | GTCTGACCATCACAGGGGCCCAGGCTGAGGACGAAGCTGATTACTATTGTGGC |
| | AATGCAGATTCCAGCTCTGGAATTTTCGGGTCCGGTACTAAAGTCACCGTCCT |
| | AGG (SEQ ID NO: 235) |
| 12865.15377 | GGCGCGCCGAGGTGCAGCTGCTGGAATCCGGAGGAGGACTGGTCCAGCCAGGT |
| VH | GGATCCCTGCGACTGAGCTGCGCCGCTTCTGGATTCGACTTTAGCGATCACGG |
| | GATGCAGTGGGTGAGACAGGCACCAGGCAAGGGACTGGAGTACGTGGGTGTCA |
| | TCGACACCACAGGCCGCTATACATACTATGCACCTGCCGTCAAGGGCAGGGCT |
| | ACCATTAGTCGGGACAACTCAAAAAATACACTGTACCTGCAGATGAACTCTCT |
| | GAGGGCTGAAGATACTGCAGTGTACTATTGCGCCAAAACTACCTGCGTGGGAG |
| | GGTACCTGTGCAATACCGTCGGAAGTATCGATGCTTGGGGACAGGGGACACTG |
| | GTGACTGTCTCGAG (SEQ ID NO: 244) |
| 12865.15377 | GCTAGCCTCCTACGAGCTGACTCAGGACCCAGCAGTGAGCGTCGCCCTGGGCC |
| VL | AGACAGTGAGAATCACTTGCTCTGGCGGAGGGTCCAGCTCTTACTATGGTTGG |
| | TACCAGCAGAAGCCCGGCCAGGCTCCTGTGACCGTCATCTATGACGATACAAA |
| | CAGGCCAAGTGGAATTCCCGATCGGTTCTCAGGTAGTTCATCCGGCAATACAG |
| | CTTCTCTGACCATCACAGGGGCCCAGGCTGAGGACGAAGCAGATTACTATTGT |
| | GGTGGCTATGAAGGAAGCTCTCACGCCGGGATTTTTGGAAGTGGGACTAAAGT |
| | CACCGTCCTAGG (SEQ ID NO: 245) |
| 12892.15378 | GGCGCGCCGAGGTGCAGCTGCTGGAAAGTGGAGGAGGACTGGTCCAGCCAGGT |
| VH | GGAAGCCTGAGACTGTCTTGCGCCGCTAGTGGCTTCGACTTTTCCAGCTACAC |
| | CATGCAGTGGGTGAGGCAGGCACCAGGCAAGGGACTGGAGTGGGTGGGCGTCA |
| | TCTCTAGTACTGGAGGGTCTACCGGATACGGGCCTGCTGTGAAGGGAAGGGCA |
| | ACAATTTCACGGGATAACTCCAAAAATACTCTGTATCTGCAGATGAACAGCCT |
| | GAGGGCAGAAGACACAGCCGTGTACTATTGCGTGAAATCAATCTCCGGAGATG |
| | CCTGGTCTGTGGACGGGCTGGATGCTTGGGGTCAGGGCACCCTGGTCACAGTC |
| | TCGAG (SEQ ID NO: 254) |
| 12892.15378 | GCTAGCCTCATACGAGCTGACCCAGGACCCAGCAGTGTCCGTCGCCCTGGGAC |
| VL | AGACAGTGAGAATCACTTGCTCCGGAGGAGGATCCGCCTACGGTTGGTATCAG |
| | CAGAAGCCCGGCCAGGCACCTGTGACCGTCATCTACTATAACAATCAGAGGCC |
| | ATCTGGCATTCCCGACCGGTTCAGTGGATCCAGCTCTGGGAACACAGCAAGTC |
| | TGACCATCACAGGCGCCCAGGCTGAGGACGAAGCCGATTACTATTGTGGAAGC |
| | TATGATAGTTCAGCTGTGGGGATTTTTGGTTCTGGCACTAAAGTCACCGTCCT |
| | AGG (SEQ ID NO: 255) |
| 12796.15376 | GGCGCGCCGAGGTGCAGCTGCTGGAAAGTGGAGGAGGACTGGTCCAGCCAGGT |
| VH | GGAAGCCTGAGACTGTCTTGCGCCGCTAGTGGCTTCGACTTTTCCAGCTACAC |
| | CATGCAGTGGGTGAGGCAGGCACCAGGCAAGGGACTGGAGTGGGTGGGCGTCA |
| | TCTCTAGTACTGGAGGGTCTACCGGATACGGGCCTGCTGTGAAGGGAAGGGCA |
| | ACAATTTCACGGGATAACTCCAAAAATACTCTGTATCTGCAGATGAACAGCCT |
| | GAGGGCAGAAGACACAGCCGTGTACTATTGCGTGAAATCAGTCTCCGGAGATG |
| | CCTGGTCTGTGGACGGGCTGGATGCTTGGGGTCAGGGCACCCTGGTCACAGTC |
| | TCGAG (SEQ ID NO: 264) |
| 12796.15376 | GCTAGCCTCATACGAGCTGACCCAGGACCCAGCAGTGTCCGTCGCCCTGGGCC |
| VL | AGACAGTGAGAATCACTTGCTCCGGAGGAGGATCCGCCTACGGTTGGTATCAG |
| | CAGAAGCCCGGCCAGGCACCTGTGACCGTCATCTACTATAACAATCAGAGGCC |
| | ATCTGACATTCCCGATCGGTTCAGTGGATCCAGCTCTGGGAACACAGCAAGTC |
| | TGACCATCACAGGCGCCCAGGCTGAGGACGAAGCCGATTACTATTGTGGAAGC |
| | TATGATAGTTCAGCTGTGGGGATTTTTGGTTCTGGCACTAAAGTCACCGTCCT |
| | AGG (SEQ ID NO: 265) |
| 12777.15382 | GGCGCGCCGAGGTGCAGCTGCTGGAATCCGGAGGAGGACTGGTCCAGCCAGGT |
| VH | GGAAGCCTGCGACTGTCTTGCGCCGCTAGTGGATTCGACTTTTCCAGCTACGG |
| | AATGCAGTGGGTGAGGCAGGCACCAGGCAAGGGACTGGAGTGGGTGGGCGTCA |
| | TCTCTGGAAGTGGGATTACCACACTGTACGCACCTGCCGTCAAGGGAAGGGCT |
| | ACTATCTCACGGGACAACTCTAAAAATACAGTGTATCTGCAGATGAACTCCCT |
| | GAGAGCTGAAGATACCGCAGTCTACTATTGTACACGCTCACCCTCCATCACAG |
| | ACGGCTGGACTTATGGAGGGGCCTGGATTGATGCTTGGGGTCAGGGCACTCTG |
| | GTGACCGTCTCGAG (SEQ ID NO: 274) |
| 12777.15382 | GCTAGCCAGCTACGAGCTGACCCAGGACCCAGCAGTGTCCGTCGCCCTGGGCC |
| VL | AGACAGTGAGAATCACTTGCAGTGGCGGAGATGGGTCATACGGTTGGTTCCAG |
| | CAGAAGCCCGGACAGGCCCCTGTGACCGTCATCTATGACAACGATAATAGGCC |
| | ATCTGACATTCCCGATCGGTTTAGTGGCTCCAGCTCTGGAAACACAGCTTCTC |
| | TGACCATCACAGGGGCCCAGGCTGAGGACGAAGCTGATTACTATTGTGGCAAT |
| | GCAGACCTGTCCGGGGGTATTTTCGGCAGCGGAACTAAAGTCACCGTCCTAGG |
| | (SEQ ID NO: 275) |
| 12760.15375 | GGCGCGCCGAGGTGCAGCTGCTGGAATCTGGAGGAGGACTGGTCCAGCCAGGT |
| VH | GGATCCCTGAGACTGAGCTGCGCCGCTTCTGGATTCACCTTTAGTACATTCAA |
| | CATGGTGTGGGTCAGGCAGGCACCTGGAAAGGGACTGGAGTACGTGGCTGAAA |
| | TCTCCAGCGACGGCTCTTTTACATGGTATGCAACTGCCGTCAAGGGCAGGGCC |
| | ACCATTAGTCGGGATAACTCAAAAAATACAGTGTACCTGCAGATGAATTCCCT |
| | GAGGGCTGAGGACACCGCAGTCTACTATTGCGCAAAATCCGATTGTTCTAGTT |
| | CATACTATGGATATAGCTGTATCGGGATCATTGACGCTTGGGGTCAGGGCACT |
| | CTGGTGACCGTCTCGAG (SEQ ID NO: 284) |
| 12760.15375 | GCTAGCCTCCTATGAGCTGACCCAGGACCCAGCAGTGAGCGTCGCCCTGGGCC |
| VL | AGACAGTGAGAATCACTTGCTCCGGCGGAATTAGCGACGATGGCTCTTACTAT |
| | TACGGATGGTTCCAGCAGAAGCCCGGACAGGCCCCTGTGACCGTCATCTATAT |
| | TAACGACAGGCGGCCAAGTAATATCCCCGATAGGTTTTCAGGGTCCAGCTCTG |
| | GTAACACAGCTTCTCTGACCATTACAGGGGCCCAGGCTGAGGACGAAGCTGAT |
| | TATTACTGTGGCTCTTACGATAGTTCAGCAGGGGTGGGTATCTTCGGCAGTGG |
| | AACTAAAGTCACCGTCCTAGG (SEQ ID NO: 285) |
| 13112.15380 | GGCGCGCCGAGGTGCAGCTGCTGGAAAGTGGAGGAGGACTGGTCCAGCCAGGT |
| VH | GGATCACTGAGACTGTCCTGCGCCGCCTCCGGCTTCACCTTTTCCAGCTACAA |
| | CATGTTCTGGGTGCGCCAGGCACCAGGAAAGGGACTGGAGTTTGTCGCTGAAA |
| | TCTCTGGTAGTAATACTGGAAGCCGAACCTGGTACGCACCTGCCGTGAAGGGC |
| | AGGGCTACAATTTCTCGGGACAACAGTAAAAATACTCTGTATCTGCAGATGAA |
| | CTCTCTGAGGGCTGAGGATACAGCAGTGTACTATTGTGCAAAATCAATCTACG |
| | GAGGGTATTGCGCCGGTGGCTATTCCTGTGGTGTGGGCCTGATTGACGCATGG |
| | GGACAGGGGACCCTGGTCACAGTCTCGAG (SEQ ID NO: 294) |
| 13112.15380 | GCTAGCCTCATACGAGCTGACCCAGGACCCAGCAGTGTCCGTCGCCCTGGGCC |
| VL | AGACAGTGAGAATCACTTGCAGTGGCGGATCCAGCGATTACTATGGGTGGTTC |
| | CAGCAGAAGCCCGGTCAGGCCCCTGTGACCGTCATCTACTATAACAACAAGAG |
| | GCCATCTGACATTCCCGATCGGTTTAGTGGCTCTAGTTCAGGAAACACAGCCT |
| | CCCTGACCATTACAGGGGCCCAGGCTGAGGACGAAGCTGATTACTATTGTGGC |
| | AATGCAGACTCCAGCGTGGGAGTCTTCGGGTCTGGTACTAAGGTGACCGTCCT |
| | AGG (SEQ ID NO: 295) |
| 12748.16124 | GCTAGCCTCTTACGAGCTGACTCAGCCACCTTCCGTGTCCGTGTCCCCAGGAC |
| VL | AGACCGCAAGAATCACATGCAGTGGCGGATCCAGCTACTCATATGGGTGGTTC |
| | CAGCAGAAGCCTGGTCAGGCCCCCGTGACAGTCATCTATGAGAGCAACAATAG |
| | GCCTTCTGACATTCCAGAACGGTTTAGTGGCTCTAGTTCAGGAACCACAGTGA |
| | CTCTGACCATCAGCGGGGTCCAGGCCGAGGACGAAGCTGATTACTATTGTGGC |
| | AACGCTGATTCCAGCTCTGGAATTTTCGGGTCCGGTACAAAAGTGACTGTCCT |
| | AGG (SEQ ID NO: 391) |

TABLE 7
Anti-PD-1 antibody heavy and light chain variable domain amino acid
sequences

| Ab | Sequence (N-terminal to C-terminal) |
|---|---|
| 12819.15384 | EVQLLESGGGLVQPGGSLRLSCAAS [symbol] MVWVRQAPGKGLEWVAG [symbol] |
| VH | [symbol] RYAPAVKGRATISRDNSKNTLYLQMNSLRAEDTAVYY [symbol] |
| | [symbol] GQGTLVTVSS |
| | (SEQ ID NO: 226) |
| 12819.15384 | SYELTQDPAVSVALGQTVRITCSGG [symbol] YGWYQQKPGQAPVTVIY [symbol] |
| VL | NRPSDIPDRFSGSSSGNTASLTITGAQAEDEADYY [symbol] G |
| | SGTKVTVL |
| | (SEQ ID NO: 227) |
| 12748.15381 | EVQLLESGGGLVQPGGSLRLSCAAS [symbol] MNWVRQAPGKGLEWVAG [symbol] |
| 12748.16124 | [symbol] NYGAAVKGRATISRDNSKNTLYLQMNSLRAEDTAVYY [symbol] |
| VH | [symbol] GQGTLVTVSS |
| | (SEQ ID NO: 236) |
| 12748.15381 | SYELTQDPAVSVALGQTVRITCSGG [symbol] YGWFQQKPGQAPVTVIY [symbol] NRPS |
| VL | DIPDRFSGSSSGNTASLTITGAQAEDEADYY [symbol] GSGTKVTVL |
| | (SEQ ID NO: 237) |
| 12865.15377 | EVQLLESGGGLVQPGGSLRLSCAAS [symbol] MQWVRQAPGKGLEYVGV [symbol] |
| VH | [symbol] YYAPAVKGRATISRDNSKNTLYLQMNSLRAEDTAVYY [symbol] |
| | [symbol] GQGTLVTVSS |
| | (SEQ ID NO: 246) |
| 12865.15377 | SYELTQDPAVSVALGQTVRITCSGG [symbol] YGWYQQKPGQAPVTVIY [symbol] NRP |
| | SGIPDRFSGSSSGNTASLTITGAQAEDEADYY [symbol] GSGTKVTV |
| VL | L |
| | (SEQ ID NO: 247) |
| 12892.15378 | EVQLLESGGGLVQPGGSLRLSCAASG [symbol] MQWVRQAPGKGLEWVGV [symbol] |
| VH | [symbol] GYGPAVKGRATISRDNSKNTLYLQMNSLRAEDTAVYY [symbol] |
| | [symbol] GQGTLVTVSS |
| | (SEQ ID NO: 256) |
| 12892.15378 | SYELTQDPAVSVALGQTVRITCSGG [symbol] YGWYQQKPGQAPVTVIY [symbol] QRPSG |
| VL | IPDRFSGSSSGNTASLTITGAQAEDEADYY [symbol] GSGTKVTVL |
| | (SEQ ID NO: 257) |
| 12796.15376 | EVQLLESGGGLVQPGGSLRLSCAAS [symbol] MQWVRQAPGKGLEWVGV [symbol] |
| VH | [symbol] GYGPAVKGRATISRDNSKNTLYLQMNSLRAEDTAVYY [symbol] |
| | [symbol] GQGTLVTVSS |
| | (SEQ ID NO: 266) |
| 12796.15376 | SYELTQDPAVSVALGQTVRITCSGG [symbol] YGWYQQKPGQAPVTVIY [symbol] QRPSD |
| VL | IPDRFSGSSSGNTASLTITGAQAEDEADYY [symbol] GSGTKVTVL |
| | (SEQ ID NO: 267) |
| 12777.15382 | EVQLLESGGGLVQPGGSLRLSCAAS [symbol] MQWVRQAPGKGLEWVGV [symbol] |
| VH | [symbol] LYAPAVKGRATISRDNSKNTVYLQMNSLRAEDTAVYY [symbol] |
| | [symbol] GQGTLVTVSS |
| | (SEQ ID NO: 276) |
| 12777.15382 | SYELTQDPAVSVALGQTVRITCSGG [symbol] YGWFQQKPGQAPVTVIY [symbol] NRPSD |
| VL | IPDRFSGSSSGNTASLTITGAQAEDEADYY [symbol] GSGTKVTVL |
| | (SEQ ID NO: 277) |
| 12760.15375 | EVQLLESGGGLVQPGGSLRLSCAAS [symbol] MVWVRQAPGKGLEYVAE [symbol] |
| VH | [symbol] WYATAVKGRATISRDNSKNTVYLQMNSLRAEDTAVYY [symbol] |
| | [symbol] GQGTLVTVSS |
| | (SEQ ID NO: 286) |
| 12760.15375 | SYELTQDPAVSVALGQTVRITCSGG [symbol] YGWFQQKPGQAPVTVIY [symbol] |
| VL | RRPSNIPDRFSGSSSGNTASLTITGAQAEDEADYY [symbol] GSGTK |
| | VTVL |
| | (SEQ ID NO: 287) |
| 13112.15380 | EVQLLESGGGLVQPGGSLRLSCAAS [symbol] MFWVRQAPGKGLEFVAE [symbol] |
| VH | [symbol] WYAPAVKGRATISRDNSKNTLYLQMNSLRAEDTAVYY [symbol] |
| | [symbol] GQGTLVTVSS |
| | (SEQ ID NO: 296) |
| 13112.15380 | SYELTQDPAVSVALGQTVRITCSGG [symbol] YGWFQQKPGQAPVTVIY [symbol] KRPS |
| VL | DIPDRFSGSSSGNTASLTITGAQAEDEADYY [symbol] GSGTKVTVL |
| | (SEQ ID NO: 297) |
| 12748.16124 | SYELTQPPSVSVSPGQTARITCSGG [symbol] YGWFQQKPGQAPVTVIY [symbol] NRPS |
| VL | DIPERFSGSSSGTTVTLTISGVQAEDEADYY [symbol] GSGTKVTVL |
| | (SEQ ID NO: 392) |

(CDRs are bolded and italicized)

TABLE 8
Anti-LAG-3 antibody heavy and light chain variable domain
nucleotide sequences

| Ab | Sequence (5′ to 3′) |
|---|---|
| 15646 VH | CAGGTGCAGCTGCAGCAGTGGGGTGCCGGTCTGCTGAAGCCTTCTGAAACTCTGTC |
| | TCTGACTTGTGCCGTCTATGGTGGATCATTCAGCGGCTACTATTGGTCCTGGATCA |
| | GGCAGCCCCCTGGCAAGGGCCTGGAGTGGATCGGCGAGATCAACCACCGGGGCTCT |
| | ACCAACTACAATCCCTCTCTGAAGAGCAGGGTGACCATCTCCGTGGACACATCTAA |
| | GAATCAGTTCAGCCTGAAGCTGAGCTCCGTGACCGCCGCTGATACAGCCGTGTACT |
| | ATTGCACAAGGGGGGAGGAATGGGAGTCACTGTTCTTTGATTACTGGGGGCAGGGG |
| | ACACTGGTCACAGTCTCGAGT (SEQ ID NO: 304) |
| 15646 VL | GAAATCGTCCTGACCCAGTCCCCCGCCACCCTGAGCCTGAGCCCCGGAGAAAGAGC |
| | CACCCTGTCCTGCCGAGCAAGCCAGTCCATCAGCTCCTATCTCGCCTGGTATCAGC |
| | AGAAACCAGGCCAGGCTCCCCGGCTGCTGATCTACGGCGCCTCCAACAGAGCTACA |
| | GGAATCCCAGCCCGCTTCAGCGGCTCCGGCTCTGGCACAGACTTTACCCTGACAAT |
| | CTCTAGCCTGGAGCCTGAGGATTTCGCCGTGTACTATTGCCAGCAGAGATCTAATT |
| | GGCCACTGACATTCGGCGGCGGCACACGGGTGGAGATCAAG (SEQ ID NO: |
| | 305) |
| 15532 VH | CAGGTTCAGCTGCAGCAGTGGGGCGCCGGCCTGCTGAGACCAAGCGAGACCCTGTC |
| | CCTGACATGCGCCGTGTATGGCGAGAGCTTCTCCGGCTATTACTGGAACTGGATCC |
| | GGCAGCCTCCCGGCAAGGGCCTGGAGTGGATCGGCGAGATCAATCACTCCGGCTCC |
| | ACCAATTACAACCCATCCCTGAAGTCTCGGGTGACAATCAGCGTGGATACAAGCAA |
| | GACCCAGTTCAGCCTGAAGCTGAGCTCCGTGACAGCTGCCGATACCGCCGTGTATT |
| | ACTGCGCCAGAGGCTGGGACCTGCTGGATTGGAATGACTACTGGAATGAGTACTGG |
| | GGCCAGGGGACCCTGGTGACCGTCTCGAGT (SEQ ID NO: 314) |
| 15532 VL | GAGATCGTGCTGACCCAGTCCCCTGCCACCCTGTCTCTGTCCCCTGGCGAGCGGGC |
| | CACCCTGTCCTGTAGAGCTTCTCAGTCCGTGTCTTCCTACCTGGCTTGGTACCAGC |
| | AGAAGCCAGGACAGGCCCCAAGACTGCTGATCTATGACGCTTCCAATCGGGCTACC |
| | GGCATCCCAGCTCGCTTTAGCGGCTCCGGCTCCGGCACCGACTTCACCCTGACAAT |
| | CAGCTCCCTGGAGCCAGAGGATTTTGCCGTGTATTACTGTCAGCAGAGGTCCAATT |
| | GGCCACTGACATTTGGCGGCGGCACAAAGGTTGAGATCAAG (SEQ ID NO: |
| | 315) |
| 15723 VH | CAGGTTCAGCTGCAGCAGTGGGGCGCTGGCCTGCTGAAGCCCTCTGAGACCCTGTC |
| | TCTGACCTGTGCCGTGTATGGCGGCAGCTTCTCCGGCTATTACTGGAACTGGATCC |
| | GCCAGCCCCCCGGCAAGGGCCTGGAGTGGATCGGCGAGATCAACCACTCCGGCTCT |
| | ACCAACTACAATCCTTCTCTGAAGTCCAGGGTGACAATCAGCGTGGACACCAGCAA |
| | GAACCAGTTTAGCCTGAAGCTGTCCAGCGTGACAGCTGCCGATACAGCCGTGTATT |
| | ACTGCGCCAGAGGCGAGGATTGGGGCGAGAGCTTCTTTGATTACTGGGGCCAGGGG |
| | ACCCTGGTGACCGTCTCGAGT (SEQ ID NO: 324) |
| 15723 VL | GAGATCGTGCTGACCCAGTCCCCTGCCACCCTGTCTCTGTCCCCTGGCGAGCGGGC |
| | CACCCTGAGCTGTCGGGCCTCCCAGTCCGTGAGCTCCTACCTGGCTTGGTATCAGC |
| | AGAAGCCAGGACAGGCCCCAAGACTGCTGATCTACGCCGCTTCCAATCGGGCCACC |
| | GGCATCCCCGCCAGATTTTCCGGCTCTGGCTCCGGCACCGATTTCACCCTGACAAT |
| | CAGCTCCCTGGAGCCAGAGGACTTCGCCGTGTACTACTGCCAGCAGAGGTCTAATT |
| | GGCCACTGACATTTGGCGGCGGCACCAAGGTTGAGATCAAG (SEQ ID NO: |
| | 325) |
| 15595 VH | CAGGTTCAGCTGGTGCAGTCCGGCGCTGAGGTGAAGAAGCCTGGAGCTTCTGTGAA |
| | GGTTTCCTGTAAGGTTTCCGGCTATAGCCTGACCGAGATCTCTATGCACTGGGTAC |
| | GGCAAGCCCCCGGCAAGGGCCTGGAGTGGATGGGCGGCTTTGACCCAGAGGATGGC |
| | GAGACCATCTACGCTCAGAGGTTTCAGGGGCGCGTGATCATGACCGAGGATACCAG |
| | CACCGATACCGCCTACATGGAGCTGTCCAGCCTGAGATCCGAGGATACCGCCGTGT |
| | ATTACTGCGCTACTGGTGGCTGGGGCCCCAATTGGTTCGATCCTTGGGGCCAGGGG |
| | ACCCTGGTGACCGTCTCGAGT (SEQ ID NO: 334) |
| 15595 VL | GACATCCAGATGACACAGTCTCCTTCCTCTGTGAGCGCCTCTGTGGGCGACCGCGT |
| | GACCATCACATGCCGCGCTTCCCAGGGGATCTCTTCCTGGCTGGCTTGGTACCAGC |
| | AGAAGCCCGGCAAGGCCCCCAAGCTGCTGATCTATGCTGCCAGCAGCCTGCAGTCC |
| | GGCGTGCCTTCCAGGTTTAGCGGCTCCGGCTCCGGCACCGACTTTACACTGACAAT |
| | CAGCTCCCTGCAGCCCGAGGATTTTGCCACCTATTACTGTCAGCAGGCGAATTCCT |
| | TCCCTTTTACATTCGGCCCTGGCACCAAGGTTGATATCAAG (SEQ ID NO: |
| | 335) |
| 15431 VH | CAGGTTCAGCTGCAGCAGTCCGGCCCCGGCCTGGTGAAGCCAAGCCAGACCCTGTC |
| | TCTGACATGCGCCATCTCCGGCGACAGCGTGTCCTCTAACTCCGCCGCTTGGAATT |
| | GGATCCGGCAGTCTCCATCCAGAGGCCTGGAGTGGCTGGGCAGAACCTATTACCGG |
| | TCCAAGTGGTACAACGATTATGCCGTGTCCGTGAAGAGCAGAATCACCATCAACCC |
| | TGATACCAGCAAGAACCAGTTCAGCCTGCAGCTGAATTCCGTGACCCCAGAGGATA |
| | CAGCCGTGTATTACTGTGCTAGGGACGATGATTGGAATGACTTCGATTACTGGGGC |
| | CAGGGGACCCTGGTGACAGTCTCGAGT (SEQ ID NO: 344) |
| 15431 VL | GAGATCGTGCTGACCCAGTCCCCAGCTACACTGTCCCTGTCTCCCGGCGAGCGGGC |
| | CACCCTGAGCTGTAGAGCTTCCCAGTCCGTGTCTTCCTATCTGGCTTGGTATCAGC |
| | AGAAGCCAGGACAGGCCCCAAGGCTGCTGATCTACGACGCCTCCAATAGAGCCACC |
| | GGCATCCCAGCTAGATTTTCTGGCTCCGGCTCCGGCACCGATTTCACACTGACCAT |
| | CTCTAGCCTGGAGCCAGAGGATTTTGCTGTATATTACTGCCAGCAGCGCAGCAACT |
| | GGCCCCTGACATTTGGCGGCGGCACCAAGGTTGAGATCAAG (SEQ ID NO: |
| | 345) |
| 15572 VH | CAGCTGCAGCTGCAGGAAAGCGGCCCCGGCCTGGTGAAGCCCTCTGAGACCCTGTC |
| | CCTGACATGCACCGTGAGCGGCGATTCCATCAGCTCTTCCAGCTATTACTGGGGCT |
| | GGATCCGGCAGCCCCCCGGCAAGGGCCTGGAGTGGATCGGCAGCATCTTCTACTCC |
| | GGCAATACATATTATAATCCTTCTCTGAAGAGCAGGGTGACAATCAGCGTGGATAC |
| | CTCCAAGAATCAGTTTAGCCTGAAGCTGAGCTCCGTGACAGCTGCCGATACAGCCG |
| | TGTATTACTGCGCTAGGGAGGACGATTTTCTGACCGATTATTACGGCGCTTTCGAC |
| | ATCTGGGGCCAGGGGACAATGGTGACAGTCTCGAGT (SEQ ID NO: 354) |
| 15572 VL | GATATCCAGATGACCCAGTCTCCAAGCACCCTGAGCGCCTCTGTGGGCGATCGGGT |
| | GACCATCACATGTCGGGCTTCTCAGTCCATCAGCAGCTGGCTGGCTTGGTATCAGC |
| | AGAAGCCCGGCAAGGCCCCAAAGCTGCTGATCTACAAGGCCTCTTCCAGCGAGAGC |
| | GGCGTGCCATCCAGGTTTAGCGGCTCCGGCTCCGGCACCGAGTTTACCCTGACCAT |
| | CTCTTCCCTGCAGCCCGATGACTTTGCCACCTACTACTGTCAGCAGTACAATTCCT |
| | ATCTGACATTCGGCGGCGGCACCAAGGTTGAGATCAAG (SEQ ID NO: 355) |
| 15011 VH | GAGGTGCAGCTGCTGGAATCCGGAGGAGGACTGGTCCAGCCAGGTGGATCCCTGCG |
| | ACTGAGCTGCGCCGCTTCTGGCTTCGACTTTAGAAGCTACGCAATGATGTGGGTCC |
| | GCCAGGCACCAGGAAAGGGACTGGAGTGGGTGGGAGGGATCAACGGTGAAGTCGGT |
| | GGCTCTAATACATACTATGCACCTGCCGTCAAGGGAAGGGCTACTATTAGTCGGGA |
| | CAACTCAAAAAATACCCTGTATCTGCAAATGAACAGTCTGAGGGCCGAGGATACCG |
| | CCGTGTACTATTGCGTGAAAGGTGCTGGCGCATGCGGCATCTGTAATGACGATATT |
| | GATGCATGGGGACAGGGGACCCTGGTGACAGTCTCGAGT (SEQ ID NO: |
| | 364) |
| 15011 VL | AGTTATGAGCTGACTCAGGACCCAGCAGTGTCAGTCGCCCTGGGCCAGACAGTGAG |
| | AATCACTTGCAGTGGGGCTGGTTCATATGCAGGCTCCTACTATTACGGATGGCACC |
| | AGCAGAAGCCCGGACAGGCACCTGTGACAGTCATCTACGACAACGATAAAAGGCCA |
| | AGCAATATTCCCGACCGGTTCTCTGGGTCCAGCTCTGGTAACACCGCCTCCCTGAC |
| | CATTACTGGGGCCCAGGCTGAGGACGAAGCTGATTATTACTGTGGCTCTACAAACG |
| | ATAATGACGATGGCGGACTGTTTGGCTCCGGAACTAAGGTCACCGTCCTA (SEQ |
| | ID NO: 365) |

TABLE 9
Anti-LAG-3 antibody heavy and light chain variable
domain amino acid sequences

| Ab | Sequence (N-terminal to C-terminal) |
|---|---|
| 15646 VH | QVQLQQWGAGLLKPSETLSLTCAVY [symbol] WSWIRQPPGKGLEWIGE [symbol] |
| | NYNPSLKSRVTISVDTSKNQFSLKLSSVTAADTAVYY [symbol] GQGTL |
| | VTVSS |
| | (SEQ ID NO: 306) |
| 15646 VL | EIVLTQSPATLSLSPGERATLSCRAS [symbol] LAWYQQKPGQAPRLLIY [symbol] NRATG |
| | IPARFSGSGSGTDFTLTISSLEPEDFAVYY [symbol] SNWPLTFGGGTRVEIK |
| | (SEQ ID NO: 307) |
| 15532 VH | QVQLQQWGAGLLRPSETLSLTCAVY [symbol] WNWIRQPPGKGLEWIGE [symbol] |
| | NYNPSLKSRVTISVDTSKTQFSLKLSSVTAADTAVYY [symbol] GQ |
| | GTLVTVSS |
| | (SEQ ID NO: 316) |
| 15532 VL | EIVLTQSPATLSLSPGERATLSCRAS [symbol] LAWYQQKPGQAPRLLIY [symbol] NRATG |
| | IPARFSGSGSGTDFTLTISSLEPEDFAVYY [symbol] GGGTKVEIK |
| | (SEQ ID NO: 317) |
| 15723 VH | QVQLQQWGAGLLKPSETLSLTCAVY [symbol] WNWIRQPPGKGLEWIGE [symbol] |
| | NYNPSLKSRVTISVDTSKNQFSLKLSSVTAADTAVYY [symbol] GQGTL |
| | VTVSS |
| | (SEQ ID NO: 326) |
| 15723 VL | EIVLTQSPATLSLSPGERATLSCRAS [symbol] LAWYQQKPGQAPRLLIY [symbol] NRATG |
| | IPARFSGSGSGTDFTLTISSLEPEDFAVYY [symbol] GGGTKVEIK |
| | (SEQ ID NO: 327) |
| 15595 VH | QVQLVQSGAEVKKPGASVKVSCKVS [symbol] MHWVRQAPGKGLEWMGG [symbol] |
| | [symbol] IYAQRFQGRVIMTEDTSTDTAYMELSSLRSEDTAVYY [symbol] GQGTL |
| | VTVSS |
| | (SEQ ID NO: 336) |
| 15595 VL | DIQMTQSPSSVSASVGDRVTITCRAS [symbol] LAWYQQKPGKAPKLLIY [symbol] SLQSG |
| | VPSRFSGSGSGTDFTLTISSLQPEDFATYY [symbol] GPGTKVDIK |
| | (SEQ ID NO: 337) |
| 15431 VH | QVQLQQSGPGLVKPSQTLSLTCAIS [symbol] AWNWIRQSPSRGLEWLGR [symbol] |
| | [symbol] YNDYAVSVKSRITINPDTSKNQFSLQLNSVTPEDTAVYY [symbol] GQG |
| | TLVTVSS |
| | (SEQ ID NO: 346) |
| 15431 VL | EIVLTQSPATLSLSPGERATLSCRAS [symbol] LAWYQQKPGQAPRLLIY [symbol] NRATG |
| | IPARFSGSGSGTDFTLTISSLEPEDFAVYY [symbol] GGGTKVEIK |
| | (SEQ ID NO: 347) |
| 15572 VH | QLQLQESGPGLVKPSETLSLTCTVS [symbol] WGWIRQPPGKGLEWIGS [symbol] |
| | [symbol] YYNPSLKSRVTISVDTSKNQFSLKLSSVTAADTAVYY [symbol] |
| | GQGTMVTVSS |
| | (SEQ ID NO: 356) |
| 15572 VL | DIQMTQSPSTLSASVGDRVTITCRAS [symbol] LAWYQQKPGKAPKLLIY [symbol] SSESG |
| | VPSRFSGSGSGTEFTLTISSLQPDDFATYY [symbol] GGGTKVEIK |
| | (SEQ ID NO: 357) |
| 15011 VH | EVQLLESGGGLVQPGGSLRLSCAAS [symbol] MWVRQAPGKGLEWVGG [symbol] |
| | [symbol] YYAPAVKGRATISRDNSKNTLYLQMNSLRAEDTAVYY [symbol] |
| | [symbol] GQGTLVTVSS |
| | (SEQ ID NO: 366) |
| 15011 VL | SYELTQDPAVSVALGQTVRITCSGA [symbol] YYGWHQQKPGQAPVTVIY [symbol] KRPS |
| | NIPDRFSGSSSGNTASLTITGAQAEDEADYY [symbol] GSGTKVTVL |
| | (SEQ ID NO: 367) |

(CDRs are bolded and italicized)

1-53. (canceled)

54. A method of enhancing immunity in a human patient in need thereof, comprising administering to the patient
an anti-PD-1 antibody or an antigen-binding portion thereof that competes for binding to human PD-1 with, or binds to the same epitope of human PD-1 as, an antibody comprising the heavy and light chain variable domain amino acid sequences of:
a) SEQ ID NOs: 226 and 227, respectively;
b) SEQ ID NOs: 236 and 237, respectively;
c) SEQ ID NOs: 236 and 392, respectively;
d) SEQ ID NOs: 246 and 247, respectively;
e) SEQ ID NOs: 256 and 257, respectively;
f) SEQ ID NOs: 266 and 267, respectively;
g) SEQ ID NOs: 276 and 277, respectively;
h) SEQ ID NOs: 286 and 287, respectively; or
i) SEQ ID NOs: 296 and 297, respectively; and
an anti-TIM-3 antibody or an antigen-binding portion thereof, an anti-LAG-3 antibody or an antigen-binding portion thereof, or both.

55. The method of claim 54, wherein the anti-PD-1 antibody has at least one of the following properties:
a) binds to human PD-1 with a K_{D} of 750 pM or less;
b) binds to cynomolgus PD-1 with a K_{D} of 7 nM or less;
c) binds to mouse PD-1 with a K_{D} of 1 nM or less;
d) does not bind to rat PD-1;
e) increases IL-2 secretion in a Staphylococcal enterotoxin B (SEB) whole blood assay;
f) increases IFN-γ secretion in a one-way mixed lymphocyte reaction assay;
g) inhibits the interaction of PD-1 with PD-L1 by at least 60% at a concentration of 10 μg/mL in a flow cytometric competition assay;
h) blocks binding of PD-L1 and PD-L2 to PD-1 by at least 90% at a concentration of 10 μg/mL as determined by Bio-Layer Interferometry analysis; and
i) inhibits tumor growth in vivo.

56. The method of claim 54, wherein the anti-PD-1 antibody comprises:
a) heavy chain complementarity-determining regions (H-CDR) 1-3 and light chain complementarity-determining regions (L-CDR) 1-3 comprising the amino acid sequences of:
i) SEQ ID NOs: 228-233, respectively;
ii) SEQ ID NOs: 238-243, respectively;
iii) SEQ ID NOs: 248-253, respectively;
iv) SEQ ID NOs: 258-263, respectively;
v) SEQ ID NOs: 268-273, respectively;
vi) SEQ ID NOs: 278-283, respectively;
vii) SEQ ID NOs: 288-293, respectively; or
viii) SEQ ID NOs: 298-303, respectively;
b) a heavy chain variable domain (VH) and a light chain variable domain (VL) comprising the amino acid sequences of:
i) SEQ ID NOs: 226 and 227, respectively;
ii) SEQ ID NOs: 236 and 237, respectively;
iii) SEQ ID NOs: 236 and 392, respectively;
iv) SEQ ID NOs: 246 and 247, respectively;
v) SEQ ID NOs: 256 and 257, respectively;
vi) SEQ ID NOs: 266 and 267, respectively;
vii) SEQ ID NOs: 276 and 277, respectively;
viii) SEQ ID NOs: 286 and 287, respectively; or
ix) SEQ ID NOs: 296 and 297, respectively; or
c) a heavy chain (HC) and a light chain (LC) that:
i) comprise the amino acid sequences of SEQ ID NOs: 226 and 375, and SEQ ID NOs: 227 and 379, respectively;
ii) comprise the amino acid sequences of SEQ ID NOs: 236 and 375, and SEQ ID NOs: 237 and 379, respectively;
iii) comprise the amino acid sequences of SEQ ID NOs: 236 and 375, and SEQ ID NOs: 392 and 379, respectively;
iv) comprise the amino acid sequences of SEQ ID NOs: 246 and 375, and SEQ ID NOs: 247 and 379, respectively;
v) comprise the amino acid sequences of SEQ ID NOs: 256 and 375, and SEQ ID NOs: 257 and 379, respectively;
vi) comprise the amino acid sequences of SEQ ID NOs: 266 and 375, and SEQ ID NOs: 267 and 379, respectively;
vii) comprise the amino acid sequences of SEQ ID NOs: 276 and 375, and SEQ ID NOs: 277 and 379, respectively;
viii) comprise the amino acid sequences of SEQ ID NOs: 286 and 375, and SEQ ID NOs: 287 and 379, respectively;
ix) comprise the amino acid sequences of SEQ ID NOs: 296 and 375, and SEQ ID NOs: 297 and 379, respectively;
x) are the HC and LC, respectively, of pembrolizumab; or
xi) are the HC and LC, respectively, of nivolumab.

57. The method of claim 54, wherein the anti-TIM-3 antibody competes for binding to human TIM-3 with, or binds to the same epitope of human TIM-3 as, an antibody comprising the heavy and light chain variable domain amino acid sequences of:
a) SEQ ID NOs: 7 and 4, respectively;
b) SEQ ID NOs: 3 and 4, respectively;
c) SEQ ID NOs: 16 and 17, respectively;
d) SEQ ID NOs: 26 and 27, respectively;
e) SEQ ID NOs: 36 and 37, respectively;
f) SEQ ID NOs: 46 and 47, respectively;
g) SEQ ID NOs: 56 and 57, respectively;
h) SEQ ID NOs: 66 and 67, respectively;
i) SEQ ID NOs: 76 and 77, respectively;
j) SEQ ID NOs: 86 and 87, respectively;
k) SEQ ID NOs: 96 and 97, respectively;
l) SEQ ID NOs: 106 and 107, respectively;
m) SEQ ID NOs: 116 and 117, respectively;
n) SEQ ID NOs: 126 and 127, respectively;
o) SEQ ID NOs: 136 and 137, respectively;
p) SEQ ID NOs: 146 and 147, respectively;
q) SEQ ID NOs: 156 and 157, respectively;
r) SEQ ID NOs: 166 and 167, respectively;
s) SEQ ID NOs: 176 and 177, respectively;
t) SEQ ID NOs: 186 and 187, respectively;
u) SEQ ID NOs: 196 and 197, respectively;
v) SEQ ID NOs: 206 and 207, respectively; or
w) SEQ ID NOs: 216 and 217, respectively.

58. The method of claim 57, wherein the anti-TIM-3 antibody has at least one of the following properties:
a) binds to human TIM-3 with a K_{D} of 23 nM or less as measured by surface plasmon resonance;
b) binds to cynomolgus TIM-3 with a K_{D} of 22 nM or less as measured by surface plasmon resonance;
c) binds to human TIM-3 with an EC50 of 1.2 nM or less as measured by ELISA;
d) binds to cynomolgus TIM-3 with an EC50 of 46 nM or less as measured by ELISA;
e) increases IFN-γ secretion in a one-way mixed lymphocyte reaction assay;
f) increases IFN-γ secretion in a two-way mixed lymphocyte reaction assay;
g) increases TNF-α secretion in a one-way mixed lymphocyte reaction assay;
h) increases TNF-α secretion from dendritic cells; and
i) inhibits interaction of TIM-3 with phosphatidylserine.

59. The method of claim 57, wherein the anti-TIM-3 antibody comprises:
a) H-CDR1-3 and L-CDR1-3 comprising the amino acid sequences of:
i) SEQ ID NOs: 8-13, respectively;
ii) SEQ ID NOs: 18-23, respectively;
iii) SEQ ID NOs: 28-33, respectively;
iv) SEQ ID NOs: 38-43, respectively;
v) SEQ ID NOs: 48-53, respectively;
vi) SEQ ID NOs: 58-63, respectively;
vii) SEQ ID NOs: 68-73, respectively;
viii) SEQ ID NOs: 78-83, respectively;
ix) SEQ ID NOs: 88-93, respectively;
x) SEQ ID NOs: 98-103, respectively;
xi) SEQ ID NOs: 108-113, respectively;
xii) SEQ ID NOs: 118-123, respectively;
xiii) SEQ ID NOs: 128-133, respectively;
xiv) SEQ ID NOs: 138-143, respectively;
xv) SEQ ID NOs: 148-153, respectively;
xvi) SEQ ID NOs: 158-163, respectively;
xvii) SEQ ID NOs: 168-173, respectively;
xviii) SEQ ID NOs: 178-183, respectively;
xix) SEQ ID NOs: 188-193, respectively;
xx) SEQ ID NOs: 198-203, respectively;
xxi) SEQ ID NOs: 208-213, respectively; or
xxii) SEQ ID NOs: 218-223, respectively;
b) a VH and a VL comprising the amino acid sequences of:
i) SEQ ID NOs: 7 and 4, respectively;
ii) SEQ ID NOs: 3 and 4, respectively;
iii) SEQ ID NOs: 16 and 17, respectively;
iv) SEQ ID NOs: 26 and 27, respectively;
v) SEQ ID NOs: 36 and 37, respectively;
vi) SEQ ID NOs: 46 and 47, respectively;
vii) SEQ ID NOs: 56 and 57, respectively;
viii) SEQ ID NOs: 66 and 67, respectively;
ix) SEQ ID NOs: 76 and 77, respectively;
x) SEQ ID NOs: 86 and 87, respectively;
xi) SEQ ID NOs: 96 and 97, respectively;
xii) SEQ ID NOs: 106 and 107, respectively;
xiii) SEQ ID NOs: 116 and 117, respectively;
xiv) SEQ ID NOs: 126 and 127, respectively;
xv) SEQ ID NOs: 136 and 137, respectively;
xvi) SEQ ID NOs: 146 and 147, respectively;
xvii) SEQ ID NOs: 156 and 157, respectively;
xviii) SEQ ID NOs: 166 and 167, respectively;
xix) SEQ ID NOs: 176 and 177, respectively;
xx) SEQ ID NOs: 186 and 187, respectively;
xxi) SEQ ID NOs: 196 and 197, respectively;
xxii) SEQ ID NOs: 206 and 207, respectively; or
xxiii) SEQ ID NOs: 216 and 217, respectively; or
c) an HC and an LC comprising the amino acid sequences of:
i) SEQ ID NO: 7 and SEQ ID NO: 374, 375, 376, or 377; and SEQ ID NOs: 4 and 378; respectively;
ii) SEQ ID NO: 3 and SEQ ID NO: 374, 375, 376, or 377; and SEQ ID NOs: 4 and 378; respectively;
iii) SEQ ID NO: 16 and SEQ ID NO: 374, 375, 376, or 377; and SEQ ID NOs: 17 and 378; respectively;
iv) SEQ ID NO: 26 and SEQ ID NO: 374, 375, 376, or 377; and SEQ ID NOs: 27 and 378; respectively;
v) SEQ ID NO: 36 and SEQ ID NOs: 374, 375, 376, or 377; and SEQ ID NOs: 37 and 378; respectively;
vi) SEQ ID NO: 46 and SEQ ID NO: 374, 375, 376, or 377; and SEQ ID NOs: 47 and 378; respectively;
vii) SEQ ID NO: 56 and SEQ ID NO: 374, 375, 376, or 377; and SEQ ID NOs: 57 and 378; respectively;
viii) SEQ ID NO: 66 and SEQ ID NO: 374, 375, 376, or 377; and SEQ ID NOs: 67 and 378; respectively;
ix) SEQ ID NO: 76 and SEQ ID NO: 374, 375, 376, or 377; and SEQ ID NOs: 77 and 378; respectively;
x) SEQ ID NO: 86 and SEQ ID NO: 374, 375, 376, or 377; and SEQ ID NOs: 87 and 378; respectively;
xi) SEQ ID NO: 96 and SEQ ID NO: 374, 375, 376, or 377; and SEQ ID NOs: 97 and 378; respectively;
xii) SEQ ID NO: 106 and SEQ ID NO: 374, 375, 376, or 377; and SEQ ID NOs: 107 and 378; respectively;
xiii) SEQ ID NO: 116 and SEQ ID NO: 374, 375, 376, or 377; and SEQ ID NOs: 117 and 378; respectively;
xiv) SEQ ID NO: 126 and SEQ ID NO: 374, 375, 376, or 377; and SEQ ID NOs: 127 and 378; respectively;
xv) SEQ ID NO: 136 and SEQ ID NO: 374, 375, 376, or 377; and SEQ ID NOs: 137 and 378; respectively;
xvi) SEQ ID NO: 146 and SEQ ID NO: 374, 375, 376, or 377; and SEQ ID NOs: 147 and 378; respectively;
xvii) SEQ ID NO: 156 and SEQ ID NO: 374, 375, 376, or 377; and SEQ ID NOs: 157 and 378; respectively;
xviii) SEQ ID NO: 166 and SEQ ID NO: 374, 375, 376, or 377; and SEQ ID NOs: 167 and 378; respectively;
xix) SEQ ID NO: 176 and SEQ ID NO: 374, 375, 376, or 377; and SEQ ID NOs: 177 and 378; respectively;
xx) SEQ ID NO: 186 and SEQ ID NO: 374, 375, 376, or 377; and SEQ ID NOs: 187 and 378; respectively;
xxi) SEQ ID NO: 196 and SEQ ID NO: 374, 375, 376, or 377; and SEQ ID NOs: 197 and 378; respectively;
xxii) SEQ ID NO: 206 and SEQ ID NO: 374, 375, 376, or 377; and SEQ ID NOs: 207 and 378; respectively; or
xxiii) SEQ ID NO: 216 and SEQ ID NO: 374, 375, 376, or 377; and SEQ ID NOs: 217 and 378; respectively.

60. The method of claim 54, wherein the anti-LAG-3 antibody competes for binding to human LAG-3 with, or binds to the same epitope of human LAG-3 as, an antibody comprising the heavy and light chain variable domain amino acid sequences of:
a) SEQ ID NOs: 316 and 317, respectively;
b) SEQ ID NOs: 306 and 307, respectively;
c) SEQ ID NOs: 326 and 327, respectively;
d) SEQ ID NOs: 336 and 337, respectively;
e) SEQ ID NOs: 346 and 347, respectively;
f) SEQ ID NOs: 356 and 357, respectively; or
g) SEQ ID NOs: 366 and 367, respectively.

61. The method of claim 60, wherein the anti-LAG-3 antibody has at least one of the following properties:
a) at a concentration of 20 μg/mL, reduces the binding of human LAG-3 to human MHC class II on A375 cells by greater than 85% compared to a negative control antibody as determined by a flow cytometric competition assay;
b) at a concentration of 20 μg/mL, reduces the binding of human LAG-3 to human MHC class II on A375 cells to between 35% and 85% compared to a negative control antibody as determined by a flow cytometric competition assay;
c) blocks binding between human LAG-3 expressed on Jurkat cells and human MHC class II expressed on Raji cells;
d) binds to human LAG-3 with an EC50 of 0.1 nM or less as measured by flow cytometry;
e) binds to cynomolgus LAG-3 with an EC50 of 0.3 nM or less as measured by flow cytometry;
f) binds to human LAG-3 with a K_{D} of 3.0×10⁻⁸ M or less as measured by surface plasmon resonance;
g) binds to cynomolgus LAG-3 with a K_{D} of 1.5×10⁻⁷ M or less as measured by surface plasmon resonance;
h) binds to mouse LAG-3 with a K_{D} of 3.5×10⁻⁸ M or less as measured by surface plasmon resonance;
i) stimulates IL-2 production in Staphylococcal enterotoxin B (SEB) treated human peripheral blood mononuclear cells (PBMCs);
j) reduces cellular levels of LAG-3 in human T cells;
k) reduces soluble levels of LAG-3 in the culture of human T cells;
l) induces tumor growth regression in vivo;
m) delays tumor growth in vivo; and
n) does not bind to the same epitope of human LAG-3 as antibody 25F7-Lag3.5.

62. The method of claim 60, wherein the anti-LAG-3 antibody comprises:
a) H-CDR1-3 and L-CDR1-3 comprising the amino acid sequences of:
i) SEQ ID NOs: 318-323, respectively;
ii) SEQ ID NOs: 308-313, respectively;
iii) SEQ ID NOs: 328-333, respectively;
iv) SEQ ID NOs: 338-343, respectively;
v) SEQ ID NOs: 348-353, respectively;
vi) SEQ ID NOs: 358-363, respectively; or
vii) SEQ ID NOs: 368-373, respectively;
b) a VH and a VL comprising the amino acid sequences of:
i) SEQ ID NOs: 316 and 317, respectively;
ii) SEQ ID NOs: 306 and 307, respectively;
iii) SEQ ID NOs: 326 and 327, respectively;
iv) SEQ ID NOs: 336 and 337, respectively;
v) SEQ ID NOs: 346 and 347, respectively;
vi) SEQ ID NOs: 356 and 357, respectively; or
vii) SEQ ID NOs: 366 and 367, respectively; or
c) an HC and an LC comprising the amino acid sequences of
i) SEQ ID NOs: 316 and 375, and SEQ ID NOs: 317 and 378, respectively;
ii) SEQ ID NOs: 306 and 375, and SEQ ID NOs: 307 and 378, respectively;
iii) SEQ ID NOs: 326 and 375, and SEQ ID NOs: 327 and 378, respectively;
iv) SEQ ID NOs: 336 and 375, and SEQ ID NOs: 337 and 378, respectively;
v) SEQ ID NOs: 346 and 375, and SEQ ID NOs: 347 and 378, respectively;
vi) SEQ ID NOs: 356 and 375, and SEQ ID NOs: 357 and 378, respectively; or
vii) SEQ ID NOs: 366 and 375, and SEQ ID NOs: 367 and 379, respectively.

63. The method of claim 54, comprising administering to the patient:
a) an anti-PD-1 antibody whose H-CDR1-3 and L-CDR1-3 comprise the amino acid sequences of SEQ ID NOs: 228-233, respectively; and an anti-TIM-3 antibody whose H-CDR1-3 and L-CDR1-3 comprise the amino acid sequences of SEQ ID NOs: 8-13, respectively;
b) an anti-PD-1 antibody whose VH and VL comprise the amino acid sequences of SEQ ID NOs: 226 and 227, respectively; and an anti-TIM-3 antibody whose VH and VL comprise the amino acid sequences of SEQ ID NOs: 7 and 4, respectively;
c) an anti-PD-1 antibody comprising an HC that comprises the amino acid sequences of SEQ ID NOs: 226 and 375 and an LC that comprises the amino acid sequences of SEQ ID NOs: 227 and 379; and an anti-TIM-3 antibody comprising an HC that comprises the amino acid sequences of SEQ ID NOs: 7 and 377 and an LC that comprises the amino acid sequences of SEQ ID NOs: 4 and 378;
d) pembrolizumab and an anti-TIM-3 antibody comprising an HC that comprises the amino acid sequences of SEQ ID NOs: 7 and 377 and an LC that comprises the amino acid sequences of SEQ ID NOs: 4 and 378; or
e) nivolumab and an anti-TIM-3 antibody comprising an HC that comprises the amino acid sequences of SEQ ID NOs: 7 and 377 and an LC that comprises the amino acid sequences of SEQ ID NOs: 4 and 378.

64. The method of claim 54, comprising administering to the patient:
a) an anti-PD-1 antibody whose H-CDR1-3 and L-CDR1-3 comprise the amino acid sequences of SEQ ID NOs: 228-233, respectively; and an anti-LAG-3 antibody whose H-CDR1-3 and L-CDR1-3 comprise the amino acid sequences of SEQ ID NOs: 318-323, respectively;
b) an anti-PD-1 antibody whose VH and VL comprise the amino acid sequences of SEQ ID NOs: 226 and 227, respectively; and an anti-LAG-3 antibody whose VH and VL comprise the amino acid sequences of SEQ ID NOs: 316 and 317, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; and an anti-LAG-3 antibody comprising an HC that comprises the amino acid sequences of SEQ ID NOs: 316 and 375 and an LC that comprises the amino acid sequences of SEQ ID NOs: 317 and 378.

65. The method of claim 54, comprising administering to the patient:
a) an anti-PD-1 antibody whose H-CDR1-3 and L-CDR1-3 comprise the amino acid sequences of SEQ ID NOs: 228-233, respectively; an anti-TIM-3 antibody whose H-CDR1-3 and L-CDR1-3 comprise the amino acid sequences of SEQ ID NOs: 8-13, respectively; and an anti-LAG-3 antibody whose H-CDR1-3 and L-CDR1-3 comprise the amino acid sequences of SEQ ID NOs: 318-323, respectively;
b) an anti-PD-1 antibody whose VH and VL comprise the amino acid sequences of SEQ ID NOs: 226 and 227, respectively; an anti-TIM-3 antibody whose VH and VL comprise the amino acid sequences of SEQ ID NOs: 7 and 4, respectively; and an anti-LAG-3 antibody whose VH and VL comprise the amino acid sequences of SEQ ID NOs: 316 and 317, respectively; or
c) an anti-PD-1 antibody comprising an HC that comprises the amino acid sequences of SEQ ID NOs: 226 and 375 and an LC that comprises the amino acid sequences of SEQ ID NOs: 227 and 379; an anti-TIM-3 antibody comprising an HC that comprises the amino acid sequences of SEQ ID NOs: 7 and 377 and an LC that comprises the amino acid sequences of SEQ ID NOs: 4 and 378; and an anti-LAG-3 antibody comprising an HC that comprises the amino acid sequences of SEQ ID NOs: 316 and 375 and an LC that comprises the amino acid sequences of SEQ ID NOs: 317 and 378.

66. The method of claim 54, wherein the patient has cancer.

67. The method of claim 66, wherein the cancer is selected from the group consisting of leukemia, Hodgkin's lymphoma, non-Hodgkin's lymphoma, myeloma, melanoma, non-small cell lung cancer, bladder cancer, head and neck squamous cell carcinoma, ovarian cancer, colorectal cancer, renal cell carcinoma, Merkel-cell carcinoma, fibrosarcoma, gliosarcoma, or glioblastoma.

68. The method of claim 66, further comprising administering to the patient radiation therapy, or at least one of a chemotherapeutic agent, an anti-neoplastic agent, and an anti-angiogenic agent.

69. A multi-specific antibody that specifically binds to:
a) human PD-1 and human TIM-3;
b) human PD-1 and human LAG-3; or
c) human PD-1, human anti-TIM-3, and human LAG-3,
comprising an antigen-binding portion of an anti-PD-1 antibody as defined in claim 54.

70. A pharmaceutical composition comprising (1) an anti-PD-1 antibody or an antigen-binding portion thereof as defined in claim 54, (2) an anti-TIM-3 antibody or an antigen-binding portion thereof and/or an anti-LAG-3 antibody or an antigen-binding portion thereof, and (3) a pharmaceutically acceptable excipient.

71. The pharmaceutical composition of claim 70, wherein the anti-TIM-3 antibody competes for binding to human TIM-3 with, or binds to the same epitope of human TIM-3 as, an antibody comprising the heavy and light chain variable domain amino acid sequences of:
a) SEQ ID NOs: 7 and 4, respectively;
b) SEQ ID NOs: 3 and 4, respectively;
c) SEQ ID NOs: 16 and 17, respectively;
d) SEQ ID NOs: 26 and 27, respectively;
e) SEQ ID NOs: 36 and 37, respectively;
f) SEQ ID NOs: 46 and 47, respectively;
g) SEQ ID NOs: 56 and 57, respectively;
h) SEQ ID NOs: 66 and 67, respectively;
i) SEQ ID NOs: 76 and 77, respectively;
j) SEQ ID NOs: 86 and 87, respectively;
k) SEQ ID NOs: 96 and 97, respectively;
l) SEQ ID NOs: 106 and 107, respectively;
m) SEQ ID NOs: 116 and 117, respectively;
n) SEQ ID NOs: 126 and 127, respectively;
o) SEQ ID NOs: 136 and 137, respectively;
p) SEQ ID NOs: 146 and 147, respectively;
q) SEQ ID NOs: 156 and 157, respectively;
r) SEQ ID NOs: 166 and 167, respectively;
s) SEQ ID NOs: 176 and 177, respectively;
t) SEQ ID NOs: 186 and 187, respectively;
u) SEQ ID NOs: 196 and 197, respectively;
v) SEQ ID NOs: 206 and 207, respectively; or
w) SEQ ID NOs: 216 and 217, respectively.

72. The pharmaceutical composition of claim 70, wherein the anti-LAG-3 antibody competes for binding to human LAG-3 with, or binds to the same epitope of human LAG-3 as, an antibody comprising the heavy and light chain variable domain amino acid sequences of:
a) SEQ ID NOs: 316 and 317, respectively;
b) SEQ ID NOs: 306 and 307, respectively;
c) SEQ ID NOs: 326 and 327, respectively;
d) SEQ ID NOs: 336 and 337, respectively;
e) SEQ ID NOs: 346 and 347, respectively;
f) SEQ ID NOs: 356 and 357, respectively; or
g) SEQ ID NOs: 366 and 367, respectively.

73. A pharmaceutical composition comprising:
a) an anti-PD-1 antibody whose H-CDR1-3 and L-CDR1-3 comprise the amino acid sequences of SEQ ID NOs: 228-233, respectively; and an anti-TIM-3 antibody whose H-CDR1-3 and L-CDR1-3 comprise the amino acid sequences of SEQ ID NOs: 8-13, respectively;
b) an anti-PD-1 antibody whose VH and VL comprise the amino acid sequences of SEQ ID NOs: 226 and 227, respectively; and an anti-TIM-3 antibody whose VH and VL comprise the amino acid sequences of SEQ ID NOs: 7 and 4, respectively;
c) an anti-PD-1 antibody comprising an HC that comprises the amino acid sequences of SEQ ID NOs: 226 and 375 and an LC that comprises the amino acid sequences of SEQ ID NOs: 227 and 379; and an anti-TIM-3 antibody comprising an HC that comprises the amino acid sequences of SEQ ID NOs: 7 and 377 and an LC that comprises the amino acid sequences of SEQ ID NOs: 4 and 378;
d) pembrolizumab and an anti-TIM-3 antibody comprising an HC that comprises the amino acid sequences of SEQ ID NOs: 7 and 377 and an LC that comprises the amino acid sequences of SEQ ID NOs: 4 and 378; or
e) nivolumab and an anti-TIM-3 antibody comprising an HC that comprises the amino acid sequences of SEQ ID NOs: 7 and 377 and an LC that comprises the amino acid sequences of SEQ ID NOs: 4 and 378.


SEQUENCE LISTING (397 sequences)

SEQ ID NO: 1 - DNA, 360 bp, synthetic construct
  misc_feature 1..360: note = source = /note="Description of Artificial Sequence: Synthetic polynucleotide"
  source 1..360: mol_type = other DNA
caggtgcagctacagcagtcgggcccaggactggtgaagccttcacagaccctgtccctcacctgcactgtctctggtggctccatcagcagtggtggttactactggagttggacccgtcagcacccagggatgggcctggagtggattggatacatctcttacagtgggagtatctattacactccgtccctcaagagtcgacttaccatatcagtggacacgtctaagaaccagttctccctgaagctgagctctgtgactgccgcggacacggccgtatattactgtgcgagtttggattcctggggatctaaccgtgactactggggccagggaaccctggtcaccgtctcgagt

SEQ ID NO: 2 - DNA, 321 bp, synthetic construct
  misc_feature 1..321: note = source = /note="Description of Artificial Sequence: Synthetic polynucleotide"
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagattaag

SEQ ID NO: 3 - protein, 120 aa, synthetic construct
  REGION 1..120: note = source = /note="Description of Artificial Sequence: Synthetic polypeptide"
QVQLQQSGPGLVKPSQTLSLTCTVSGGSISSGGYYWSWTRQHPGMGLEWIGYISYSGSIYYTPSLKSRLTISVDTSKNQFSLKLSSVTAADTAVYYCASLDSWGSNRDYWGQGTLVTVSS

SEQ ID NO: 4 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 5 - DNA, 360 bp, synthetic construct
  misc_feature 1..360: note = source = /note="Description of Artificial Sequence: Synthetic polynucleotide"
  source 1..360: mol_type = other DNA
caggtgcagctgcaggagagtggccccggactggtcaagccttcacagactctgagcctgacctgcacagtgtctggcggaagtatcagctccgggggttactattggagctggacccgacagcacccaggaatgggtctggaatggatcgggtacatttcatatagcggctccatctactatacaccctcactgaaaagcaggctgaccatttccgtggacacatctaagaaccagttcagcctgaaactgtctagtgtgacagccgctgatactgcagtctactattgtgcctccctggactcttggggcagtaatagagattactggggccagggaactctggtcaccgtctcgagt

SEQ ID NO: 6 - DNA, 321 bp, synthetic construct
  misc_feature 1..321: note = source = /note="Description of Artificial Sequence: Synthetic polynucleotide"
  source 1..321: mol_type = other DNA
gagatcgtgctgactcagtccccagccaccctgtcactgagcccaggagaacgagcaaccctgtcttgcagggcctcccagtctgtcagctcctacctggcttggtatcagcagaagcccgggcaggcacctcgactgctgatctacgacgccagtaacagagctaccggtattcccgcccgcttcagtggttcaggcagcggaacagactttaccctgacaatctctagtctggagcctgaagatttcgccgtgtactattgtcagcagaggtctaattggccactgacatttggcggagggactaaggtcgagatcaag

SEQ ID NO: 7 - protein, 120 aa, synthetic construct
  REGION 1..120: note = source = /note="Description of Artificial Sequence: Synthetic polypeptide"
QVQLQESGPGLVKPSQTLSLTCTVSGGSISSGGYYWSWTRQHPGMGLEWIGYISYSGSIYYTPSLKSRLTISVDTSKNQFSLKLSSVTAADTAVYYCASLDSWGSNRDYWGQGTLVTVSS

SEQ ID NO: 8 - protein, 10 aa, synthetic construct
  REGION 1..10: note = source = /note="Description of Artificial Sequence: Synthetic peptide"
GGSISSGGYY

SEQ ID NO: 9 - protein, 7 aa, synthetic construct
  REGION 1..7: note = source = /note="Description of Artificial Sequence: Synthetic peptide"
ISYSGSI

SEQ ID NO: 10 - protein, 14 aa, synthetic construct
  REGION 1..14: note = source = /note="Description of Artificial Sequence: Synthetic peptide"
CASLDSWGSNRDYW

SEQ ID NO: 11 - protein, 6 aa, synthetic construct
  REGION 1..6: note = source = /note="Description of Artificial Sequence: Synthetic peptide"
QSVSSY

SEQ ID NO: 12 - skipped

SEQ ID NO: 13 - protein, 11 aa, synthetic construct [sequence omitted]

SEQ ID NO: 14 - DNA, 345 bp, synthetic construct
  misc_feature 1..345: note = source = /note="Description of Artificial Sequence: Synthetic polynucleotide"
  source 1..345: mol_type = other DNA
caggtcaccttgaaggagtggggcgcaggactgttgaggccctcggagaccctgtccctcacctgcgctgtctatggtgggtccttcagtggttactactggagctggatccgccagcccccagggaaggggctggagtggataggggaaatcaatcatagtggaagcaccaactacaacccgtccctcaagagtcgagtcaccatatcagtagacgcgaccaagaaacaattctccctgaagctgacctctgtgaccgccgcggacacggctgtgtattactgtgcgagatattgggagctccctgactactggggccagggcaccctggtcaccgtctcgagt

SEQ ID NO: 15 - DNA, 321 bp, synthetic construct
  misc_feature 1..321: note = source = /note="Description of Artificial Sequence: Synthetic polynucleotide"
  source 1..321: mol_type = other DNA
gacatccagttgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctccgacgttcggccaagggaccaaggtggaaatcaag

SEQ ID NO: 16 - protein, 115 aa, synthetic construct
  REGION 1..115: note = source = /note="Description of Artificial Sequence: Synthetic polypeptide"
QVTLKEWGAGLLRPSETLSLTCAVYGGSFSGYYWSWIRQPPGKGLEWIGEINHSGSTNYNPSLKSRVTISVDATKKQFSLKLTSVTAADTAVYYCARYWELPDYWGQGTLVTVSS

SEQ ID NO: 17 - protein, 107 aa, synthetic construct
  REGION 1..107: note = source = /note="Description of Artificial Sequence: Synthetic polypeptide"
DIQLTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIK

SEQ ID NO: 18 - protein, 8 aa, synthetic construct
  REGION 1..8: note = source = /note="Description of Artificial Sequence: Synthetic peptide"
GGSFSGYY

SEQ ID NO: 19 - protein, 7 aa, synthetic construct
  REGION 1..7: note = source = /note="Description of Artificial Sequence: Synthetic peptide"
INHSGST

SEQ ID NO: 20 - protein, 11 aa, synthetic construct
  REGION 1..11: note = source = /note="Description of Artificial Sequence: Synthetic peptide"
CARYWELPDYW

SEQ ID NO: 21 - protein, 6 aa, synthetic construct
  REGION 1..6: note = source = /note="Description of Artificial Sequence: Synthetic peptide"
QGIRND

SEQ ID NO: 22 - skipped

SEQ ID NO: 23 - protein, 11 aa, synthetic construct [sequence omitted]

SEQ ID NO: 24 - DNA, 345 bp, synthetic construct
  misc_feature 1..345: note = source = /note="Description of Artificial Sequence: Synthetic polynucleotide"
  source 1..345: mol_type = other DNA
cagatgcagctggtgcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatgtcagttgacacgtccaagcaccagttctccctgaagctgagctctgtgaccgccgcggacacggctgtgtattactgtgcgagatggtgggagcttcctgactactggggccagggaaccctggtcaccgtctcgagt

SEQ ID NO: 25 - DNA, 321 bp, synthetic construct
  misc_feature 1..321: note = source = /note="Description of Artificial Sequence: Synthetic polynucleotide"
  source 1..321: mol_type = other DNA
gaaattgtgttgacgcagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaag

SEQ ID NO: 26 - protein, 115 aa, synthetic construct
  REGION 1..115: note = source = /note="Description of Artificial Sequence: Synthetic polypeptide"
QMQLVQWGAGLLKPSETLSLTCAVYGGSFSGYYWSWIRQPPGKGLEWIGEINHSGSTNYNPSLKSRVTMSVDTSKHQFSLKLSSVTAADTAVYYCARWWELPDYWGQGTLVTVSS

SEQ ID NO: 27 - protein, 107 aa, synthetic construct
  REGION 1..107: note = source = /note="Description of Artificial Sequence: Synthetic polypeptide"
EIVLTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 28 - protein, 8 aa, synthetic construct
  REGION 1..8: note = source = /note="Description of Artificial Sequence: Synthetic peptide"
GGSFSGYY

SEQ ID NO: 29 - protein, 7 aa, synthetic construct
  REGION 1..7: note = source = /note="Description of Artificial Sequence: Synthetic peptide"
INHSGST

SEQ ID NO: 30 - protein, 11 aa, synthetic construct
  REGION 1..11: note = source = /note="Description of Artificial Sequence: Synthetic peptide"
CARWWELPDYW

SEQ ID NO: 31 - protein, 6 aa, synthetic construct
  REGION 1..6: note = source = /note="Description of Artificial Sequence: Synthetic peptide"
QGIRND

SEQ ID NO: 32 - skipped

SEQ ID NO: 33 - protein, 11 aa, synthetic construct [sequence omitted]

SEQ ID NO: 34 - DNA, 351 bp, synthetic construct
  misc_feature 1..351: note = source = /note="Description of Artificial Sequence: Synthetic polynucleotide"
  source 1..351: mol_type = other DNA
cagatgcagctggtgcaatggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgaggttttactatgctccgaactttgactactggggccagggcaccctggtcaccgtctcgagt

SEQ ID NO: 35 - DNA, 318 bp, synthetic construct
  misc_feature 1..318: note = source = /note="Description of Artificial Sequence: Synthetic polynucleotide"
  source 1..318: mol_type = other DNA
gaaattgtgttgacgcagtctccttccaccctgtctgcatctgtaggagacagagtcaccatcacttgccgggccagtcagagtattagtagctggttggcctggtatcagcagaaaccagggacagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattccactttcggcggagggaccaaggtggagatcaaa

SEQ ID NO: 36 - protein, 117 aa, synthetic construct
  REGION 1..117: note = source = /note="Description of Artificial Sequence: Synthetic polypeptide"
QMQLVQWGAGLLKPSETLSLTCAVYGGSFSGYYWSWIRQPPGKGLEWIGEINHSGSTNYNPSLKSRVTISVDTSKNQFSLKLSSVTAADTAVYYCARFYYAPNFDYWGQGTLVTVSS

SEQ ID NO: 37 - protein, 106 aa, synthetic construct
  REGION 1..106: note = source = /note="Description of Artificial Sequence: Synthetic polypeptide"
EIVLTQSPSTLSASVGDRVTITCRASQSISSWLAWYQQKPGTAPKLLIYKASSLESGVPSRFSGSGSGTEFTLTISSLQPDDFATYYCQQYNSYSTFGGGTKVEIK

SEQ ID NO: 38 - protein, 8 aa, synthetic construct
  REGION 1..8: note = source = /note="Description of Artificial Sequence: Synthetic peptide"
GGSFSGYY

SEQ ID NO: 39 - protein, 7 aa, synthetic construct
  REGION 1..7: note = source = /note="Description of Artificial Sequence: Synthetic peptide"
INHSGST

SEQ ID NO: 40 - protein, 13 aa, synthetic construct
  REGION 1..13: note = source = /note="Description of Artificial Sequence: Synthetic peptide"
CARFYYAPNFDYW

SEQ ID NO: 41 - protein, 6 aa, synthetic construct
  REGION 1..6: note = source = /note="Description of Artificial Sequence: Synthetic peptide"
QSISSW

SEQ ID NO: 42 - skipped

SEQ ID NO: 43 - protein, 10 aa, synthetic construct [sequence omitted]

SEQ ID NO: 44 - DNA, 360 bp, synthetic construct
  misc_feature 1..360: note = source = /note="Description of Artificial Sequence: Synthetic polynucleotide"
  source 1..360: mol_type = other DNA
caggtgcagctgcagcagtcgggcccaggactggtgaagccttcggagaccctgtccctcacctgcactgtctctggtggctccatcagcagtagtaattactactggggctggatccgccagcccccagggaaggggctggagtggattgggagtatctattatagtgggaacacctactacaacccgtccctcaagagtcgagtcaccatatccgtagacacgtccaagaaccagttctccctgaagctgagttctgtgaccgccgcagacacggctgtgtattactgtgcgagacagacagtggctggccccctctttgactactggggccagggaaccctggtcaccgtctcgagt

SEQ ID NO: 45 - DNA, 321 bp, synthetic construct
  misc_feature 1..321: note = source = /note="Description of Artificial Sequence: Synthetic polynucleotide"
  source 1..321: mol_type = other DNA
gaaattgtgatgacgcagtctccttccaccctgtctgcatctgtaggagacagagtcaccatcacttgccgggccagtcagagtattagtagctggttggcctggtatcagcagaaaccagggaaagcccctaaggtcctgatctataaggcgtctagtttagaaagtggggtcccatcaaggttcagcggcagtggatctgggacagaactcactctcaccatcagcagcctgcagcctgatgattttgcaacttattactgccaacagtataatagttattcattcactttcggccctgggaccaaagtggatatcaag

SEQ ID NO: 46 - protein, 120 aa, synthetic construct
  REGION 1..120: note = source = /note="Description of Artificial Sequence: Synthetic polypeptide"
QVQLQQSGPGLVKPSETLSLTCTVSGGSISSSNYYWGWIRQPPGKGLEWIGSIYYSGNTYYNPSLKSRVTISVDTSKNQFSLKLSSVTAADTAVYYCARQTVAGPLFDYWGQGTLVTVSS

SEQ ID NO: 47 - protein, 107 aa, synthetic construct
  REGION 1..107: note = source = /note="Description of Artificial Sequence: Synthetic polypeptide"
EIVMTQSPSTLSASVGDRVTITCRASQSISSWLAWYQQKPGKAPKVLIYKASSLESGVPSRFSGSGSGTELTLTISSLQPDDFATYYCQQYNSYSFTFGPGTKVDIK

SEQ ID NO: 48 - protein, 10 aa, synthetic construct
  REGION 1..10: note = source = /note="Description of Artificial Sequence: Synthetic peptide"
GGSISSSNYY

SEQ ID NO: 49 - protein, 7 aa, synthetic construct
  REGION 1..7: note = source = /note="Description of Artificial Sequence: Synthetic peptide"
IYYSGNT

SEQ ID NO: 50 - protein, 14 aa, synthetic construct
  REGION 1..14: note = source = /note="Description of Artificial Sequence: Synthetic peptide"
CARQTVAGPLFDYW

SEQ ID NO: 51 - protein, 6 aa, synthetic construct
  REGION 1..6: note = source = /note="Description of Artificial Sequence: Synthetic peptide"
QSISSW

SEQ ID NO: 52 - skipped

SEQ ID NO: 53 - protein, 11 aa, synthetic construct [sequence omitted]

SEQ ID NO: 54 - DNA, 375 bp, synthetic construct
  misc_feature 1..375: note = source = /note="Description of Artificial Sequence: Synthetic polynucleotide"
  source 1..375: mol_type = other DNA
caggtccagctggtgcaatctggggctgaggtgaagaagcctggggcctcagtgaaggtctcctgcaaggctgctggatacaccttaaccggctactatatgcactgggtgcgacaggcccctggacaaggccttgagtggatgggacggatcaaccctaacagtggtggctcaaacaatgcacagaagtttcagggcagggtcaccatgaccagggacacgtccatcagcacagcctacatggagctgagcaggctgagatctgacgacacggccgtgtattactgtgcgagagagggtcccctgtatagcagtggctggtacgagggtgcttttgatatctggggccaagggacaatggtcaccgtctcgagt

SEQ ID NO: 55 - DNA, 327 bp, synthetic construct
  misc_feature 1..327: note = source = /note="Description of Artificial Sequence: Synthetic polynucleotide"
  source 1..327: mol_type = other DNA
gaaattgtgatgacgcagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattctccggggctcactttcggcggagggaccaaggtggagatcaag

SEQ ID NO: 56 - protein, 125 aa, synthetic construct
  REGION 1..125: note = source = /note="Description of Artificial Sequence: Synthetic polypeptide"
QVQLVQSGAEVKKPGASVKVSCKAAGYTLTGYYMHWVRQAPGQGLEWMGRINPNSGGSNNAQKFQGRVTMTRDTSISTAYMELSRLRSDDTAVYYCAREGPLYSSGWYEGAFDIWGQGTMVTVSS

SEQ ID NO: 57 - protein, 109 aa, synthetic construct
  REGION 1..109: note = source = /note="Description of Artificial Sequence: Synthetic polypeptide"
EIVMTQSPSTLSASVGDRVTITCRASQSISSWLAWYQQKPGKAPKLLIYKASSLESGVPSRFSGSGSGTEFTLTISSLQPDDFATYYCQQYNSYSPGLTFGGGTKVEIK

SEQ ID NO: 58 - protein, 8 aa, synthetic construct
  REGION 1..8: note = source = /note="Description of Artificial Sequence: Synthetic peptide"
GYTLTGYY

SEQ ID NO: 59 - protein, 8 aa, synthetic construct
  REGION 1..8: note = source = /note="Description of Artificial Sequence: Synthetic peptide"
INPNSGGS

SEQ ID NO: 60 - protein, 20 aa, synthetic construct
  REGION 1..20: note = source = /note="Description of Artificial Sequence: Synthetic peptide"
CAREGPLYSSGWYEGAFDIW

SEQ ID NO: 61 - protein, 6 aa, synthetic construct
  REGION 1..6: note = source = /note="Description of Artificial Sequence: Synthetic peptide"
QSISSW

SEQ ID NO: 62 - skipped

SEQ ID NO: 63 - protein, 13 aa, synthetic construct [sequence omitted]

SEQ ID NO: 64 - DNA, 372 bp, synthetic construct
  misc_feature 1..372: note = source = /note="Description of Artificial Sequence: Synthetic polynucleotide"
  source 1..372: mol_type = other DNA
cagatgcagctacagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtattctgcttttgcagtatctgtgaaaagtcgaataaccatcaacccagacacatccaagaaccagttctccctgcagctgaactctgtgactcccgaggacacggctgtgtattactgtgcaagagagggtagcagtggctggtacggatacgtccaccactggggccagggcaccctggtcaccgtctcgagt

SEQ ID NO: 65 - DNA, 321 bp, synthetic construct
  misc_feature 1..321: note = source = /note="Description of Artificial Sequence: Synthetic polynucleotide"
  source 1..321: mol_type = other DNA
gaaattgtgttgacgcagtctccagcttccctgtctgtatctctgggagaaactgtcaccatcgaatgtcgagcaagtgaggacatttacaatggtttagcatggtatcagcagaagccagggaaatctcctcagctcctgatctataatgcaaatagcttgcatactggggtcccatcacggttcagtggcagtggatctggtacacagtattctctcaagataaacagcctgcaatctgaagatgtcgcaagttatttctgtcaacagtattacgattatcctccgacgttcggccaagggaccaaggtggaaatcaaa

SEQ ID NO: 66 - protein, 124 aa, synthetic construct
  REGION 1..124: note = source = /note="Description of Artificial Sequence: Synthetic polypeptide"
QMQLQQSGPGLVKPSQTLSLTCAISGDSVSSNSAAWNWIRQSPSRGLEWLGRTYYRSKWYSAFAVSVKSRITINPDTSKNQFSLQLNSVTPEDTAVYYCAREGSSGWYGYVHHWGQGTLVTVSS

SEQ ID NO: 67 - protein, 107 aa, synthetic construct
  REGION 1..107: note = source = /note="Description of Artificial Sequence: Synthetic polypeptide"
EIVLTQSPASLSVSLGETVTIECRASEDIYNGLAWYQQKPGKSPQLLIYNANSLHTGVPSRFSGSGSGTQYSLKINSLQSEDVASYFCQQYYDYPPTFGQGTKVEIK

SEQ ID NO: 68 - protein, 10 aa, synthetic construct
  REGION 1..10: note = source = /note="Description of Artificial Sequence: Synthetic peptide"
GDSVSSNSAA

SEQ ID NO: 69 - protein, 9 aa, synthetic construct
  REGION 1..9: note = source = /note="Description of Artificial Sequence: Synthetic peptide"
TYYRSKWYS

SEQ ID NO: 70 - protein, 16 aa, synthetic construct
  REGION 1..16: note = source = /note="Description of Artificial Sequence: Synthetic peptide"
CAREGSSGWYGYVHHW

SEQ ID NO: 71 - protein, 6 aa, synthetic construct
  REGION 1..6: note = source = /note="Description of Artificial Sequence: Synthetic peptide"
EDIYNG

SEQ ID NO: 72 - skipped

SEQ ID NO: 73 - protein, 11 aa, synthetic construct
  REGION 1..11: note = source = /note="Description of Artificial Sequence: Synthetic peptide"
CQQYYDYPPTF

SEQ ID NO: 74 - DNA, 369 bp, synthetic construct
  misc_feature 1..369: note = source = /note="Description of Artificial Sequence: Synthetic polynucleotide"
  source 1..369: mol_type = other DNA
caggtgcagctgcaggagtcgggcccaggactggtgaagccttcggagaccctgtccctcacctgcactgtctctggtggctccttcagcagtagtagttactactggggctggatccgccagccccctgggaaggggctggagtggattgggatcttctattatagtgggaccacctactacaacccgtccctcaagagtcgagtcaccatatccgcacacacgtccaagagccagttctccctgaagctgagctctgtgaccgccgcagacacggctgtgtattactgtgcgagagggggagaatattttgaccggttactcccctttgactactggggccagggaaccctggtcaccgtctcgagt

SEQ ID NO: 75 - DNA, 321 bp, synthetic construct
  misc_feature 1..321: note = source = /note="Description of Artificial Sequence: Synthetic polynucleotide"
  source 1..321: mol_type = other DNA
gaaattgtgatgacgcagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttggaaagtggggtcccatcaaggttcagcggcagtggatctgggacagaattcactctcacaatcagcagcctgcagcctgaagattttgcaacttattactgtcaacagcttaatagttacccattcactttcggccctgggaccaaagtggatatcaaa

SEQ ID NO: 76 - protein, 123 aa, synthetic construct
  REGION 1..123: note = source = /note="Description of Artificial Sequence: Synthetic polypeptide"
QVQLQESGPGLVKPSETLSLTCTVSGGSFSSSSYYWGWIRQPPGKGLEWIGIFYYSGTTYYNPSLKSRVTISAHTSKSQFSLKLSSVTAADTAVYYCARGGEYFDRLLPFDYWGQGTLVTVSS

SEQ ID NO: 77 - protein, 107 aa, synthetic construct
  REGION 1..107: note = source = /note="Description of Artificial Sequence: Synthetic polypeptide"
EIVMTQSPSFLSASVGDRVTITCRASQGISSYLAWYQQKPGKAPKLLIYAASTLESGVPSRFSGSGSGTEFTLTISSLQPEDFATYYCQQLNSYPFTFGPGTKVDIK

SEQ ID NO: 78 - protein, 10 aa, synthetic construct
  REGION 1..10: note = source = /note="Description of Artificial Sequence: Synthetic peptide"
GGSFSSSSYY

SEQ ID NO: 79 - protein, 7 aa, synthetic construct
  REGION 1..7: note = source = /note="Description of Artificial Sequence: Synthetic peptide"
FYYSGTT

SEQ ID NO: 80 - protein, 17 aa, synthetic construct
  REGION 1..17: note = source = /note="Description of Artificial Sequence: Synthetic peptide"
CARGGEYFDRLLPFDYW

SEQ ID NO: 81 - protein, 6 aa, synthetic construct
  REGION 1..6: note = source = /note="Description of Artificial Sequence: Synthetic peptide"
QGISSY

SEQ ID NO: 82 - skipped

SEQ ID NO: 83 - protein, 11 aa, synthetic construct [sequence omitted]

SEQ ID NO: 84 - DNA, 351 bp, synthetic construct
  misc_feature 1..351: note = source = /note="Description of Artificial Sequence: Synthetic polynucleotide"
  source 1..351: mol_type = other DNA
caggtgcagctggtggagtctgggggaggcttggtacagccgggggggtccctgagactctcctgtacagcctctggattcacctttagcagctatgccatgagctgggtccgccaggctccagggaaggggctggagtgggtctcagctattggtggtagtggtggtagcacatactacgcagactccgtgaagggccggttcaccatctccagagacaattccaagaacacgctgtatctgcaaatgaacagcctgagagccgaggacacggccgtatattactgtgtgaaagatggggcaggaggctttgactactggggccagggaaccctggtcaccgtctcgagt

SEQ ID NO: 85 - DNA, 321 bp, synthetic construct
  misc_feature 1..321: note = source = /note="Description of Artificial Sequence: Synthetic polynucleotide"
  source 1..321: mol_type = other DNA
gatattgtgatgacgcagtcttcatcctccctgtctgcatctgtaggagacagagtcaccatcacttgccgggcaagtcagggcattataaatcatttaggctggtatcagcataaaccagggaaagcccctaatcgcctaatctatgctgcatccagtttgcaaagtggggtcccatcaaggttcagcggcagtggatctgggacagaattcactctcacaatcagcagcctgcagcctgaagattttgcaacttattactgtctacggcataatagttaccctccgacgttcggccaagggaccaaggtggaaatcaag

SEQ ID NO: 86 - protein, 117 aa, synthetic construct
  REGION 1..117: note = source = /note="Description of Artificial Sequence: Synthetic polypeptide"
QVQLVESGGGLVQPGGSLRLSCTASGFTFSSYAMSWVRQAPGKGLEWVSAIGGSGGSTYYADSVKGRFTISRDNSKNTLYLQMNSLRAEDTAVYYCVKDGAGGFDYWGQGTLVTVSS

SEQ ID NO: 87 - protein, 107 aa, synthetic construct
  REGION 1..107: note = source = /note="Description of Artificial Sequence: Synthetic polypeptide"
DIVMTQSSSSLSASVGDRVTITCRASQGIINHLGWYQHKPGKAPNRLIYAASSLQSGVPSRFSGSGSGTEFTLTISSLQPEDFATYYCLRHNSYPPTFGQGTKVEIK

SEQ ID NO: 88 - protein, 8 aa, synthetic construct
  REGION 1..8: note = source = /note="Description of Artificial Sequence: Synthetic peptide"
GFTFSSYA

SEQ ID NO: 89 - protein, 8 aa, synthetic construct
  REGION 1..8: note = source = /note="Description of Artificial Sequence: Synthetic peptide"
IGGSGGST

SEQ ID NO: 90 - protein, 12 aa, synthetic construct
  REGION 1..12: note = source = /note="Description of Artificial Sequence: Synthetic peptide"
CVKDGAGGFDYW

SEQ ID NO: 91 - protein, 6 aa, synthetic construct
  REGION 1..6: note = source = /note="Description of Artificial Sequence: Synthetic peptide"
QGIINH

SEQ ID NO: 92 - skipped

SEQ ID NO: 93 - protein, 5 aa, synthetic construct
  REGION 1..5: note = source = /note="Description of Artificial Sequence: Synthetic peptide"
YPPTF

SEQ ID NO: 94 - DNA, 366 bp, synthetic construct
  misc_feature 1..366: note = source = /note="Description of Artificial Sequence: Synthetic polynucleotide"
  source 1..366: mol_type = other DNA
caggtgcagctacagcagtcgggcccaggactggtgaagccttcacagaccctgtccctcacctgcactgtctctggtggctccatcaacagtggtggtcactactggagctggatccgccagcacccagggaggggcctggagtggattgggtacatctattacagtgggagcatctactacaacccgtccctcaagagtcgacttaccatatcagtagacacgtctaagaaccagttctccctgaagctgagctctgtgactgccgcggacacggccgtgtattactgtgcgagttattactatgccagtagtggtgatgcttttgatatctggggccaagggacaatggtcaccgtctcgagt

SEQ ID NO: 95 - DNA, 321 bp, synthetic construct
  misc_feature 1..321: note = source = /note="Description of Artificial Sequence: Synthetic polynucleotide"
  source 1..321: mol_type = other DNA
gaaacgacactcacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaa

SEQ ID NO: 96 - protein, 122 aa, synthetic construct
  REGION 1..122: note = source = /note="Description of Artificial Sequence: Synthetic polypeptide"
QVQLQQSGPGLVKPSQTLSLTCTVSGGSINSGGHYWSWIRQHPGRGLEWIGYIYYSGSIYYNPSLKSRLTISVDTSKNQFSLKLSSVTAADTAVYYCASYYYASSGDAFDIWGQGTMVTVSS

SEQ ID NO: 97 - protein, 107 aa, synthetic construct
  REGION 1..107: note = source = /note="Description of Artificial Sequence: Synthetic polypeptide"
ETTLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 98 - protein, 10 aa, synthetic construct
  REGION 1..10: note = source = /note="Description of Artificial Sequence: Synthetic peptide"
GGSINSGGHY

SEQ ID NO: 99 - protein, 7 aa, synthetic construct
  REGION 1..7: note = source = /note="Description of Artificial Sequence: Synthetic peptide"
IYYSGSI

SEQ ID NO: 100 - protein, 16 aa, synthetic construct
  REGION 1..16: note = source = /note="Description of Artificial Sequence: Synthetic peptide"
CASYYYASSGDAFDIW

SEQ ID NO: 101 - protein, 6 aa, synthetic construct
  REGION 1..6: note = source = /note="Description of Artificial Sequence: Synthetic peptide"
QSVSSY

SEQ ID NO: 102 - skipped

SEQ ID NO: 103 - protein, 11 aa, synthetic construct [sequence omitted]

SEQ ID NO: 104 - DNA, 372 bp, synthetic construct
  misc_feature 1..372: note = source = /note="Description of Artificial Sequence: Synthetic polynucleotide"
  source 1..372: mol_type = other DNA
caggtgcagctgcaggagtctgggggaggcttggtacagccgggggggtccctgagactctcctgtacagcctctggattcacctttagtaattatgccatgagctgggtccgccaggctccagggaaggggctggagtgggtctcagctattagtggtcgtggtggtagcacattcttcgcagactccgtgaagggccggttcaccatctccagagacaattccaagagcacgctgtatctgcaaacgaacagcctgagagccgaggacacggccgtatattactgtgcgaaagggggcccgttgtataactggaacgacggtgatggttttgatatctggggccaagggaccacggtcacagtctcgagt

SEQ ID NO: 105 - DNA, 324 bp, synthetic construct
  misc_feature 1..324: note = source = /note="Description of Artificial Sequence: Synthetic polynucleotide"
  source 1..324: mol_type = other DNA
gaaattgtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcactgggtctgggacagagttcactctcaccatcagcagcctgcagtctgaagattttgcactttattactgtcagcagtatgataactggcctccgtggacgttcggccaagggaccaaggtggaaatcaaa

SEQ ID NO: 106 - protein, 124 aa, synthetic construct
  REGION 1..124: note = source = /note="Description of Artificial Sequence: Synthetic polypeptide"
QVQLQESGGGLVQPGGSLRLSCTASGFTFSNYAMSWVRQAPGKGLEWVSAISGRGGSTFFADSVKGRFTISRDNSKSTLYLQTNSLRAEDTAVYYCAKGGPLYNWNDGDGFDIWGQGTTVTVSS

SEQ ID NO: 107 - protein, 108 aa, synthetic construct
  REGION 1..108: note = source = /note="Description of Artificial Sequence: Synthetic polypeptide"
EIVLTQSPATLSVSPGERATLSCRASQSVSSNLAWYQQKPGQAPRLLIYGASTRATGIPARFSGTGSGTEFTLTISSLQSEDFALYYCQQYDNWPPWTFGQGTKVEIK

SEQ ID NO: 108 - protein, 8 aa, synthetic construct
  REGION 1..8: note = source = /note="Description of Artificial Sequence: Synthetic peptide"
GFTFSNYA

SEQ ID NO: 109 - protein, 8 aa, synthetic construct
  REGION 1..8: note = source = /note="Description of Artificial Sequence: Synthetic peptide"
ISGRGGST

SEQ ID NO: 110 - protein, 19 aa, synthetic construct
  REGION 1..19: note = source = /note="Description of Artificial Sequence: Synthetic peptide"
CAKGGPLYNWNDGDGFDIW

SEQ ID NO: 111 - protein, 6 aa, synthetic construct
  REGION 1..6: note = source = /note="Description of Artificial Sequence: Synthetic peptide"
QSVSSN

SEQ ID NO: 112 - skipped

SEQ ID NO: 113 - protein, 12 aa, synthetic construct [sequence omitted]

SEQ ID NO: 114 - DNA, 357 bp, synthetic construct
  misc_feature 1..357: note = source = /note="Description of Artificial Sequence: Synthetic polynucleotide"
  source 1..357: mol_type = other DNA
caggtgcagctgcaggagtctgggggaggcttggtcaagcctggagggtccctgagactctcctgtgcagcctctggattcaccttcagtgactactacatgacctggatccgccaggctccagggaaggggctggagtggatttcatacattagtggtggtggtggttccatatactacgcagactctgtgaagggccgattcaccatctccagggacaacgccaagaactcactgtatctgcaaatgaacagcctgagagccgaggacacggccgtttatttctgtgcgagagggaactggggatcggcggctcttgatatctggggccaagggacaatggtcacggtctcgagt

SEQ ID NO: 115 - DNA, 321 bp, synthetic construct
  misc_feature 1..321: note = source = /note="Description of Artificial Sequence: Synthetic polynucleotide"
  source 1..321: mol_type = other DNA
gaaattgtgttgacgcagtctccatcctcactgtctgcatctgtaggagacagagtcaccatcacttgtcgggcgagtcagggcattaacaattatttagcctggtttcagcagaaaccagggagagcccctaagtccctgatctatgctgcatccagtttgcaaagtggggtcccatcgaagttcagcggcagtggatctgggacagatttcactctcaccatcagcagcctgcagcctgaagattttgcaacttattactgccaacagtataatagttaccctccaactctcggccctgggaccaacgtggatatcaaa

SEQ ID NO: 116 - protein, 119 aa, synthetic construct
  REGION 1..119: note = source = /note="Description of Artificial Sequence: Synthetic polypeptide"
QVQLQESGGGLVKPGGSLRLSCAASGFTFSDYYMTWIRQAPGKGLEWISYISGGGGSIYYADSVKGRFTISRDNAKNSLYLQMNSLRAEDTAVYFCARGNWGSAALDIWGQGTMVTVSS

SEQ ID NO: 117 - protein, 107 aa, synthetic construct
  REGION 1..107: note = source = /note="Description of Artificial Sequence: Synthetic polypeptide"
EIVLTQSPSSLSASVGDRVTITCRASQGINNYLAWFQQKPGRAPKSLIYAASSLQSGVPSKFSGSGSGTDFTLTISSLQPEDFATYYCQQYNSYPPTLGPGTNVDIK

SEQ ID NO: 118 - protein, 8 aa, synthetic construct
  REGION 1..8: note = source = /note="Description of Artificial Sequence: Synthetic peptide"
GFTFSDYY

SEQ ID NO: 119 - protein, 11 aa, synthetic construct
  REGION 1..11: note = source = /note="Description of Artificial Sequence: Synthetic peptide"
ISYISGGGGSI

SEQ ID NO: 120 - protein, 14 aa, synthetic construct
  REGION 1..14: note = source = /note="Description of Artificial Sequence: Synthetic peptide"
CARGNWGSAALDIW

SEQ ID NO: 121 - protein, 6 aa, synthetic construct
  REGION 1..6: note = source = /note="Description of Artificial Sequence: Synthetic peptide"
QGINNY

SEQ ID NO: 122 - skipped

SEQ ID NO: 123 - protein, 11 aa, synthetic construct
  REGION 1..11: note = source = /note="Description of Artificial Sequence: Synthetic peptide"
CQQYNSYPPTL

SEQ ID NO: 124 - DNA, 351 bp, synthetic construct
  misc_feature 1..351: note = source = /note="Description of Artificial Sequence: Synthetic polynucleotide"
  source 1..351: mol_type = other DNA
caggtgcagctggtggagtctgggggaggcttggtacagccgggggggtccctgagactctcctgtacagcctctggattcacctttagcagctatgccatgagctgggtccgccaggctccagggaaggggctggagtgggtctcagctattggtggtagtggtggtagcgcatactacgcagactccgtgaagggccggttcaccatctccagagacaattccaagaacacgctgtatctgcaaatgaacagcctgagagccgaggacacggccgtatattactgtgtgaaagatggggcaggaggctttgactactggggccagggcaccctggtcaccgtctcgagt

SEQ ID NO: 125 - DNA, 321 bp, synthetic construct
  misc_feature 1..321: note = source = /note="Description of Artificial Sequence: Synthetic polynucleotide"
  source 1..321: mol_type = other DNA
gacatccagttgacccagtccccatcctccctgtctgcatctgtaggagacagagtcaccatcacttgccgggcaagtcagggcattagaaatcatttaggctggtatcagcagaaaccagggaaagcccctaagcgcctaatctatgctgcatccagtttgcaaagtggggtcccatcaaggttcagcggcagtggatctgggacagaattcactctcacaatcagcagcctgcagcctgaagattttgcaacttattactgtctacagcataatagttaccctccgacgttcggccaagggaccaaggtggaaatcaag

SEQ ID NO: 126 - protein, 117 aa, synthetic construct
  REGION 1..117: note = source = /note="Description of Artificial Sequence: Synthetic polypeptide"
QVQLVESGGGLVQPGGSLRLSCTASGFTFSSYAMSWVRQAPGKGLEWVSAIGGSGGSAYYADSVKGRFTISRDNSKNTLYLQMNSLRAEDTAVYYCVKDGAGGFDYWGQGTLVTVSS

SEQ ID NO: 127 - protein, 107 aa, synthetic construct
  REGION 1..107: note = source = /note="Description of Artificial Sequence: Synthetic polypeptide"
DIQLTQSPSSLSASVGDRVTITCRASQGIRNHLGWYQQKPGKAPKRLIYAASSLQSGVPSRFSGSGSGTEFTLTISSLQPEDFATYYCLQHNSYPPTFGQGTKVEIK

SEQ ID NO: 128 - protein, 8 aa, synthetic construct
  REGION 1..8: note = source = /note="Description of Artificial Sequence: Synthetic peptide"
GFTFSSYA

SEQ ID NO: 129 - protein, 8 aa, synthetic construct
  REGION 1..8: note = source = /note="Description of Artificial Sequence: Synthetic peptide"
IGGSGGSA

SEQ ID NO: 130 - protein, 12 aa, synthetic construct
  REGION 1..12: note = source = /note="Description of Artificial Sequence: Synthetic peptide"
CVKDGAGGFDYW

SEQ ID NO: 131 - protein, 6 aa, synthetic construct
  REGION 1..6: note = source = /note="Description of Artificial Sequence: Synthetic peptide"
QGIRNH

SEQ ID NO: 132 - skipped

SEQ ID NO: 133 - protein, 11 aa, synthetic construct [sequence omitted]

SEQ ID NO: 134 - DNA, 354 bp, synthetic construct
  misc_feature 1..354: note = source = /note="Description of Artificial Sequence: Synthetic polynucleotide"
  source 1..354: mol_type = other DNA
cagatgcagctacagcagtctgggggaggcttggtacagccgggggggtccctgagactctcctgtgcagcctctggattcaccgttagcagctatgccatgagctgggtccgccaggctctagggaaggggctggagtgggtctcaggtattagtggtagtggtggtagcacatactacgcagactccgtgaagggccggttcaccatctccagagacaattccaagaatacgctgtatctgcaaatgaacagcctgagagccgaggacacggccgtatattactgtgcgaagatagtgggagctacccactttgactactggggccagggaaccctggtcacggtctcgagt

SEQ ID NO: 135 - DNA, 339 bp, synthetic construct
  misc_feature 1..339: note = source = /note="Description of Artificial Sequence: Synthetic polynucleotide"
  source 1..339: mol_type = other DNA
gaaattgtgatgacacagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtggtccgatcaccttcggccaagggacacgactggagattaag

SEQ ID NO: 136 - protein, 118 aa, synthetic construct
  REGION 1..118: note = source = /note="Description of Artificial Sequence: Synthetic polypeptide"
QMQLQQSGGGLVQPGGSLRLSCAASGFTVSSYAMSWVRQALGKGLEWVSGISGSGGSTYYADSVKGRFTISRDNSKNTLYLQMNSLRAEDTAVYYCAKIVGATHFDYWGQGTLVTVSS

SEQ ID NO: 137 - protein, 113 aa, synthetic construct
  REGION 1..113: note = source = /note="Description of Artificial Sequence: Synthetic polypeptide"
EIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSGPITFGQGTRLEIK

SEQ ID NO: 138 - protein, 8 aa, synthetic construct
  REGION 1..8: note = source = /note="Description of Artificial Sequence: Synthetic peptide"
GFTVSSYA

SEQ ID NO: 139 - protein, 8 aa, synthetic construct
  REGION 1..8: note = source = /note="Description of Artificial Sequence: Synthetic peptide"
ISGSGGST

SEQ ID NO: 140 - protein, 13 aa, synthetic construct
  REGION 1..13: note = source = /note="Description of Artificial Sequence: Synthetic peptide"
CAKIVGATHFDYW

SEQ ID NO: 141 - protein, 12 aa, synthetic construct
  REGION 1..12: note = source = /note="Description of Artificial Sequence: Synthetic peptide"
QSVLYSSNNKNY

SEQ ID NO: 142 - skipped

SEQ ID NO: 143 - protein, 11 aa, synthetic construct [sequence omitted]

SEQ ID NO: 144 - DNA, 366 bp, synthetic construct
  misc_feature 1..366: note = source = /note="Description of Artificial Sequence: Synthetic polynucleotide"
  source 1..366: mol_type = other DNA
caggtgcagctggtggagtcgggcccaggactggtgaagccttcacagaccctgtccctcacctgcactgtctctggtggctccatcaacagtggtggttactactggagctggatccgccagcacccagggaagggcctggagtggattgggtacatctattacagtgggagcatctactacaacccgtccctcaggagtcgacttaccatatcagtagacacgtctaagaaccagttctccctgaagctgagctctgtgactgccgcggacacggccgtttattactgtgcgactccttattactatggttcggggagttatggggactactggggccagggcaccctggtcactgtctcgagt

SEQ ID NO: 145 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtctccagccaccctgtctttgtctccaggggaaagagccaccctctcctgcagggccagtcagagtgttaacaactacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcccatcaccttcggccaagggacacgactggagattaaa

SEQ ID NO: 146 - protein, 122 aa, synthetic construct
  REGION 1..122: note = source = /note="Description of Artificial Sequence: Synthetic polypeptide"
QVQLVESGPGLVKPSQTLSLTCTVSGGSINSGGYYWSWIRQHPGKGLEWIGYIYYSGSIYYNPSLRSRLTISVDTSKNQFSLKLSSVTAADTAVYYCATPYYYGSGSYGDYWGQGTLVTVSS

SEQ ID NO: 147 - protein, 107 aa, synthetic construct
  REGION 1..107: note = source = /note="Description of Artificial Sequence: Synthetic polypeptide"
DIQMTQSPATLSLSPGERATLSCRASQSVNNYLAWYQQKPGQAPRLLIYDASNRATGIPARFSGSGSGTDFTLTISSLEPEDFAVYYCQQRSNWPITFGQGTRLEIK

SEQ ID NO: 148 - protein, 10 aa, synthetic construct
  REGION 1..10: note = source = /note="Description of Artificial Sequence: Synthetic peptide"
GGSINSGGYY

SEQ ID NO: 149 - protein, 7 aa, synthetic construct
  REGION 1..7: note = source = /note="Description of Artificial Sequence: Synthetic peptide"
IYYSGSI

SEQ ID NO: 150 - protein, 16 aa, synthetic construct
  REGION 1..16: note = source = /note="Description of Artificial Sequence: Synthetic peptide"
CATPYYYGSGSYGDYW

SEQ ID NO: 151 - protein, 6 aa, synthetic construct
  REGION 1..6: note = source = /note="Description of Artificial Sequence: Synthetic peptide"
QSVNNY

SEQ ID NO: 152 - skipped

SEQ ID NO: 153 - protein, 11 aa, synthetic construct [sequence omitted]

SEQ ID NO: 154 - DNA, 363 bp, synthetic construct
  misc_feature 1..363: note = source = /note="Description of Artificial Sequence: Synthetic polynucleotide"
  source 1..363: mol_type = other DNA
cagatgcagctgcaggagtcgggcccaggactggtgaagccttctcagaccctgtccctcacctgcactgtgtctggtggctccatcagcagtgttggttactactggaactggatccgccagcacccagggaagggcctggagttcattgggtacatctattacagtgggagcatctactacaatccgtccctcaagagtcgagttaccatatccgtagacacgtctaagaaccagttctccctgaagctgagctctgtgactgccgcggacacggccctatattactgtgcgagcgtcggtatagtgggagcctcctactttgagtactggggccagggaaccctggtcacagtctcgagt

SEQ ID NO: 155 - DNA, 321 bp, synthetic construct
  misc_feature 1..321: note = source = /note="Description of Artificial Sequence: Synthetic polynucleotide"
  source 1..321: mol_type = other DNA
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctatcaccttcggccaagggacacgactggagatcaag

SEQ ID NO: 156 - protein, 121 aa, synthetic construct
  REGION 1..121: note = source = /note="Description of Artificial Sequence: Synthetic polypeptide"
QMQLQESGPGLVKPSQTLSLTCTVSGGSISSVGYYWNWIRQHPGKGLEFIGYIYYSGSIYYNPSLKSRVTISVDTSKNQFSLKLSSVTAADTALYYCASVGIVGASYFEYWGQGTLVTVSS

SEQ ID NO: 157 - protein, 107 aa, synthetic construct
  REGION 1..107: note = source = /note="Description of Artificial Sequence: Synthetic polypeptide"
EIVMTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 158 - protein, 10 aa, synthetic construct
  REGION 1..10: note = source = /note="Description of Artificial Sequence: Synthetic peptide"
GGSISSVGYY

SEQ ID NO: 159 - protein, 7 aa, synthetic construct
  REGION 1..7: note = source = /note="Description of Artificial Sequence: Synthetic peptide"
IYYSGSI

SEQ ID NO: 160 - protein, 15 aa, synthetic construct
  REGION 1..15: note = source = /note="Description of Artificial Sequence: Synthetic peptide"
CASVGIVGASYFEYW

SEQ ID NO: 161 - protein, 6 aa, synthetic construct
  REGION 1..6: note = source = /note="Description of Artificial Sequence: Synthetic peptide"
QSVSSY

SEQ ID NO: 162 - skipped

SEQ ID NO: 163 - protein, 11 aa, synthetic construct [sequence omitted]

SEQ ID NO: 164 - DNA, 354 bp, synthetic construct
  misc_feature 1..354: note = source = /note="Description of Artificial Sequence: Synthetic polynucleotide"
  source 1..354: mol_type = other DNA
caggtgcagctacagcagtctgggggaggcttggtacagcctggggggtccctgagactctcctgtgcagcctctggattcaccttaagcagctatgccatgagctgggtccgccaggctccagggaaggggctggagtgggtctcaggtattagtggtagtggtggtagcacatacaacgcagactccgtgaagggccggttcaccatctccagagacaattccaagaacacgctgtttctgcaaatgaacagcctgagagccgaggacacggccgtatattactgtgcgaaaatttttgggtcctactactttgactactggggccagggaaccctggtcacagtctcgagt

SEQ ID NO: 165 - DNA, 339 bp, synthetic construct
  misc_feature 1..339: note = source = /note="Description of Artificial Sequence: Synthetic polynucleotide"
  source 1..339: mol_type = other DNA
gaaattgtgatgacacagtctccagactccctggctgtgtctctgggcgagagggccaccatcaactgcaagtccagccagagtgttttatacagctccaacaataagaactacttagcttggtaccagcagaaaccaggacagcctcctaagctgctcatttactggacatctacccgggaatccggggtccctaaccgattcagtggcagcgggtctgggacagatttcactctcaccatcagcagcctgcaggctgaagatgtggcagtttattactgtcagcaatattatagtggtcctccgacgttcggccaagggaccaaggtggaaatcaaa

SEQ ID NO: 166 - protein, 118 aa, synthetic construct
  REGION 1..118: note = source = /note="Description of Artificial Sequence: Synthetic polypeptide"
QVQLQQSGGGLVQPGGSLRLSCAASGFTLSSYAMSWVRQAPGKGLEWVSGISGSGGSTYNADSVKGRFTISRDNSKNTLFLQMNSLRAEDTAVYYCAKIFGSYYFDYWGQGTLVTVSS

SEQ ID NO: 167 - protein, 113 aa, synthetic construct
  REGION 1..113: note = source = /note="Description of Artificial Sequence: Synthetic polypeptide"
EIVMTQSPDSLAVSLGERATINCKSSQSVLYSSNNKNYLAWYQQKPGQPPKLLIYWTSTRESGVPNRFSGSGSGTDFTLTISSLQAEDVAVYYCQQYYSGPPTFGQGTKVEIK

SEQ ID NO: 168 - protein, 8 aa, synthetic construct
  REGION 1..8: note = source = /note="Description of Artificial Sequence: Synthetic peptide"
GFTLSSYA

SEQ ID NO: 169 - protein, 8 aa, synthetic construct
  REGION 1..8: note = source = /note="Description of Artificial Sequence: Synthetic peptide"
ISGSGGST

SEQ ID NO: 170 - protein, 13 aa, synthetic construct
  REGION 1..13: note = source = /note="Description of Artificial Sequence: Synthetic peptide"
CAKIFGSYYFDYW

SEQ ID NO: 171 - protein, 12 aa, synthetic construct
  REGION 1..12: note = source = /note="Description of Artificial Sequence: Synthetic peptide"
QSVLYSSNNKNY

SEQ ID NO: 172 - skipped

SEQ ID NO: 173 - protein, 11 aa, synthetic construct [sequence omitted]

SEQ ID NO: 174 - DNA, 354 bp, synthetic construct
  misc_feature 1..354: note = source = /note="Description of Artificial Sequence: Synthetic polynucleotide"
  source 1..354: mol_type = other DNA
caggtgcagctggtggagtctgggggaggcttggttcggcctggggggtccctgagactctcctgtgcagtctctggattcacctttagcagctatgccatgagctgggtccgccaggctccagggaaggggctggagtgggtctcaggtattagtggtagtggtggtagcacatacaacgcagactccgtgaagggccggttcaccatctccagagacaattccaagaacacgctgtatcttcaaatgaacagcctgagagccgaggacacggccgtatattactgtgcgaaaatttttgggtcctactactttgactactggggccagggaaccctggtcaccgtctcgagt

SEQ ID NO: 175 - DNA, 339 bp, synthetic construct
  misc_feature 1..339: note = source = /note="Description of Artificial Sequence: Synthetic polynucleotide"
  source 1..339: mol_type = other DNA
gaaattgtgatgacacagtctccagactccctggctgtgtctctgggcgagagggccaccatcaactgcaagtccagccagagtgttttatacagctccaataataagaactacttagcttggtaccagcagaaatcaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtggtccaccgacgttcggccaagggaccaaggtggaaatcaaa

SEQ ID NO: 176 - protein, 118 aa, synthetic construct
  REGION 1..118: note = source = /note="Description of Artificial Sequence: Synthetic polypeptide"
QVQLVESGGGLVRPGGSLRLSCAVSGFTFSSYAMSWVRQAPGKGLEWVSGISGSGGSTYNADSVKGRFTISRDNSKNTLYLQMNSLRAEDTAVYYCAKIFGSYYFDYWGQGTLVTVSS

SEQ ID NO: 177 - protein, 113 aa, synthetic construct
  REGION 1..113: note = source = /note="Description of Artificial Sequence: Synthetic polypeptide"
EIVMTQSPDSLAVSLGERATINCKSSQSVLYSSNNKNYLAWYQQKSGQPPKLLIYWASTRESGVPDRFSGSGSGTDFTLTISSLQAEDVAVYYCQQYYSGPPTFGQGTKVEIK

SEQ ID NO: 178 - protein, 8 aa, synthetic construct
  REGION 1..8: note = source = /note="Description of Artificial Sequence: Synthetic peptide"
GFTFSSYA

SEQ ID NO: 179 - protein, 8 aa, synthetic construct
  REGION 1..8: note = source = /note="Description of Artificial Sequence: Synthetic peptide"
ISGSGGST

SEQ ID NO: 180 - protein, 13 aa, synthetic construct
  REGION 1..13: note = source = /note="Description of Artificial Sequence: Synthetic peptide"
CAKIFGSYYFDYW

SEQ ID NO: 181 - protein, 12 aa, synthetic construct
  REGION 1..12: note = source = /note="Description of Artificial Sequence: Synthetic peptide"
QSVLYSSNNKNY

SEQ ID NO: 182 - skipped

SEQ ID NO: 183 - protein, 11 aa, synthetic construct [sequence omitted]

SEQ ID NO: 184 - DNA, 377 bp, synthetic construct
  misc_feature 1..377: note = source = /note="Description of Artificial Sequence: Synthetic polynucleotide"
  source 1..377: mol_type = other DNA
acgtgacagggcgcgcccaggtccagctgcaggagagcggtcccggactggtgaagccatcccagacactgagcctgacttgtactgtgagcggcggtagcatctccagcggcggctactattggtcctggatcaggcagcacccaggcaagggcctggagtggatcggctacatctactatagcggctctatctactataacccttccctgaagagccgggtgaccatctctgtggacacatccaagaatcagttctatctgaagctgtcttccgtgaccgccgctgatacagccgtgtactattgcgcctcactgatggtctggggggtcatgggcgattactgggggcagggcacactggtcacagtctcgagt

SEQ ID NO: 185 - DNA, 321 bp, synthetic construct
  misc_feature 1..321: note = source = /note="Description of Artificial Sequence: Synthetic polynucleotide"
  source 1..321: mol_type = other DNA
gagattgtgctgacccagtctcccgccaccctgtctctgagtcctggcgagagagccaccctgagctgcagagcctctcagtccgtgtccagctatctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagcggtccgactggcctcctacatttggccaaggcaccaaggtggaaatcaag

SEQ ID NO: 186 - protein, 120 aa, synthetic construct
  REGION 1..120: note = source = /note="Description of Artificial Sequence: Synthetic polypeptide"
QVQLQESGPGLVKPSQTLSLTCTVSGGSISSGGYYWSWIRQHPGKGLEWIGYIYYSGSIYYNPSLKSRVTISVDTSKNQFYLKLSSVTAADTAVYYCASLMVWGVMGDYWGQGTLVTVSS

SEQ ID NO: 187 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSDWPPTFGQGTKVEIK

SEQ ID NO: 188 - protein, 10 aa, synthetic construct
  REGION 1..10: note = source = /note="Description of Artificial Sequence: Synthetic peptide"
GGSISSGGYY

SEQ ID NO: 189 - protein, 7 aa, synthetic construct
  REGION 1..7: note = source = /note="Description of Artificial Sequence: Synthetic peptide"
IYYSGSI

SEQ ID NO: 190 - protein, 14 aa, synthetic construct
  REGION 1..14: note = source = /note="Description of Artificial Sequence: Synthetic peptide"
CASLMVWGVMGDYW

SEQ ID NO: 191 - protein, 6 aa, synthetic construct
  REGION 1..6: note = source = /note="Description of Artificial Sequence: Synthetic peptide"
QSVSSY

SEQ ID NO: 192 - skipped

SEQ ID NO: 193 - protein, 11 aa, synthetic construct [sequence omitted]

SEQ ID NO: 194 - DNA, 354 bp, synthetic construct
  misc_feature 1..354: note = source = /note="Description of Artificial Sequence: Synthetic polynucleotide"
  source 1..354: mol_type = other DNA
caggtccagctacagcagtctgggggaggcttggttcatcctggggggtccctaagactctcctgtgcagcctctggattcaccgttgacacctatgccatgacctgggtccgccaggctccagggaaggggctggagtgggtctcaggtattagcggtagtggtggtagcacatactacgcagactccgtgaagggccggttcaccatctccagagacaactccaagaacacgctgtatctgcaaatgaacagcctgagagacgaggacacggccgtatattactgtgcgaagatagtgggagttacccactttgactactggggccagggcaccctggtcacggtctcgagt

SEQ ID NO: 195 - DNA, 339 bp, synthetic construct
  misc_feature 1..339: note = source = /note="Description of Artificial Sequence: Synthetic polynucleotide"
  source 1..339: mol_type = other DNA
gaaattgtgatgacgcagtctccagactccctggctgtgtctctgggcgagagggccaccatcaactgcaagtccagccagagtgttttatacaggtccaacaataagaactatttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtggtccgatcaccttcggccaagggacacgactggagattaag

SEQ ID NO: 196 - protein, 118 aa, synthetic construct
  REGION 1..118: note = source = /note="Description of Artificial Sequence: Synthetic polypeptide"
QVQLQQSGGGLVHPGGSLRLSCAASGFTVDTYAMTWVRQAPGKGLEWVSGISGSGGSTYYADSVKGRFTISRDNSKNTLYLQMNSLRDEDTAVYYCAKIVGVTHFDYWGQGTLVTVSS

SEQ ID NO: 197 - protein, 113 aa, synthetic construct
  REGION 1..113: note = source = /note="Description of Artificial Sequence: Synthetic polypeptide"
EIVMTQSPDSLAVSLGERATINCKSSQSVLYRSNNKNYLAWYQQKPGQPPKLLIYWASTRESGVPDRFSGSGSGTDFTLTISSLQAEDVAVYYCQQYYSGPITFGQGTRLEIK

SEQ ID NO: 198 - protein, 8 aa, synthetic construct
  REGION 1..8: note = source = /note="Description of Artificial Sequence: Synthetic peptide"
GFTVDTYA

SEQ ID NO: 199 - protein, 8 aa, synthetic construct
  REGION 1..8: note = source = /note="Description of Artificial Sequence: Synthetic peptide"
ISGSGGST

SEQ ID NO: 200 - protein, 13 aa, synthetic construct
  REGION 1..13: note = source = /note="Description of Artificial Sequence: Synthetic peptide"
CAKIVGVTHFDYW

SEQ ID NO: 201 - protein, 12 aa, synthetic construct
  REGION 1..12: note = source = /note="Description of Artificial Sequence: Synthetic peptide"
QSVLYRSNNKNY

SEQ ID NO: 202 - skipped

SEQ ID NO: 203 - protein, 11 aa, synthetic construct [sequence omitted]

SEQ ID NO: 204 - DNA, 369 bp, synthetic construct
  misc_feature 1..369: note = source = /note="Description of Artificial Sequence: Synthetic polynucleotide"
  source 1..369: mol_type = other DNA
caggtcaccttgaaggagtcgggcccaggactggtgaagccttcacagaccctgtccctcacctgcactgtctctggtggctccatcagcagtggtggtcattactggagctggatccgccagcacccagggaagggcctggagtggattgggtacatctcttacagtgggagcacctactacaacccgtccctcaagagtcgacttaccatatcagtagacacgtctaagaaccagttctccctgaagctgagctctgtgactgccgcggacacggccgtgtattactgtgcgaccgcgtattacgatattttgactggttacccttttgactactggggccagggaaccctggtcacggtctcgagt

SEQ ID NO: 205 - DNA, 321 bp, synthetic construct
  misc_feature 1..321: note = source = /note="Description of Artificial Sequence: Synthetic polynucleotide"
  source 1..321: mol_type = other DNA
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccgacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagatcaag

SEQ ID NO: 206 - protein, 123 aa, synthetic construct
  REGION 1..123: note = source = /note="Description of Artificial Sequence: Synthetic polypeptide"
QVTLKESGPGLVKPSQTLSLTCTVSGGSISSGGHYWSWIRQHPGKGLEWIGYISYSGSTYYNPSLKSRLTISVDTSKNQFSLKLSSVTAADTAVYYCATAYYDILTGYPFDYWGQGTLVTVSS

SEQ ID NO: 207 - protein, 107 aa, synthetic construct
  REGION 1..107: note = source = /note="Description of Artificial Sequence: Synthetic polypeptide"
EIVMTQSPATLSLSPGERATLSCRASQSVSSYLAWYQQKPGQAPRLLIYDASDRATGIPARFSGSGSGTDFTLTISSLEPEDFAVYYCQQRSNWPITFGQGTRLEIK

SEQ ID NO: 208 - protein, 10 aa, synthetic construct
  REGION 1..10: note = source = /note="Description of Artificial Sequence: Synthetic peptide"
GGSISSGGHY

SEQ ID NO: 209 - protein, 7 aa, synthetic construct
  REGION 1..7: note = source = /note="Description of Artificial Sequence: Synthetic peptide"
ISYSGST

SEQ ID NO: 210 - protein, 17 aa, synthetic construct
  REGION 1..17: note = source = /note="Description of Artificial Sequence: Synthetic peptide"
CATAYYDILTGYPFDYW

SEQ ID NO: 211 - protein, 6 aa, synthetic construct
  REGION 1..6: note = source = /note="Description of Artificial Sequence: Synthetic peptide"
QSVSSY

SEQ ID NO: 212 - skipped

SEQ ID NO: 213 - protein, 11 aa, synthetic construct [sequence omitted]

SEQ ID NO: 214 - DNA, 369 bp, synthetic construct
  misc_feature 1..369: note = source = /note="Description of Artificial Sequence: Synthetic polynucleotide"
  source 1..369: mol_type = other DNA
caggtgcagctacagcagtcgggcccaggactggtgaagccttcacagaccctgtccctcacctgcactgtctctggtggctccatcagcagtggtggttactactggagctggatccgccagcacccagggaagggcctggagtggattgggtacatctcttatagtgggagtatctactacaacccgtccctcaagagtcgagttaccatatcagtagacacgtctaagaaccagttctccctgaagctgagctctgtgactgccacggacacggccgtgtattactgtgcgaccgcgtattacgatcttttgactggttacccttttgactactggggccagggaaccctggtcacggtctcgagt

SEQ ID NO: 215 - DNA, 321 bp, synthetic construct
  misc_feature 1..321: note = source = /note="Description of Artificial Sequence: Synthetic polynucleotide"
  source 1..321: mol_type = other DNA
gaaattgtgatgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaag

SEQ ID NO: 216 - protein, 123 aa, synthetic construct
  REGION 1..123: note = source = /note="Description of Artificial Sequence: Synthetic polypeptide"
QVQLQQSGPGLVKPSQTLSLTCTVSGGSISSGGYYWSWIRQHPGKGLEWIGYISYSGSIYYNPSLKSRVTISVDTSKNQFSLKLSSVTATDTAVYYCATAYYDLLTGYPFDYWGQGTLVTVSS

SEQ ID NO: 217 - protein, 107 aa, synthetic construct; identical to SEQ ID NO: 157
  REGION 1..107: note = source = /note="Description of Artificial Sequence: Synthetic polypeptide"

SEQ ID NO: 218 - protein, 10 aa, synthetic construct
  REGION 1..10: note = source = /note="Description of Artificial Sequence: Synthetic peptide"
GGSISSGGYY

SEQ ID NO: 219 - protein, 7 aa, synthetic construct
  REGION 1..7: note = source = /note="Description of Artificial Sequence: Synthetic peptide"
ISYSGSI

SEQ ID NO: 220 - protein, 17 aa, synthetic construct
  REGION 1..17: note = source = /note="Description of Artificial Sequence: Synthetic peptide"
CATAYYDLLTGYPFDYW

SEQ ID NO: 221 - protein, 6 aa, synthetic construct
  REGION 1..6: note = source = /note="Description of Artificial Sequence: Synthetic peptide"
QSVSSY

SEQ ID NO: 222 - skipped

SEQ ID NO: 223 - protein, 11 aa, synthetic construct [sequence omitted]

SEQ ID NO: 224 - DNA, 379 bp, synthetic construct
  misc_feature 1..379: note = source = /note="Description of Artificial Sequence: Synthetic polynucleotide"
  source 1..379: mol_type = other DNA
ggcgcgccgaggtgcagctgctggaatctggaggaggactggtccagccaggtggatccctgcgactgagctgcgccgcttctggattcacctttacaagatacgacatggtgtgggtccgccaggcaccaggaaagggactggagtgggtggctggtatcggcgatagtaacaagatgacccgctacgcacctgccgtcaaagggagggcaacaattagtcgggacaactcaaagaatactctgtatctgcagatgaattccctgcgagctgaggatacagcagtgtactattgtgccaaaggtagctgcatcgcctgttgggacgaagctggccgtattgatgcatggggacaggggactctggtgaccgtctcgag

SEQ ID NO: 225 - DNA, 351 bp, synthetic construct
  misc_feature 1..351: note = source = /note="Description of Artificial Sequence: Synthetic polynucleotide"
  source 1..351: mol_type = other DNA
gctagcctcttacgagctgactcaggaccctgcagtgagtgtcgccctgggccagacagtgagaatcacttgctccggcggagggagctacgatggttccagctactatggctggtatcagcagaagccaggacaggcacctgtgaccgtcatctataacaataacaataggccatctgacattcccgatcggttcagtggatctagttcagggaacacagcttctctgaccattacaggagcccaggctgaggacgaagcagattactattgtgggtcatacgacaggccagaaacaaattccgattatgtgggaatgtttggtagcggcactaaagtcaccgtcctagg

SEQ ID NO: 226 - protein, 124 aa, synthetic construct
  REGION 1..124: note = source = /note="Description of Artificial Sequence: Synthetic polypeptide"
EVQLLESGGGLVQPGGSLRLSCAASGFTFTRYDMVWVRQAPGKGLEWVAGIGDSNKMTRYAPAVKGRATISRDNSKNTLYLQMNSLRAEDTAVYYCAKGSCIACWDEAGRIDAWGQGTLVTVSS

SEQ ID NO: 227 - protein, 114 aa, synthetic construct
  REGION 1..114: note = source = /note="Description of Artificial Sequence: Synthetic polypeptide"
SYELTQDPAVSVALGQTVRITCSGGGSYDGSSYYGWYQQKPGQAPVTVIYNNNNRPSDIPDRFSGSSSGNTASLTITGAQAEDEADYYCGSYDRPETNSDYVGMFGSGTKVTVL

SEQ ID NO: 228 - protein, 8 aa, synthetic construct
  REGION 1..8: note = source = /note="Description of Artificial Sequence: Synthetic peptide"
GFTFTRYD

SEQ ID NO: 229 - protein, 8 aa, synthetic construct
  REGION 1..8: note = source = /note="Description of Artificial Sequence: Synthetic peptide"
IGDSNKMT

SEQ ID NO: 230 - protein, 19 aa, synthetic construct
  REGION 1..19: note = source = /note="Description of Artificial Sequence: Synthetic peptide"
CAKGSCIACWDEAGRIDAW

SEQ ID NO: 231 - protein, 8 aa, synthetic construct
  REGION 1..8: note = source = /note="Description of Artificial Sequence: Synthetic peptide"
GSYDGSSY

SEQ ID NO: 232 - skipped

SEQ ID NO: 233 - protein, 17 aa, synthetic construct
  REGION 1..17: note = source = /note="Description of Artificial Sequence: Synthetic peptide"
CGSYDRPETNSDYVGMF

SEQ ID NO: 234 - DNA, 400 bp, synthetic construct
  misc_feature 1..400: note = source = /note="Description of Artificial Sequence: Synthetic polynucleotide"
  source 1..400: mol_type = other DNA
ggcgcgccgaggtgcagctgctggaaagcggaggaggactggtccagccaggtggatctctgcgactgagttgcgccgcttcaggcttcacattttctgactacgccatgaactgggtgaggcaggctcctggcaagggactggagtgggtcgcaggaatcgggaacgatggaagttacactaattatggagcagccgtgaaggggagagctactatttcccgcgacaacagcaaaaataccctgtacctgcagatgaactcactgagagctgaagataccgcagtgtactattgtgcctctgacatcaggagtcggaatgattgctcctatttcctgggagggtgttccagcggctttattgacgtgtggggtcagggcaccctggtcacagtctcgag

SEQ ID NO: 235 - DNA, 321 bp, synthetic construct
  misc_feature 1..321: note = source = /note="Description of Artificial Sequence: Synthetic polynucleotide"
  source 1..321: mol_type = other DNA
gctagcctcttacgagctgacccaggacccagcagtgtccgtcgccctgggccagacagtgagaatcacttgctccggcggatccagctacagctatgggtggttccagcagaagcccggtcaggcccctgtgaccgtcatctatgaaagtaacaataggccatcagacattcccgatcggttttctggctctagttcaggaaacacagctagtctgaccatcacaggggcccaggctgaggacgaagctgattactattgtggcaatgcagattccagctctggaattttcgggtccggtactaaagtcaccgtcctagg

SEQ ID NO: 236 - protein, 131 aa, synthetic construct
  REGION 1..131: note = source = /note="Description of Artificial Sequence: Synthetic polypeptide"
EVQLLESGGGLVQPGGSLRLSCAASGFTFSDYAMNWVRQAPGKGLEWVAGIGNDGSYTNYGAAVKGRATISRDNSKNTLYLQMNSLRAEDTAVYYCASDIRSRNDCSYFLGGCSSGFIDVWGQGTLVTVSS

SEQ ID NO: 237 - protein, 104 aa, synthetic construct
  REGION 1..104: note = source = /note="Description of Artificial Sequence: Synthetic polypeptide"
SYELTQDPAVSVALGQTVRITCSGGSSYSYGWFQQKPGQAPVTVIYESNNRPSDIPDRFSGSSSGNTASLTITGAQAEDEADYYCGNADSSSGIFGSGTKVTVL

SEQ ID NO: 238 - protein, 8 aa, synthetic construct
  REGION 1..8: note = source = /note="Description of Artificial Sequence: Synthetic peptide"
GFTFSDYA

SEQ ID NO: 239 - protein, 8 aa, synthetic construct
  REGION 1..8: note = source = /note="Description of Artificial Sequence: Synthetic peptide"
IGNDGSYT

SEQ ID NO: 240 - protein, 26 aa, synthetic construct
  REGION 1..26: note = source = /note="Description of Artificial Sequence: Synthetic peptide"
CASDIRSRNDCSYFLGGCSSGFIDVW

SEQ ID NO: 241 - protein, 4 aa, synthetic construct
  REGION 1..4: note = source = /note="Description of Artificial Sequence: Synthetic peptide"
SSYS

SEQ ID NO: 242 - skipped

SEQ ID NO: 243 - protein, 11 aa, synthetic construct
  REGION 1..11: note = source = /note="Description of Artificial Sequence: Synthetic peptide"
CGNADSSSGIF

SEQ ID NO: 244 - DNA, 385 bp, synthetic construct
  misc_feature 1..385: note = source = /note="Description of Artificial Sequence: Synthetic polynucleotide"
  source 1..385: mol_type = other DNA
ggcgcgccgaggtgcagctgctggaatccggaggaggactggtccagccaggtggatccctgcgactgagctgcgccgcttctggattcgactttagcgatcacgggatgcagtgggtgagacaggcaccaggcaagggactggagtacgtgggtgtcatcgacaccacaggccgctatacatactatgcacctgccgtcaagggcagggctaccattagtcgggacaactcaaaaaatacactgtacctgcagatgaactctctgagggctgaagatactgcagtgtactattgcgccaaaactacctgcgtgggagggtacctgtgcaataccgtcggaagtatcgatgcttggggacaggggacactggtgactgtctcgag

SEQ ID NO: 245 - DNA, 330 bp, synthetic construct
  misc_feature 1..330: note = source = /note="Description of Artificial Sequence: Synthetic polynucleotide"
  source 1..330: mol_type = other DNA
gctagcctcctacgagctgactcaggacccagcagtgagcgtcgccctgggccagacagtgagaatcacttgctctggcggagggtccagctcttactatggttggtaccagcagaagcccggccaggctcctgtgaccgtcatctatgacgatacaaacaggccaagtggaattcccgatcggttctcaggtagttcatccggcaatacagcttctctgaccatcacaggggcccaggctgaggacgaagcagattactattgtggtggctatgaaggaagctctcacgccgggatttttggaagtgggactaaagtcaccgtcctagg

SEQ ID NO: 246 - protein, 126 aa, synthetic construct
  REGION 1..126: note = source = /note="Description of Artificial Sequence: Synthetic polypeptide"
EVQLLESGGGLVQPGGSLRLSCAASGFDFSDHGMQWVRQAPGKGLEYVGVIDTTGRYTYYAPAVKGRATISRDNSKNTLYLQMNSLRAEDTAVYYCAKTTCVGGYLCNTVGSIDAWGQGTLVTVSS

SEQ ID NO: 247 - protein, 107 aa, synthetic construct
  REGION 1..107: note = source = /note="Description of Artificial Sequence: Synthetic polypeptide"
SYELTQDPAVSVALGQTVRITCSGGGSSSYYGWYQQKPGQAPVTVIYDDTNRPSGIPDRFSGSSSGNTASLTITGAQAEDEADYYCGGYEGSSHAGIFGSGTKVTVL

SEQ ID NO: 248 - protein, 8 aa, synthetic construct
  REGION 1..8: note = source = /note="Description of Artificial Sequence: Synthetic peptide"
GFDFSDHG

SEQ ID NO: 249 - protein, 8 aa, synthetic construct
  REGION 1..8: note = source = /note="Description of Artificial Sequence: Synthetic peptide"
IDTTGRYT

SEQ ID NO: 250 - protein, 21 aa, synthetic construct
  REGION 1..21: note = source = /note="Description of Artificial Sequence: Synthetic peptide"
CAKTTCVGGYLCNTVGSIDAW

SEQ ID NO: 251 - protein, 5 aa, synthetic construct
  REGION 1..5: note = source = /note="Description of Artificial Sequence: Synthetic peptide"
GSSSY

SEQ ID NO: 252 - skipped

SEQ ID NO: 253 - protein, 13 aa, synthetic construct
  REGION 1..13: note = source = /note="Description of Artificial Sequence: Synthetic peptide"
CGGYEGSSHAGIF

SEQ ID NO: 254 - DNA, 376 bp, synthetic construct
  misc_feature 1..376: note = source = /note="Description of Artificial Sequence: Synthetic polynucleotide"
  source 1..376: mol_type = other DNA
ggcgcgccgaggtgcagctgctggaaagtggaggaggactggtccagccaggtggaagcctgagactgtcttgcgccgctagtggcttcgacttttccagctacaccatgcagtgggtgaggcaggcaccaggcaagggactggagtgggtgggcgtcatctctagtactggagggtctaccggatacgggcctgctgtgaagggaagggcaacaatttcacgggataactccaaaaatactctgtatctgcagatgaacagcctgagggcagaagacacagccgtgtactattgcgtgaaatcaatctccggagatgcctggtctgtggacgggctggatgcttggggtcagggcaccctggtcacagtctcgag

SEQ ID NO: 255 - DNA, 321 bp, synthetic construct
  misc_feature 1..321: note = source = /note="Description of Artificial Sequence: Synthetic polynucleotide"
  source 1..321: mol_type = other DNA
gctagcctcatacgagctgacccaggacccagcagtgtccgtcgccctgggacagacagtgagaatcacttgctccggaggaggatccgcctacggttggtatcagcagaagcccggccaggcacctgtgaccgtcatctactataacaatcagaggccatctggcattcccgaccggttcagtggatccagctctgggaacacagcaagtctgaccatcacaggcgcccaggctgaggacgaagccgattactattgtggaagctatgatagttcagctgtggggatttttggttctggcactaaagtcaccgtcctagg

SEQ ID NO: 256 - protein, 123 aa, synthetic construct
  REGION 1..123: note = source = /note="Description of Artificial Sequence: Synthetic polypeptide"
EVQLLESGGGLVQPGGSLRLSCAASGFDFSSYTMQWVRQAPGKGLEWVGVISSTGGSTGYGPAVKGRATISRDNSKNTLYLQMNSLRAEDTAVYYCVKSISGDAWSVDGLDAWGQGTLVTVSS

SEQ ID NO: 257 - protein, 104 aa, synthetic construct
  REGION 1..104: note = source = /note="Description of Artificial Sequence: Synthetic polypeptide"
SYELTQDPAVSVALGQTVRITCSGGGSAYGWYQQKPGQAPVTVIYYNNQRPSGIPDRFSGSSSGNTASLTITGAQAEDEADYYCGSYDSSAVGIFGSGTKVTVL

SEQ ID NO: 258 - protein, 8 aa, synthetic construct
  REGION 1..8: note = source = /note="Description of Artificial Sequence: Synthetic peptide"
GFDFSSYT

SEQ ID NO: 259 - protein, 8 aa, synthetic construct
  REGION 1..8: note = source = /note="Description of Artificial Sequence: Synthetic peptide"
ISSTGGST

SEQ ID NO: 260 - protein, 18 aa, synthetic construct
  REGION 1..18: note = source = /note="Description of Artificial Sequence: Synthetic peptide"
CVKSISGDAWSVDGLDAW

SEQ ID NO: 261-262 - skipped (2 entries)

SEQ ID NO: 263 - protein, 12 aa, synthetic construct
  REGION 1..12: note = source = /note="Description of Artificial Sequence: Synthetic peptide"
CGSYDSSAVGIF

SEQ ID NO: 264 - DNA, 376 bp, synthetic construct
  misc_feature 1..376: note = source = /note="Description of Artificial Sequence: Synthetic polynucleotide"
  source 1..376: mol_type = other DNA
ggcgcgccgaggtgcagctgctggaaagtggaggaggactggtccagccaggtggaagcctgagactgtcttgcgccgctagtggcttcgacttttccagctacaccatgcagtgggtgaggcaggcaccaggcaagggactggagtgggtgggcgtcatctctagtactggagggtctaccggatacgggcctgctgtgaagggaagggcaacaatttcacgggataactccaaaaatactctgtatctgcagatgaacagcctgagggcagaagacacagccgtgtactattgcgtgaaatcagtctccggagatgcctggtctgtggacgggctggatgcttggggtcagggcaccctggtcacagtctcgag

SEQ ID NO: 265 - DNA, 321 bp, synthetic construct
  misc_feature 1..321: note = source = /note="Description of Artificial Sequence: Synthetic polynucleotide"
  source 1..321: mol_type = other DNA
gctagcctcatacgagctgacccaggacccagcagtgtccgtcgccctgggccagacagtgagaatcacttgctccggaggaggatccgcctacggttggtatcagcagaagcccggccaggcacctgtgaccgtcatctactataacaatcagaggccatctgacattcccgatcggttcagtggatccagctctgggaacacagcaagtctgaccatcacaggcgcccaggctgaggacgaagccgattactattgtggaagctatgatagttcagctgtggggatttttggttctggcactaaagtcaccgtcctagg

SEQ ID NO: 266 - protein, 123 aa, synthetic construct
  REGION 1..123: note = source = /note="Description of Artificial Sequence: Synthetic polypeptide"
EVQLLESGGGLVQPGGSLRLSCAASGFDFSSYTMQWVRQAPGKGLEWVGVISSTGGSTGYGPAVKGRATISRDNSKNTLYLQMNSLRAEDTAVYYCVKSVSGDAWSVDGLDAWGQGTLVTVSS

SEQ ID NO: 267 - protein, 104 aa, synthetic construct
  REGION 1..104: note = source = /note="Description of Artificial Sequence: Synthetic polypeptide"
SYELTQDPAVSVALGQTVRITCSGGGSAYGWYQQKPGQAPVTVIYYNNQRPSDIPDRFSGSSSGNTASLTITGAQAEDEADYYCGSYDSSAVGIFGSGTKVTVL

SEQ ID NO: 268 - protein, 8 aa, synthetic construct
  REGION 1..8: note = source = /note="Description of Artificial Sequence: Synthetic peptide"
GFDFSSYT

SEQ ID NO: 269 - protein, 8 aa, synthetic construct
  REGION 1..8: note = source = /note="Description of Artificial Sequence: Synthetic peptide"
ISSTGGST

SEQ ID NO: 270 - protein, 18 aa, synthetic construct
  REGION 1..18: note = source = /note="Description of Artificial Sequence: Synthetic peptide"
CVKSVSGDAWSVDGLDAW

SEQ ID NO: 271-272 - skipped (2 entries)

SEQ ID NO: 273 - protein, 12 aa, synthetic construct
  REGION 1..12: note = source = /note="Description of Artificial Sequence: Synthetic peptide"
CGSYDSSAVGIF

SEQ ID NO: 274 - DNA, 385 bp, synthetic construct
  misc_feature 1..385: note = source = /note="Description of Artificial Sequence: Synthetic polynucleotide"
  source 1..385: mol_type = other DNA
ggcgcgccgaggtgcagctgctggaatccggaggaggactggtccagccaggtggaagcctgcgactgtcttgcgccgctagtggattcgacttttccagctacggaatgcagtgggtgaggcaggcaccaggcaagggactggagtgggtgggcgtcatctctggaagtgggattaccacactgtacgcacctgccgtcaagggaagggctactatctcacgggacaactctaaaaatacagtgtatctgcagatgaactccctgagagctgaagataccgcagtctactattgtacacgctcaccctccatcacagacggctggacttatggaggggcctggattgatgcttggggtcagggcactctggtgaccgtctcgag

SEQ ID NO: 275 - DNA, 318 bp, synthetic construct
  misc_feature 1..318: note = source = /note="Description of Artificial Sequence: Synthetic polynucleotide"
  source 1..318: mol_type = other DNA
gctagccagctacgagctgacccaggacccagcagtgtccgtcgccctgggccagacagtgagaatcacttgcagtggcggagatgggtcatacggttggttccagcagaagcccggacaggcccctgtgaccgtcatctatgacaacgataataggccatctgacattcccgatcggtttagtggctccagctctggaaacacagcttctctgaccatcacaggggcccaggctgaggacgaagctgattactattgtggcaatgcagacctgtccgggggtattttcggcagcggaactaaagtcaccgtcctagg

SEQ ID NO: 276 - protein, 126 aa, synthetic construct
  REGION 1..126: note = source = /note="Description of Artificial Sequence: Synthetic polypeptide"
EVQLLESGGGLVQPGGSLRLSCAASGFDFSSYGMQWVRQAPGKGLEWVGVISGSGITTLYAPAVKGRATISRDNSKNTVYLQMNSLRAEDTAVYYCTRSPSITDGWTYGGAWIDAWGQGTLVTVSS

SEQ ID NO: 277 - protein, 103 aa, synthetic construct
  REGION 1..103: note = source = /note="Description of Artificial Sequence: Synthetic polypeptide"
SYELTQDPAVSVALGQTVRITCSGGDGSYGWFQQKPGQAPVTVIYDNDNRPSDIPDRFSGSSSGNTASLTITGAQAEDEADYYCGNADLSGGIFGSGTKVTVL

SEQ ID NO: 278 - protein, 8 aa, synthetic construct
  REGION 1..8: note = source = /note="Description of Artificial Sequence: Synthetic peptide"
GFDFSSYG

SEQ ID NO: 279 - protein, 8 aa, synthetic construct
  REGION 1..8: note = source = /note="Description of Artificial Sequence: Synthetic peptide"
ISGSGITT

SEQ ID NO: 280 - protein, 21 aa, synthetic construct
  REGION 1..21: note = source = /note="Description of Artificial Sequence: Synthetic peptide"
CTRSPSITDGWTYGGAWIDAW

SEQ ID NO: 281-282 - skipped (2 entries)

SEQ ID NO: 283 - protein, 11 aa, synthetic construct
  REGION 1..11: note = source = /note="Description of Artificial Sequence: Synthetic peptide"
CGNADLSGGIF

SEQ ID NO: 284 - DNA, 388 bp, synthetic construct
  misc_feature 1..388: note = source = /note="Description of Artificial Sequence: Synthetic polynucleotide"
  source 1..388: mol_type = other DNA
ggcgcgccgaggtgcagctgctggaatctggaggaggactggtccagccaggtggatccctgagactgagctgcgccgcttctggattcacctttagtacattcaacatggtgtgggtcaggcaggcacctggaaagggactggagtacgtggctgaaatctccagcgacggctcttttacatggtatgcaactgccgtcaagggcagggccaccattagtcgggataactcaaaaaatacagtgtacctgcagatgaattccctgagggctgaggacaccgcagtctactattgcgcaaaatccgattgttctagttcatactatggatatagctgtatcgggatcattgacgcttggggtcagggcactctggtgaccgtctcgag

SEQ ID NO: 285 - DNA, 339 bp, synthetic construct
  misc_feature 1..339: note = source = /note="Description of Artificial Sequence: Synthetic polynucleotide"
  source 1..339: mol_type = other DNA
gctagcctcctatgagctgacccaggacccagcagtgagcgtcgccctgggccagacagtgagaatcacttgctccggcggaattagcgacgatggctcttactattacggatggttccagcagaagcccggacaggcccctgtgaccgtcatctatattaacgacaggcggccaagtaatatccccgataggttttcagggtccagctctggtaacacagcttctctgaccattacaggggcccaggctgaggacgaagctgattattactgtggctcttacgatagttcagcaggggtgggtatcttcggcagtggaactaaagtcaccgtcctagg

SEQ ID NO: 286 - protein, 127 aa, synthetic construct
  REGION 1..127: note = source = /note="Description of Artificial Sequence: Synthetic polypeptide"
EVQLLESGGGLVQPGGSLRLSCAASGFTFSTFNMVWVRQAPGKGLEYVAEISSDGSFTWYATAVKGRATISRDNSKNTVYLQMNSLRAEDTAVYYCAKSDCSSSYYGYSCIGIIDAWGQGTLVTVSS

SEQ ID NO: 287 - protein, 110 aa, synthetic construct
  REGION 1..110: note = source = /note="Description of Artificial Sequence: Synthetic polypeptide"
SYELTQDPAVSVALGQTVRITCSGGISDDGSYYYGWFQQKPGQAPVTVIYINDRRPSNIPDRFSGSSSGNTASLTITGAQAEDEADYYCGSYDSSAGVGIFGSGTKVTVL

SEQ ID NO: 288 - protein, 8 aa, synthetic construct
  REGION 1..8: note = source = /note="Description of Artificial Sequence: Synthetic peptide"
GFTFSTFN

SEQ ID NO: 289 - protein, 8 aa, synthetic construct
  REGION 1..8: note = source = /note="Description of Artificial Sequence: Synthetic peptide"
ISSDGSFT

SEQ ID NO: 290 - protein, 22 aa, synthetic construct
  REGION 1..22: note = source = /note="Description of Artificial Sequence: Synthetic peptide"
CAKSDCSSSYYGYSCIGIIDAW

SEQ ID NO: 291 - protein, 8 aa, synthetic construct
  REGION 1..8: note = source = /note="Description of Artificial Sequence: Synthetic peptide"
ISDDGSYY

SEQ ID NO: 292 - skipped

SEQ ID NO: 293 - protein, 13 aa, synthetic construct
  REGION 1..13: note = source = /note="Description of Artificial Sequence: Synthetic peptide"
CGSYDSSAGVGIF

SEQ ID NO: 294 - DNA, 400 bp, synthetic construct
  misc_feature 1..400: note = source = /note="Description of Artificial Sequence: Synthetic polynucleotide"
  source 1..400: mol_type = other DNA
ggcgcgccgaggtgcagctgctggaaagtggaggaggactggtccagccaggtggatcactgagactgtcctgcgccgcctccggcttcaccttttccagctacaacatgttctgggtgcgccaggcaccaggaaagggactggagtttgtcgctgaaatctctggtagtaatactggaagccgaacctggtacgcacctgccgtgaagggcagggctacaatttctcgggacaacagtaaaaatactctgtatctgcagatgaactctctgagggctgaggatacagcagtgtactattgtgcaaaatcaatctacggagggtattgcgccggtggctattcctgtggtgtgggcctgattgacgcatggggacaggggaccctggtcacagtctcgag

SEQ ID NO: 295 - DNA, 321 bp, synthetic construct
  misc_feature 1..321: note = source = /note="Description of Artificial Sequence: Synthetic polynucleotide"
  source 1..321: mol_type = other DNA
gctagcctcatacgagctgacccaggacccagcagtgtccgtcgccctgggccagacagtgagaatcacttgcagtggcggatccagcgattactatgggtggttccagcagaagcccggtcaggcccctgtgaccgtcatctactataacaacaagaggccatctgacattcccgatcggtttagtggctctagttcaggaaacacagcctccctgaccattacaggggcccaggctgaggacgaagctgattactattgtggcaatgcagactccagcgtgggagtcttcgggtctggtactaaggtgaccgtcctagg

SEQ ID NO: 296 - protein, 131 aa, synthetic construct
  REGION 1..131: note = source = /note="Description of Artificial Sequence: Synthetic polypeptide"
EVQLLESGGGLVQPGGSLRLSCAASGFTFSSYNMFWVRQAPGKGLEFVAEISGSNTGSRTWYAPAVKGRATISRDNSKNTLYLQMNSLRAEDTAVYYCAKSIYGGYCAGGYSCGVGLIDAWGQGTLVTVSS

SEQ ID NO: 297 - protein, 104 aa, synthetic construct
  REGION 1..104: note = source = /note="Description of Artificial Sequence: Synthetic polypeptide"
SYELTQDPAVSVALGQTVRITCSGGSSDYYGWFQQKPGQAPVTVIYYNNKRPSDIPDRFSGSSSGNTASLTITGAQAEDEADYYCGNADSSVGVFGSGTKVTVL

SEQ ID NO: 298 - protein, 8 aa, synthetic construct
  REGION 1..8: note = source = /note="Description of Artificial Sequence: Synthetic peptide"
GFTFSSYN

SEQ ID NO: 299 - protein, 10 aa, synthetic construct
  REGION 1..10: note = source = /note="Description of Artificial Sequence: Synthetic peptide"
ISGSNTGSRT

SEQ ID NO: 300 - protein, 24 aa, synthetic construct
  REGION 1..24: note = source = /note="Description of Artificial Sequence: Synthetic peptide"
CAKSIYGGYCAGGYSCGVGLIDAW

SEQ ID NO: 301 - protein, 4 aa, synthetic construct
  REGION 1..4: note = source = /note="Description of Artificial Sequence: Synthetic peptide"
SSDY

SEQ ID NO: 302 - skipped

SEQ ID NO: 303 - protein, 11 aa, synthetic construct
  REGION 1..11: note = source = /note="Description of Artificial Sequence: Synthetic peptide"
CGNADSSVGVF

SEQ ID NO: 304 - DNA, 357 bp, synthetic construct
  misc_feature 1..357: note = source = /note="Description of Artificial Sequence: Synthetic polynucleotide"
  source 1..357: mol_type = other DNA
caggtgcagctgcagcagtggggtgccggtctgctgaagccttctgaaactctgtctctgacttgtgccgtctatggtggatcattcagcggctactattggtcctggatcaggcagccccctggcaagggcctggagtggatcggcgagatcaaccaccggggctctaccaactacaatccctctctgaagagcagggtgaccatctccgtggacacatctaagaatcagttcagcctgaagctgagctccgtgaccgccgctgatacagccgtgtactattgcacaaggggggaggaatgggagtcactgttctttgattactgggggcaggggacactggtcacagtctcgagt

SEQ ID NO: 305 - DNA, 321 bp, synthetic construct
  misc_feature 1..321: note = source = /note="Description of Artificial Sequence: Synthetic polynucleotide"
  source 1..321: mol_type = other DNA
gaaatcgtcctgacccagtcccccgccaccctgagcctgagccccggagaaagagccaccctgtcctgccgagcaagccagtccatcagctcctatctcgcctggtatcagcagaaaccaggccaggctccccggctgctgatctacggcgcctccaacagagctacaggaatcccagcccgcttcagcggctccggctctggcacagactttaccctgacaatctctagcctggagcctgaggatttcgccgtgtactattgccagcagagatctaattggccactgacattcggcggcggcacacgggtggagatcaag

SEQ ID NO: 306 - protein, 119 aa, synthetic construct
  REGION 1..119: note = source = /note="Description of Artificial Sequence: Synthetic polypeptide"
QVQLQQWGAGLLKPSETLSLTCAVYGGSFSGYYWSWIRQPPGKGLEWIGEINHRGSTNYNPSLKSRVTISVDTSKNQFSLKLSSVTAADTAVYYCTRGEEWESLFFDYWGQGTLVTVSS

SEQ ID NO: 307 - protein, 107 aa, synthetic construct
  REGION 1..107: note = source = /note="Description of Artificial Sequence: Synthetic polypeptide"
EIVLTQSPATLSLSPGERATLSCRASQSISSYLAWYQQKPGQAPRLLIYGASNRATGIPARFSGSGSGTDFTLTISSLEPEDFAVYYCQQRSNWPLTFGGGTRVEIK

SEQ ID NO: 308 - protein, 8 aa, synthetic construct
  REGION 1..8: note = source = /note="Description of Artificial Sequence: Synthetic peptide"
GGSFSGYY

SEQ ID NO: 309 - protein, 7 aa, synthetic construct
  REGION 1..7: note = source = /note="Description of Artificial Sequence: Synthetic peptide"
INHRGST

SEQ ID NO: 310 - protein, 15 aa, synthetic construct
  REGION 1..15: note = source = /note="Description of Artificial Sequence: Synthetic peptide"
CTRGEEWESLFFDYW

SEQ ID NO: 311 - protein, 6 aa, synthetic construct
  REGION 1..6: note = source = /note="Description of Artificial Sequence: Synthetic peptide"
QSISSY

SEQ ID NO: 312 - skipped

SEQ ID NO: 313 - protein, 11 aa, synthetic construct [sequence omitted]

SEQ ID NO: 314 - DNA, 366 bp, synthetic construct
  misc_feature 1..366: note = source = /note="Description of Artificial Sequence: Synthetic polynucleotide"
  source 1..366: mol_type = other DNA
caggttcagctgcagcagtggggcgccggcctgctgagaccaagcgagaccctgtccctgacatgcgccgtgtatggcgagagcttctccggctattactggaactggatccggcagcctcccggcaagggcctggagtggatcggcgagatcaatcactccggctccaccaattacaacccatccctgaagtctcgggtgacaatcagcgtggatacaagcaagacccagttcagcctgaagctgagctccgtgacagctgccgataccgccgtgtattactgcgccagaggctgggacctgctggattggaatgactactggaatgagtactggggccaggggaccctggtgaccgtctcgagt

SEQ ID NO: 315 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtcccctgccaccctgtctctgtcccctggcgagcgggccaccctgtcctgtagagcttctcagtccgtgtcttcctacctggcttggtaccagcagaagccaggacaggccccaagactgctgatctatgacgcttccaatcgggctaccggcatcccagctcgctttagcggctccggctccggcaccgacttcaccctgacaatcagctccctggagccagaggattttgccgtgtattactgtcagcagaggtccaattggccactgacatttggcggcggcacaaaggttgagatcaag

SEQ ID NO: 316 - protein, 122 aa, synthetic construct
  REGION 1..122: note = source = /note="Description of Artificial Sequence: Synthetic polypeptide"
QVQLQQWGAGLLRPSETLSLTCAVYGESFSGYYWNWIRQPPGKGLEWIGEINHSGSTNYNPSLKSRVTISVDTSKTQFSLKLSSVTAADTAVYYCARGWDLLDWNDYWNEYWGQGTLVTVSS

SEQ ID NO: 317 - protein, 107 aa, synthetic construct; identical to SEQ ID NO: 4
  REGION 1..107: note = source = /note="Description of Artificial Sequence: Synthetic polypeptide"

SEQ ID NO: 318 - protein, 8 aa, synthetic construct
  REGION 1..8: note = source = /note="Description of Artificial Sequence: Synthetic peptide"
GESFSGYY

SEQ ID NO: 319 - protein, 7 aa, synthetic construct
  REGION 1..7: note = source = /note="Description of Artificial Sequence: Synthetic peptide"
INHSGST

SEQ ID NO: 320 - protein, 18 aa, synthetic construct
  REGION 1..18: note = source = /note="Description of Artificial Sequence: Synthetic peptide"
CARGWDLLDWNDYWNEYW

SEQ ID NO: 321 - protein, 6 aa, synthetic construct
  REGION 1..6: note = source = /note="Description of Artificial Sequence: Synthetic peptide"
QSVSSY

SEQ ID NO: 322 - skipped

SEQ ID NO: 323 - protein, 11 aa, synthetic construct [sequence omitted]

SEQ ID NO: 324 - DNA, 357 bp, synthetic construct
  misc_feature 1..357: note = source = /note="Description of Artificial Sequence: Synthetic polynucleotide"
  source 1..357: mol_type = other DNA
caggttcagctgcagcagtggggcgctggcctgctgaagccctctgagaccctgtctctgacctgtgccgtgtatggcggcagcttctccggctattactggaactggatccgccagccccccggcaagggcctggagtggatcggcgagatcaaccactccggctctaccaactacaatccttctctgaagtccagggtgacaatcagcgtggacaccagcaagaaccagtttagcctgaagctgtccagcgtgacagctgccgatacagccgtgtattactgcgccagaggcgaggattggggcgagagcttctttgattactggggccaggggaccctggtgaccgtctcgagt

SEQ ID NO: 325 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtcccctgccaccctgtctctgtcccctggcgagcgggccaccctgagctgtcgggcctcccagtccgtgagctcctacctggcttggtatcagcagaagccaggacaggccccaagactgctgatctacgccgcttccaatcgggccaccggcatccccgccagattttccggctctggctccggcaccgatttcaccctgacaatcagctccctggagccagaggacttcgccgtgtactactgccagcagaggtctaattggccactgacatttggcggcggcaccaaggttgagatcaag

SEQ ID NO: 326 - protein, 119 aa, synthetic construct
  REGION 1..119: note = source = /note="Description of Artificial Sequence: Synthetic polypeptide"
QVQLQQWGAGLLKPSETLSLTCAVYGGSFSGYYWNWIRQPPGKGLEWIGEINHSGSTNYNPSLKSRVTISVDTSKNQFSLKLSSVTAADTAVYYCARGEDWGESFFDYWGQGTLVTVSS

SEQ ID NO: 327 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYAASNRATGIPARFSGSGSGTDFTLTISSLEPEDFAVYYCQQRSNWPLTFGGGTKVEIK

SEQ ID NO: 328 - protein, 8 aa, synthetic construct
  REGION 1..8: note = source = /note="Description of Artificial Sequence: Synthetic peptide"
GGSFSGYY

SEQ ID NO: 329 - protein, 7 aa, synthetic construct
  REGION 1..7: note = source = /note="Description of Artificial Sequence: Synthetic peptide"
INHSGST

SEQ ID NO: 330 - protein, 15 aa, synthetic construct
  REGION 1..15: note = source = /note="Description of Artificial Sequence: Synthetic peptide"
CARGEDWGESFFDYW

SEQ ID NO: 331 - protein, 6 aa, synthetic construct
  REGION 1..6: note = source = /note="Description of Artificial Sequence: Synthetic peptide"
QSVSSY

SEQ ID NO: 332 - skipped

SEQ ID NO: 333 - protein, 11 aa, synthetic construct [sequence omitted]

SEQ ID NO: 334 - DNA, 357 bp, synthetic construct
  misc_feature 1..357: note = source = /note="Description of Artificial Sequence: Synthetic polynucleotide"
  source 1..357: mol_type = other DNA
caggttcagctggtgcagtccggcgctgaggtgaagaagcctggagcttctgtgaaggtttcctgtaaggtttccggctatagcctgaccgagatctctatgcactgggtacggcaagcccccggcaagggcctggagtggatgggcggctttgacccagaggatggcgagaccatctacgctcagaggtttcaggggcgcgtgatcatgaccgaggataccagcaccgataccgcctacatggagctgtccagcctgagatccgaggataccgccgtgtattactgcgctactggtggctggggccccaattggttcgatccttggggccaggggaccctggtgaccgtctcgagt

SEQ ID NO: 335 - DNA, 321 bp, synthetic construct
  misc_feature 1..321: note = source = /note="Description of Artificial Sequence: Synthetic polynucleotide"
  source 1..321: mol_type = other DNA
gacatccagatgacacagtctccttcctctgtgagcgcctctgtgggcgaccgcgtgaccatcacatgccgcgcttcccaggggatctcttcctggctggcttggtaccagcagaagcccggcaaggcccccaagctgctgatctatgctgccagcagcctgcagtccggcgtgccttccaggtttagcggctccggctccggcaccgactttacactgacaatcagctccctgcagcccgaggattttgccacctattactgtcagcaggcgaattccttcccttttacattcggccctggcaccaaggttgatatcaag

SEQ ID NO: 336 - protein, 119 aa, synthetic construct
  REGION 1..119: note = source = /note="Description of Artificial Sequence: Synthetic polypeptide"
QVQLVQSGAEVKKPGASVKVSCKVSGYSLTEISMHWVRQAPGKGLEWMGGFDPEDGETIYAQRFQGRVIMTEDTSTDTAYMELSSLRSEDTAVYYCATGGWGPNWFDPWGQGTLVTVSS

SEQ ID NO: 337 - protein, 107 aa, synthetic construct
  REGION 1..107: note = source = /note="Description of Artificial Sequence: Synthetic polypeptide"
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIK

SEQ ID NO: 338 - protein, 8 aa, synthetic construct
  REGION 1..8: note = source = /note="Description of Artificial Sequence: Synthetic peptide"
GYSLTEIS

SEQ ID NO: 339 - protein, 8 aa, synthetic construct
  REGION 1..8: note = source = /note="Description of Artificial Sequence: Synthetic peptide"
FDPEDGET

SEQ ID NO: 340 - protein, 14 aa, synthetic construct
  REGION 1..14: note = source = /note="Description of Artificial Sequence: Synthetic peptide"
CATGGWGPNWFDPW

SEQ ID NO: 341 - protein, 6 aa, synthetic construct
  REGION 1..6: note = source = /note="Description of Artificial Sequence: Synthetic peptide"
QGISSW

SEQ ID NO: 342 - skipped

SEQ ID NO: 343 - protein, 11 aa, synthetic construct [sequence omitted]

SEQ ID NO: 344 - DNA, 363 bp, synthetic construct
  misc_feature 1..363: note = source = /note="Description of Artificial Sequence: Synthetic polynucleotide"
  source 1..363: mol_type = other DNA
caggttcagctgcagcagtccggccccggcctggtgaagccaagccagaccctgtctctgacatgcgccatctccggcgacagcgtgtcctctaactccgccgcttggaattggatccggcagtctccatccagaggcctggagtggctgggcagaacctattaccggtccaagtggtacaacgattatgccgtgtccgtgaagagcagaatcaccatcaaccctgataccagcaagaaccagttcagcctgcagctgaattccgtgaccccagaggatacagccgtgtattactgtgctagggacgatgattggaatgacttcgattactggggccaggggaccctggtgacagtctcgagt

SEQ ID NO: 345 - DNA, 321 bp, synthetic construct
  misc_feature 1..321: note = source = /note="Description of Artificial Sequence: Synthetic polynucleotide"
  source 1..321: mol_type = other DNA
gagatcgtgctgacccagtccccagctacactgtccctgtctcccggcgagcgggccaccctgagctgtagagcttcccagtccgtgtcttcctatctggcttggtatcagcagaagccaggacaggccccaaggctgctgatctacgacgcctccaatagagccaccggcatcccagctagattttctggctccggctccggcaccgatttcacactgaccatctctagcctggagccagaggattttgctgtatattactgccagcagcgcagcaactggcccctgacatttggcggcggcaccaaggttgagatcaag

SEQ ID NO: 346 - protein, 121 aa, synthetic construct
  REGION 1..121: note = source = /note="Description of Artificial Sequence: Synthetic polypeptide"
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDDDWNDFDYWGQGTLVTVSS

SEQ ID NO: 347 - protein, 107 aa, synthetic construct; identical to SEQ ID NO: 4
  REGION 1..107: note = source = /note="Description of Artificial Sequence: Synthetic polypeptide"

SEQ ID NO: 348 - protein, 9 aa, synthetic construct
  REGION 1..9: note = source = /note="Description of Artificial Sequence: Synthetic peptide"
GDSVSSNSA

SEQ ID NO: 349 - protein, 7 aa, synthetic construct
  REGION 1..7: note = source = /note="Description of Artificial Sequence: Synthetic peptide"
TYYRSKW

SEQ ID NO: 350 - protein, 13 aa, synthetic construct
  REGION 1..13: note = source = /note="Description of Artificial Sequence: Synthetic peptide"
CARDDDWNDFDYW

SEQ ID NO: 351 - protein, 6 aa, synthetic construct
  REGION 1..6: note = source = /note="Description of Artificial Sequence: Synthetic peptide"
QSVSSY

SEQ ID NO: 352 - skipped

SEQ ID NO: 353 - protein, 11 aa, synthetic construct [sequence omitted]

SEQ ID NO: 354 - DNA, 372 bp, synthetic construct
  misc_feature 1..372: note = source = /note="Description of Artificial Sequence: Synthetic polynucleotide"
  source 1..372: mol_type = other DNA
cagctgcagctgcaggaaagcggccccggcctggtgaagccctctgagaccctgtccctgacatgcaccgtgagcggcgattccatcagctcttccagctattactggggctggatccggcagccccccggcaagggcctggagtggatcggcagcatcttctactccggcaatacatattataatccttctctgaagagcagggtgacaatcagcgtggatacctccaagaatcagtttagcctgaagctgagctccgtgacagctgccgatacagccgtgtattactgcgctagggaggacgattttctgaccgattattacggcgctttcgacatctggggccaggggacaatggtgacagtctcgagt

SEQ ID NO: 355 - DNA, 318 bp, synthetic construct
  misc_feature 1..318: note = source = /note="Description of Artificial Sequence: Synthetic polynucleotide"
  source 1..318: mol_type = other DNA
gatatccagatgacccagtctccaagcaccctgagcgcctctgtgggcgatcgggtgaccatcacatgtcgggcttctcagtccatcagcagctggctggcttggtatcagcagaagcccggcaaggccccaaagctgctgatctacaaggcctcttccagcgagagcggcgtgccatccaggtttagcggctccggctccggcaccgagtttaccctgaccatctcttccctgcagcccgatgactttgccacctactactgtcagcagtacaattcctatctgacattcggcggcggcaccaaggttgagatcaag

SEQ ID NO: 356 - protein, 124 aa, synthetic construct
  REGION 1..124: note = source = /note="Description of Artificial Sequence: Synthetic polypeptide"
QLQLQESGPGLVKPSETLSLTCTVSGDSISSSSYYWGWIRQPPGKGLEWIGSIFYSGNTYYNPSLKSRVTISVDTSKNQFSLKLSSVTAADTAVYYCAREDDFLTDYYGAFDIWGQGTMVTVSS

SEQ ID NO: 357 - protein, 106 aa, synthetic construct
  REGION 1..106: note = source = /note="Description of Artificial Sequence: Synthetic polypeptide"
DIQMTQSPSTLSASVGDRVTITCRASQSISSWLAWYQQKPGKAPKLLIYKASSSESGVPSRFSGSGSGTEFTLTISSLQPDDFATYYCQQYNSYLTFGGGTKVEIK

SEQ ID NO: 358 - protein, 10 aa, synthetic construct
  REGION 1..10: note = source = /note="Description of Artificial Sequence: Synthetic peptide"
GDSISSSSYY

SEQ ID NO: 359 - protein, 7 aa, synthetic construct
  REGION 1..7: note = source = /note="Description of Artificial Sequence: Synthetic peptide"
IFYSGNT

SEQ ID NO: 360 - protein, 18 aa, synthetic construct
  REGION 1..18: note = source = /note="Description of Artificial Sequence: Synthetic peptide"
CAREDDFLTDYYGAFDIW

SEQ ID NO: 361 - protein, 6 aa, synthetic construct
  REGION 1..6: note = source = /note="Description of Artificial Sequence: Synthetic peptide"
QSISSW

SEQ ID NO: 362 - skipped

SEQ ID NO: 363 - protein, 10 aa, synthetic construct [sequence omitted]

SEQ ID NO: 364 - DNA, 375 bp, synthetic construct
  misc_feature 1..375: note = source = /note="Description of Artificial Sequence: Synthetic polynucleotide"
  source 1..375: mol_type = other DNA
gaggtgcagctgctggaatccggaggaggactggtccagccaggtggatccctgcgactgagctgcgccgcttctggcttcgactttagaagctacgcaatgatgtgggtccgccaggcaccaggaaagggactggagtgggtgggagggatcaacggtgaagtcggtggctctaatacatactatgcacctgccgtcaagggaagggctactattagtcgggacaactcaaaaaataccctgtatctgcaaatgaacagtctgagggccgaggataccgccgtgtactattgcgtgaaaggtgctggcgcatgcggcatctgtaatgacgatattgatgcatggggacaggggaccctggtgacagtctcgagt

SEQ ID NO: 365 - DNA, 330 bp, synthetic construct
  misc_feature 1..330: note = source = /note="Description of Artificial Sequence: Synthetic polynucleotide"
  source 1..330: mol_type = other DNA
agttatgagctgactcaggacccagcagtgtcagtcgccctgggccagacagtgagaatcacttgcagtggggctggttcatatgcaggctcctactattacggatggcaccagcagaagcccggacaggcacctgtgacagtcatctacgacaacgataaaaggccaagcaatattcccgaccggttctctgggtccagctctggtaacaccgcctccctgaccattactggggcccaggctgaggacgaagctgattattactgtggctctacaaacgataatgacgatggcggactgtttggctccggaactaaggtcaccgtccta

SEQ ID NO: 366 - protein, 125 aa, synthetic construct
  REGION 1..125: note = source = /note="Description of Artificial Sequence: Synthetic polypeptide"
EVQLLESGGGLVQPGGSLRLSCAASGFDFRSYAMMWVRQAPGKGLEWVGGINGEVGGSNTYYAPAVKGRATISRDNSKNTLYLQMNSLRAEDTAVYYCVKGAGACGICNDDIDAWGQGTLVTVSS

SEQ ID NO: 367 - protein, 110 aa, synthetic construct
  REGION 1..110: note = source = /note="Description of Artificial Sequence: Synthetic polypeptide"
SYELTQDPAVSVALGQTVRITCSGAGSYAGSYYYGWHQQKPGQAPVTVIYDNDKRPSNIPDRFSGSSSGNTASLTITGAQAEDEADYYCGSTNDNDDGGLFGSGTKVTVL

SEQ ID NO: 368 - protein, 8 aa, synthetic construct
  REGION 1..8: note = source = /note="Description of Artificial Sequence: Synthetic peptide"
GFDFRSYA

SEQ ID NO: 369 - protein, 10 aa, synthetic construct
  REGION 1..10: note = source = /note="Description of Artificial Sequence: Synthetic peptide"
INGEVGGSNT

SEQ ID NO: 370 - protein, 18 aa, synthetic construct
  REGION 1..18: note = source = /note="Description of Artificial Sequence: Synthetic peptide"
CVKGAGACGICNDDIDAW

SEQ ID NO: 371 - protein, 7 aa, synthetic construct
  REGION 1..7: note = source = /note="Description of Artificial Sequence: Synthetic peptide"
GSYAGSY

SEQ ID NO: 372 - skipped

SEQ ID NO: 373 - protein, 13 aa, synthetic construct
  REGION 1..13: note = source = /note="Description of Artificial Sequence: Synthetic peptide"
CGSTNDNDDGGLF

SEQ ID NO: 374 - protein, 330 aa, synthetic construct
  REGION 1..330: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 330 aa, synthetic construct
  REGION 1..330: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 327 aa, synthetic construct
  REGION 1..327: note = source = /note="Description of Artificial Sequence: Synthetic polypeptide"
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 377 - protein, 326 aa, synthetic construct
  REGION 1..326: note = source = /note="Description of Artificial Sequence: Synthetic polypeptide"
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 378 - protein, 107 aa, synthetic construct [sequence omitted]

SEQ ID NO: 379 - protein, 106 aa, synthetic construct
  REGION 1..106: note = source = /note="Description of Artificial Sequence: Synthetic polypeptide"
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 380 - DNA, 990 bp, synthetic construct
  misc_feature 1..990: note = source = /note="Description of Artificial Sequence: Synthetic polynucleotide"
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 381 - DNA, 1596 bp, synthetic construct
  misc_feature 1..1596: note = source = /note="Description of Artificial Sequence: Synthetic polynucleotide"
  source 1..1596: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 382 - DNA, 981 bp, synthetic construct
  misc_feature 1..981: note = source = /note="Description of Artificial Sequence: Synthetic polynucleotide"
  source 1..981: mol_type = other DNA
gcttccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 383 - DNA, 1588 bp, synthetic construct
  misc_feature 1..1588: note = source = /note="Description of Artificial Sequence: Synthetic polynucleotide"
  source 1..1588: mol_type = other DNA
gcttccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcaggccccatctgtctcctcacccggaggcctctgaccaccccactcatgctcagggagagggtcttctggatttttccaccaggctccgggcagccacaggctggatgcccctaccccaggccctgagcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 384 - DNA, 978 bp, synthetic construct
  misc_feature 1..978: note = source = /note="Description of Artificial Sequence: Synthetic polynucleotide"
  source 1..978: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 385 - DNA, 1585 bp, synthetic construct
  misc_feature 1..1585: note = source = /note="Description of Artificial Sequence: Synthetic polynucleotide"
  source 1..1585: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 386 - DNA, 321 bp, synthetic construct
  misc_feature 1..321: note = source = /note="Description of Artificial Sequence: Synthetic polynucleotide"
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 387 - DNA, 325 bp, synthetic construct
  misc_feature 1..325: note = source = /note="Description of Artificial Sequence: Synthetic polynucleotide"
  source 1..325: mol_type = other DNA
cctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataa

SEQ ID NO: 388 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 389 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 390 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 391 - DNA, 321 bp, synthetic construct
  misc_feature 1..321: note = source = /note="Description of Artificial Sequence: Synthetic polynucleotide"
  source 1..321: mol_type = other DNA
gctagcctcttacgagctgactcagccaccttccgtgtccgtgtccccaggacagaccgcaagaatcacatgcagtggcggatccagctactcatatgggtggttccagcagaagcctggtcaggcccccgtgacagtcatctatgagagcaacaataggccttctgacattccagaacggtttagtggctctagttcaggaaccacagtgactctgaccatcagcggggtccaggccgaggacgaagctgattactattgtggcaacgctgattccagctctggaattttcgggtccggtacaaaagtgactgtcctagg

SEQ ID NO: 392 - protein, 104 aa, synthetic construct
  REGION 1..104: note = source = /note="Description of Artificial Sequence: Synthetic polypeptide"
SYELTQPPSVSVSPGQTARITCSGGSSYSYGWFQQKPGQAPVTVIYESNNRPSDIPERFSGSSSGTTVTLTISGVQAEDEADYYCGNADSSSGIFGSGTKVTVL

SEQ ID NO: 393 - protein, 4 aa, synthetic construct
  REGION 1..4: note = source = /note="Description of Artificial Sequence: Synthetic peptide"
SSYS

SEQ ID NO: 394 - skipped

SEQ ID NO: 395 - protein, 11 aa, synthetic construct
  REGION 1..11: note = source = /note="Description of Artificial Sequence: Synthetic peptide"
CGNADSSSGIF

SEQ ID NO: 396 - protein, 15 aa, synthetic construct
  REGION 1..15: note = source = /note="Description of Artificial Sequence: Synthetic peptide"
GGGGSGGGGSGGGGS

SEQ ID NO: 397 - protein, 6 aa, Homo sapiens
KIEELE